(19) United States
(12) Patent Application Publication
(10) Pub. No.: US 2004/0156920 A1
(43) Pub. Date: Aug. 12, 2004

(54) Extracts from plant and non-plant biomass and uses thereof

(76) Inventor: Shantaram Govind Kane, Pune (IN)

Correspondence Address: Brinks Hofer Gilson & Lione, NBC Tower, Suite 3600, P.O. Box 10395, Chicago, IL, 60610, US

(21) Appl. No.: 10/338,405

(22) Filed: Jan. 7, 2003

(30) Foreign Application Priority Data
Jul. 14, 2000 (IN) 653/MUM/2000
Jul. 14, 2000 (IN) 654/MUM/2000
Jul. 4, 2001 (WO) PCT/IN01/00132

(51) Int. Cl. A61K 35/78; A01N 65/00

(52) U.S. Cl. 424/725; 424/750; 424/756; 424/753; 424/754; 424/734; 424/770; 424/757; 424/761

(57) ABSTRACT

Novel oil extracts from Angiosperm and Gymnosperm plants and other-plant biomass from human, veterinary, birds, aquatic species, microbial and mycological sources useful in human, veterinary and agricultural, mycological and microbiological applications are described. Methods of preparation of these extracts in oil and methods of application and administration are also described.

### RELATED APPLICATIONS

This application claims priority to India application Serial Nos. 653/MUM/2000 and 654/MUM/2000, both filed Jul. 14, 2000 and PCT application PCT/IN01/00132, filed Jul. 04, 2001, International Publication Number WO 02/05830 A2, which are incorporated herein by reference in their entirety.

### BACKGROUND OF THE INVENTION

Introduction

The use of plants and herbs for general welfare and effectively treating a variety of conditions and ailments dates back to ancient times. Use of a variety of all other type of materials of biological origin (biomass), both prokaryotes and eukaryotes, has also been known for treating a variety of conditions and ailments. The biomass source materials can be from bacteria, fungi, mushroom, yeast, poultry, birds, marine, amphibian, animal and human products.

However, using these materials of biological origin can also be dangerous because of endogenous toxins. Effective doses are often achieved when large volumes of these materials are used, thus aggravating toxic effects.

The plants of different genera and species of the Crassulaceae family have been used to combat inflammation, promote healing, and improve overall well being. The Crassulaceae fix carbon via Crassulacean Acid Metabolism (CAM)—in the dark. CAM, so called because originally found in the Crassulaceae family (stonecrops, comprising mostly succulents such as cacti), plants temporally separate the two pathways of carbon fixation, C₃ and C₄, while using both cycles within the same cells. The initial fixation of carbon dioxide, the C₄ pathway, occurs at night (via cytosolic PEP carboxylase), while the C₃ pathway functions during the day. Consequently, the stomata of CAM plants are closed during the day, thus enabling them to withstand brutal environmental conditions, such as drought and low temperatures, and are open at night to take in carbon dioxide. In contrast, C₄ plants have open stomata during the day and closed during night. CAM plants have been reported in at least 23 families of flowering plants, mostly eudicots, including maternity plant, wax plant, snake plant. Less succulent CAM plants include pineapple and Spanish moss. Interestingly, some nonflowering plants also show CAM activity, including the gymnosperm Welwitschia mirabilisi, quillwort (lsoetes), and some ferns (Raven et al., 1999).

CAM plants are adapted to high stress conditions such as arid zones, including hot and cold deserts, and high altitudes. CAM plants can be found in many genera and are not limited to succulents; these include Kalanchoe, Bryophyllum, Sedum, Sempervium, Rhodiola, Crassulaceae, Aloe, and Cissus sp. CAM plants have been used for many human applications. Most often, plant parts, such as leaves, or plant juices are orally administered. However, the typical dosage is very high, above 100 mg/kg body weight per day (Blazovics et al., 1993; Boikova and Akulova, 1995; Botha et al., 1997; Da Silva et al., 1995; Da Silva et al., 1999; Lans and Brown, 1998; Nadkarni, 1982; Nassis et al., 1992; Obaseiki-Ebor, 1985; Pal et al., 1992; Sendl et al., 1993; Verma et al., 1986; Yoshikawa et al., 1997). Whenever fresh juice or decoction of a particular plant is given internally according to traditional or folk medicine, the useful medicinal agent is believed to be released in the juice from ground leaves, or the decoction of other plant parts; the plant residues remaining after extraction were not added to the composition.

Kalanchoe is perhaps the most widely known genus in folk medicine. Kalanchoe pinnata (Lam.) pers; Bryophyllum calicinum Salis; Cotyledon pinnata and Bryophullum pinnatum are synomyous, and have been extensively studied. Folk medicine has bestowed nicknames, such as “Wonder Plant” and descriptions such as “Divine”, thus illustrating their importance.

Sedum and Sempervivum are more commonly known in folk medicine of Europe whereas Rhodiola and other genera are known more in China and the Far East. Although the uses of these genera are not as far-reaching as that of Kalanchoe, specific uses have been reported, such as for protecting the liver and lowering lipids for Sempervivum which are not reported so far for Kalanchoe.

Aloe sp. has been used to promote health worldwide for thousands of years. Aloe vera is the most commonly used species throughout the world. The plant is used both by external topical application and by internal dose. These applications include promotion of general health; specifically, wound and burn healing, surgery recovery, bone growth, immunoprotection against cancer, health in HIV-infected subjects, protection against frostbite, reducing arthritic swelling, bowel inflammation, blood sugar, and protection of superoxide dismutase and glutathione from radiation.

The effective dosage for Aloe preparations required is typical for many herbal reparations. Dosage is high: an oral dose of 100 mg/kg per day in animal studies or wound healing (Davies et al., 1989) and 150 mg/kg per day has been prescribed to treat arthritis (Davies et al., 1992). For humans, the reported dose of the extract or juice ranges from 2 g/day (½ teaspoon) to over 100 g/day. Direct topical application also requires several grams per application. Anthraquinones and other low molecular weight compounds in Aloe are also reported to have cytotoxicity (Avila et al., 1997; Mueller and Stopper, 1999).

Cissus quadrangularis is the most commonly used species throughout Asia and Africa. It has been used to promote fracture healing both by external topical application and by internal dose. Other uses include treating rheumatic back pain and body pain, irregular menstruation, stomachache and whooping cough.

Most parts of the plant have been used, including the stem, leaves, and tender shoots. In addition, the entire plant, dried, is used in Arabic countries as a “Cure-All” medicine. Pastes made from leaves, stem or entire plants have been used for external applications.

The dosage levels required are typical for many herbal preparations. Oral dose of juice is 10 to 20 grams/day. Typical dosages of dry stem powder are 2 to 4 grams. Topical applications in the form of paste of dried parts is usually applied at least 10 grams or more. However, the usefulness of this plant is diminished: the various previously known compositions are reported to have mutagenic (including clastogenic) activity (Balachandran et al., 1991; Sivaswamy et al., 1991).

Table 1 summarizes the common uses of CAM plants; for a comprehensive review of Bryophyllum, Kalanchoe, Aloe and Cissus regarding uses, see (Nadkarni, 1982; Gogte, 2000).

TABLE 1
CAM plants and their uses

| Plant | Application | Reference |
|---|---|---|
| Kalanchoe pinnata (Lam.) | Anti-inflammatory | (Pal et al., 1992) |
| pers; (also known as | Anti-bacterial | (Obaseiki-Ebor, |
| Bryophullum calicinum Salis; | | 1985; Verma et al., |
| Cotyledon pinnata and | | 1985) |
| Bryophullum pinnatum) and | Neurotoxin (animal | (Botha et al., 1997) |
| other Kalanchoe sp. | husbandry study) | |
| | Anti-parasite (e.g. | (Da Silva et al., |
| | Leishmania) | 1995; Da Silva et |
| | | al., 1999) |
| | Antihistamine (anti- | (Nassis et al., 1992) |
| | inflammatory) | |
| | Pharmaceutical | (Verma et al., 1986) |
| | (general) | |
| Sempervivum sp. | Lipid reduction | (Blazovics et al., |
| | (circulation) | 1993) |
| Sedum sp. | Anti-inflammatory | (Sendl et al., 1993) |
| Rhodiula sacra | Antihistamine (anti- | (Yoshikawa et al., |
| | inflammatory) | 1997) |
| Aloe sp. | Macrophage | (Djeraba and Quere, |
| | activation | 2000) |
| | Wound healing, | (Davies et al., 1989; |
| | circulation | Paturmaj, 2000) |
| | Immunoregulation | (Qiu et al., 2000) |
| | General (reviews) | (Reynolds and |
| | | Dweck, 1999; |
| | | Vogler and Ernst, |
| | | 1999) |
| | Inflammation | (Davies et al., 1992) |
| | Anti-fungal | (Ali et al., 1999) |
| | Toxicity | (Avila et al., 1997; |
| | | Mueller and |
| | | Stopper, 1999) |
| Cissus sp. | Animal health | (Barakat et al., |
| | (general) | 1985) |
| | Animal health- | (Hifny et al., 1984) |
| | nervous system | |
| | (brain) | |
| | Fracture healing | (Chopra et al., 1975; |
| | | Chopra et al., 1976) |
| | Toxicity | (Balachandran et al., |
| | | 1991; Sivaswamy et |
| | | al., 1991) |
| General Crassulaceae | Ovulation regulation | (Boikova and |
| references | | Akulova, 1995) |
| | Uses in poultry | (Lans and Brown, |
| | | 1998) |

Many other plants front different families and genera have also been used for the treatment of a wide variety of conditions and ailments all over the world. This range includes:

Angiosperm-Monocotyledon Plants

Angiosperm-Dicotyledon Plants

Gymnosperm Plants

Many parts of the plants have been used, including the stem, leaves, rhizomes, seeds, tender shoots. In addition, the entire plant, dried, is also used in some cases. Pastes made from leaves, stem or entire plants have been used for external applications.

The dosage levels required are typical for many herbal preparations. Oral dose of juice or decoction of plant parts is 2 to 50 ml/day. Typical dosages of dry powder are 1 to 4 grams. Topical applications in the form of paste of dried parts is usually applied at least 10 grams or more.

A few illustrative examples are described. For a comprehensive review regarding human dosage and uses, see (Nadkarni, 1982; Gogte, 2000). For typical human dosage, see Tables 1A, 1B and 1C.

Angiosperm-Monocotyledon Plants

Cyperous rotundus is well known to folk medicine in the Orient and Africa. Rhizomes or tuberous roots of cyperous are used as anti-inflammatory, anti-pyretic and analgesic particularly for stomach and bowel disorders (Gupta et al., 1971; Seo et al., 2001). Inhibition of NO and superoxide prodution is also reported. Decoctions of tubers or ground powder are given in fever, diarrhoea, dysentery. Paste is applied on the breasts as a galactogouge. (Nadkarni 1982). The human dose of powder is 1 to 3 gm per day. Antimalarial compounds have been isolated (Thebarononth et al., 1995; Weenan et al., 1990). The most active compounds have an IC50 of 5.4 micrograms/ml.

Andropogon muricatus is known (Nadkarni 1982; Gogte, 2000) as a coolent and anti-pyretic. It is particularly useful to reduce burning sensation. The root is given as powder or in the form of an infusion. It controls diaphoresis.

Avena sativa or the common cereal, oat, is regarded as a nutritious cereal. Oat bran contains hemicellulose and its intake reduces LDL (Low Density Lipoproteins) and increases HDL (High Density Lipoproteins) levels in the blood.

Triticum vulgare or the common cereal, wheat, is generally used as a food ingredient. However, general tonic properties of wheat components and preparations are known in folk practice. In “wheat grass therapy” juice of one week old wheat grass is is ingested as a general tonic. Wheat germ is also used in folk practice as a special tonic for general well-being. Wheat germ agglutinin (0.5-1.0 mg/ml) promoted phagocytosis by human blood PMN cells 1.8 times compared to Zymosan-A control (Stoika et al., 2001). However, at higher concentration (5-10 ng/ml) it strongly inhibited phagocytosis.

Allium cepa (Onion) and Allium sativum (Garlic) are the two most commonly used substances of the genus Allium. A variety of uses of onion and garlic have been reported in folk medicinal literature (Nadkarni 1982; Gogte, 2000). Garlic is used particularly for stinulating the digestive and circulatory system. Human dose of bulb paste is 1 to 6 gm per day. Onion is useful in arthritis, sciatica, digestion and cardiac debility (Nadkarni 1982; Gogte, 2000). One (1) to three (3) grams powder of the dry onion bulb is used.

Curcuma genus contains a number of species of medicinal importance such as Curcuma amadal Curcuma longa, Curcuma angustifolia, Curcuma aromatica, Curcuma zedoaria and others. Infusion and paste of the rhizome of Curcuma amada is used (Nadkarni 1982). Internally, the infusion is given for worms and for purification of the blood. Externally, the paste is applied for a variety of skin diseases, often in combination with other medicines. Antifungal activity of the rhizomes has been reported (Ghosh et al., 1980). Human dose of the rhizome powder is 1 to 3 gm per day. Curcuma longa is used (Nadkarni, 1982; Gogte, 2000) as analgesic, anti-inflammatory and chologogouge.

Zingiber genus contains a number of species of medicinal importance. Zingiber officinale (ginger, the common spice) is the most commonly used species throughout the world. Ginger is taken both internally and applied as a paste externally. Traditional medicine lists many applications of both the juice of fresh rhizome and the powder of dried rhizome. Ginger is used for flatulence, dyspepsia, colic and other painful conditions of the stomach. Other applications of ginger include anti-ulcer, promotion of antioxidant enzymes, stimulation of humoral immunity, lipid lowering, skin tumour protection, rheumatism, gastroprotective and antifilarial.

The effective dose required is quite high. In animal studies, aqueous decoctions and ethanolic extracts are used. Oral dose of 50 mg/kg per day for anti-ulcer (Agarwal et al. 2000), 100 mg/kg per day for antioxidant enzyme protection (Ahmed et al., 2000), 200 mg/kg per day for lipid lowering (Bhandari et al., 1998), 500 mg/kg per day for gastroprotection (Al-Yahya et al., 1989) and 100 mg/Kg per day for antifilarial (Datta et al., 1987) was used. For humans, the dose of rhizome powder is from 0.75 to 1.5 g/day (½ teaspoon juice).

Crop health applications of Zingiber officinale extracts or active components include IGR, anti-feedant, antifungal and molluscicidal activity.

The dosage requirements for isolated/derived compounds are quite high. The maximum EC50 activity reported for any compound as IGR is 3.55 mg/ml and as antifungal is 86.46 mg/liter (Agarwal M. et al., 2000).

Table 1A summarizes the common uses and typical human dosage of a variety of Angiosperm-Monocotyledon plants; for a comprehensive review regarding human dosage and uses, see (Nadkarni, 1982; Gogte, 2000; and the references given below).

TABLE 1A
Other Angiosperm - Monocotyledon plants, their uses
and human dosage

| Plant - Angiosperm - | | |
|---|---|---|
| Monocotyledon | Application | Reference |
| Family - Cyperaceae | Antimalarial | (Thebtaranonth et al., |
| Cyperous rotundus Linn. | | 1995; Weenan et al., |
| Human Dose - Root | | 1990) |
| Powder 1 to 3 gm per day | Anti-inflammatory, | (Gupta et al., 1971) |
| | anti-pyretic, | |
| | analgesic | |
| | Inhibition of Nitric | (Seo et al., 2001) |
| | Oxide and | |
| | Superoxide | |
| | production | |
| Family - Gramineae | Avoids burning | (Nadkarni 1982; |
| Andropogon muricatus | micturation and | Gogte, 2000) |
| Retz. | dysurea, Anti- | pyretic |
| Human Dose - Root powder | | |
| 3 to 6 gm per day | | |
| Family - Gramineae | Lowers LDL and | (Nadkarni 1982; |
| Avena sativa, Linn. | Increases HDL | Gogte, 2000) |
| Human Dose - Oat Bran | | |
| 5 to 20 gm per day | | |
| Family - Gramineae | Phagocytosis | (Stoika et al., 2001) |
| Triticum vulgare | promoter | |
| Human Dose - Seeds | | |
| 10 to 100 gm per day | | |
| Family - Liliaceae, | Stimulant, Anti- | (Nadkarni 1982; |
| Allium sp. Human Dose - | pyretic, | Gogte, 2000) |
| Alliun sativumLinn. | Diaphoretic, Skin, | |
| Bud Paste | Anti-inflammatory | |
| 1 to 6 gm per day | | |
| Allium cepa Linn. - Bulb | Arthritis, Sciatica, | (Nadkarni, 1982; |
| powder: 1 to 3 gm per day | Digestion, Tonic | Gogte, 2000) |
| Family - Liliaceae | Galactogouge, | (Nadkarni, 1982; |
| Asperagus racemosus | Aphrodisiac, | Gogte, 2000) |
| Willd. Human Dose - Tuber | Alleviates bleeding | |
| powder 3-6 gm per day | disorders | |
| Family - Zingiberaceae | Skin disease, | (Nadkarni, 1982; |
| Curcuma amada Roxb. | Itching, sprains | Gogte, 2000) |
| Human Dose - Rhizome | Anti-fungal | (Ghosh et al., 1980) |
| Powder 1 to 3 gm per day | | |
| Family - Zingiberaceae | Anti-inflammatory, | (Nadkarni, 1982; |
| Curcuma longa Linn. | Analgesic, | Gogte, 2000 |
| Human Dose - Rhizome | Chologouge | |
| Powder 1 to 3 gm per day | | |
| Family - Zingiberaceae | Anti-ulcer | (Agarwal et al., 2000) |
| Zingiber officinale Roscoe. | Antioxidant | (Ahmed et al., 2000) |
| Human/Animal Health | protection | |
| Human Dose - Rhizome | Humoral Immunity | (Puri et al., 2000) |
| Powder (Juice): 0.75 to | Lipid Lowering | (Bhandari et al., 1998) |
| 1.5 gm (2-4 ml) per day. | Skin Tumor | (Katiyar et al., 1996) |
| Zingiber officinale-Crop | Rheumatism | (Srivastava et al., |
| Health | | 1992) |
| | Gastroprotective | (Al-Yahya et al., |
| | | 1989) |
| | Antifilarial | (Datta et al., 1987) |
| | IGR, Antifeedant, | (Agarwal M. et al., |
| | Antifungal | 2001) |
| | Fusarium wilt | (Singh R. et al., 2000) |
| | Molluscicidal | (Singh K. et al., 2000) |

Angiosperm-Dicotyledon Plants

Momordica charanta juice is used (Nadkarni, 1982; Gogte, 2000) as a galactogouge, and in amenorrhoea and dysmenorrhoea. Juice is also given for sugar control. Extenally, the juce is used for skin disease and for healing of ulcers. The human dose of juice internally is 10 to 30 ml per day.

The family Apocynaceae has several genera of medicinal importance such as Holarrhena, Rauwolia and Vinca.

Holarrrhena genus has several medicinally active species. H. floribunda and H. antidysenterica are the most commonly known to traditional medicine. The bark and seed are both used and the applications include antiamoebic, antimicrobial and antitumour. Both are used for toning of vaginal tissues after delivery.

Total alkaloids from H. antidysenterica showed an MIC of 95 microg/ml against S. aureus (Chakraborty et al., 1999). IC50 values of H. fioribunda for a variety of tumors are 3.4 to 9.8 microg/ml. Human dose of the seed powder is typically 1-2 g/day.

Rauwolfia serpentine is best known for use on irritated nervous system and as hyotensive agent. Human dose of the root powder is 0.5 to 3 gm per day depending on the severity of symptoms.

Vinca rosea root powder is used in anti-cancer applications. The human dose of root powder is 1 to 3 gm per day.

The family Asclepiadaceae has several genera of medicinal importance such as Hemidesmus and Gymnema.

Hemidesmus indicus is used as a blood purifier, anti-infalmmatory, and as an alterative and tonic. The human dose is 3 to 6 gm of root bark.

Gymnema sylvestree is used as a hepatic stimulant. Extract of the leaves works on pancreas and adrenal glands and helps regulate sugar in blood and urine. The human dose is 1 to 2 gm of leaf powder.

Swertia genus has several medicinally active species. Swertia chirata is the most common one. It is used for promoting appetite, as an expectorant and as a blood purifier. It is particularly useful in chronic fevers also malarial fevers.

Methanolic extracts of whole dried plants are reported to have tyrosinase and PEP inhibitory (Khanom et al., 2000) activity. Methanolic extract at a dose of 100 mg/kg i.p. given to rats was found to be hepatoprotective (Karan et al., 1999). The ethanolic extract given to rats was found to have protective effect against ulcerogenic agents.

Plyllanthus genus has several plants of medicinal importance. Among these, Phyllanthus emblica Linn. and Phyllanthus amarus (P. niruri Linn.) are the most widely known. Phyllanthus amarus is known to be useful for viral hepatitis. Phyllanthus emblica Linn. is used for disorders of eyes, liver, spleen and lungs. Paste is applied externally for bruning and headache. Juice is added to eyes for eye disorders. The fruit is a rejuvenating agent and is used in cough, asthma, tuberculosis and also as a brain tonic.

A review of the genus Phyllanthus (Calixto et al., 1998) is available. Hyaluronidase inhibitory activity (Ishizaki et al., 1999) and superoxide scavenging and prolyl endopeptidase inhibitory activities (Khanom et al., 2000) of P. emblica have been reported. Antiviral tannins (Liu et al., 1999) are also reported. Antitumor activity against leukemic cell lines (Sur et al., 1998) for P. emblica has been reported. Being a dietary product, human dosage of P. emblica juice is 12 ml per day and that of fruit powder is 3 to 6 gm per day. Higher doses are not harmful. In case of P. niruri Linn. the total plant is used and the typical dose is 3 to 4 gm powder of total plant per day.

Linum usitatissimum is a dietary oilseed (Flaxseed or Linseed) rich in omega-3 fatty acids and lignans. The decoction of seeds is useful as an expectorant and is used in cough, pleuritis, pneumonia and whooping cough in children.

The regular usage of seeds in the diet (25 to 50 gm per day) can lower cholesterol and post-prandial sugar (Cunnane et al., 1993; Harris, 1997). The seeds are also a strong regulator of inflammatory mediators and alleviate conditions such as eczema, psoriasis and arthritis (James et al., 2000).

Azadiracta indica or the Neem tree has many uses both for human and crop protection applications. Bark, leaves and seed oil are used both for external and internal use. The range of activities and applications include antimicrobial, ant-leprotic, analgesic, wound healing, psoriasis. Human dosage is 1 to 2 gm per day of powder. Cake is generally not given to humans.

Crop applications of leaves, bark and oil include anti-feedant, IGR, anti-fungal, and anti-nematocidal. Neem cake can be used as a molluscicide (Singh K. et al., 1996). Use of cake mixed with urea and and germinated barley has been suggested as a slow ammonia release (Virk et al., 1989) product. Application of 500 kg/ha of neem cake powder (Rao et al., 1992) caused a striking reduction in the culicine larvae, a vector for Japanese encephalitis virus, in a rice field.

Terminalia genus has many species with a wide range of medicinal properties. Some of the most widely used species are Terminalia arjuna, Terminalia bellerica and Terminalia chebula.

T. chebula fruits are used. T. chebula acts as a rejuvenator and is useful in loss of appetite, constipation, ascites, hepatomegaly, splenomegaly, and parasites. It is also useful in disorders of the respiratory system and reproductive system. Application of the fruit or oil extracted from fruit pulp of T. bellerica is useful in painful inflammatory conditions. Oil is used in skin disease, leucorrhoea, and in premature graying of the hair. The rind of the fruit is kept in the mouth acts as a mucolyte. The fruit taken internally is useful in disorders of the respiratory, circulatory and reproductive system.

Human dosage is 1 gm of fruit powder per day as a general tonic. For purgation, 10 gm dose is used. Mouthwash with a 10% solution of aqueous extract of T. chebula significantly inhibited salivary total streptococcal counts and glycolysis of salivary bacteria post-rinsing. T. chebula increased gastric emptying in rats (Jagtap et al., 1999) at a dose of 100 mg/kg/day given orally for 15 days. Alcoholic extracts of T. chebula and T. bellerica at 200 mg/ml showed interesting activity against a number of pathogenic and opportunistic microorganisms.

Eugenia jambolana seeds, bark, fruit and leaves are used. Human dose is 12 to 25 nl for juice and 1 to 3 gm for powder. The plant is used for diabetes, diarrhoea and menorrhgia with bleeding, and dysentery.

Piper genus has several species of wide ranging medeicinal properties. Piper nigrum and Piper longum are the most commonly used species. Both are stimulants of appetite, liver, spleen and nerves. They promote bioabsorption of other drugs and are useful in cough and asthma.

Embelia ribes has many applications. In rhinitis and migraine, a fine powder is used for nasal adminstration. It is a nervine tonic and is used for abdominal colic, flatulence and particularly against round worm, thread worm and tape worm infestations. Extermally it is used on skin diseases.

Administration of Embelin from Embelia ribes produces (Chitra et al) a dose-dependant decrease in labeled thymidine uptake, lipid peroxidaation and glutathione levels. Anti-spermatogenic and retinotoxic effects have also been noted.

Human dosage of fruit powder is 1 to gm per day. For use against worms, a dose of 10 gm is used. Daily subcutaneous administration of embelin (Gupta et al., 1989) at a dose of 20 mg/kg body weight to male albino rats revealed an inhibition of sperm count and other fertility parameters. Chicks fed Embelia ribes at the rate of 0.5 gm/kg per day (Low et al., 1985) showed a dose-dependant degeneration of the retina. Defects were noted above a cumulative dose of 0.25 gm.

Tinospora cordifolia is used internally for chronic fevers, diabetes, as a restorative, anti-inflammatory and antacid. The human dose of root powder is 1 to 3 gm per day.

Glycine max (soybean) is used as an oilseed. It is a lactagouge, emenagouge and aphrodisiac. The human dose is 10 to 20 gm seeds per day.

Glycyrrhiza glabra is used externally as analgesic, helps in hair growth and promotes skin smoothness and complexion. The root powder is used internally in cough, hoarseness of voice, anaemia, bleeding disorders, disurea, pyorrhoea, and for increasing sperm count.

Oral administration of Lacrinat (Konovalova et al., 2000) containing liquorice root powder for 1 month markedly increased antioxidant activity of the liver. All crude polysaccharide fractions from the shoot and hairy roots of G. glabra induced nitric oxide production by murine peritoneal macrophages in vitro.

Human dose is 1 to 2 gm of root powder per day. Methanolic extract of G. glabra had a 50% tyrosinase-inhibitory concentration of 21.2 microg/ml (Khanom et al., Sept. 2000). Beta-glycyrrhetinic acid is a potent inhibitor (Kroes et al., 1997) of the classical complement pathway (IC50=35 microM).

Mucuna pruriens roots and seeds are tonic for neurons. Root is used in facial palsey and hemiparesis. Seeds are used in Parkinson's disease. The roots are diuretic and seeds are useful in impotence ans oligospermia.

Anti-snake venom (Guerranti et al., 2001) properties of M. pruriens extract are a result of its ability to increase precoagulant activity.

Human dose is 3 to gm of seed powder per day. Higher doses are also used. In a clinical study on Parkinson's disease patients, a concoction in cow's milk containing M. pruriens seed powder was found to give a good response in tremor, bradykinesia, stiffness and cramps (Nagashayana et al., 2000). The daily dose contained 200 mg of L-DOPA. In alloxan-diabetic rabbits (Akhtar et al., 1990) 1 gm/kg per day root powder caused a significant fall in blood glucose levels.

Phaseolus radiatus and Phaseolus mungo are pulses and are used as a source of high protein food. They are consumed in the range of 5 to 50 gm per day. They are also useful for the health of hair and skin and are anti-inflammatory when applied externally as a paste.

Pongamia glabra bark, leaves and seeds are antiseptic, antipruritic and analgesic externally. Oil from seeds has antiparasitic, wound healing and analgesic properties. Internally the various parts are used for helminthasis, and as a liver tonic.

Human dose is 1 to 3 gm of seeds per day. Solvent extracted P. glabra cake (Ravi et al., 2000) can be fed to lambs (20% of feed) without affecting the performance for a period of 98 days. However, expressed Karanj (P. glabra) cake may not be recommended as it adversely affects the intake and digestibility of nutrients. Aqueous extract of deoiled kernels (Sagar et al., 1996) at 100 ppm causes 100% mortality in 4^{th} in star larvae and pupae of Culex mosquito.

Trigonella foenum-graecum intake is advised to women in the post natal period to improve excretory and menstrual functions and as a galactogogue. Seed powder is used locally to reduce inflammation and to reduce hair fall. It is also used as a lipolytic agent. Hypoglycaemic and Antiulcer activity has been reported (Zia et al., 201) and Trypsin/Chymotrypsin inhibitor (Weder et al., 1991) activity has also been noted.

Human dose is 3 to 6 gm of seed powder per day. Methanolic extract given to mice produced antidiabetic effect at a dose of 1 gm/kg per day.

Santalum album heartwood (2 to 5 gm) and oil (5 to 20 drops) are used for external applications and for internal use. It is used to reduce burning and thirst, dusurea and for the treatment of purulent menstuation. It cures dermatoses and pruritis.

Ocimum genus has several important medicinal plants. Ocimum sanctum is the most commonly used plant. The juice (10 to 20 ml) and seed (1 to 2 teaspoons) are taken internally. Its main action is on the respiratory system. It is a common remedy for cold, cough and fever. The paste of leaves is also used for ringworm and scabies. Seed is a diuretic and tonic.

Sesamum indicum seeds and oil are used as food. The intake of seeds is useful for gum and dental health, hair and skin. It is useful in dysmenorrhoea, diabetes and bleeding piles. Oil is used for massage in paralysis, fractures and wounds.

As a common dietary item, human dose is 10 to 20 gm per day.

Herpestis monnieri is a valuable plant for brain and the entire nervous system. It is given in mania, epilepsy and retardation. It is also useful in cough and cold. The human dose is 10 ml of leaf juice.

Withania somnifera is well known for its aphrodisiac property. It is an immunomodulator and also given to reduce pain in rheumatoid arthritis and for abdominal pain. The human dose is 5 gm of the root powder.

Carum roxburghianum is used as a spice in cooking. It is useful as a carminative, analgesic, anti-inflammatory, and antihelminthic. It is an aphrodisiac and is used for treating amenorrhoea and dysmenorrhoea. The human dose is 5 to 7 gm of seed powder.

Cuminum cyminum is used as a spice in cooking. It is useful as a carminative, analgesic, anti-inflammatory, and antihelminthic. It also improves lactation. The human dose is 5 to 7 gm of seed powder.

Ficus genus has several plants of medicinal importance. Ficus bengalensis fruit, bark, aerial roots and latex are all used. Latex is applied on wounds, synovitis, arthritis, toothache, conjunctivitis and piles. Latex, bark and fruit act as antidiabetic. In leucorrhoea and menorrhagia, decoction of bark is used as douche.

The fruit extracts have antitumour activity in the potato disc bioassay and also antibacterial activity (Mousa et al., 1994). These results support the use in respiratory and certain skin disorders. Leucocyanidin derivatives from the bark have antidiabetic activity (Kumar et al., 1989), and they promote hexokinase and HMGCOA reductase levels in tissues.

Human dose is 50 to 100 ml decoction of the bark or 3 to 6 gm of powder per day. Leucocyanidin derivates (Kumar et al., 1989) give a significant effect at 100 mg/kg body weight.

Table 1B summarizes the common uses and typical human doses of a variety of other Angiosperm-Dicotyledon plants; for a comprehensive review regarding human dosage and uses, see (Nadkarni, 1982; Gogte, 2000; and references given below).

TABLE 1B
Other Angiosperm - Dicotyledon plants, their uses and
human dosage

| Plant - Angiosperm - | | |
|---|---|---|
| Dicotyledon | Application | Reference |
| Family - Cucurbitaceae | Diabetes, | (Nadkarni, 1982; |
| Momordica charantia, Linn. | healing, dysmenorrhoea, | Gogte, 2000) |
| Human Dosage - Juice of plant or | amenorrhoea, skin | |
| fruit 10 to 30 nl per day | galactogouge | |
| Family - Apocynaceae | Antitumor | (Loukaci A. et al., |
| Holarrhena floribunda | | 2000; Abreu et al., |
| Human Dosage - Decoction of | | 1999) |
| Seeds | Antileschmania, | (Abreu et al., 1999) |
| 0.5 to 1 gm per day | antimicrobial | |
| Holarrhena | Antibacterial, | (Chakraborty et al., |
| antidysenterica, Roxb. | Antimicrobial | 1999; Ahmed et al., |
| | | 1998) |
| | Stimulate phagocytosis | (Atal et al., 1986) |
| | Hepatotoxicity | (Arseculeratne et al., |
| | | 1981) |
| Family - Apocynaceae | Sedative, Lowering of | (Nadkarni, 1982; |
| Rauwolfia serpentina, Benth Ez | B.P., Promotes Uterine | Gogte, 2000) |
| Kurazaa Human Dosage - Root | contractions, | |
| 0.5 to 3 gm per day | | |
| Fanily - Apocynaceae | Anti-cancer | (Nadkarni, 1982; |
| Vinca rosea, Linn. | | Gogte 2000) |
| Human Dosage - Roots | | |
| 1 to 3 gm per day | | |
| Family - Asclepiadaceae | Blood purifier, Tonic, | (Nadkarni, 1982; |
| Hemidesmus indicus, R. Br. | Alterative, | Gogte, 2000) |
| Human Dosage - Root Bark paste | Anti-inflammatory | |
| 3 to 6 gm | | |
| Family - Asclepiadaceae | Hepatic stimulant, | (Nadkarni, 1982; |
| Gymnema sylvestree, R. Br. | Anti-diabetic | Gogte, 2000) |
| Human Dosage - Leaf powder | | |
| 1 to 2 gm | | |
| Family - Gentianaceae | Liver Toxicity | (Karan et al., 1999; |
| Swertia chirata, Ham. | | Reen et al., 2001) |
| Human Dosage - Whole Plant | Tyrosinase and Prolyl | (Khanom et al., |
| 2 to 6 gm per day | Endopeptidase | April and Sept. |
| | Inhibitor | 2000) |
| | Gastric Ulcer | (Rafatullah et al., |
| | Protection | 1993) |
| Family - Euphorbiaceae | General Review | (Calixto et al., 1998) |
| Phyllanthu niruri, Linn. | Superoxide scavenging | (Khanom et al., |
| Human Dosage: Total Plant | and PEP Inhibition | April 2000) |
| Powder | Hyaluronidase | (Ishizaki et al., |
| 3 to 6 gm per day | Inhibition | 1999) |
| Phyllanthus emblica, Linn. | Anti-tumor | (Sur et al., 1998) |
| Human Dosage - Fruit | Anti-viral | (Liu et al., 1999) |
| Powder: 3 to 6 gm per day | | |
| Juice: 12 ml per day | | |
| Family - Linaceae | Cholesterol | (Harris, 1997) |
| Linum usitatssiimum, Linn. | Post prandial blood | (Cunnane et al., |
| Human Dosage - seeds | glucose | 1993) |
| 5 to 50 gm per day | Inflammatory Mediator | (James et al., 2000) |
| | Production | |
| Family - Meliaceae | Slow ammonia release | (Virk et al., 1989) |
| Azadiracta indica, A. Juss. | Molluscicidal | (Singh K. et al., |
| Human Dosage - Bark Powder: 1 | | 1996) |
| to 2 gm per day. Leaf Juice: 12 ml | Culex Mosquito Larvae | (Rao et al., 1992) |
| per day | in Rice Fields(vector | |
| Cake is not used for humans. | for encephalitis virus) | |
| Family - Conbretaceae | Antimicrobial | (Ahmad et al., 1998) |
| Terminalia sp. | Gastric Emptying | (Tamhane et al., |
| Terminalia bellericaa, Roxb. | | 1997) |
| Erminalia chebula, Retz. | Anticaries agent | (Jagtap et al., 1999) |
| Human Dosage - Fruit Powder | HIV Reverse | (el-Mekkawy et al., |
| 1 gm per day | Transcriptase | 1995) |
| 3 to 6 gm for purgation | | |
| Family - Myrtaceae | Diabetes, | (Nadkarni, 1982; |
| Eugenia jambolana, Lam. | Liver Funcction | Gogte, 2000) |
| Human Dosage - Fruit, Seed, | Diarrhoea, Dysentry, | |
| Bark, Leaves: | Menorrhagia with | |
| Juice 12 to 25 ml, Powder 1 to 3 gm | bleeding | |
| Family - Piperaceae | Stimulant for appetite, | (Nadkarni, 1982; |
| Piper nigrum, Linn. and | liver, spleen, nerves | Gogte, 2000) |
| Piper longum, Linn. | Promote bioabsorption | |
| Human Dosage - Fruit | of other drugs | |
| 0.25 to 0.5 gm for P. nigrum | cough and asthma | |
| 5 to 10 gm for P. longum | | |
| Family - Myrsinaceae | Anti-neoplastic | (Chitra et al.) |
| Embelia ribes, Burm. | Anti-spermatogenic | (Gupta et al., 1989) |
| Human Dosage - Fruit | Retinotoxic | (Low et al., 1985) |
| 1 to 2 gm per day. 10 gm for | | |
| worms. | | |
| Family - Menispermaceae | Chronic fevrs, | (Nadkarni, 1982; |
| Tinospora cordifolia, Willd | Diabetes, Anti- | Gogte, 2000) |
| Meirs. | inflammatory, Antacid, | |
| Human Dosage - Bark powder | Restorative | |
| 1 to 3 gm | | |
| Family - Leguminosae | Lactagouge and | (Nadkarni, 1982; |
| Glycine max, Merr. | emengouge, | Gogte, 2000) |
| Human Dosage - Seeds | aphrodisiac | |
| 10 to 20 gm per day | | |
| Family - Leguminosae | Tyrosinase Inhibitor | (Khanom et al., |
| Glycyrrhiza glabra, Linn. | | Sept. 2000) |
| Human Dosage - Root Powder | Macrophage Activation | (Nose et al., 1998) |
| 1 to 2 gm per day | Anti-inflammatory | (Kroes et al., 1997) |
| | Antioxidant | (Konovalova et al., |
| | | 2000) |
| Family - Legyminosae | Anti-snake venom | (Guerranti et al., |
| Mucuna pruriens, Bak. | | 2001) |
| Human Dosage - | Parkinson's Disease | (Nagashayana et al., |
| Seed Powder: 3 to 6 gm per day | | 2000) |
| Root Extract: 50 to 100 ml per | Antidiabetic | (Akhtar et al., 1990) |
| day | | |
| Family - Leguminosae | Anti-inflammatory | (Nadkarni, 1982; |
| Phaseolus radiatus | Complexion Promoter | Gogte, 2000) |
| Human Dosage - Seeds | | |
| 5 to 50 gm per day | | |
| Family - Leguminosae | Anti-mosquito | (Sagar et al., 1996) |
| Pongamia glabra, Vent. | Solvent Extracted Cake | (Ravi et al., 2000) |
| Human Dosage - | As Feed Supplement to | |
| Juice of Bark or Leaves: 6-12 ml | Lambs | |
| per day | | |
| Seeds: 1 to 3 gm per day | | |
| Family - Leguminosae | Hypoglycaemic | (Zia et al., 2001) |
| Trigonella foenum - graeceum, | Trypsin/Chymotrypsin | (Weder et al., 1991) |
| Linn. | Inhibitor | |
| Human Dosage - Seed Powder | | |
| 2 to 6 gm per day. | | |
| Family - Santalaceae | Burning sensation and | (Nadkarni, 1982; |
| Santalum slba Linn. | thirst, Dermatoses and | Gogte, 2000) |
| Human Dosage - Heartwood, Oil | Pruritis, purulent | |
| Powder 2 to 5 gm, Oil 5 to 20 | menstruation | |
| drops | | |
| Family - Labiatae | Expectorant, Fever, | (Nadkarni, 1982; |
| Ocimum sanctum Linn. | Cough, Cold, | Gogte, 2000) |
| Human Dosage - Juice 10 to ml | Diaphoretic, Ringworm | |
| Seeds - 1 to 2 teaspoon | | |
| Family - Pedaliaceae | Lactagouge and | (Nadkarni, 1982; |
| Sesamum indicum | emenagouge, | Gogte, 2000) |
| Human Dosage - Seeds | aphrodisiac | |
| 10 to 20 gm per day | | |
| Family - Scrophulariaceae | Epilepsy, Depresion, | (Nadkarni, 1982; |
| Herpestis monnieri, H. B.K. | Retardation, | Gogte, 2000) |
| Human Dosage - Leaf Juice | Cough and Cold | |
| 2 teaspoon (10 ml) | | |
| Family - Solanaceae | Aphrodisiac, | (Nadkarni, 1982; |
| Withania somnifera, Dunal. | Immune modulator, | Gogte, 2000) |
| Human Dosage - Root Powder | Rhematic Arthritis, | |
| 5 gm per day | Abdominal Pain | |
| Family - Umbelliferae | Anti-inflammatory, | (Nadkarni, 1982; |
| Carum roxburghianum, Benth | Analgesic, Carminative, | Gogte, 2000) |
| Human Dosage - Seeds | Antihelminthic, | |
| | Amenorrhoea, | |
| | Dysmenorrhoea | |
| Family - Umbelliferae | Anti-inflammatory, | (Nadkarni, 1982; |
| Cuminum cyminum, Linn. | Analgesic, Carminative, | Gogte, 2000) |
| Human Dosage - Seeds | Antihelminthic, | |
| 3 to 6 gm per day | Galactogouge | |
| Family - Moraceae | Antitumor, | (Mousa et al., 1994) |
| Ficus bengalensis, Linn. | Antibacterial | |
| Human Dosage - | Antidiabetic | (Kumar et al., 1989) |
| Decoction of Bark: 50 to 100 ml | | |
| per day. | | |
| Powder: 3 to 6 gm per day. | | |

Gymnosperm Plants

There are three species known by the name Talispatra. They are: Abies webbiana Lindle, Taxus baccata and Rheododendron anthropogon. One of them is highly poisonous, Taxus baccata a species from which taxol, an anticancer drug, has been isolated. Leaves are used as an appetizer and in cough, asthma, general debility and pthisis. The dose of leaf powder is 0.5 to 1 gm.

Pinus deodar Roxb. Sergent is used widely. Leaf powder, oil and latex are all used. Externally the latex is used on abscesses. The oil (turpentine oil) is useful for pulmonary edema, arthritis, flatulence. Internally, the plant is used as hepatostimulant, on acute and chronic bronchitis and a variety of skin diseases.

Human dosage powder and oil is 1-3 gm and 1-2 gm, respectively. Overdose causes, diarrhoea, vomiting, paralysis and sensory loss.

Table 1C summarizes the common uses and typical human doses of a variety of Gymnosperm plants; for a comprehensive review regarding human dosage and uses, see (Nadkarni, 1982; Gogte, 2000; and references given below).

TABLE 1C
Gymnosperm plants, their uses and human dosage

| Plant - Gymnosperm | Application | Reference |
|---|---|---|
| Order - Conifereae | Anti-tumor | (Mantle et al., 2001) |
| Taxus baccata(also Abies | Toxicity | (Kite et al., 2000) |
| webbiana Lindle) | | |
| Human Dosage - Leaf powder | | |
| 0.5 to 1.0 gm per day | | |

Non-Plant Biomass Sources

A wide range of many other biomass sources have also been used for the treatment of a wide range of ailments all over the world. This range of biomass includes:

Microbial, Mycological, Marine, Poultry, Animal and Human.

The preparations start with many different parts of biomass such as blood, mik, urine, organ meat, cartilage and chitin, skin. Total cell mass may also be used. As many of these are food materials, typical human dosage levels required on the basis of fresh part is 10 to 50 gm per day. In the case of dry powders of mushroom, yeast or lactobacilli, the dosage may be 1 to 10 gm.

The total list of non-plant biomass sources used includes the entire set of life forms and is therefore too exhaustive to be cited here. General use and nutritional applications of meat, milk and eggs from poultry, seafood and animals are also well known and are not repeated here. Only a few illustrative examples of non-plant biologicals are described.

Mushrooms have been used medicinally for centuries, particularly in traditional Chinese and Japanese medicine (Lombardi R. M. 2002). They are considered as promoters of health and vitality and are adaptogens. The constituents show, immunomodulatory, amtobacterial, antiviral, antitumour, antiparasitic, cardiovascular hypercholesteromiac properties. Active substances include beta-glucans and polysaccharide-protein complexes Button mushroom (Agaricus bisporus) is a very common type of mushroom, and is used as a source of high protein food ingredient. It is also known to promote digestion and help lower blood pressure.

Beta-glucans are also isolated from the cell walls of yeast (Bacon et al. 1969) and oat and barley bran. Beta-glucans activate the anti-infection and antitumour activity of macrophages. Hence, yeast and oat and barley bran also have immune modulating activities. Beta-glucan dose is typically 2 mg/kg per day.

Shrimp(Prawn) outer shell and shark fin are used as a source of glucosamine and chondroitin in commercial preparations and are given internally for rebuilding cartilage in joints. No specific medicinal activity has been claimed for them.

Table 1D summarizes the common uses and typical human dose of a variety of non-plant biomass.

TABLE 1D
Non-plant Biomass Sources, their uses and human dosage

| Biomass Source | Application | Reference |
|---|---|---|
| Yeast | Digestion, | Bacon et al. 1969 |
| Active dried Baker's Yeast | Immunomodulation | |
| Saccharomyces cerevisiae | | |
| Human Dosage - dry powder 2 | | |
| to 6 gm | | |
| Beta-glucan - 200 mg | | |
| Mushroom | Immunomodulation, | Lombardi, 2001 |
| Agaricus bispores (Button | Digestion, Blood | |
| Mushroom) | Pressure Lowering | |
| Human Dosage - dry powder 2 | | |
| to 6 gm | | |
| Beta-glucan 200 mg | | |
| Prawn (Shrimp) | Source for | |
| | glucosamine | |
| Shark cartillage | Source for | |
| | chondroitin | |

Toxicity

Because of the relatively high effective doses and the traditional methods of preparation, toxins from CAM plants that might be otherwise negligible can adversely affect a subject. For example, Crassulaceae juices and aqueous extracts from various plants have cytotoxic substances (Avila et al., 1997; Balachandran et al., 1991; Botha et al., 1997; Mueller and Stopper, 1999; Sivaswamy et al., 1991). High therapeutic doses of leaf juice for internal use (more than 100 mg/kg bbody weight daily) coupled with high mammalian toxicity close to the habitual level of use (LD₅₀ in mice is 230 mg/kg and in rat is 560 mg/kg, respectively (Verma et al., 1986)) render these compositions less useful.

Toxicity issues rendering compositions less useful is not restricted to CAM plants. Many other plants and other biologicals also have toxic substances and their toxic effects are known. As an example, Embelia ribes used for the treatment of worms is known to be retinotoxic (Low et al., 1985). Holarrhena antydysenterica used for amoebic dysentery is known to have hepatotoxicity (Arsecularatne, 1981).

Shelf Life and Potency

The traditional methods of preparation, juice extraction and ground leaf, suffer from poor shelf life, especially fresh plant juice, which ferments readily if not sterilized or stored properly. Even when dry leaf or stem powder is used, the shelf life of such products is 6 months to one year. Because preparation methods have not been optimized, potency varies by preparation, and thus each preparation may have different effective doses.

Previous Oil Extraction Methods

Medicated oils using herbal materials are known in Indian traditional medicine. The base oils used for such preparations are sesame oil and ghee (clarified butter). In South Indian practice coconut oil may replace sesame oil. Sharangdharsamhita, an ancient treatise by Sharangdharacharya (1961), a standard reference treatise of traditional medicine describes a standard method of preparing such “medicated oils.”

In traditional practice, such extracts are made by boiling together a mixture of kalka (ground paste or homogenate), oil and other liquid substances. The recommended ratio of kalka:oil:liquid substances changes with the nature of liquid substance used. Water, plant derived liquids and juices are the three types of liquid substance described. Plant derived liquids used for making oil extracts are decoctions of plant parts in water. The decoctions are the filtrates prepared by boiling plant part in water, and filtering to obtain a clear liquid or decoction. The ratios of kalka:oil:liquid substance for these three cases are 1:4:16; 1:6:24; and 1:8:32, respectively. Therefore, in these three cases the overall ratio of water to kalka, plant derived liquids to kalka and juice to kalka are recommended to be 16:1, 24:1 and 32:1.

Juice based or decoction based preparation is the commonly used preparation in practice for fresh or dried succulents. Thus, the standard preparations for succulents call for a very high ratio of juice to ground paste (32:1) or decoction to ground paste (24:1) in making the medicated oil extracts. Traditionally, the effective substances were thought to be present only in the fresh juice or decoction; the bulky residue from ground paste was considered unimportant. Furthermore, the typical recommended dose of such medicated oils is as high as 4 tola (1 tola=11.4 g). This standard procedure is practiced in Ayurveda, the traditional medicine of India. These oil preparations are thus characterized by predominant use of juice or decoction and a high dose. Classical treatises and other references specify particular plants for such methods since the belief is that the various healing substances are liberated from the plants in very specific ways (Nanal, 1995).

However, Nanal (1995), in reviewing the use of Kalanchoe in the context of theory and practice, remarks that Parnabeeja (Kalanchoe) is not mentioned in any Ayurvedic texts. Nanal mentions several different preparations from Kalanchoe that includes oils, both in sesame and in clarified butter, but does not specify the usefulness of such preparations, and he does not recommend dosages. Such Kalanchoe preparations are usually prepared from the juice of the plant; with only a small amount of leafy residue. These juice-based preparations are seldom used because of serious toxic side effects at very low doses, and when used, are only topically (as opposed to internally) administered.

Aloe extracts are used commercially in hair oils, often as part of multi-herb medicated oils. Such preparations use extracts prepared from fresh Aloe juice or decoctions of dried Aloe pulp. Aloe oils, by themselves, are not generally recommended for topical or internal use; instead, Aloe liquids, gels or pulp are used.

The use of oil extracts of Cissus is unknown.

In a more general way, oil or ghee (clarified butter) extracts of plants are more commonly used in multi-herb compositions used for both external and internal applications. Specific detailed description for individual plants is difficult to come by. The generalized procedures described above are therefore the guidelines to go by.

Oil extract preparations for non-herbal biomass are not described in traditional medicine.

The use of paste or residue of total plant or plant parts as the predominant component compared to the use of juice or decoction as the dominant component in extractions is contrary to the teachings of traditional medicine. However, surprisingly, the methods of the invention allow for the preparation of compositions that have an enormous potential to improve health by mining the beneficial effects and minimizing toxicity of plants. These methods also produce compositions of high potency at very low doses, thus further reducing any potential for toxicity.

The methods of this invention also produce compositions of high potency and low toxicity from all non-herbal biomass sources including microbial, mycological, veterinary, human and aquatic.

The invention circumvents the problems of toxicity and shelf life by providing a general way to make compositions that incorporate oil extracts of a wide variety of plants and other non-plant biomass. These compositions have a very high useful activity (on the basis of biomass material dose) which allows extremely low dosage and as a result, the toxicity is considerably reduced. These compositions bring out the activity characteristic of each biomass. As a result, these compositions are useful for a wide variety of applications, including human, veterinary and plant applications, for both known and novel uses. These applications include broad general effects such as disease resistance, stress resistance, general promotion in health and growth, delaying senescence and special effects such as wound healing, skin repair, stimulation of hair growth, bone repair and lipid lowering.

### BRIEF SUMMARY OF THE INVENTION

This invention relates to compositions comprising biomass extracts to be used in human, veterinary and agricultural aquatic, mycological and microbial applications.

The novel compositions are prepared from biomass by taking the entire biomass or appropriate parts, washing them with water, soaking them (in case of hard seeds or dry material), pounding them (in case of hard materials such as seeds), or cutting them into pieces (in case of soft plant material), mixing them with water, homogenizing the mixture, and filtering the homogenate to obtain two fractions: juice (J) fraction (as the filtrate) and the leafy residue (or stem, biomass part, etc.; LR) fraction. The fractions may be mixed together, or kept separate as J or LR fractions. The total homogenate or any form and any proportion of the fractions may be mixed with oil or fat, adding water, bringing the mixture to boil, stirring the mixture to provide good contact of biomass with both water and oil and to minimize sticking of biomass to the vessel bottom, removing the water by boiling, cooling the mixture, and filtering the mixture to separate the oil extract from the residue, which are referred to as the first oil extract and first residue, respectively. A second extract from the particular fraction may be obtained by washing the corresponding first residue with oil and filtering to obtain a second extract. Subsequently, the first and second extracts may be combined. The composition can be used to treat a variety of human and animal ailments, and has manifold applications in agriculture, using exceptionally low doses and without toxic side effects. These uses will become apparent as the various embodiments of the invention are discussed.

### DETAILED DESCRIPTION

The novel herbal compositions of the invention, is prepared by a method wherein the total biomass or one or more biomass parts are first reduced in size by soaking, pounding and cutting as required, and are then homogenized, adding water as required. The total homogenate (kalka) is filtered to separate the juice fraction (J) from the concentrated stem/leaf/plant parts residue (LR). The total homogenate may be added or the two fractions may be added separately (mixed in any proportion) to oil with additional water as required; the water is then removed by boiling. Stirring is provided to promote good contact between the biomass, oil and water.

Total biomass or any part or parts of the biomass can be used to prepare a range of extracts. The concentrated seed/rhizome/stem/leaf/biomass parts residue may be used to prepare LR fraction; or, only the juice fraction may be used to obtain J fraction. Thus, the proportion of the biomass residue fraction to the juice fraction may be 1:0 or 0:1. The combined extract comprises both the J and LR fractions. By varying the starting biomass materials (including plant, non-plant materials and their parts, etc.), the admixing of J and LR fractions, and filtration provides the preparation of extracts with a variable biological activity that are suitable for specific applications (see Examples). Because various factors can be adjusted during the preparation of the compositions of the invention (biomass parts, ratio of leaf, stem, biomass parts, J fraction, LR fraction, oil, etc.), the drawbacks of traditional methods of preparation, such as cytotoxicity and excessively high doses, are circumvented.

In addition, the compositions of the invention have unexpected and useful results, including high potency coupled with low toxicity, an exceptionally long shelf life, and a wide range of usefulness.

The two important features of this invention in the context of plant extracts are the ratio of oil to homogenate or kalka of biomass/biomass parts and the ratio of liquid substances to homogenate or kalka of biomass/biomass parts taken for boiling.

The traditional methods recommend an oil to kalka ratio of 4:1, 6:1 and 8:1 for use depending upon the use of water, plant decoction or juice as the liquid substance used. This invention uses the lowest possible ratio consistent with ease of processing. Thus, with succulent leaves and fruit as the plant part, the typical ratio of oil to starting plant part is 1 to 2 in the presence of water. A ratio of homogenate to the oil of 0.1 to 1.5 can also be used. In the case of dried material and particularly where the material forms a pasty mass after homogenization, oil to starting plant part ratio of 4:1 may be used in the presence of water.

The traditional methods recommend the overall ratio of water to kalka, plant derived liquids to kalka and juice to kalka to be 16:1, 24:1 and 32:1, respectively.

This invention uses ratios which are significantly different. In general, present invention uses overall ratio of water to kalka, plant derived liquigds to kalka and juice to kalka less than 16:1, less than 24:1 and less than 32:1, respectively. This is best understood in the context of one of three cases.

The first case is where the total homogenate is used for boiling without separating the LR and J fractions. In this case, unlike the traditional method, there is no additional decoction or juice is used at all. Thus, it is equivalent to using a ratio of decoction or juice to kalka or homogenate of zero instead of 16:1 and 24:1 and 32:1.

The second case is where the LR fraction is taken further for boiling. In this case, not only no additional decoction or juice is used but most of the juice inherently present in the succulent starting material is also washed away. Thus, this method is equivalent to having a negative ratio of juice to initial plant material.

In both these cases, some water inevitably gets added for homogenization and also to control the time of boiling. The total water to initial plant material is typically in the ratio of 1:1 to 8:1. The low ratio is likely to be used with succulents where total homogenate is being used. The higher ratio is required when starting with dry materials which tend to form a thick paste. Even the higher ratio is much lower than the 16:1 ratio recommended in traditional literature.

Thus, for total homogenate or for the LR fraction, considerably lower total liquid quantities are used. This drastically cuts down the processing time, avoiding loss of activity caused by excessive heating for a long time. At the same time, this still provides an ability to adjust the ratio of water and helps in controlling and optimizing the boiling time to the desired level as required for each specific extract.

This invention also incorporates another novel feature as an “Extraction Concept.” Traditionally, plants are typically extracted in one solvent. The filtrate is then concentrated and evaporated to dryness to obtain the active concentrate. U.S. Pat. No. 5,529,778 (1996) describes a composition made by such a process where the plant material is extracted in distilled water and the filtrate is evaporated to dryness. Sometimes, the residue from the first solvent extraction may be further extracted by a series of different solvents, all the filtrates combined together and then evaporated to obtain the active material. In some other cases, the plant is extracted in one solvent and then the filtrate is back-extracted into another solvent.

However, the concept of fractionating the total homogenate into LR and J fractions and separately processing them in oil to produce two separate compositions with widely different biological activity is novel and forms a special feature of this invention. This can be explained in the following manner. The step of homogenization brings the plant material with intimate contact with a solvent (e.g. cold water). After filtration, the two fractions, LR and J, contain a selective fractionation of plant components in them. Subsequent oil extraction, therefore, yields two separate extracts with widely differing biological activity in many cases. In some cases one fraction gets enhanced activity and reduced toxicity than the total homogenate for a particular application. In some other cases, one of the fractions becomes stronger in one activity while the other fraction also develops strong activity for a different application. Thus, this scheme of fractionation/extraction provides a general method to greatly enhance the utility of many extracts and therefore the utility of many plants as per this invention. In the oil extract of the present invention, the ratio of the first residue to the oil may be from 0.05:0.5. Alternatively, in the oil extract of the present invention, the ratio of the biomass juice to the oil may be 0.5 to 10.

The key features, methods and the ‘Extraction Concept’ outlined above are also applicable to making compositions from all non-plant biomass materials as per this invention.

Potency

Doses of less than 1 mg/kg body weight/day on the basis of total fresh leaf or stem or plant parts or non-plant biomass weight for human (and mammalian) internal use is sufficient to produce significant therapeutic effects compared to greater than 50 mg/kg body weight therapeutic dosage traditionally used. A 5 to 50 mg plant or other biological material equivalent is sufficient for topical applications, compared to the traditional use of 5 to 10 g of juice or homogenate. A dose level of less than 1 mg/kg body weight per day of plant equivalent is effective in poultry applications, compared to approximately 100 mg/kg body weight per day as traditionally used.

Low Toxicity

Oil extracts of Kalanchoe pinnata (Lam.), when prepared according to the methods of the invention, are not toxic when given in doses of 50 mg/kg/day for 6 months to Sprague-Dawley rats. Even at doses of 500 mg/kg/day, changes in mortality rates or histopathology are not observed. The compositions are not cytotoxic in vitro when administered to 60 different tumor cell lines at doses up to 250 ppm. Thus, compared to the toxicity levels reported for the traditionally prepared compositions, the toxicity of the compositions of the present invention is negligible, even at high doses.

Similar enhancement in the efficacy/toxicity ratio is expected for other plants and non-plant biologicals as well.

Shelf Life

Fresh juice or extracts prepared by traditional methods ferments rapidly. However, the compositions of the invention remain potent much longer. In case of Crassulaceae, the extracts have remained potent even after at least 7 years.

### I. EMBODIMENTS

A. Human

The compositions of this invention can be made from a wide variety of plants and non-plant biomass. For each biomass, the key activities as reported in the literature can be incorporated in the compositions of this invention. Hence, the compositions of this invention have a wide variety of human applications. An illustrative summary of examples of the many embodiments for selected CAM plants is given in Table 2.

Thus, in the context of CAM plants alone, the compositions of the invention may be used to treat respiratory disorders and skin conditions, modulate the immune system, lower blood lipid levels, improve digestion, promote healing, regulate menstruation and ovulation, and may be used as an anti-inflammatory agent. Dosages are unexpectedly low when compared to traditional applications, from 100 to 1000 times less.

The compositions may also be used prophylactically.

TABLE 2
Human embodiments of uses for the compositions of
the invention

| General | |
|---|---|
| embodiment | Specific embodiments |
| Respiratory | Treating coughs, colds and congestion |
| | Treating asthma, including allergy and stress-induced |
| Circulatory | Promoting circulation in feet |
| | Lowering low density lipoproteins (LDL)/cholesterol |
| | Lowering triglycerides |
| Digestive | Treating ulcers from Diabetes |
| | Reducing stomach acidity |
| | Reducing stomach upsets |
| | Promoting appetite |
| Growth | Promoting weight gain |
| | Promoting height growth in children |
| Healing/Wound | Promoting healing of bruises and cuts |
| repair | Promoting healing of ulcers from leprosy |
| | Promoting healing of bedsores |
| | Promoting healing of burns |
| | Promoting healing of piles (hemorrhoidal tumors) |
| | Treating fistulas |
| Stress and energy | Promoting sound sleep |
| levels | Promoting lowered stress and tension |
| | Promoting higher energy level in elderly |
| Inflammation | Reducing general pain and swelling |
| | Treating spondylitis (inflammation of the vertebrae) |
| | Treating arthritis |
| | Treating gingivitis |
| | Treating toothaches |
| Reproduction | Treating oligospermia |
| | Promoting sperm motility |
| | Regulating ovulation |
| | Regulating menstruation |
| | Managing menstruation pain |
| | Treating irregular, especially prolonged (menorrhagia), |
| | menses |
| Dermatology | Treating pimples |
| | Treating sunburn and tan |
| | Treating lichenplanus |
| | Treating hyperpigmentation |
| | Treating eczema/dermatitis |
| | Treating psoriasis |
| | Preventing hair loss |
| | Promoting hair growth |
| Vision | Promoting vision recover after macular surgery |
| | Treating dry cornea |
| | Treating styes |

Utility of the compositions of this invention are not restricted to the embodiments listed in Table 2. Innumerable other embodiments for particular plant and non-plant biomass are possible as the invention is applicable to a wide variety of plant and non-plant biomass. The range of embodiments can be further increased by using appropriate mixtures of biomass. A few illustrative examples of such applications are given below:

reduce sciatica pain,

improve handgrip post paralytic stroke,

restore ‘Foot Drop’ condition post viral polyneuritis attack,

restore concentration and memory post brain haemorrage,

alleviate colitis,

reduce hyperpigmentation,

regulate sugar om blood and

alleviate headache.

B. Veterinary

The compositions of the invention may also be used to improve livestock productivity, treat animals for a variety of conditions, and improve animal health. Additionally, other benefits may be realized, such as an early onset of maturity, improvement in the shelf life of buffalo milk, an improvement in feed conversion efficiency (more production for less feed), and a decrease in mortality. Table 3 summarizes examples of embodiments with CAM plants in which the compositions of the invention may be used on animals. Utility of the compositions of this invention are not restricted to the embodiments listed in Table 3. Innumerable other embodiments for particular biomass are possible as the invention is applicable to a wide variety of plant and non-plant biomass.

TABLE 3
Veterinary embodiments of uses for the compositions of
the invention

| General embodiment | Specific embodiments |
|---|---|
| Growth | Increasing weight gain |
| | Increasing growth rate |
| | Decreasing mortality (overall |
| | improving health) |
| | Hastening maturity |
| Productivity | Increasing egg laying with less feed |
| | (egg-laying birds) |
| | Improving quality of milk (buffalo) |

C. Agricultural

The utility of the compositions, of the present invention extends to all areas of the Plant Kingdom. For example, the compositions of the invention have beneficial effects on vegetables, ornamentals, flowers, fruits, trees, cereals, legumes, herbs and medicinal plants. Table 4 summarizes examples of embodiments in which the compositions of the invention may be used in plants. The utility of the compositions of this invention are not restricted to the embodiments listed in Table 4. Innumerable other embodiments for particular biomass are possible as the invention is applicable to a wide variety of plants.

TABLE 4
Embodiments of uses for the compositions of the
invention

| General embodiment | Specific embodiments |
|---|---|
| Germination | Promoting vigorous rooting and |
| | shooting and germination vigour |
| Vegetative growth | Promoting branching |
| | Promoting growth (especially height) |
| Leaf production | Promoting increased chlorophyll levels |
| | Promoting larger leaves and more leaf |
| | area per plant |
| | Promoting higher carbohydrate content |
| | Promoting higher number of leaf active |
| | days (LAD) |
| | Extending leaf life |
| | Delaying senescence |
| Flowering | Promoting early onset |
| | Reducing flower drop |
| | Promoting larger bloom size |
| | Promoting uniform bloom size |
| | Increasing production |
| Fruit | Reducing fruit drop |
| | Promoting larger sized fruits |
| | Promoting fruit appearance (e.g. |
| | “shine”) |
| | Promoting production |
| Productivity | Promoting increased yields, whether |
| | fruit, flower, or vegetable |
| | Increasing primary metabolites (e.g., |
| | sugars, proteins, and oil content) |
| | Increasing secondary metabolites (e.g., |
| | anti-oxidants, aromatics, and medicinal |
| | substances) |
| Herbicide | Eliminating unwanted plants/grasses |
| | Controlling growth of plants/grasses |
| | Acting as a synergist with pre-emergent |
| | herbicides |
| Shelf life | Promoting shelf life of fruit and flowers |
| Pest defenses | Promoting higher levels of defense |
| | chemicals (e.g., polyphenols and |
| | alkaloids) |
| | Reducing damage by pests (e.g., white |
| | fly, aphid, jassid, fruit fly, fruit borer, |
| | mite, stem borer, millibug) |
| | Reducing incidence of viral attacks |
| | Reducing incidence of fungal damage |
| Environmental stress | Promoting frost resistance |
| | Promoting drought tolerance |
| | Increasing osmolyte levels (e.g., |
| | proline) |
| | Allowing co-existence of insects while |
| | decreasing insect damage |
| Qualitative | Decreasing thorny habits |
| | Promoting natural plant colors (e.g., |
| | ornamentals) and shiny leaves/fruit |

Other embodiments of the invention will be apparent to those of skill in the art.

### II. DEFINITIONS

Crassulacean Acid Metabolism (CAM)

“CAM” involves the use of both the C₃ and C₄ pathways of carbon fixation. However, unlike C₄ plants, CAM plants temporally separate, as opposed to spatially separate, the C₃ and C₄ cycles.

The C₃ cycle (Calvin cycle) takes place in the stroma of the chloroplasts, starts and ends with the five carbon sugar, ribulose 1,5-bisphosphate (RuBP). The Calvin cycle occurs in three stages. (1) Carbon dioxide enters the cycle and is enzymatically combined (fixed) to RuBP. The resultant six-carbon compound, an unstable enzyme-bound intermediate, is immediately hydrolyzed to generate two molecules of 3-phosphoglycerate or 3-phosphoglyceric acid (PGA). Each PGA molecule contains three carbon atoms. RuBP carboxylase/oxygenase (Rubisco) catalyzes this reaction. (2) In the second stage, 3-phosphoglycerate is reduced to glyceraldehydes 3-phosphate, or 3-phosphoglyceraldhyde (PGAL), requiring NADPH as the nucleotide cofactor for reduction. (3) In the third stage, five of the six molecules of clyceraldehyde 3-phosphate are used to regenerate three molecules of ribulose 1,5-bisphosphate. Many plants use only the C₃ cycle.

The C₄ cycle (Hatch-Slack pathway) involves a first step of fixing carbon dioxide to phosphoenolpyruvate (PEP) by the enzyme PEP carboxylase. PEP carboxylase uses the hydrated form of carbon dioxide, bicarbonate ion. Depending on the species, the resulting oxaloacetate is either reduced to malate or transaminated to aspartate through the addition of an amino group. The malate or aspartate then releases the carbon dioxide for use in the Calvin cycle. Plants that are C₄ spatially separate the different steps of carbon fixation: oxaloacetate and malate (or aspartate) are produced in the mesophyll cells, but then the malate (or aspartate) moves to bundle-sheath cells, where decarboxylation occurs and the Calvin cycle. Hence, C₄ plants spatially separate the C₃ and C₄ cycles. Kranz leaf anatomy clearly identifies most C₄ plants, wherein mesophyll cells are orderly arranged around a layer of large bundle-sheath cells, so that together, the two form concentric layers around the vascular bundle.

CAM plants are distinguished by their ability to fix carbon dioxide in the dark through the activity of PEP carboxylase in the cytosol. The initial carboxylation product is oxaloacetate, which is immediately reduced to malate. The malate is stored as malic acid in the vacuole. During the following light period, the malic acid is recovered from the vacuole, decarboxylated, and the carbon dioxide transferred to RuBP of the Calvin cycle within the same cells. Structurally, CAM plants have cells with large vacuoles (for aqueous storage of malic acid), and chloroplasts, where the carbon dioxide obtained from the malic acid can be transformed into carbohydrates.

CAM plants are largely dependent upon nighttime accumulation of carbon dioxide for their photosynthesis because their stomata are closed during the day to retard water loss. In general, CAM plants, while able to survive harsh environmental conditions, grow more slowly and if forced to compete with C₃ and C₄ species (in favorable environments), will compete poorly (Raven et al., 1999).

Examples of CAM plants include Crassula sp., Faucaria sp., Lithops sp. Rhodia sp., Cactaceae, Euphorbiaceae, Agave sp., Spanish moss, epiphytic bromeliads, pineapple, and vanilla orchids. Other examples are given in Table 5.

TABLE 5
Examples of CAM plants

| Family | Genera |
|---|---|
| Agavaceae | Agave, Yucca |
| Aizoaceae | Aptenia, Bergeranthus, Carpobrotus, |
| | Conophytum, Drossanthenum, |
| | Faucaria, Lithops, |
| | Mesembryanthemum, Tetragonia, |
| | Titanopsis, Trichodeadema |
| Asclepiadaceae | Caralluma, Hoya, Stapelia |
| Asteraceae | Aster, Kleinia, Notonia, Senecio |
| Bromeliaceae | Acanthostachys, Aechmia, |
| | Ananas, Araeocassus, Billbergia, |
| | Bromelia, Canistrum, Dyckia, |
| | Guzmania, Hoplophytum, |
| | Neoregelia, Nidularium, Orthophytum, |
| | Puya, Quesnelia, |
| | Tillandsia, |
| Cactaceae | Bergerocactus, Carnegiea, Cereus, |
| | Cephalocereus, Echinocereus, |
| | Echinopsis, Eulychnia, Ferocactus, |
| | Lobivia, Lophocereus, |
| | Machaerocereus, Mammillaria, |
| | Metacactus, Myrtillocactus, Neichilena, |
| | Nopalea, Notocactus, |
| | Opuntia, Pachycereus, Phyllocactus, |
| | Pilocopiapoe, Trichocereus, Zygocactus |
| Crassulaceae | Aeonium, Bryophyllum, Cotyledon, |
| | Crassula, Dudleya, Echeveria, |
| | Kalanchoe, Rochea, Sedum, |
| | Sempervivum, |
| Cucurbitaceae | Xerosicyos |
| Didiereaceae | Alluaudia, Didieria |
| Euphorbiaceae | Euphorbia, Monodenium, Synadenimum |
| Geraniaceae | Geranium, Pelargonium |
| Labiateae | Plectranthus |
| Lilliaceae | Aloe, Gasteria, Haworthia, |
| | Sanservieria |
| Orchidaceae | Arachnis, Aranda, Aranthera, |
| | Brassovora, Brassolaeliocattleya |
| | Bulbophyllum, Cattleya, Dendrobium, |
| | Encyclia, Epidendrum Laelia, Lanium, |
| | Oncidium, Phalaenopsis, Pleurothris, |
| | Schomburgkia, Sophrontis, Vanilla |
| Oxalidaceae | Oxalis |
| Piperaceae | Peperomia |
| Polypodiaceae | Drymoglossum, Pyrrosia |
| Portulacaceae | Portulacaria, Calandrinia |
| Vitaceae | Cissus |
| Welwitschiaceae | Wewitschia |

A more general complete list of plants can be considered to be in three categories: Angiosperm-Monocotyledon, Angiosperm-Dicotyledon, and Gymnosperm. Table 5A, 5B, and 5C below give just some illustrative examples of each category of plants.

TABLE 5A
Examples of Angiosperm - Monocotyledon Plants

| Order | Family | Genera |
|---|---|---|
| Graminales | Cyperaceae | Cyperus |
| Graminales | Gramineae | Triticum, Andropogon, |
| | | Avena, Cynodon |
| Liliales | Liliaceae | Allium, Asperagus |
| Schitaminales | Zingiberaceae | Curcuma |
| Schitaminales | Zingiberaceae | Zingiber |

TABLE 5B
Examples of Angiosperm - Dicotyledon Plants

| Order | Family | Genera |
|---|---|---|
| Campanulales | Cucurbitaceae | Momordica |
| Gentianales | Apocynaceae | Holarrhena, Rauwolfia, |
| | | Vinca |
| Gentianales | Asclepiadaceae | Hemidesmus, Gymnema |
| Gentianales | Gentianaceae | Swertia |
| Geraniales | Euphorbiaceae | Phyllanthus |
| Geraniales | Linaceae | Linum |
| Geraniales | Meliaceae | Azadiracta |
| Myrtales | Combretaceae | Terminalia |
| Myrtales | Myrtaceae | Eugenia |
| Piperales | Piperaceae | Piper |
| Primulales | Myrsinaceae | Embelia |
| Ranales | Menispermaceae | Tinospora |
| Rosales | Leguminosae | Glycine,Glycyrrhiza, |
| | | Mucuna, Phaseolus, |
| | | Pongamia, Trigonella |
| Santales | Santalaceae | Santalum |
| Tubiflorales | Labiatae | Ocimum |
| Tubiflorales | Pedaliaceae | Sesamum |
| Tubiflorales | Scrophulariaceae | Bacopa |
| Tubiflorales | Solanaceae | Withania |
| Umbellales | Umbelliferae | Carum, Cuminum |
| Urticales | Moraceae | Ficus |

TABLE 5C
Examples of Gymnosperm Plants

| Order | Family | Genera |
|---|---|---|
| Conifereae | | Pinus, Taxus |

An illustrative list of non-plant biomass includes mushroom, yeast, shrimp, shark fin, milk, organ meat and human hair.

Extract

An “extract” is most simply a preparation that is in a different form than its source. A cell extract may be as simple as mechanically lysed cells. Such preparations may be clarified by centrifugation or filtration to remove insoluble debris.

Extracts also comprise those preparations that involve the use of a solvent. Examples of solvents are water, a detergent, an oil or an organic compound. Extracts may be concentrated, removing most of the solvent and/or water; and may also be fractionated, using any method common to those of skill in the art (such as a second extraction, filtration, size fractionation by gel filtration or gradient centrifugation, etc.). In addition, extracts may also contain substances added to the mixture to preserve some components, such as the case with protease inhibitors to prolong protein life, or sodium azide to prevent microbial contamination.

When oils are used as a solvent, generally all oils that are appropriate for the application can be used. Examples include vegetable (corn, hempnut, mustard, rapeseed, safflower, sesame, sunflower, flaxseed, canola, soybean, olive, grape seed, walnut, peanut, anise, balm, bay, bergamont, borage, cajeput, castor (including Turkey Red (sulfated castor)), cedarwood, cinnamon, clove, coconut, cottonseed, evening primrose, jojoba bean, linseed (boiled or not), macadeamia, orignaum (thyme), Tea Tree, wheat germ, Neem (Azadirachta indica), Karanj (Pongamia glabra) and almond), animal (lard, fish, and butterfat from milk from various species), and those produced by the extraction industries (mineral, immersion and halocarbon). Purified oil components (lipids) may also be used. While all combinations of such oils and fats can be used, it is preferred to avoid those oils and oil combinations that polymerize or form gum during the extraction procedure that would interfere with extraction and fractionation.

Often, cell or tissue extracts are made to isolate a component from the intact source; for example, growth factors, surface proteins, nucleic acids, lipids, polysaccharides, etc., or even different cellular compartments, including Golgi vesicles, lysosomes, nuclei, mitochondria and chloroplasts may be extracted from cells.

A biomass extract may be made from any part of, or the entire, biomass. Plant parts include leaves, stems, flowers, inflorescences, shoots, cotyledons, etc. Non-plant biomass parts include milk, organ meat, blood, fruiting bodies, mycelium, hair, horn etc. The various parts may be dehydrated or used fresh. Often, the biomass parts are washed before processing. Fractionation with organic solvents may be desired to separate out organic-soluble components, such as chlorophyll.

The term “biomass extract” in the context of the current invention refers to any extract, made from a plant or non-plant biomass, that has at least one activity of the biomass extracts and compositions of the invention. A biomass extract activity is one that is evident throughout the description of the invention, including, but not limited to, Tables 1, 1A, 1B, 1C, 1D and Tables 2, 3, and 4.

Vigor

“Vigor” refers to the active, healthy, and well-balanced growth of plants or animals. For example, a “vigorous” plant has a fast growth rate coupled with non-etiolated habit and copious reproduction (seed or spore). A vigorous animal also has a fast growth rate coupled with adequate body strength.

Resistance

Resistance is of two types. A plant or animal may resist pests or opportunistic infections.

A plant or animal may also show resistance or tolerance to environmental stresses, such as heat, drought, frost, osmotic stresses and sudden fluctuations in the environment.

Production, Yield, and Feed Conversion

Production refers to the aspect of a plant or animal that is used for human purposes. For example, tomato plants are grown for their tomatoes; a tomato variety that produces many fruits per plant is more “productive” than one that produces few fruit but many leaves. On the other hand, a lettuce plant with many leaves is more productive than one that bolts early.

Yield refers the actual production per unit, unit referring to an organism such as a plant or animal.

Feed conversion is tied into production and yield. Feed conversion refers to the ability of an animal to efficiently produce per amount of feed.

Quality

“Quality” refers to subjective criteria that are used commercially to distinguish goods. For example, a high “quality” apple is one of a certain weight, certain, shape, free of blemishes, ripened and has a desired coloration, flavor, and texture. Qualitative assessments are well known to those of skill in the various arts.

Longevity

“Longevity” refers to criteria that define delaying of senescence such as a longer green life of a leaf or longer shelf life of flower or fruit.

### III. USING OF THE INVENTION

A. Extraction

The following describes the preparation of extract from Angiosperm-monocotyledon, Angiosperm-dicotyledon and Gymnosperm plants and from non-plant biomass. General extraction methods are defined both for fresh, soft, easily homogenizable biomass and biomass parts and for dry, hard, difficult to homogenize biomass and biomass parts.

A1: Fresh, Soft, Easy to Homogenize Biomass and Biomass Parts:

The following describes the preparation of an extract prepared from Kalanchoe pinnata (Lam.). Also see Examples. It will be apparent to one of skill in the art that many variations of the following procedure may yield extracts with similar activities. In general, any extract produced from Kalanchoe pinnata (Lam.) or any other whole plant or non-plant biomass or parts of plant or non-plant biomass with soft, easy to homogenize material that has at least one of the activities of the extract (see examples) is contemplated by the inventors.

However, any extract comprising regeneration and other specific activities can be similarly prepared from any CAM plant, such as Aloe vera or Cissue quadrangularis or from any other whole plant or parts of plant or with any other whole non-plant biomass or parts of non-plant biomass with fresh, soft, easy to homogenize material. Such extracts will have at least one activity of the compositions of the invention (see Examples). Such a procedure can be used for a wide variety of fresh, soft plant and non-plant materials including but not restricted to Allium cepa, Allium sativum, Bacopa monnieri, Fresh Ocimum santum, Phyllanthus indica (Fresh Fruit), and Zingiber officinalis (Fresh Rhizome), baker's yeast, fresh mushroom, ground prawn paste, milk, bacterial and fungal cell mass, and organ meat.

A mixture of small, medium and large leaves (1205 g) of Kalanchoe pinnata (Lam.) is plucked. After washing in water, the leaves are blended in a household blender, adding water to the mixture to allow the blades of the blender to contact the leaves such that the leaves are reduced to a pulp. Generally, water equal to half the weight of fresh leaves suffices. Next, 1205 g of sesame oil is heated to 100-120° C., but well below the smoke point of the oil in a stainless steel pot. The leaf mixture is charged to the pot and brought to boil. Thus, the ratio of oil to total homogenate for this soft material was 1:1. Stirring is provided to bring about an intimate contact of the plant material with oil and water and to minimize sticking on the walls of the vessel or charring. Boiling is continued until only fine bubbles or fine foam is formed, and bubbling nearly ceases. When the oil just starts to smoke, the extract is sufficiently free of water and is ready for filtration. The boiling time may be anywhere from 15 minutes to over 6 hours, depending on a variety of variables, including the starting material, volumes of water, etc. Heating is then stopped, the mixture cooled and filtered through cheesecloth to separate the first extract from the leafy residue. The leafy residue is mixed with sesame oil, 0 to 1 times the weight of the filtrate and filtered through a double layer of cheesecloth to obtain a second extract. The two extracts are combined, and additional sesame oil is added to adjust the total weight to 1205 g to obtain a final oil strength of 100, i.e., R-100. The composition is based on 100 g of leaf equivalent per 100 g of total final extract.

A2: Dry, Hard, Difficult to Homogenize Biomass and Biomass Parts:

The following describes the preparation of an extract prepared from Curcuma amada Roxb. Also see Examples. It will be apparent to one of skill in the art that many variations of the following procedure may yield extracts with similar activities. In general, any extract produced from Curcuma amada or any other whole plant or parts of plant or any other whole non-plant biomass or parts of non-plant biomass with dry, hard, difficult to homogenize material that has at least one of the activities of the extract (see examples) is contemplated by the inventors.

However, any extract comprising regeneration and other specific activities can be similarly prepared from any other whole plant or parts of plant with dry, hard, difficult to homogenize material including bark, rhizomes and seeds, bones, cartilage. Such extracts will have at least one activity of the compositions of the invention (see Examples). Such a procedure can be used for a variety of plants with hard, difficult to homogenize material including but not restricted to Azadirachta indica cake, Carum copticum, Cuminum cyminum, Curcuma longa, Ficus bengalensis, Embelia ribes, Eugenia jambolana, Hemidesmus indicus, Gymnema sylvestree, Glycine max, Glycyrrhiza glabra, Holarrhena antidysenterica, Momordica charantia, Phaeolus radiatus, Piper longum, Piper nigrum, Pongamia glabra cake, Rauwolfia serpentina, Santalum alba, Trigonella foecum-graecum, Terminalia chebula, Terminalia bellerica, Taxus baccata, Tinospora cordifolia, Mucuna pruriens, Sesamum indicum, Triticum vulgare, Swertia chirata, Cyperous rotundus, Vinca rosea, Withania somnifera.

Dry rhizomes (500 g) of Curcuma amada Roxb were obtained from the market. After washing in water, the rhizomes were pounded in a mortar to break them up into small pieces below 3 mm in diameter. These pieces were then soaked in 2 litres of water for 2 hours. All of the material, including the soak water, was blended by adding some more water to the mixture to allow the blades of the blender to contact the pieces such that the pieces are reduced to a very fine grind. Generally, for such dry, hard materials, water equal to 4 to 8 times the weight of dry starting material suffices. The homogenate was filtered to obtain 1180 g of wet residue. Next, 1840 g of sesame oil is heated to 100-120° C., but well below the smoke point of the oil in a stainless steel pot. With very hard biomass from seeds and dry bark/roots, about 4 kg oil per kg biomass is needed to keep the mass stirrable to the end. The wet residue is charged to the pot along with an additional 800 ml water and brought to boil. Stirring is provided to bring about an intimate contact of the plant material with oil and water and to minimize sticking on the walls of the vessel or charring. Boiling is continued until only fine bubbles or fine foam is formed, and bubbling nearly ceases. When the oil just starts to smoke, the extract is sufficiently free of water and is ready for filtration. The boiling time may be anywhere from 25 minutes to over 6 hours, depending on a variety of variables, including the starting material, volumes of water, etc. Heating is then stopped, the mixture cooled and filtered through cheesecloth to separate the first extract from the grind residue. The grind residue is mixed with sesame oil, 0 to 1 times the weight of the filtrate and filtered through a double layer of cheesecloth to obtain a second extract. The two extracts are combined, and additional sesame oil is added to adjust the total weight to 2000 g. The composition is based on 25 g of rhizome equivalent per 100 g of total final extract.

In both of above general procedures, several variables can be adjusted to achieve extracts of desired potency. For example, the starting material may consist of leaves, stems, shoots, seeds, bark, rhizome or the entire plant. Alternatively, juice that has been manually extracted, or expressed, from the plant or plant parts may also be used. Pounding can be done in a mortar or with any other device that can reduce the size of the starting material. Instead of a blender to homogenize the plant tissues, a mortar and pestle, or any other device or method that can destroy the integrity of the plant tissue, may be used. Boiling time may range from 25 minutes to 6 hours without losing efficacy. The oil may be any known in the art, including coconut, sesame, mineral and butterfat. It will be apparent to one of skill in the art to adjust other variables as appropriate, as, for example, when large-scale preparations are desired.

The compositions thus made may also be further diluted with oils to achieve extracts of different strengths that are suitable for various applications. Dilution serves important functions, including reducing any irritants and providing convenient doses. General penetrants and absorption aides such as Isopropyl myristate or MCT (Medium Chain Tryglyceride fraction) oil may be admixed.

B. Pharmaceutical Compositions

The compositions of the invention can be incorporated into pharmaceutical compositions. Such compositions typically comprise the plant extracts of the invention.

A “pharmaceutically acceptable carrier” includes any and all solvents, dispersion media, coatings, antibacterial and antifungal agents, isotonic and absorption delaying agents, and the like, compatible with pharmaceutical administration (Gennaro, 2000). Preferred examples of such carriers or diluents include, but are not limited to, water, saline, Finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

The pharmaceutical compositions for the administration of the active compounds, such as those of any of the plant extracts, may conveniently be presented in dosage unit form and may be prepared by any of the methods well known in the art of pharmacy. All methods include the step of bringing the active compound or plant extracts into association with the carrier that constitutes one or more accessory ingredients. In general, the pharmaceutical compositions are prepared by uniformly and intimately bringing the active compound into association with a liquid carrier or a finely divided solid carrier or both, and then, if necessary, shaping the product into the desired formulation. In the pharmaceutical composition the active compound is included in an amount sufficient to produce the desired effect upon the process or condition of diseases.

General Considerations

A pharmaceutical composition of the invention is formulated to be compatible with its intended route of administration, including intravenous, intradermal, subcutaneous, oral (e.g., inhalation), transdermal (i.e., topical), transmucosal and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Injectable Formulations

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, paraffin oils such as CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents, for example, parabens, chlorobutanol, phenol, ascorbic acid, and thimerosal, can contain microorganism contamination. Isotonic agents, for example, sugars, polyalcohols such as manitol, sorbitol, and sodium chloride can be included in the composition. Compositions that can delay absorption include agents such as aluminum monostearate and gelatin.

Sterile injectable solutions can be prepared by incorporating the active compound or composition, such as plant extracts, in the required amount in an appropriate solvent with one or a combination of ingredients as required, followed by sterilization. Generally, dispersions are prepared by incorporating the active compound into a sterile vehicle that contains a basic dispersion medium, and the other required ingredients as discussed. Sterile powders for the preparation of sterile injectable solutions, methods of preparation include vacuum drying and freeze-drying that yield a powder containing the active ingredient and any desired ingredient from a sterile solution.

Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

Compositions for Inhalation

For administration by inhalation, the compounds are delivered as an aerosol spray from a nebulizer or a pressurized container that contains a suitable propellant, e.g., a gas such as carbon dioxide.

Systemic Administration, Including Patches

Systemic administration can also be transmucosal or transdermal. For transmucosal or transdermal administration, penetrants that can permeate the target barrier(s) are selected. Transmucosal penetrants include: detergents, bile salts, and fusidic acid derivatives. Nasal sprays or suppositories can be used for transmucosal administration. For transdermal administration, the active compounds are formulated into ointments, salves, gels, or creams.

Creams are useful for a variety of external applications such as on chapped lips, cracked feet, heat rash, face cream, pimples, hand and body lotion to restore darkened skin after sun exposure, etc.

The compounds can also be prepared in the form of suppositories (e.g. with bases such as cocoa butter and other glycerides) or retention enemas for rectal delivery.

Carriers

In one embodiment, plant extracts are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such materials can be obtained commercially from ALZA Corporation (Mountain View, Calif.) and NOVA Pharmaceuticals, Inc. (Lake Elsinore, Calif.), or prepared by one of skill in the art. Liposomal suspensions can also be used as pharmaceutically acceptable carriers. These can be prepared according to methods known to those skilled in the art, such as in (Eppstein et al., U.S. Pat. No. 4,522,811, 1985).

Unit Dosage

Oral formulations or parenteral compositions in unit dosage form can be created to facilitate administration and dosage uniformity. Unit dosage form refers to physically discrete units suited as single dosages for the subject to be treated, containing a therapeutically effective quantity of active compound in association with the required pharmaceutical carrier. The specification for the unit dosage forms of the invention are dictated by, and directly dependent on, the unique characteristics of the active compound and the particular desired therapeutic effect, and the inherent limitations of compounding the active compound.

Dosage

The pharmaceutical composition and method of the present invention may further comprise other therapeutically active compounds, such as plant compositions, as noted herein that are usually applied in the treatment of wounds or other associated pathological conditions.

In the treatment of human conditions which require the compositions of the invention, an appropriate dosage level will generally be about 0.01 to 10 mg per kg patient body weight per day which can be administered in single or multiple doses. Preferably, the dosage level will be about 0.01 to about 10 mg/kg per day; more preferably about 0.01 to about 2.0 mg/kg per day, and most preferably 0.01 to about 0.4 mg/kg per day. A suitable dosage level may be about 0.001 to 10 mg/kg per day, about 0.01 to 2 mg/kg per day, or about 0.01 to 50 mg/kg per day. Within this range the dosage may be 0.05 to 0.5, 0.5 to 5 or 5 to 50 mg/kg per day.

For oral administration, the compositions are preferably provided in the form of tablets containing 0.1 to 10 milligrams of the active ingredient, particularly 0.1, 0.2, 0.5, 1.0, 1.5, 2.0, 2.5, 5.0, 7.5 and 10.0 milligrams of the active ingredient. The compounds may be administered 1 to 4 times per day, preferably once or twice per day.

For topical applications, the composition may have a dosage of about 0.001% to 50%, more preferably 0.01% to 10%, delivering 0.1 mg to 100 mg per 1 g application. The compositions may be administered 1 to 8 times per day, preferably once or twice per day. Alternatively, pads and other materials may be impregnated with such compositions and held in contact to the surface of the subject for chronic application.

The dosages outlined above are also suitable for veterinary applications. It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. In addition, the site of delivery will also impact dosage and frequency. Also understood, however, is that dosage for livestock may also differ. A skilled artisan will know how to adjust the unit dosage.

Kits for Pharmaceutical Compositions

The pharmaceutical compositions can be included in a kit, container, pack, or dispenser together with instructions for administration. When the invention is supplied as a kit, the different components of the composition may be packaged in separate containers and admixed immediately before use. Such packaging of the components separately may permit long-term storage without losing the active components' functions.

Containers or Vessels

The reagents included in the kits can be supplied in containers of any sort such that the life of the different components are preserved, and are not adsorbed or altered by the materials of the container. For example, sealed glass ampoules may contain lyophilized plant extracts or buffer that have been packaged under a neutral, non-reacting gas, such as nitrogen. Ampoules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampoules, and envelopes, that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, etc. Detailed instructions may not be physically associated with the kit; instead, a user may be directed to an internet web site specified by the manufacturer or distributor of the kit, or supplied as electronic mail.

Delivery Methods

Interstitial Delivery

The composition of the invention, such as plant extracts, may be delivered to the interstitial space of tissues of the animal body, including those of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. They may be conveniently delivered by injection into the tissues comprising these cells. They are preferably delivered to sites of injury, preferably to live cells and extracellular matrices directly adjacent to dead and dying tissue.

Any apparatus known to the skilled artisan in the medical arts may be used to deliver the compositions of the invention to the site of injury interstitially. These include, but are not limited to, syringes, stents and catheters.

Systemic Delivery

Any apparatus known to the skilled artisan in the medical arts may be used to deliver the compositions of the invention to the circulation system. These include, but are not limited to, syringes, stents and catheters. One convenient method is delivery via intravenous drip. Another approach would comprise implants, such as transdermal patches, that deliver the compositions of the invention over prolonged periods of time. Such implants may or may not be absorbed by the subject over time.

Surgical Delivery

The compositions of the invention may be delivered in a way that is appropriate for the surgery, including by bathing the area under surgery, implantable drug delivery systems, and matrices (absorbed by the body over time) impregnated with the compositions of the invention.

Superficial Delivery

Direct application of the compositions of the invention, such as plant extracts, may be used. For example, gauze impregnated with plant extracts or active components may be directly applied to the site of damage, and may be held in place, such as by a bandage or other wrapping. Alternatively, the compositions of the invention may be applied in salves, creams, or other pharmaceutical compositions known in the art meant for topical application.

C. Agricultural/Horticultural Compositions

Compositions Suitable for Application to Plants

In its simplest form, plant extract compositions that are suitable for agricultural compositions are simply diluted in water. Oil, powder and tablets of the CAM plant extract compositions may be used.

It is also possible to prepare combinations with other pesticidally active substances, fertilizers and/or growth regulators, for example in the form of a ready mix or a tank mix. These can be thought of us to be “carriers” for the plant extracts.

Wettable powders are preparations which are uniformly dispersible in water and which, besides the active substance, also comprise ionic and/or nonionic surfactants (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane 6,6′-disulfonate, sodium dibutylnaphthalene-sulfonate, or else sodium oleoylmethyltaurinate, in addition to a diluent or inert substance.

Emulsifiable concentrates are prepared by dissolving the plant extracts in an organic solvent, for example butanol, cyclohexanone, dimethylformamide, xylene, or else higher-boiling aromatics or hydrocarbons, or mixtures of the organic solvents with the addition of one or more ionic and/or nonionic surfactants (emulsifiers). Examples of substances which can be used as emulsifiers are: calcium alkylarylsulfonates such as calcium dodecylbenzenesulfonate, or nonionic emuslifiers such as fatty acid polyglycol esters, alkylaryl polyglycol ethers, fatty alcohol polyglycol ethers, propylene oxide/ethylene oxide condensates, alkyl polyethers, sorbitan esters, for example sorbitan fatty acid esters, or polyoxyethylene sorbitan esters, for example polyoxyethylene sorbitan fatty acid esters.

Dusts are obtained by grinding or mixing the plant extracts with finely distributed solid substances, for example, talc, natural clays such as kaolin, bentonite and pyrophyllite, or diatomaceous earth.

Suspension concentrates can be water-based or oil-based. They can be prepared, for example, by wet grinding using commercially available bead mills with or without an addition of surfactants, for example those that have already been mentioned above in the case of the other formulation types.

Emulsions, for example oil-in-water emulsions (EW), can be prepared, for example, by means of stirrers, colloid mills and/or static mixers using aqueous organic solvents in the presence or absence of surfactants, for example, in the case of the other formulation types.

Granules can be prepared either by spraying the active substance onto adsorptive, granulated inert material or by applying active substance concentrates to the surface of carriers such as sand, kaolinites or granulated inert material with the aid of binders, for example polyvinyl alcohol, sodium polyacrylate or else mineral oils. Suitable active substances can also be granulated in the manner that is conventional for the preparation of fertilizer granules, if desired as a mixture with fertilizers.

As a rule, water-dispersible granules are prepared by the customary processes such as spray drying, fluidized-bed granulation, disk granulation, mixing with high-speed mixers, and extrusion without solid inert material.

For the preparation of disk, fluidized-bed, extruder and spray granules see, for example (1973; 1979).

In wettable powders, the concentration of active substance is, for example, approximately 0.01% to 90% by weight, more preferably 0.01% to 0.5%, the remainder to 100% by weight being composed of customary formulation components. In the case of emulsifiable concentrates, the concentration, of active substance may be approximately 0.01% to 90%, preferably 0.01% to 0.5% by weight. Formulations in the form of dusts comprise 0.01% to 30% by weight of active substance, in most cases preferably 0.01% to 0.5% by weight of active substance; sprayable solutions comprise approximately 0.01% to 80%, preferably 0.01% to 0.5% by weight of active substance. In the case of water-dispersible granules, the active substance content depends partly on whether the active compound is in liquid or solid form and on which granulation auxiliaries, fibers and the like are being used. The active substance content of the water-dispersible granules is, for example, between 0.01% and 95% by weight, preferably between 0.01% and 0.5% by weight.

Alternatively, the rate of application of an active biomass extract is 2 to 100 g per hectare per year, applied in 4 to 20 sprays per year (or 2-5 sprays per season). More preferably, 3 to 30 g per hectare per year is applied. For herbicidal effects or for control of excessive growth mediated by biomass extracts, the extract concentration is increased to 25 to 500 g per hectare per year.

Besides, the above mentioned formulations of active substances may comprise, if appropriate, the adhesives, wetting agents, dispersants, emulsifiers, penetrants, preservatives, antifreeze agents, solvents, fillers, carriers, colorants, antifoams, evaporation inhibitors and pH and viscosity regulators which are customary in each case.

For use, the formulations that are in commercially available form are, if desired, diluted in the customary manner, for example using water in the case of wettable powders, emulsifiable concentrates, dispersions and water-dispersible granules. Preparations in the form of dusts, granules and sprayable solutions are usually not diluted any further with other inert substances prior to use. The necessary rate of application of the safeners varies with the external conditions such as temperature and humidity.

Components that can also be present in biomass extract compositions suitable for plant (agricultural) application, such as fertilizers or pesticides, include natural enzymes, growth hormones such as the gibberellins (gibberellic acid and gibberellin plant growth hormones), and control agents including pesticides such as acaracides and molluskicides, insecticides, fungicides, nematocides, and the like, depending of course on their compatibility with particular plant extracts. Examples of control agents that can be used in the compositions of the invention, depending on particular biomass extract compatibility, include inorganic compounds such as elementary sulfur and inorganic sulfur compounds, e.g., calcium polysulfide and sodium thiosulfate, which are effective fungicides, copper, zinc, and other metal in organics such as copper carbonate copper oxychloride, copper sulfate, and copper zinc sulfate. Organometallic compounds such as iron and tin compounds, e.g., triphenyl tin hydroxide exhibit both insecticidal and pesticidal activity. Saturated higher alkyl alcohols, either straight or branched chain, such as nonyl and decyl alcohol, can be present as insecticides. Aldehydes such as metaldehyde, are effective molluskicides, e.g., useful against snails. Carbonic acid derivatives, especially their mixed esters, are potent acaracides and fungicides; when sulfur is also present, e.g., mixed esters of thio- and di-thiocarbonic acids, activity is enhanced. 6-methylquinoxaline-2,3-dithiocyclocarbonate is an effective acaricide, fungicide, and insecticide. Carbamic acid derivatives such as aryl esters of N-methylcarbamnic acid, e.g., 1-naphthyl-N-methylcarbamate can also be used. Halogen substituted aliphatic monobasic and dibasic carboxylic acids are effective pesticides. Natural pyrethrins and their synthetic analogs are also effective pesticides. Salicylanilide is effective against leaf mold and tomato brown spot. Hetercyclic compounds possessing insecticidal and/or fungicidal activity can also be used. Halogen derivatives of benzene, such as paradichlorobenzene, are effective pesticides, often used against the sugarbeet weevil. Chitin-containing products are effective menatocides. Other compounds that can be used include aliphatic mercaptans having four or fewer carbon atoms, organic sulfides and thioacetals, nitro compounds such as chloropicrin dichloronitroethane, and chloronitropropane, copper and zinc inorganic and organic compounds, e.g., copper linoleate, copper naphthenate, etc., organophosphorous compounds of which there are well over a hundred, e.g., DDVP, tris-(2,4-diphenoxyethyl) phosphite, derivatives of mono- and dithiophosphoric acids, such as 0,0-diethyl S (2-ethylthio)-ethyl)phosphorodithioate, phosphoric acid derivatives, pyrophosphoric acid derivatives and phosphonic acid derivatives, quinones, sulfonic acid derivatives, thiocyanates and isocyanates, phytoalexins, insect killing soaps such as potassium fatty acid salts, and antiallatotropins such as 7-methoxy-2,2-dimethylchromene and the 6,7-dimethoxy analog. Diatomaceous earth can be used, which kills crawling insects.

These components can comprise from 0.001 to 10% or more by weight of the biomass extract compositions suitable for plant application. Also, alkalizing agents such as ground limestone and acidifying agents such as inorganic acids or acid salts can be added as needed or desired.

The biomass extract compositions suitable for plant application can be in solid form or in the form of an aqueous solution. Solid forms include powders and larger particulate forms, e.g., from 20 to 200 mesh. Where the biomass extract compositions are in solid form and biomass extracts are sensitive to light, air, or compounds in the composition, or to optional added components, the biomass extract compositions can be separately encapsulated in water soluble coatings, e.g., dyed or undyed gelatin spheres or capsules, or by micro-encapsulation to a free flowing powder using one or more of gelatin, polyvinyl alcohol, ethylcellulose, cellulose acetate phthalate, or styrene maleic anhydride. The separately encapsulated biomass extracts can then be mixed with the powder or larger particulates of another unencapsulated component and any optional components.

The presence of biomass extracts in the compositions suitable for agricultural use provides further enhancement of plant growth, and where applicable, crop production, i.e., by further enhancement is meant benefits in plant growth and crop production in addition to the benefits provided by the components other than plant extracts, and/or provides control of pest damage and resistance to stress. Biomass extracts also improve the effectiveness of beneficial microorganisms, and promote nutrient absorption and assimilation.

Particular biomass extracts may be added to herbicides, known in the art, to increase their effectiveness; as such, biomass extracts can also be used to control unwanted proliferation of weeds and other vegetative growth.

### EXAMPLES

The following examples are included to demonstrate preferred embodiments of the present invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing form the spirit and scope of the invention. For example, heating could be provided with steam or hot heat transfer mediaum to reduce degradation due to wall hot spots. Use of nitrogen or other inert gas blanketing may also be used to reduce degradation. The extraction process can also be carried out in a continuous manner by using the well established engineering methods of continuously introducing biomass, oil and water at one end and continuously withdrawing the liquid slurry at the other end of a suitably designed vessel.

### I. EXAMPLES OF BIOMASS EXTRACTION

The following examples illustrate biomass extractions; however, one of skill in the art will know how to vary the various variables to obtain extracts with the activity of the biomass extracts of the invention. Table A summarizes the designations for the various extracts used throughout the following examples.

In several of the following examples of biomass extraction, final extract weight is the same as the starting fresh biomass weight; hence all extracts are equivalent on fresh weight basis and have equivalent potencies. This, however, is not essential. As illustrated in Table A, any proportion of biomass weight to final extract can be used and suitabley designated as explained herein.

Using Kalanchoe pinnata

### Example 1

### General Extract Procedure

A mixture of small, medium and large leaves (1205 g) of Kalanchoe pinnata (Lam.) pers. was harvested. The leaves were washed with water, and blended in a household blender by addition of water, approximately half the weight of plant material, 600 g (or 600 ml). Separately, an equal weight of sesame oil was heated in a stainless steel pot. The blended mixture of leaves and water was charged to the pot and boiled for about 2 hours and 45 minutes until a very fine foam appeared. Heating was stopped, the mixture cooled and filtered through a once-folded (double) layer of cheesecloth to separate the first extract from leafy residue. The leafy residue was stirred with sesame oil equal to half the weight of the wet residue and filtered through a double layer of cheesecloth to obtain a second extract. The two extracts were combined and sesame oil was added to adjust the total weight of the final extract to 1205 g. This composition is based on 100 g of leaf equivalent per 100 g of total extract. The final extract was named R-100.

### Example 2

### Illustrating Smaller Starting Amounts of Materials and Shorter Boiling Times

Large, thick leaves (380 g) of Kalanchoe pinnata (Lam.) pers. were harvested and processed as in Example 1, except for a boiling time of 25 minutes. The final extract weight was adjusted with sesame oil to 380 g. This extract was named R-100.

### Example 3

### Illustrating Smaller Amounts of Starting Materials and Longer Boiling Times

Leaves of Kalanchoe pinnata (Lam) pers. (2000 g) were harvested. Procedure as outlined in Example 1 was followed, but with a boiling time of 6 hours. The final extract weight was adjusted with sesame oil to 2000 g. This extract was named R-100

### Example 4

### Illustrating the Use of Different Oils

Coconut Oil

Leaves (1380 g) of Kalanchoe pinnata (Lam.) pers. were harvested and washed with water. Leaves were blended as in Example 1. Separately, 2000 g of refined coconut oil was heated in a stainless steel pot. The total leaf homogenate was charged to the pot and brought to boil. After boiling for 4 hours and 45 minutes, until a very fine foam formed and started to subside, heating was stopped. The mixture was cooled and filtered through a double layer of cheesecloth to separate the first extract from leafy residue. The extract was adjusted to 1380 g by adding coconut oil. This composition, based on 100 g of leaf equivalent per 100 g of total final extract, was named R-100.

Safflower Oil; also Demonstrating Different Homogenization

Leaves (600 g) of Kalanchoe pinnata (Lam.) pers. were harvested and washed with water. Leaves were then ground in a pestle and mortar, adding water as in Example 1 to produce a leaf homogenate. Separately, 400 g of safflower oil was heated in a stainless steel pot. The total leaf homogenate was charged to the pot and boiled for 45 minutes until a fine foam formed and subsided. The mixture was cooled and filtered a double layer of cheesecloth to separate the first oil extract. The leafy residue was stirred with an equal amount by weight of safflower oil. The two extracts were combined, and additional safflower oil was added to a final total weight of 600 g. This composition, based on 100 g of leaf equivalent per 100 g of total final extract, was named R-100.

Using Aloe vera

### Example 5

### General Procedure for Aloe vera Extraction

800 g of Aloe vera, Linn. leaves were plucked and washed with water. Leaves were blended in a household blender by addition of 200 ml water as in Example 1 to produce a total homogenate. This homogenate was filtered over a cloth to separate the juice (J fraction) from the leafy residue concentrate (LR fraction). Separately, sesame oil was heated in two separate stainless steel pots.

The LR fraction was charged to one of the pots containing 800 g of sesame oil; 400 ml of water was then added, and the mixture was boiled for 45 minutes until a fine foam formed and subsided. The mixture was cooled and filtered through double layer of cheesecloth to separate the first extract. The residue was stirred with an equal weight of sesame oil. The two extracts were combined and additional sesame oil was added to a final total weight of 800 g. This composition from the LR fraction based on 100 g of initial leaf equivalent per 100 g of total final extract was named A-100 PLUS.

The J fraction was charged to a second stainless steel pot containing 800 g of sesame oil and the mixture was boiled for 1 hour and 20 minutes until the foam subsided. This extract was filtered through a double layer of cheesecloth and additional sesame oil added to a final weight of 800 g. This composition from the J fraction based on 100 g of initial total leaf equivalent per 100 g of final extract is designated as A-100 MINUS.

Using Cissus quadrangularis

### Example 6

### General Procedure for Cissus quadrangularis Extraction

The stem portion of Cissus quadrangularis (650 g) was harvested and washed with water. The stems were then blended in a household blender by addition of water as in Example 1 to produce a total homogenate. The homogenate was filtered over a double layer of cheesecloth to separate the juice (J fraction) from the fibrous stem residue concentrate (LR fraction). Separately, sesame oil was heated in two separate stainless steel pots.

The LR fraction was charged to one of the pots containing 650 g of sesame oil. 600 ml of water was then added, and the mixture boiled for 30 minutes until a fine foam formed and subsided. The mixture was cooled and filtered a double layer of cheesecloth to separate the extract. Additional sesame oil was added to a final total weight of 650 g. This composition from the LR fraction, based on 100 g of initial stem equivalent per 100 g of total final extract, was named C-100 PLUS.

The J fraction was charged to a second stainless steel pot containing 640 g of sesame oil, and the mixture was boiled for 1 hour and 20 minutes until the foam subsided. This extract was filtered, and additional sesame oil added to a final weight of 650 g. This composition from the J fraction, based on 100 g of initial total stem equivalent per 100 g of final extract, is designated as C-100 MINUS.

### Example 7

### Illustrating use of Animal Fat

Leaves of Kalanchoe pinnata were harvested and washed with water. Leaves were blended in a household blender by adding water as in Example 1 to produce a leaf homogenate. Separately, 2000 g of butterfat (ghee) was heated in a stainless steel pot. The total leaf homogenate was charged to the pot and brought to boil. Boiling was continued for 1 hour and 30 minutes to drive off the water. The mixture was cooled and filtered through a double layer of cheesecloth while warm to recover butterfat. The final composition, weighing 1600 g and based on 50 g of leaf equivalent per 100 g of total final extract was named R-50.

In all of these extract preparations, stirring was provided while boiling in oil or fat.

Extracts by using the general procedures outlined in Examples 1 to 7 can also be made from all other CAM plants. The same general procedures can also be used to make extracts from other soft, fresh plants and their parts. These can be any plants from the Angiosperm-monocotyledon, Angiosperm-dicotyledon and Gymnosperm plants including but not limited to fruit pulp of Phyllanthus emblica Linn., and rhizome of Zingiber officinalis Roscoe.

The following examples 1A to 4A, illustrate extractions of dry, hard biomass and their parts.

### Example 1A

### Illustrating Use of Bark for Extraction

The bark portion of Ficus bengalensis Linn. (500 g) was procured and washed with water. The bark pieces were then soaked in 1 litre water for 2 hours. The soaked pieces were then blended in a household blender by addition of another 500 ml water to produce a total homogenate. The homogenate was filtered over a double layer of cheesecloth to separate the juice (J fraction) from the fibrous bark residue concentrate (LR fraction). Separately, sesame oil was heated in two separate stainless steel pots.

The LR fraction was charged to one of the pots containing 1840 g of sesame oil. Then, 200 ml of water was added, and the mixture boiled with continuous stirring for 50 minutes until a fine foam formed and subsided. The mixture was cooled and filtered a double layer of cheesecloth to separate the extract. The oil wet residue was stirred with 940 g of sesame oil and filtered again to obtain the second oil extract. The two oil extracts were combined to have a final total extract of 1708 g. This composition from the LR fraction, based on 29.3 g of initial bark equivalent per 100 g of total final extract, was named FEBNG-29.3 PLUS.

The J fraction was charged to a second stainless steel pot containing 485 g of sesame oil, and the mixture was boiled for 35 minutes until the foam subsided. This extract was filtered, and additional sesame oil added to a final weight of 500 g. This composition from the J fraction, based on 100 g of initial bark equivalent per 100 g of final extract, is designated as FBENG-100 MINUS.

### Example 2A

### Illustrating Use of Dry Water Absorbing Seeds for Extraction

Seeds of Taxus baccata Linn. were procured (500 g) and washed with water. They were blended in a household blender by adding water. The homogenate becomes very viscous and thick. Hence, 3 litres of water was used to produce a homogenate. Separately, 1840 g of sesame oil was heated in a stainless steel pot. The total homogenate was charged to the pot and brought to boil. Boiling with continuous stirring was continued for 55 minutes to drive off the water. The mixture was cooled and filtered through a double layer of cheesecloth to separate the first oil extract from the oil wet cake. The oil wet cake was stirred with 920 g sesame oil, and filtered to obtain a second oil extract. The two extracts were combined to produce a final composition, weighing 1858 g and based on 27 g of seed equivalent per 100 g of total final extract was named TABA-27.

### Example 3A

### Illustrating Use of Dry, Hard Water Absorbing Tuberous Roots for Extraction

Dry, tuberous roots of Cyperous rotundus Linn. (500 g) were procured and washed with water. These roots do not easily get pulverized in a mortar and pestle. They were soaked in 2 litres of water for 4 hours. The soaked material was blended in a household blender by using an additional 1.2 litres of water to produce a homogenate. The homogenate was filtered over a double layer of cheesecloth to separate the juice (J fraction) from the fibrous residue concentrate (LR fraction). Separately, sesame oil was heated in two separate stainless steel pots.

The LR fraction (947 g water wet residue) was charged to one of the pots containing 1840 g of sesame oil. Then, 400 ml of water was added and the mixture boiled with continuous stirring for 45 minutes until a fine foam formed and subsided. The mixture was cooled and filtered a double layer of cheesecloth to separate the first oil extract from oil wet residue. The oil wet residue was stirred with 464 g of sesame oil and filtered again to obtain the second extract. The two oil extracts were combined to have a final total extract of 1773 g. This composition from the LR fraction, based on 28.2 g of initial tuberous root equivalent per 100 g of total final extract, was named CROT-28.2 PLUS.

The J fraction was charged to a second stainless steel pot containing 920 g of sesame oil, and the mixture was boiled for 50 minutes until the foam subsided. This extract was filtered to obtain 798 g of extract. This composition from the J fraction, based on 62.7 g of initial tuberous root equivalent per 100 g of final extract, is designated as CROT-62.7 MINUS.

### Example 4A

### Illustrating Use of Dry, Hard Seeds for Extraction

Seeds of Mucuna pruriens Bak were procured (500 g) and washed with water. The seeds were then pounded in a mortar and pestle to produce fines and pieces smaller than 5 mm. The larger pieces from the pounded mass were then soaked in 400 ml water for 4 hours. The soaked mass was blended in a household blender you produce a very thick paste. Separately, 1840 g of sesame oil was heated in a stainless steel pot. The thick paste and fines were charged to the pot along with an additional 800 ml water and brought to boil. Boiling with continuous stirring was continued for 55 minutes to drive off the water. The mixture was cooled and filtered through a double layer of cheesecloth to separate the first oil extract from the oil wet cake. The oil wet cake was stirred with 467 g sesame oil, and filtered to obtain a second oil extract. The two extracts were combined to produce a final composition, weighing 1843 g and based on 27.1 g of seed equivalent per 100 g of total final extract was named MPRU-27.1.

Extracts by using the general procedures outlined in Examples 1A to 4A can also be made from all other dry, hard plants and plant parts. These can be any plants from Angiosperm-monocotyledon, Angiosperm-dicotyledon and Gymnosperm including but not restricted to Azadirachta indica cake, Ficus bengalensis, Embelia ribes, Glycyrrhiza glabra, Holarrhena antidysenterica, Pongamia glabra cake, Trigonella foecum-graecum, Terminalia chebula, Terminalia bellerica, Taxus baccata, Mucuna pruriens, Sesamum indicum, Triticum vulgare, Swertia chirata, Cyperous rotundus.

Table A illustrates the method of designationg extracts from each plant. Each plant extract is designated by the following:

A number of alphabets which indicate the plant name, e.g. C stands for Cissus quadrangularis, CROT stands for Cyperous rotundus etc.

A further set of alphabets which may designate the particular part.

A number from 0 to 100 which designates the potency of the extract. Potency is designated on the basis of starting biomass material per 100 g of final extract.

The words, PLUS or MINUS which indicate use of LR and J fraction, respectively.

Thus, as illustrative examples,

TBEL-R-35 means an extract of the seed outer rind of Terminalia bellerica prepared such that 35 g of rind ends up as 100 g of final extract.

PE-100 PLUS means an extract of the LR fraction of the fruit of Phyllanthus emblica prepared such that 100 g of fruit ends up as 100 g of final extract.

TABLE A
Designations of the various extracts as illustrative
examples

| | Total | | |
|---|---|---|---|
| Plant source | homogenate | LR fraction | J fraction |
| Kalanchoe | R-100 | R-100 PLUS | R-100 MINUS |
| pinnata | | | |
| LEAF | | | |
| Kalanchoe | RS-10 | RS-10 PLUS | RS-10 MINUS |
| pinnata | | | |
| LEAF STEM | | | |
| Kalanchoe | KPMS-100 | KPMS-100 PLUS | KPMS-100 |
| pinnata | | | MINUS |
| MAIN STEM | | | |
| Aloe vera | A-100 | A-100 PLUS | A-100 MINUS |
| LEAF | | | |
| Cissus | C-100 | C-100 PLUS | C-100 MINUS |
| quadrangularis | | | |
| STEM | | | |
| Cyperous | CROT-25 | CROT-25 PLUS | CROT-100 |
| rotundus ROOT | | | MINUS |
| Triticum vulgare | TVUL-25 | TVUL-25 PLUS | TVUL-100 |
| SEED | | | MINUS |
| Allium cepa | ACEP-76 | ACEP-76 PLUS | ACEP-76 MINUS |
| BULB | | | |
| Allium sativum | ASAT-100 | ASAT-100 PLUS | ASAT-100 |
| BULB | | | MINUS |
| Asperagus | ASRA-50 | ASRA-50 PLUS | ASRA-50 |
| racemosus - | | | MINUS |
| ROOT | | | |
| Curcuma amada | CAMA-25 | CAMA-25 PLUS | CAMA-100 |
| RHIZOME | | | MINUS |
| Curcuma longa | CLNG-I-34 | CLNG-I-34 | CLNG-I-34 |
| | | PLUS | |
| RHIZOME | CLNG-II-28 | CLNG-II-28 | MINUS |
| | | PLUS | CLNG-II-28 |
| | | | MINUS |
| Zingiber officinale | ZOFF-50 | ZOFF-50 PLUS | ZOFF-100 |
| RHIZOME | | | MINUS |
| Momordica | MOCH-35 | MOCH-35 PLUS | MOCH-35 |
| charantia | | | MINUS |
| FRUIT | | | |
| Holarrhena | HA-35 | HA-35 PLUS | HA-100 MINUS |
| antidysenterica | | | |
| SEED | | | |
| Rauwolfia | RSER-75 | RSER-75 PLUS | RSER-75 MINUS |
| serpentina | | | |
| ROOT | | | |
| Vinca rosea | VR-37 | VR-37 PLUS | VR-37 MINUS |
| ROOT | | | |
| Gymnema | GYSY-26.4 | GYSY-25 PLUS | GYSY-25 |
| sylvestree | | | MINUS |
| LEAF | | | |
| Swertia chirata | SCHIR-35 | SCHIR-35 PLUS | SCHIR-100 |
| TOTAL PLANT | | | MINUS |
| Phyllanthus | PE-100 | PE-100 PLUS | PE-100 MINUS |
| emblica FRUIT | | | |
| Linum | LUST-35 | LUST-35 PLUS | LUST-100 |
| usitatisimum | | | MINUS |
| SEEDS | | | |
| Azadiricta indica | AZIN-C-35 | AZIN-C-35 | AZIN-C-100 |
| | | PLUS | |
| CAKE | N-50 | N-50 PLUS | MINUS |
| LEAF | | | N-50 MINUS |
| Terminalia | TCHEB-35 | TCHEB-35 PLUS | TCHEB-100 |
| chebula FRUIT | | | MINUS |
| Terminalia | TBEL-35 | TBEL-35-PLUS | TBEL-100 |
| bellerica | TBEL(R)-35 | TBEL(R)-35 | MINUS |
| T. bellerica RIND | TBEL(P)-35 | PLUS | TBEL(R)-35 |
| T. bellerica PULP | | TBEL(P)-35 | MINUS |
| | | PLUS | TBEL(P)-35 |
| | | | MINUS |
| Eugenia | EJAM-28 | EJAM-28 PLUS | EJAM-28 |
| jambolana | | | MINUS |
| SEED | | | |
| Piper longum | PILO-29 | PILO-29 PLUS | PILO-29 MINUS |
| FRUIT | | | |
| Piper nigrum | PINI-32 | PINI-32 PLUS | PINI-32 MINUS |
| FRUIT | | | |
| Embelia ribes | ER-35 | ER-35 PLUS | ET-100 MINUS |
| FRUIT | | | |
| Tinospora | TICO-34 | TICO-34 PLUS | TICO-34 MINUS |
| cordifolia | | | |
| STEM | | | |
| Glycine max | GLMX-54 | GLMX-54 PLUS | GLMX-54 |
| SEED | | | MINUS |
| Glycyrrhiza | GGLAB-35 | GGLAB-35 | GGLAB-35 |
| glabra ROOT | | PLUS | MINUS |
| Mucuna pruriens | MPRU-35 | MPRU-35 PLUS | MPRU-100 |
| SEED | | | MINUS |
| Phaseolus | PRAD-28 | PRAD-28 PLUS | PRAD-29 |
| radiatus | | | MINUS |
| SEED | | | |
| Pongamia glabra | PGL-C-35 | PGL-C-35 PLUS | PGL-C-100 |
| CAKE | | | MINUS |
| Trigonella | TRIGF-35 | TRIGF-35 PLUS | TRIG-35 MINUS |
| foecum-graecum | | | |
| SEED | | | |
| Santalum alba | SAAL-65 | SAAL-65 PLUS | SAAL-65 |
| HEARTWOOD | | | MINUS |
| Ocimum sanctum | OSAT-36 | OSAT-36 PLUS | OSAT-36 |
| TOTAL PLANT | | | MINUS |
| Sesamum | SIND-35 | SIND-35 PLUS | SIND-100 |
| indicaum SEEDS | | | MINUS |
| Bacopa monnieri | BAMO-37 | BAMO-37 PLUS | BAMO-37 |
| LEAF | | | MINUS |
| Withania somifera | WISO-43 | WISO-43 PLUS | WISO-43 |
| STEM | | | MINUS |
| Carum copticum | CACO-42 | CACO-42 PLUS | CACO-42 |
| SEED | | | MINUS |
| Cuminum | CUCY-29 | CUCY-29 PLUS | CUCY-29 |
| cyminum | | | MINUS |
| SEED | | | |
| Ficus bengalensis | FBENG-35 | FBENG-35 PLUS | FBENG-100 |
| BARK | | | MINUS |
| Baker's Yeast - | YBD-45 | YBD-45 PLUS | YBD-45 MINUS |
| granulated | | | |
| Button Mushroom | MUSH-A-67 | MUSH-A-57 | MUSH-A- |
| Agaricus bisporus | | PLUS | 57 MINUS |
| Prawn - ground - | PRWN-54 | PRWN-54 PLUS | PRWN-54 |
| whole | | | MINUS |

### II. USEFUL PREPARATIONS OF BIOMASS EXTRACTS

The following examples illustrate compositions of plant extracts from CAM plants for a variety of applications; however, they are not meant to be limiting. The same principles and methods can be used to prepare useful compositions from any Angiosperm-monocotyledon, Angiosperm-dicotyledon and Gymnosperm plant and from any non-plant biomass. It will be apparent to one of skill in the art how to modify the various preparations for specific applications.

### Example 8

### Dilution of Extracts

Extracts made by the general procedure described in Examples 1-7 were further diluted with sesame oil to make extracts of different strengths. For example, extract made by mixing one part of R-100 with 9 parts of sesame oil was named R-10. Similarly, one part of R-100 with 19 parts of sesame oil, and one part of R-100 with 49 parts of sesame oil were designated R-5 and R-2, respectively. Thus, one can make diluted oil extracts of any desired strength such as R-1, R-2, R-5, R-10, etc. as convenient and stable dosage forms.

R-5 can be used effectively on simple burns and open wounds. However, applications in more sensitive skin application require a much lower concentration such as R-1 or one may have to go even lower and use skin lotions as described in subsequent examples. R-5 to R-1 range can also be conveniently given in the form of one or more drops as such or in drinking water.

In veterinary applications, R-10 to R-1 constitute more convenient dosage forms for addition to feed or drinking water.

In crop health applications, too, R-5 to R-1 can be conveniently added to the root zone or added to the water for spraying purposes.

Extracts from other plants and from J or LR fractions were also diluted to different strengths and designated by the degree of dilution and the fraction used, such as A-5, A-5 PLUS, A-5 MINUS, C-2, C-2 PLUS, C-2 MINUS.

### Example 9

### Admixing CAM Plant Extracts with Pharmaceutical Carriers

R-100 extract made by the general procedure described in Example 1 is further diluted by mixing it with carriers such as sucrose, lactose, or other sugars. Alternatively, R-100 can be absorbed on porous supports such as precipitated calcium carbonate, talc, precipitated silica, etc. Powders made by mixing one part of R-10 with 9 parts of solids were named R-10(P). Similarly, one part of R-100 with 19 parts of solids and one part of R-100 with 49 parts of solids are named R-5(P) and R-2(P), respectively. Thus, one can make these diluted powders of any desired strength such as R-1(P), R-2(P), R-5(P), R-10(P), etc. as convenient and stable dosage forms.

R-1(P) and lower strengths up to R-0.1(P) can be used effectively in talcum powder formulations, dental preparations or other powder formulations for dusting applications on skin.

For internal veterinary applications, the quantities required are so small that R-10(P) to R-1(P) dilutions can be conveniently used directly for feed mixing in the conventional mixing equipment.

In crop health applications, too, R-5(P) to R-1(P) can be conveniently added to the root zone or added to the water for spraying purposes.

### Example 10

### Tablet Compounding

Tablets of different strength can be made as convenient and stable dosage forms for a variety of applications. A few typical recipes for tablet making are given in the examples below. However, a variety of other excipients may also be used, with or without other adjuvants, for tablet making.

Tablets of any desired strength of mg of R-100 can be made. In the following examples, they were named accordingly as T-1, T-2, T-5, T-10, etc. Tablet making also allows convenient dose metering. Thus, for internal human consumption, T-1 represents one of the convenient forms as the typical human dose is one T-1 per day for an adult.

In veterinary applications also, for the same reason, T-10 to T-1 constitute a more convenient dosage form for addition to feed.

In crop health applications, one T-5 in 5 liters water is a very convenient spray dose per 100 m² field area. Also, one T-5 at the root zone is the typical dose for a new tree sapling. Larger trees need root zone application dose in multiples of T-5.

T-1 and multiples can be conveniently used at the root zone for small potted plants and one T-1 per liter is a useful spray solution.

150 mg Tablets

100 g of R-100 was mixed with 900 g sucrose and homogenized in a pestle and mortar to make R-10(P). R-10(P) was then mixed with other components, processing aids and binding agents in the proportion of 1 kg R-10(P), 0.3 kg gum Arabic, 0.3 kg gelatine, 1.15 kg magnesium stearate, 0.3 kg talc, and 11.95 kg sucrose (for a total of 15 kg) and made into tablets weighing 150 mg in a tablet making machine.

250 mg Tablets

100 g of R-100 is mixed with 900 g sucrose and homogenized in a pestle and mortar to make R-10(P). R-10(P) was then mixed with other components, processing aids and binding agents in the proportion of 1.0 kg R-10(P), 0.1 kg gum Arabic, 0.1 kg gelatine, 0.35 kg magnesium stearate, 0.1 kg talc, and 3.35 kg potassium chloride (for a total of 5.0 kg) and made into tablets weighing 250 mg in a standard tablet making machine.

### Example 11

### Cream Formulations

The notations to describe the strength of oil, powder and tablet in terms of leaf equivalent are also applicable to creams.

All Purpose Cream

Two mixtures were prepared. Mixture A consisted of 3% stearic acid, 40% mineral oil (70 viscosity), 7% lanolin, 10% petrolatum (USP), 2% cetyl alcohol, 2% microcrystalline wax and 0.10 R-100. Mixture B consisted of 5% MgAl silicate (as a 5% dispersion), 1.78% triethanoloamine, and 29.22% water.

Mixtures A and B were heated separately to 70° C. Mixture B was then added to mixture A and stirred continuously. Then, the mixtures was cooled to 35-40° C. A negligeable amount of fragrance (such as lavendar) and preservatives (such as parabens) was then added, and the mixture mixed until dispersion was complete. Evaporation loss was replaced with water.

Hand and Body Lotion (Oil in Water Emulsion)

Two mixtures were prepared. Mixture A consisted of 2.5% stearic acid, 2% mineral oil (70 viscosity), 1% glyceryl monostearate, 2% isopropyl palmitate, 1% petrolatum (USP), 1% cetyl alcohol, 0.25% PEG 40 stearate wax and 0.10% R-100. Mixture B consisted of 7% Carbomer 934 (as a 2% dispersion), 5% glycerine, 1% triethanolamine (as 99% solution), and 77% deionized water.

Mixtures A and B were heated separately to 70° C. Mixture B was then added to mixture A and then agitated. Then, the mixtures were mixed to 35° C. A negligible amount of lavender and parabens were added for fragrance and stability, respectively, and the mixture mixed until dispersion was complete.

### III. HUMAN APPLICATIONS

Methods

Oil, powder, or tablets made as described in the preceding examples were used in all cases for internal human administration. Oils, creams or lotions were made as described in Examples 8-11 and were used in all topical applications. These formulations were based on oil extracts made according to Examples 1-7 and 1A-4A. Multi-herb combinations were made by: 1) mixing the oil extracts made as per the preceding examples and 2) further adding medium chain fatty acid tryglycerides and sesame oil as per the final stength desired. The examples given below illustrate the use of individual extract compositions as also the use of multi-herb compositions.

Results

### Example 12

### Use of CAM Plant Extracts

In all of these reported cases in Table 6, the dose administered internally was 1 to 2 drops of R-5 oil or between 1 to 4 tablets per day each containing 1 mg of leaf equivalent per day, i.e. 1 to 4 mg of R-100 per day, and in a vast majority of cases, 1 tablet per day or 1 drop of R-5 oil per day. The topical application (1 to 2 times a day) using oil or body lotion was also less than or equal to 5 mg of R-100 or leaf equivalent per day.

TABLE 6
Results of human applications of CAM plant oil
extracts

| Indication | % Positive Respondents | Number |
|---|---|---|
| Respiratory disorders (prevent/cure) | | 78 |
| Cough/cold/congestion | 70 | 66 |
| Asthma: Allergic or Stress | 80 | 12 |
| Induced | | |
| Stress/energy level | 90 | 26 |
| Sound sleep | | 3 |
| Lowering of stress/tension | | 12 |
| Higher energy level in elderly | | 9 |
| Improved circulation in feet | | 2 |
| Digestive system | 80 | 13 |
| Reduced acidity | | 4 |
| Reduced stomach upsets | | 7 |
| Improved appetite | | 2 |
| Healthy growth | 80 | 28 |
| Gain in height/weight | | 28 |
| Blood lipid levels | 80 | 10 |
| Lowering of LDL cholesterol | | 8 |
| Lowering of triglycerides | | 8 |
| Healing response | 90 | 52 |
| General bruises/cuts | | 4 |
| Diabetic ulcers | | 13 |
| Leprotic ulcers | | 7 |
| Varicose ulcers | | 1 |
| Bedsores | | 6 |
| Burns | | 12 |
| Piles | | 2 |
| Fistula | | 1 |
| Anti-inflammatory | | 35 |
| General pain/swelling | 90 | 12 |
| Spondulitis | 50 | 6 |
| Arthritis | 50 | 8 |
| Gingivitis | 80 | 5 |
| Toothache | 80 | 4 |
| Reproductive system | | 11 |
| Oligospermia/sperm motility | | 3 |
| Ovulation | | 2 |
| Menstual discomfort/pain | | 4 |
| Menorrhagia | | 2 |
| Skin/hair | | 26 |
| (inflammation/repair/recovery) | | |
| Pimples | | 4 |
| Sunburn | 100 | 2 |
| Lichenplanus | | 1 |
| Eczema/dermatitis | | 5 |
| Psoriasis | | 6 |
| Hair loss prevention | 100 | 5 |
| Hair vigorous growth | 100 | 3 |
| Eyes | | 5 |
| Vision recovery post-macular surgery | | 3 |
| Dry cornea | | 1 |
| Sty | | 1 |

As an example, none of the persons whose LDL and triglyceride levels have responded to the use of these compositions made any special changes in their diet or their lifestyle during the trial period (Table 6). At a dose level of 2 to 4 T-1 tablets/day, or at a dose level of 2 to 4 mg leaf equivalent/day, levels of serum LDL and/or serum triglyceride were reduced considerably within a few months (Table 7).

TABLE 7
Case Summary: Lipid-lowering effect of the
compositions of the invention
CASE SUMMARY - LIPID PROFILE

| | | mg/dl | mg/dl | mg/dl | mg/dl | T-1/day |
|---|---|---|---|---|---|---|
| CASE NO./Dates | Sex/Age(yrs.) | Cholesterol | HDL | LDL | Trigly | Dose/Remarks |
| 1. | | | | | | |
| 26/05/93 | Male(50) | 170 | 58 | 96 | 80 | Base Data |
| 09/12/93 | | 168 | 55 | 98 | 77 | Base Data |
| 16/11/94 | | 138 | 62 | 67 | 49 | 2 taken for two |
| | | | | | | months bef. test |
| 2. | | | | | | |
| 10/07/95 | Male(40) | 219 | 44 | | 179 | 2 dose start |
| 23/09/95 | | 177 | 40 | 125 | 123 | 2 |
| 27/02/96 | | 185 | 43 | 116 | 127 | 1-2 |
| 10/08/96 | | 185 | 41 | 118 | 127 | |
| 3. | | | | | | |
| 23/09/95 | Fem(12) | 204 | 48 | 138 | 90 | 1 dose start |
| 19/06/97 | | 151 | 46 | 90 | 78 | Taken a total |
| | | | | | | 150 tabs on/off |
| 4. | | | | | | |
| 09/10/95 | Male(45) | 268 | 37 | 176 | 273 | 1 dose start |
| 30/1/96 | | 247 | 38 | 151 | 247 | |
| 5. | | | | | | |
| 21/09/96 | Male(60) | 230 | 47 | 120 | 313 | 1 started from |
| 26/02/97 | | 225 | 41 | 130 | 269 | 4^{th} may 1996 |
| 26/03/98 | | 228 | 53 | | 194 | Intermittently |
| 6. | | | | | | |
| 27/08/93 | Male(40) | 258 | 53 | 151 | 270 | 1 dose started |
| 01/03/97 | | 245 | 40 | 169 | 180 | taken a total 150 |
| | | | | | | tabs from 5/5/96 |
| 7.. | | | | | | |
| 15/11/97 | Male(35) | 240 | | | | 4 dose started |
| 06/02/98 | | 148 | | | | from 8/12/97 |
| 8. | | | | | | |
| 06/10/98 | Fem(50) | 220 | 60 | 137 | 115 | 2 dose started |
| 18/12/98 | | 170 | 60 | 89 | 105 | |
| 9. | | | | | | |
| 06/10/98 | Fem(24) | 245 | 65 | 166 | 70 | 2 dose started |
| 18/12/98 | | 195 | 70 | 115 | 50 | |
| 10. | | | | | | |
| 06/11/98 | Male(80) | 310 | | | | |
| 01/03/99 | | 174 | | | | 2 Taken for two |
| | | | | | | months before test |

Notes on Table 6:

Stress Resistance/Energy Level

As reported in Table 6, a number of persons experienced effects such as reduction in stress-induced asthma, increase in sound sleep, etc. These effects were obtained by a daily intake of one T-1 tablet for 2 to 4 weeks. A number of elderly persons (over 70 years) found an enhanced sense of well-being, higher energy levels, a general reduction in stomach upsets and a reduction in seasonal coughs and colds by daily intake of one T-1 tablet.

Healthy Growth—Height/Weight Gain in Children

As reported in Table 6, several children who were otherwise had a lack of appetite, routine headaches, low hemoglobin, fatigue, etc, responded positively to the intake of one T-1 tablet per day and started registering healthy height and weight gain with alleviation of these other symptoms. Significant improvement was noted after treatment for more than one month.

Healing Response

Diabetic, leprotic, varicose ulcers; bedsores and burns were treated successfully with the daily application of one to four drops of R-5 oil to the ulcer (1 to 6 mg of leaf equivalent). In case of deep leprotic ulcers, the whiteness near the top of the wound changes to a healthy pink color by topical application of R-5 oil, indicating local promotion of angiogenesis. Faster growth of a tougher collagen layer in healing of diabetic ulcer was also observed. Infected diabetic wounds were cleared by topical application.

Skin Inflammation and Repair

In cases of eczema and psoriasis, topical application gave relief from the inflammatory process. In case of lichenplanus, the lesions healed readily. Tan caused by sunburn on exposed arms was eliminated by topical application of a 0.1% R-100 body lotion. Inflammation of pimples reduced by facial application of a 0.1% R-100 body lotion. In a few cases, inflammation and wound due to piles were also controlled.

Hair health

As reported in Table 6, several persons losing hair on account of ill health or poor hair health, the loss was arrested and vigorous re-growth of hair started by the intake of one T-1 tablet per day. Observable effects were noted within two weeks of starting the intake. Application of hair oil containing 0.1% by weight of R-100 to the scalp produced the same effect.

Eyes

In a few cases, rapid vision recovery post-macular surgery by oral intake of T-1 tablets was noted. Recovery from sty infection and dry corneas were achieved by topical application.

Reproductive System

A few cases of oligospermia/sperm motility were corrected by oral intake of T-1 tablets for three months.

### Example 12A

### Additional Cases of Use of CAM and Non-CAM Plant Extracts

The examples below illustrate that the compositions of the present invention produce preventive and curative therapeutic effects in many different human embodiments.

The cases described below also illustrate how a variety of compositions using combinations of biomass extracts can be used effectively in a variety of human embodiments. In a majority of cases these effects are expressions of effects expected fror the same biomass as described in the available literature. However, there are also some novel therapeutic effects which are not described in the literature. All combination extracts also contain 20 gm of MCT Oil (a 70:30 mixture of capryllic/capric tryglycerides obtained from Subhash Chemical lndustriesd, Pune)/100 gm.

The examples also illustrate the key aspect of this invention in that these effects are produced at an extremely small dose in terms of mg per day of the starting biomass.

In all of these reported cases below the dose administered internally was 1 to 10 drops of apprpriate final oil extract composition containing 2-50 mg of total plant equivalent per day. In a majority of cases, 1-2 drop of appropriate oil extract per day was adequate. The topical application (1 to 2 times a day) using oil or body lotion was also less than or equal to 10 mg of plant equivalent per day. The designations of extracts are as defined hereinabove and also as defined herein.

Digestive System/General Health

A mixture of AMAVAT-20 oil was prepared. This oil is based on the extract of 20 gms of total herbal material/100 gm of oilextract. The herbal mixture contains equal parts of: 1) an equal part mixture of Zingiber officinale, Carum copticum, Cuminum cyminum, Piper longum, 2) a mixture of Terminalia Bellerica (1 part), Terminalia chebula (3 parts), Phyllanthus emblica (6 parts), Aloe indica (10 parts), Glycyrrhiza glabra (10 parts), 3) an equal part mixture of Trigonella, Linum usitatisum, Phaseolus radiatus, Triticum vulagare and 4)an equal part mixture of Holarrhena antidysenterica, Embelia ribes, Tinospora cordifolia, Swertia chirata. Each drop of this AMAVAT-20 oil contains 8 mg equivalent of all the above herbs combined. One drop of this oil was given twice a day to 6 persons suffering from chronic stomachache and tendancy for loose bowels. All of them reported considerable reduction in stomach discomfort within 3 to 4 days. The tendancy for loose bowels also stopped.

Another case (female, 60+ years age) suffering from recurrent indigestion, diarrhea, urticaria has responded well to two drops per day of AMAVATA-20.

Menstrual Health

A 13 year old girl suffering from pain during menstruation, frequent colds and poor concentration in studies was given 2 drops per day of AMAVATA-20 and 1 tab/day of 1 mg equivalent of R-100. Over a period of 2 months, pain during menstuation has reduced considerably as also the frequency of colds. There is also an improvement in concentration in studies.

Two teenage girls suffering from delayed menstruation (35 to 40 day cycle) were given 2 drops per day of A-50(Aloe). Over a period of 2-3 months (two cycles) the menstruation cycle was restored to normalcy. The toal herbal dose was equivalent to 40 mg per day.

Arthritis, Backache and Joint Pain

A person (58 years, male) was suffering from lower back joint inflammatory pain, which was aggravated by vigourous walking. He started taking one drop per day of a composition designated as ‘RCPE-10 PLUS’ and consisting of a mixture of R-5 PLUS (2.5 parts), C-5 PLUS (2.5 parts), and PE-5 PLUS (5 parts). The total initial plant material per drop is 4 mg. After one week, there was a considerable reduction in the joint inflammation and pain.

A former jet fighter pilot (58 years old) was suffereing from chronic lower back pain and stiffness, particularly in the morning for several years. He started taking one drop per day of a composition designated ‘ARTH-10.’ The total starting plant material per drop is 4 mg. ARTH-10 contains equal parts of A-100 PLUS, R-100 PLUS, C-100 PLUS, ZOFF-55.4 PLUS, CAMA-26.2 PLUS and GGLAB-7.4 PLUS. After one week, he has found a considerable relief from his nagging back pain and stiffness.

Cough/Throat Irritation/Cold

A male (58 years old) developed a cold and early stage of a cough with throat irritation. One drop each of 1 R-5 PLUS, TABA-5 and GGLAB-5’ in a cup of hot water taken three times a day alleviated the symptoms within two days. Thus, the total daily dose was 6 mg of each of three plants.

In case of some other subjects, a drop of COUGH-5 was given thrice a day at the onset of cough, throat pain and cold. The throat pain and cough disappeared within two days as also the symptoms of cold. COUGH-10 is a mixture of: 2.4 parts of: a mixture of Zingiber officinale, Carum copticum, Cuminum cyminum, Piper longum, 0.6 parts of Glycyrrhiza glabra; and 2 parts of Piper nigrum. The total daily herbal dose in this case was 6 mg.

In cae of four persons suffering from hinitis, runy nose and headache due to congestion, one drop of OSAT-48 was applied with a cotton bud inside the nostrils. Within 15 minutes, there was a complete drainage of mucous with considerable relief from the symptoms of cold and headache.

Sciatica

A 78 year old female was suffering from throbbing pain due to sciatica. A mixture of 1 drop each of MPRU-5 and PE-5 along with 1 drop of Medium Chain Fatty Acid Tryglycerides was applied externally to the sore spot once a day. The throbbing pain completely stopped within 4 days. The total daily dose in this case was 2 mg each of Mucuna pruriens and Phyllanthus emblica.

An 80 year old male suffering from sciatica in both the legs was given a mixture of SHUKRA-5 (a mixture of Kalanchoe pinnata, Withania somnifera, Asperagas racemosus and Mucuna pruriens) and R-100 PLUS in the ratio of 3:2. Application of one drop per day to the tender spot on each leg and 1 drop internally has resulted in a considerable relief in the pain.

Neurology-Muscle Spasm

A person (60+ years, female) has been suffering from hemi-facial spasm for a long time. The spasm starts from above the left eye, travels down the nose and cheeks up to lips. The spasm lasts for several hours and causes face distortion and pain. She started applying one drop of an equal part mixture of PE-5 PLUS and ‘MPRU-5.’ When the drop is applied, the spasm subsides immediately and does not recur at least for 12 hours. Application of one drop also prevents the initiation of the spasm. The total herbal dose is just 2 mg per day.

A person (58 yeares, male) has been suffering from a chronic muscle spasm and painful area on the upper part of right foot between the fingers and ankle. Application of 2 drops of ‘MPRU-5’ (4 mg of herbal starting material) has eliminated the spasm and pain within two days.

Neurology—Post-Paralysis Recovery of Muscle Activity and Mental Concentration.

A person (58 years, male) had a stroke (left frontal MCA infarc) in 1996 with almost complete loss of speech. He was not able to concentrate on reading for more than 15 minutes at a time. Also, his handgrip was poor. Thus, recovery had been very very slow. From 1^{st} Sep., 2001, the person was given one drop per day each of PE-5 PLUS, MPRU-4.8 and GGLAB-5. The total plant material equivalent is 6 mg per day. By mid-November, 2002 (2.5 months) there was a considerable inprovement in ability to concentrate. He can now read for 2 hours at a time (compared to 15 minutes at a time before treatment) and the retention has improved. There is also a considerable improvement in the hand grip.

Another case is of a 20 year man. He had suffered a brain hemorrhage and consequently his ability to learn was severely constrained. He started taking NEURO-35 at the rate of two drops per day (an equal mixture of Phylanthus indica, Mucuna pruriens and Bacopa monnieri) equivalent to a total herbal dose of 45 mg per day. After one and a half month, there is now a noticeable improvement in his ability to learn a new language (Russian).

Neurology—Viral Polyneuritis

A person (male) was unable to lift his foot after an attack of viral polyneuritis. He was given one drop twice a day of NEURO-35 (an equal mixture of Phylanthus indica, Mucuna prurients and Bacopa monnieri) equivalent to a total herbal dose of 28 mg per day. After a few weeks, the ability of the muscles revived and he is able to lift his foot. The circumferance near the ankle also increased by 1 cm indicating a restoration of the wasted muscle.

Neurology—Speech Centres

A person suffering from heavy speech slurring for 15 years (triggered because of pesticidal neuritoxicity) was given NEURO-10 at the dose of 1 drop, twice a day equivalent to a total herbal dose of 8 mg per day. His speech clarity has improved within a few weeks.

In another case, a 50 year old person had serious speech coordination problems as a result of occupational stress particularly if he tried to speak fast. By taking two drops per day of NEURO-35 equivalent to a total herbal daily dose of 28 mg per day, his speech clarity has also improved within 2 weeks.

Reproductive System

A 13 year old girl was suffering from dysmenorrhea and did not menstruate for 3 months. She was given A-10 PLUS at the rate of 4 drops per day. This is equivalent to 16 mg/day of total plant material. Within 15 days she began to menstruate and a regular cycle was established.

### Example 12B

### Additional Cases of Use of Non-plant Biomass Extracts

PRAWN

Application of two drops of prawn extract (PRWN-5) alleviated knee pain for 1 hour. A mixture of one drop each of PRWN-5 and R-5 alleviated the pain for several hours.

MUSHROOM

A few persons suffering from a lack of appetite were given 1 drop of the button mushroom extract (MUSH-A-10) twice a day. Within four days, there was an improvement in the appetite. The total daily dose in this case was 8 mg per day of mushroom equivalent.

Agricultural Applications

Methods

In the following examples using Kalanchoe pinnata, R-5 or R-2 oil, R-10(P) powder and tablets made as per examples above were used. In the case of extracts of other plants, different potency oils were used as described in particular examples.

Plants were either grown in hydroponic or soil media.

Administration was accomplished by a variety of means, including direct application to the root zone, foliar spray, application of a solution at the root zone after dissolving/dispersing tablet/oil in water, injection in to the trunks or stems, application to terminal buds, addition to tissue culture medium, etc.

In the following examples, the typical dosage of extract for field crops was 0.5 to 1 g of R-100 oil or leaf equivalent per hectare per spray. The number of sprays can be typically at a frequency of once every one to three weeks. The dose for tree crops varied from 5 mg to 50 mg per tree of R-100 per year, depending on the size of the sapling/tree.

Multiple high dose sprays of particular preparations such as R-100 MINUS (5 to 25 g per spray per hectare) act to reduce the flower set, total seeds produced and the size of seeds. This effect can be used to control the propagation of hardy weeds such as “Congresss Grass” (Parthium sp.), Lantana sp., Cyperus sp. and others.

Toxicity

### Example 13

### R100 PLUS vs. R-100 MINUS of Kalanchoe Pinnata in Onion Root Tip Assay

The Onion Root Tip Assay was used to study genotoxicity profile of CAM plant extracts. The results of this test can be usefully related to the expected cytotological profile in animal cells or human lymphocytes (Meenakumari, 1995; Mercykutty, 1980).

Bulbs of Allium cepa, L. cv N-2-4-1 (2n=16) were used. Selected bulbs were washed and the root systems of the bulbs were kept in 100 ml of aqueous solutions in cavity blocks containing different amounts of herbal oil extract. Roots were treated for 48 hours.

At the end of 48 hours, the root tips were recovered and fixed in acetic acid-alcohol (1:3). For cytological preparations, root tips were hydrolyzed in 1 N HCl and squashed in 1% acetocarmine. Slides were examined under a microscpoe. Cells were observed and scored (Table 8), and the status of cells with respect to mitosis and various other physiological (clumping, stray and lagging chromosomes) and clastogenic (anaphase, fragments, binucleate) aberrations was recored. The total number of roots and the average length of the roots were also measured and sprouting from the tip of the bulb was also noted.

TABLE 8
Effect of R-100 PLUS vs. R-100 MINUS on Onion Root Tip

| Concentration, | No. cell | Mitotic Index | No. cells showing aberrations | | Root no./ |
|---|---|---|---|---|---|
| μl/liter | scored | - % dividing - | Physiological | Clastogenic | (length, cm) |
| 0 (CONTROL) | 1120 | 36.25 | 0 | 0 | 42 (4.3) |
| R-100 PLUS | | | | | |
| 10 | 1105 | 39.12 | 0 | 0 | 50 (4.6) |
| 30 | 1121 | 42.58 | 0 | 0 | 52 (4.7) |
| 100 | 1085 | 40.12 | 8 | 4 | 44 (3.9) |
| 300 | 1048 | 30.23 | 58 | 8 | 42 (3.6) |
| R-100 | | | | | |
| 10 | 1012 | 38.56 | 0 | 0 | 48 (4.5) |
| 30 | 1025 | 40.12 | 12 | 0 | 45 (4.1) |
| 100 | 1045 | 25.26 | 87 | 7 | 38 (3.8) |
| 300 | 1005 | 18.23 | 126 | 14 | 36 (1.6) |
| R-100 MINUS | | | | | |
| 10 | 1052 | 38.36 | 8 | 12 | 43 (4.2) |
| 30 | 930 | 25.25 | 49 | 31 | 38 (3.2) |
| 100 | 936 | 8.25 | 487 | 45 | 26 (1.2) |
| 300 | 856 | 1.22 | 671 | 69 | 12 (0.9) |

Roots that formed at higher concentrations of R-100 MINUS were short, yellowish and had curved tips. There was excellent sprouting on top of the bulb in case of control and 10 μl/liter of R-100 PLUS. Sprouting was moderate with 10 μl/liter of R-100 and 30 μl/liter of R-100 PLUS. There was no sprouting at all in any of the other sets.

In the above data, major aberrations were physiological and mainly clumping of chromosomes. However, the onset of aberrations was shown by R-100 MINUS-treated root tips at 10 μl/liter whereas R-100 PLUS-treated root tips began to show aberrations at 100 μl/liter, a tenfold higher concentration. The mitotic index, and the number and average root length also confirmed this observation.

R-100 PLUS, at least up to 30 μl liter promoted cell division, rooting and sprouting. In contrast, R-100 MINUS began to act as a mitogen and root system inhibitor beyond 10 μl/liter.

### Example 14

### CAM Plant Fractions in Onion Root Tip Assay

Experiments were carried out using A-100 PLUS, A-100 MINUS, C-100 PLUS, C-100 MINUS, R-100 PLUS, R-100 MINUS and sesame oil at the solution concentrations given in Table 9.

Bulbs of Allium cepa, L. cv N-2-4-1(2n=16) were used. Selected bulbs were washed and the root systems of the bulbs were kept in 100 ml aqueous solution in cavity blocks containing different amount of herbal oil extract. Roots were treated for 12 hours and then recovered in 10% glucose for another 12 hours.

At the end of recovery, the root tips were recovered and fixed in acetic acid-alcohol (1:3). For cytological preparations, root tips were hydrolyzed in 1 N HCl and squashed in 1% acetocarmine. Slides were examined under a microscope. Cells were observed and scored (Table 9), and the status of cells with respect to mitosis and various other physiological (clumping, stray and lagging chromosomes) and clastogenic (anaphase, fragments, binucleate) aberrations was recorded. The results are summarized in Table 9.

TABLE 9
Effect of CAM Plant Fractions on Onion Root Tip

| Concentration, | No. cell | Mitotic Index | % cells showing aberrations | |
|---|---|---|---|---|
| μl/liter | scored | -% dividing- | Physiological | Clastogenic |
| Sesame oil | | | | |
| 0 | 1552 | 30.12 | 0 | 0 |
| 100 | 1230 | 31.23 | 0.08 | 0 |
| 300 | 1452 | 32.59 | 0.34 | 0 |
| R-100 PLUS | | | | |
| 30 | 1530 | 32.24 | 0 | 0 |
| 100 | 1547 | 33.56 | 0.52 | 0 |
| 300 | 1531 | 18.63 | 1.31 | 0.2 |
| R-100 MINUS | | | | |
| 30 | 1498 | 28.27 | 0.13 | 0 |
| 100 | 1521 | 14.45 | 1.78 | 0.26 |
| 300 | 1530 | 9.63 | 3.01 | 0.92 |
| A-100 PLUS | | | | |
| 30 | 1521 | 32.21 | 0 | 0 |
| 100 | 1511 | 33.52 | 0 | 0 |
| 300 | 1505 | 31.89 | 0.8 | 0 |
| A-100 MINUS | | | | |
| 30 | 1563 | 31.25 | 0 | 0 |
| 100 | 1524 | 32.68 | 0.2 | 0 |
| 300 | 1541 | 28.30 | 0.97 | 0 |
| C-100 PLUS | | | | |
| 30 | 1621 | 32.33 | 0.06 | 0 |
| 100 | 1563 | 26.23 | 1.09 | 0 |
| 300 | 1518 | 11.14 | 1.98 | 0.4 |
| C-100 MINUS | | | | |
| 30 | 1546 | 31.28 | 0.45 | 0 |
| 100 | 1543 | 12.65 | 1.94 | 0.32 |
| 300 | 1532 | 8.25 | 3.26 | 0.91 |

In all cases, the PLUS fraction (LR) appeared to be an excellent promoter of mitosis or cell proliferation compared to sesame oil controls. In some cases 100 PLUS and A-100 PLUS), this activity was retained up to 100 μl/liter concentration in this assay. The PLUS fractions also had lower toxicity than the corresponding MINUS (J) fractions in terms of mitogenic activity inhibition and genotoxicity.

The use of the PLUS fraction and exclusion of the MINUS fraction for medicated oil preparations is contradicts the teachings of traditional medicine. Surprisingly, the method of the invention improves the overall potential of health promotion and broadens the safe operating range. This method also allows compositions of high potency at low dose; thus further reducing the toxicity potential.

These positive effects, particularly in case of Kalanchoe and Cissus, have greatly extended their safe operating range, considerably improved their efficacy/toxicity ratio and therefore extended their utility in applications that hitherto were restricted due to the toxicity of the use of juice with a high dosage.

The higher toxicity of the juice-based extract can be used in applications to eliminate unwanted vegetation or control plant growth.

### Example 14A

### Non-CAM Plant Fractions in Onion Root Tip Assay

Experiments were carried out using TABA-28, CROT-62.66 MINUS, TVUL-32 PLUS, SCHIR-55 MINUS, HA-29.7, PE-100 PLUS, PE-100 MINUS, and sesame oil at the solution concentrations given in Table 9 A. R-100 PLUS was also used as comparison.

Bulbs of Allium cepa, L. cv N-2-4-1(2n=16) were used. Selected bulbs were washed and the root systems of the bulbs were kept in 100 ml aqueous solution in cavity blocks containing different amount of herbal oil extract. Roots were treated for 48 hours.

At the end of 48 hours, the root tips were recovered and fixed in acetic acid-alcohol (1:3). For cytological preparations, root tips were hydrolyzed in 1 N HCl and squashed in 1% acetocarmine. Slides were examined under a microscope. Cells were observed and scored (Table 9 A ), and the status of cells with respect to mitosis and various other physiological (clumping, stray and lagging chromosomes) and clastogenic (anaphase, fragments, binucleate) aberrations was recorded. The results are summarized in Table 9A.

TABLE 9A
Effect of Non-CAM Plant Fractions on Onion Root Tip

| | | Number | | | | |
|---|---|---|---|---|---|---|
| | | of | Mitotic | Physiological | Clstogenic | Total |
| | Concentration | Cells | Index, % | Aberration, | Aberration, | Aberration, |
| Extract | μl/liter | Scored | Dividing | % Cells | % Cells | % Cells |
| Control | 0 | 1236 | 23.14 | 0.0 | 0.0 | 0.0 |
| Sesame | 3 | 1235 | 24.17 | 0.0 | 0.0 | |
| Oil | 10 | 1230 | 23.17 | 0.16 | 0.0 | 0.16 |
| | 30 | 1452 | 21.49 | 0.34 | 0.0 | 0.34 |
| R-100 | 3 | 1250 | 26.00 | 0.08 | 0.0 | 0.08 |
| PLUS | 10 | 1230 | 23.50 | 1.06 | 0.0 | 1.06 |
| | 30 | 1520 | 16.84 | 1.84 | 0.26 | 2.11 |
| TABA- | 3 | 1698 | 22.50 | 0.59 | 0.0 | 0.59 |
| 28 | 10 | 1615 | 20.12 | 1.67 | 0.31 | 1.98 |
| | 30 | 1602 | 17.85 | 2.56 | 0.69 | 3.25 |
| CROT- | 3 | 1629 | 23.51 | 0.74 | 0.0 | 0.74 |
| 62.66 | 10 | 1622 | 16.09 | 1.97 | 0.31 | 2.28 |
| MINUS | 30 | 1628 | 11.36 | 3.28 | 0.92 | 4.30 |
| TVUL- | 3 | 1521 | 25.97 | 0.13 | 0.0 | 0.13 |
| 32 | 10 | 1566 | 24.39 | 0.38 | 0.0 | 0.38 |
| PLUS | 30 | 1590 | 22.39 | 1.45 | 0.0 | 1.45 |
| SCHIR- | 3 | 1611 | 22.66 | 0.62 | 0.06 | 0.68 |
| 55 | 10 | 1598 | 20.03 | 1.94 | 0.38 | 2.32 |
| MINUS | 30 | 1596 | 12.84 | 3.13 | 0.69 | 3.82 |
| HA- | 3 | 1562 | 24.71 | 0.26 | 0.0 | 0.26 |
| 29.7 | 10 | 1624 | 19.77 | 1.35 | 0.12 | 1.47 |
| | 30 | 1615 | 14.98 | 2.85 | 0.74 | 3.59 |
| PE- | 3 | 1595 | 24.20 | 0.69 | 0.0 | 0.69 |
| 100 | 10 | 1622 | 17.82 | 2.10 | 0.43 | 2.53 |
| PLUS | 30 | 1659 | 12.18 | 3.01 | 0.66 | 3.68 |
| PE- | | 1652 | 21.55 | 0.85 | 0.0 | 0.85 |
| 100 | | 1623 | 17.51 | 2.45 | 0.67 | 3.12 |
| MINUS | | 1623 | 11.58 | 3.94 | 0.99 | 4.93 |

Fractions from Kalanchoe (R-100 PLUS), wheat (TVUL-32 PLUS), Holarrhena antidysenterica (HA-29.7) and Phyllanthus emblica (PE-100 PLUS) show mitogenic index increase or cell division promotion at/upto 3 μl/liter of the oil extract. At higher concentration, these and other extracts all show a decline in mitigentic index or inhibition of cell division. However, in all these cases, the % of clastogenic aberrations is rather low. Thus, these extracts, at higher concentrations can be used for contolling cell proliferation without causing significant mutagenic effects.

Applications to Dicotyledonous Plants

Germination Promotion

### Example 15

### Germination of Phaseolus Radiatus (Mung Bean) Using R-10(P)

Using R-10 (P) (batch 881128) in Phaseolus radiatus, a legume, the seeds were soaked in a solution of R-10 (P) and observations were taken at 24, 48 and 120 hours after soaking. The results are summarized in Table 10.

TABLE 10
Germination promotion

| Concentration, | | | |
|---|---|---|---|
| ppm, | ROOT LENGTH (cm), RANGE | | |
| R-10(P) | 24 Hrs | 48 Hrs | 120 Hrs |
| 0(Control) | 0.2-1.0 | 3.5-6.0 | 4.2-8.6 |
| 1 | 0.2-2.0 | 4.0-6.2 | 3.5-12.5 |
| 5 | 0.2-2.5 | 4.0-6.8 | 5.7-12.1 |
| 20 | 2.0-2.8 | 4.5-7.1 | 13.9-17.8 |
| 500 | 0.5-2.5 | 0.5-4.7 | 3.6-11.2 |

Thus, germination promotion is observed with increasing concentration up to 20 ppm of R-10(P). At 20 ppm of R-10(P) or 2 ppm of R-100 equivalent, there is a particularly strong promotion of germination.

### Example 16

### R-100 PLUS vs. R-100 MINUS of Kalanchoe pinnata on Phaseolus rasdiatus

Germination experiments were carried out with R-100 PLUS vs. R-100 MINUS and also with the standard R-100 extract. Twenty-five (25) seeds of Phaeolus mungo were placed in a plate with 5 ml of distilled water containing various concentrations of the R-100 extracts. On the b 7^{th} day after initiation of experiment, mean values of 11 seedlings were taken and reported. The results are summarized below in Table 11.

TABLE 11
Germination in Phaeolus mungo; comparison of
different forms of extract

| | | | | |
|---|---|---|---|---|
| | | | R-100 | |
| Concentration | R-100 PLUS | R-100 | MINUS | Sesame oil |

Root Length (cm)

| | | | | |
|---|---|---|---|---|
| .0 | 4.62(0.26) | 4.62(0.26) | 4.62(0.26) | 4.62(0.26) |
| 0.33 | 5.22(0.08) | 5.18(0.40) | 4.76(0.11) | 4.52(0.29) |
| 1.0 | 5.38(0.08) | 5.30((0.10) | 4.86(0.39) | 4.92(0.18) |
| 3.0 | 5.62(0.08) | 5.56(0.11) | 4.94(0.11) | 4.96(0.19) |
| 30.0 | 5.30(0.07) | 5.12(0.09) | 4.68(0.11) | 5.28(0.22) |

Shoot Length (cm)

| | | | | |
|---|---|---|---|---|
| 0.0 | 9.08(0.29) | 9.08(0.29) | 9.08(0.29) | 9.08(0.29) |
| 0.33 | 11.48(0.24) | 11.48(0.53) | 13.64(0.38) | 10.56(0.23) |
| 1.0 | 12.44(0.23) | 13.50(0.07) | 15.02(0.22) | 10.96(0.18) |
| 3.0 | 13.70(0.37) | 13.56(0.23) | 15.70(0.20) | 11.84(0.17) |
| 30.0 | 13.46(0.15) | 11.4(0.16) | 15.12(0.49) | 12.54(0.11) |

Dry weight, g (10 seedlings) - mean of two observations

| | | | | |
|---|---|---|---|---|
| 0.0 | 0.33 | 0.33 | 0.33 | 0.33 |
| 0.33 | 0.35 | 0.34 | 0.32 | 0.33 |
| 1.0 | 0.42 | 0.41 | 0.34 | 0.33 |
| 3. | 0.40 | 0.38 | 0.31 | 0.34 |
| 30 | 0.35 | 0.32 | 0.27 | 0.35 |

Note:
Concentration in the medium is in μl/50 ml distilled water.
Values in parenthesis are standard deviation.

All fractions showed significant biological activity at very low doses. Both R-100 and R-100 PLUS show significant promotion of root and shoot growth and biomass weight at the end of 7 days compared to sesame oil controls at 1 and 3 mg levels. The PLUS fraction showed the best overall promotional effect.

R-100 MINUS did show shoot growth compared to sesame oil control. However, there was no root growth and no increase in dry biomass weight up to 3 mg. At the higher dose level, R-100 also showed a sharper drop in root length and biomass retention.

Thus, this data corroborates the contrasting behavior of LR and the J fractions-based compositions from Kalanchoe described in Example 14 above.

### Example 17

### Effect of CAM Plant Fractions on Germination of Phaseolus mungo

Twenty-five (25) seeds of Phaseolus mungo were placed in a plate with 5 ml of distilled water, containing various concentrations of oil extracts or plain base oil. On the 7^{th} day after initiation of experiment, mean values of 10 seedlings were taken and reported in Table 12.

TABLE 12
Effect of CAM plant fractions on seed germination activity

| | | | | |
|---|---|---|---|---|
| Sesame Oil | A-100 | A-100 | C-100 | C-100 |
| Concentration Plain | PLUS | MINUS | PLUS | MINUS |

Root Length(cm)

| | | | | | |
|---|---|---|---|---|---|
| 0.0 | 5.09(0.065) | 5.12(0.065) | 5.12(0.065) | 5.12(0.065) | 5.12(0.065) |
| 1.0 | 5.20(0.048) | 6.33(0.068) | 6.12(0.087) | 6.75(0.055) | 5.42(0.063) |
| 3.0 | 5.30(0.052) | 6.51(0.052) | 6.39(0.047) | 4.33(0.065) | 5.23(0.071) |
| 10.0 | 5.67(0.061) | 6.69(0.061) | 6.41(0.045) | 4.67(0.062) | 4.51(0.042) |

Shoot Length(cm)

| | | | | | |
|---|---|---|---|---|---|
| 0.0 | 10.35(0.058) | 10.35(0.058) | 10.35(0.058) | 10.35(0.058) | 10.35(0.058) |
| 1.0 | 12.08(0.062) | 12.12(0.054) | 11.86(0.062) | 13.35(0.064) | 13.62(0.048) |
| 3.0 | 12.98(0.047) | 13.12(0.062) | 13.02(0.068) | 12.33(0.56) | 12.65(0.052) |
| 10.0 | 13.56(0.054) | 13.56(0.057) | 13.12(0.077) | 11.38(0.054) | 12.17(0.051) |

Dry Weight(gm/10 seedlings) mean of two observations. Numbers
in parenthesis indicate ± range of the two observations.

| | | | | | |
|---|---|---|---|---|---|
| 0.0 | 0.332(0.0023) | 0.332(0.0023) | 0.332(0.0023) | 0.332(0.0023) | 0.332(0.0023} |
| 1.0 | 0.351(0.0023) | 0.371(0.0022) | 0.384(0.0022) | 0.352(0.0022) | 0.364(0.0018) |
| 3.0 | 0.363(0.0018) | 0.408(0.0023) | 0.397(0.0021) | 0.382(0.0023) | 0.377(0.0019) |
| 10.0 | 0.362(0.0019) | 0.427(0.0019) | 0.415(0.0023) | 0.336(0.0021) | 0.322(0.0023) |

Note:
Concentration in the medium is in μl of oil/50 ml distilled water.
Values in parenthesis indicate standard deviation.

PLUS and MINUS fractions of both CAM plants promoted auxin-like (rooting promotion), gibberellin-like (shooting promotion) and cytokine-like (biomass preservation/growth) activity up to 1 μl/50 ml DW. A-100 PLUS and MINUS both promoted rooting and a higher dry biomass at the end of 7 days, even at the higher concentration of 10 μl/50ml distilled water. Thus, A-100 PLUS and MINUS promote a wide range of endogenous hormones at a low concentration and this promotional effects continue up to a high concentration. However, C-100 PLUS and MINUS had a different activity profile. They showed auxin, gibberellin and cytokine-like activities at 1 μl/50 ml DW as well. However, at higher concentrations (10 μl/50 ml distilled water), a reversal in all activities was observed. Thus, this process of inhibition of activity started earlier with C-100 than the other extracts.

### Example 17A

### Effect of Various Angiosperm and Gymnosperm Plant Extracts and Non-Plant Biomass Extracts on Germination of Phaseolus radiatus

Agar (0.8 wt. % agar in distilled water) was digested in water bath to get transperant medium and 100 ml of this solution (base medium) was poured in 300 ml culture bottles. Different doses of oil extracts of various Angiosperm-monocotyledon, Angiosperm-dicotyledon and Gymnosperm plants, non-plant biomass or plain base sesame oil were added to each bottle. Bottles were sterilized at 15 lbs for 20 mnutes in an autoclave. Surface sterilized 10 seeds of Phaseolus radiatus were added to each bottle amd germinated in dark for 5 days. On the 5^{th} day after initiation of experiment, values of shoot lenth, root lenth, and dry weight of biomass (dried in an oven at 70-80 deg. C. for constant dry weight) were measured for 10 seedlings. Mean values are reported in Table 12A-12K, 12M, 12O, 12Q, 12S, 12U, 12W. Several extracts of plants and their fractions and non-plant biomass promoted rooting, shooting and biomass mobilization at low doses.

TABLE 12A
Effect of plant extracts on P. radiatus seed germination activity

| Extract/ | | Sesame | PE 100 | PE 100 | TBEL | TBEL | TBEL |
|---|---|---|---|---|---|---|---|
| Bottle | | Oil | PLUS | MINUS | R 50 | P 50 | 27.7 |
| 0 | Shoot | 23.5 | | | | | |
| Control | (cm) | (1.09) | | | | | |
| | Root | 8.3 | | | | | |
| | (cm) | (0.74) | | | | | |
| | Total | 31.3 | | | | | |
| | (cm) | (1.38) | | | | | |
| 1 | Shoot | 24.7 | 25.8 | 25.1 | 24.9 | 24.6 | 26.0 |
| | (cm) | (1.03) | (1.04) | (1.01) | (0.70) | (0.83) | (1.04) |
| | Root | 9.4 | 11.3 | 12.1 | 10.8 | 9.9 | 9.6 |
| | (cm) | (0.51) | (0.95) | (0.61) | (0.61) | (0.62) | (0.40) |
| | Total | 34.1 | 37.1 | 37.2 | 35.6 | 34.5 | 35.6 |
| | (cm) | (0.97) | (1.50) | (0.90) | (1.09) | (0.82) | (1.08) |
| 3 | Shoot | 25.2 | 26.3 | 26.8 | 25.5 | 25.3 | 25.7 |
| | (cm) | (0.99) | (1.02) | (0.86) | (0.71) | (0.69) | (0.80) |
| | Root | 10.8 | 12.8 | 12.3 | 12.82 | 11.4 | 11.0 |
| | (cm) | (0.40) | (1.06) | (0.96) | (0.82) | (0.93) | (0.92) |
| | Total | 36.1 | 39.1 | 39.1 | 38.3 | 36.7 | 36.6 |
| | (cm) | (1.02) | (1.5) | (1.51) | (1.16) | (0.90) | (1.13) |
| 10 | Shoot | 25.0 | 26.8 | 21.7 | 24.7 | 26.0 | 26.5 |
| | (cm) | (1.01) | (1.36) | (1.05) | (1.21) | (1.27) | (0.92) |
| | Root | 10.0 | 8.8 | 12.4 | 11.4 | 10.7 | 12.4 |
| | (cm) | (0.55 | (0.74) | (0.76) | (0.70) | (0.83) | (1.32) |
| | Total | 35.1 | 35.7 | 34.1 | 36.1 | 36.7 | 38.8 |
| | (cm) | (1.39) | (1.95) | (1.23) | (1.46) | (1.20) | (1.44) |

Note:
Extract/Bottle in the medium is in μl of oil/100 ml base medium.
Values in parenthesis are standard deviation of the estimate.

Dry biomass weight per 10 seedlings at the end of 5 days is reported in Table 12B below.

TABLE 12B
Effect of plant extracts on P. radiatus germination activity

| Extract/ | | Sesame | PE 100 | PE 100 | TBEL | TBEL | TBEL |
|---|---|---|---|---|---|---|---|
| Bottle | | Oil | PLUS | MINUS | R 50 | P 50 | 27.7 |
| 0 | E.A. | 230 | | | | | |
| | (mg) | | | | | | |
| 1 | E.A. | 228 | 294 | 302 | 220 | 240 | 240 |
| | (mg) | | | | | | |
| 3 | E.A. | 226 | 268 | 306 | 246 | 210 | 232 |
| | (mg) | | | | | | |
| 10 | E.A. | 224 | 240 | 238 | 260 | 270 | 266 |
| | (mg) | | | | | | |

Note:
Extract/Bottle in the medium is in μl of oil/100 ml base medium.
E.A.—Embryo Axis (Root + Shoot)

Phyllanthus emblica (PE 100 PLUS and MINUS) fractions show strong promotion, particularly of rooting and biomass growth (E.A. value) up to 3 μl of oil/100 ml base medium. Rooting promotion is shown by the ‘TBEL R 50’ fraction (Terminalia bellerica Rind) at 3 μl of oil/100 ml base medium. Both the Rind (R) and Pulp (P) fraction of TBEL also show biomass growth at the higher concentration.

TABLE 12C
Effect of plant extracts on P. radiatus seed germination activity

| | | | FBENG | FBENG | | CAMA | ZOFF |
|---|---|---|---|---|---|---|---|
| Extract/ | | Sesame | 29 | 100 | TCHEB | 98.4 | 100 |
| Bottle | | Oil | PLUS | MINUS | 27.8 | MINUS | MINUS |
| 0 | Shoot | 22.8 | | | | | |
| Control | (cm) | (0.92) | | | | | |
| | Root | 8.1 | | | | | |
| | (cm) | (0.71) | | | | | |
| | Total | 30.9 | | | | | |
| | (cm) | (0.95) | | | | | |
| 1 | Shoot | 23.7 | 23.8 | 24.2 | 23.7 | 24.0 | 23.8 |
| | (cm) | (1.11) | (1.1) | (0.62) | (1.17) | (0.90) | (0.87) |
| | Root | 8.3 | 9.6 | 9.3 | 8.9 | 8.3 | 8.6 |
| | (cm) | (0.82) | (0.84) | (0.61) | (0.46) | (0.64) | (0.72) |
| | Total | 32.1 | 33.4 | 33.5 | 32.7 | 32.2 | 32.4 |
| | (cm) | (1.38) | (1.66) | (0.90) | (1.32) | (0.88) | (1.06) |
| 3 | Shoot | 24.2 | 24.0 | 28.3 | 23.6 | 24.5 | 24.4 |
| | (cm) | (0.98) | (0.91) | (0.84) | (0.99) | (0.74) | (0.69) |
| | Root | 8.6 | 10.1 | 9.1 | 10.0 | 9.8 | 8.8 |
| | (cm) | (1.12) | (0.83) | (0.72) | (0.79) | (0.34) | (0.69) |
| | Total | 32.8 | 34.1 | 37.3 | 33.5 | 34.3 | 33.2 |
| | (cm) | (1.68) | (0.96) | (1.28) | (1.21) | (0.64) | (0.97) |
| 10 | Shoot | 24.0 | 23.8 | 25.1 | 23.4 | 25.8 | 24.0 |
| | (cm) | (1.22) | (1.06) | (0.83) | (0.72) | (0.82) | (1.21) |
| | Root | 8.9 | 8.1 | 9.4 | 8.3 | 7.9 | 8.5 |
| | (cm) | (0.92) | (0.64) | (0.76) | (0.82) | (1.07) | (0.53) |
| | Total | 32.9 | 32.0 | 34.5 | 31.7 | 33.7 | 32.5 |
| | (cm) | (1.65) | (1.31) | (1.16) | (1.17) | (0.86) | (1.32) |

Note:
Extract/Bottle in the medium is in μl of oil/100 ml base medium.
Values in parenthesis indicate standard deviation.

Dry biomass weight/10 seedlings at the end of 5 days is reported in Table 12D below.

TABLE 12D
Effect of plant extracts on P. radiatus germination activity

| | | | FEBG | FBENG | | CAMA | ZPFF |
|---|---|---|---|---|---|---|---|
| Extract/ | | Sesame | 29 | 100 | TCHEB | 98.4 | 100 |
| Bottle | | Oil | PLUS | MINUS | 27.8 | MINUS | MINUS |
| 0 | E.A. | 322 | | | | | |
| | (mg) | | | | | | |
| 1 | E.A. | 318 | 314 | 288 | 296 | 292 | 298 |
| | (mg) | | | | | | |
| 3 | E.A. | 256 | 300 | 320 | 270 | 308 | 278 |
| | (mg) | | | | | | |
| 10 | E.A. | 290 | 288 | 312 | 294 | 322 | 284 |
| | (mg) | | | | | | |

Note:
Extract/Bottle in the medium is in μl of oil/50 ml base medium.
E.A.—Embryo Axis (Root + Shoot)

Ficus bengalensis fractions (FBENG 29 PLUS and FBENG 100 MINUS show promotion of growth (root+shoot) upto 3 μl of oil/100 ml base medium. Moderate rooting promotion is shown by Terminalia chebula (TCHEB 27.8) up to 3 μl of oil/100 ml base medium. However, promotion is not seen in biomass growth on a consistent basis in these cases.

TABLE 12E
Effect of plant extracts on P. radiatus seed germination activity

| | | | SCHIR | SCHIR | ER | ER | | | |
|---|---|---|---|---|---|---|---|---|---|
| Extract/ | | Sesame | 27.8 | 55 | 28.8 | 100 | TABA | MPRU | HA |
| Bottle | | Oil | PLUS | MINUS | PLUS | MINUS | 28 | 27.1 | 29.7 |
| 0 | Shoot | 24.0 | | | | | | | |
| Control | (cm) | (0.97) | | | | | | | |
| | Root | 8.0 | | | | | | | |
| | (cm) | (0.81) | | | | | | | |
| | Total | 32.0 | | | | | | | |
| | (cm) | (1.44) | | | | | | | |
| 1 | Shoot | | 24.2 | 25.0 | 23.4 | 26.5 | 28.3 | 26.6 | 22.8 |
| | (cm) | | (1.19) | (0.98) | (1.12) | (1.03) | (0.81) | (1.09) | (0.78) |
| | Root | | 12.1 | 11.9 | 10.3 | 10.0 | 10.4 | 9.3 | 10.3 |
| | (cm) | | (1.34) | (1.03) | (1.0) | (0.67) | (0.55) | (0.59) | (0.65) |
| | Total | | 36.3 | 36.9 | 33.8 | 36.6 | 38.7 | 35.9 | 33.1 |
| | (cm) | | (2.04) | (1.32) | (0.99) | (1.31) | (1.16) | (1.37) | (0.84) |
| 3 | Shoot | 24.2 | 25.1 | 26.5 | 23.3 | 27.1 | 28.5 | 27.9 | 29.1 |
| | (cm) | (0.98) | (0.80) | (0.97) | (1.04) | (0.96) | (1.01) | (0.51) | (0.87) |
| | Root | 10.5 | 12.7 | 10.3 | 11.3 | 11.2 | 11.9 | 11.5 | 12.1 |
| | (cm) | (0.87) | (0.70) | (1.01) | (0.93) | (0.86) | (0.74) | (0.63) | (0.85) |
| | Total | 34.7 | 37.8 | 36.8 | 34.7 | 38.3 | 40.3 | 39.3 | 41.3 |
| | (cm) | (1.23) | (1.08) | (0.99) | (0.87) | (1.59) | (0.90) | (0.76) | (1.49) |
| 10 | Shoot | | 28.5 | 24.6 | 28.7 | 23.9 | 20.3 | 24.4 | 26.2 |
| | (cm) | | (1.20) | (1.18) | (1.11) | (1.05) | (0.91) | (0.77) | (1.05) |
| | Root | | 9.2 | 7.4 | 11.0 | 10.8 | 5.0 | 8.2 | 9.7 |
| | (cm) | | (0.73) | (.79) | (1.05) | (0.82) | (0.54) | (0.72) | (0.69) |
| | Total | | 37.7 | 32.0 | 29.7 | 34.7 | 25.3 | 32.6 | 35.9 |
| | (cm) | | (1.61) | (1.35) | (1.95) | (1.33) | (1.15) | (1.09) | (1.27) |

Note:
Extract/Bottle in the medium is in μl of oil/100 ml base medium.
Values in parenthesis indicate standard deviation.

Dry biomass weight/10 seedlings at the end of 5 days is reported in Table 12F below.

TABLE 12F
Effect of plant extracts on P. radiatus germination activity

| | | | SCHIR | SCHIR | ER | ER | | | |
|---|---|---|---|---|---|---|---|---|---|
| Extract/ | | Sesame | 27.8 | 55 | 28.8 | 100 | TABA | MPRU | HA |
| Bottle | | Oil | PLUS | MINUS | PLUS | MINUS | 28 | 27.1 | 29.7 |
| 0 | E.A.(mg) | 200 | | | | | | | |
| 1 | E.A.(mg) | | 289 | 276 | | 253 | | 300 | 197 |
| 3 | E.A.(mg) | 190 | 266 | 287 | 259 | 283 | 244 | 331 | 250 |
| 10 | E.A.(mg) | | 222 | 296 | 250 | 260 | 232 | 193 | 244 |

Note:
Extract/Bottle in the medium is in μl of oil/100 ml base medium.
E.A. = Embryo Axis (Root + Shoot)

All fractions in the above Table have shown a maximum biomass growth promotion (E.A.) at 3 μl of oil/100 ml base medium. Several fractions have also shown a maximu ‘root+shoot’ growth promotion at 3 μl of oil/100 ml base medium. In most cases, this is a result of a strong shoot growth promotion. In several cases, there is a sharp reduction in ‘root+shoot’ growth at 10 μl of oil/100 ml base medium.

TABLE 12G
Effect of plant extracts on P. radiatus seed
germination activity

| Extract/ | | Sesame | AZIN- | PGL- | SIND- | TRIGF- | |
|---|---|---|---|---|---|---|---|
| Bottle | | Oil | C-29 | C-33 | 28.8 | 29.62 | |
| 0 | Shoot | 22.1 | | | | | |
| Control | (cm) | (0.75) | | | | | |
| | Root | 8.2 | | | | | |
| | (cm) | (0.87) | | | | | |
| | Total | 30.3 | | | | | |
| | (cm) | (1.05) | | | | | |
| 1 | Shoot | 23.1 | 22.3 | 23.2 | 23.7 | 22.4 | ( ) |
| | (cm) | (0.82) | (0.81) | (0.80) | (0.66) | (0.86) | |
| | Root | 8.6 | 8.6 | 9.0 | 8.8 | 8.3 | ( ) |
| | (cm) | 0.49) | (0.75) | (0.79) | (1.01) | (0.94) | |
| | Total | 31.7 | 30.9 | 32.3 | 32.6 | 30.7 | ( )( |
| | (cm) | (1.10) | (1.02) | (1.19) | (1.2) | (1.43) | |
| 3 | Shoot | 24.0 | 19.1 | 21.0 | 24.1 | 24.1 | ( ) |
| | (cm) | (0.84) | (0.59) | (0.71) | (0.72) | (0.52) | |
| | Root | 8.8 | 7.2 | 8.8 | 9.1 | 8.8 | ( ) |
| | (cm) | (1.07) | (0.85) | (0.56) | (0.92) | (0.46) | |
| | Total | 32.9 | 26.4 | 29.3 | 33.3 | 32.9 | ( ) |
| | (cm) | (1.33) | (1.33) | (0.79) | (1.55) | (0.88) | |
| 10 | Shoot | 24.1 | *13.3 | 19.0 | 24.8 | 25.9 | ( ) |
| | (cm) | (0.70) | (0.88) | (1.09) | (0.84) | (1.02) | |
| | Root | 9.0 | *6.3 | 7.6 | 9.8 | 9.2 | ( ) |
| | (cm) | (0.95) | (0.57) | (0.84) | (0.74) | (0.72) | |
| | Total | 33.1 | *19.6 | 26.5 | 34.6 | 35.16 | ( ) |
| | (cm) | (1.34) | (0.60) | (1.1) | (1.35) | (1.26) | |

Note:
Extract/Bottle in the medium is in μl of oil/100 ml base medium.
Values in parenthesis indicate standard deviation.

In this set, there was a complete liqufaction of the base medium. As result, only five seedlings grew and the others sank to the bottom. Hence, the readings are an average of 5 seedlings.

Dry biomass weight per 10 seedlings at the end of 5 days is reported in Table 12H below.

TABLE 12H
Effect of plant extracts on P. radiatus germination
activity

| Extract/ | | Sesame | AZIN- | PGL- | SIND- | TRIGF- |
|---|---|---|---|---|---|---|
| Bottle | | Oil | C-29 | C-33 | 28 | 29.62 |
| 0 | E.A.(mg) | 280 | | | | |
| Control | Root(mg) | 62 | | | | |
| | Shoot(mg) | 218 | | | | |
| 1 | E.A.(mg) | 364 | 377 | 380 | 349 | 228 |
| | Root(mg) | 70 | 78 | 62 | 60 | 63 |
| | Shoot(mg) | 294 | 299 | 318 | 289 | 165 |
| 3 | E.A.(mg) | 350 | 324 | 336 | 389 | 322 |
| | Root(mg) | 68 | 80 | 70 | 75 | 68 |
| | Shoot(mg) | 282 | 244 | 266 | 314 | 254 |
| 10 | E.A.(mg) | 314 | *287 | 312 | 334 | 307 |
| | Root(mg) | 64 | *73 | 58 | 72 | 59 |
| | Shoot(mg) | 250 | *214 | 254 | 262 | 260 |

Note:
Extract/Bottle in the medium is in μl of oil/100 ml base medium.
E.A.—Embryo Axis (Root + Shoot)

In this set, there was a complete liqufaction of the base medium. As result, only five seedlings grew and the others sank to the bottom. Hence, the weight reading for 5 seedlings per set has been multipied by 2 and recorded for comparison.

In this set, the Pongamia glabra cake (PGL-C-33) and particularly the Azadirachta indica cake (AZIN-C-29) extracts have shown a strong inhibitory activity for rooting and shooting above 3 μl of oil/100 ml base medium. This is confirmed by low biomass growth (E.A.) in case of Azadiracta indica cake. However, biomass growth in case of Pongamia glabra cake has not gone down. In case of Sesamum indicum (SIND-28) there is no significant increase in root and shoot length. However, there is a significant in crease in the biomass at 1 and 3 μl of oil/100 ml base medium. Thus, a wide range of activity has been observed.

TABLE 12I
Effect of plant extracts on P. radiatus seed
germination activity

| | | | CROT | CROT | TVUL | TVUL | ZOFF | CAMA | |
|---|---|---|---|---|---|---|---|---|---|
| Extract/ | | Sesame | 28.2 | 62.66 | 32.83 | 66.84 | 55.4 | 26.2 | GGLAB |
| Bottle | | Oil | PLUS | MINUS | PLUS | MINUS | PLUS | PLUS | 27.43 |
| 0 | Shoot | 20.0 | | | | | | | |
| Control | (cm) | (0.82) | | | | | | | |
| | Root | 9.0 | | | | | | | |
| | (cm) | (0.70) | | | | | | | |
| | Total | 29.1 | | | | | | | |
| | (cm) | (1.17) | | | | | | | |
| 1 | Shoot | 20.5 | 21.9 | 20.7 | 20.7 | 20.2 | 24.1 | 22.4 | 21.9 |
| | (cm) | (0.72) | (0.90) | (1.33) | (0.57) | (0.25) | 0.50) | (0.67) | (0.97) |
| | Root | 9.8 | 10.3 | 10.7 | 10.4 | 10.3 | 11.5 | 10.7 | 10.6 |
| | (cm) | (0.51) | (0.41) | (0.72) | (0.67) | (0.63) | (0.65) | (0.31) | (0.39) |
| | Total | 30.3 | 32.2 | 31.4 | 31.1 | 30.5 | 35.6 | 33.1 | 32.5 |
| | (cm) | (0.83) | (0.92) | (1.58) | (0.77) | (0.57) | (0.82) | (0.72) | (1.06) |
| 3 | Shoot | 20.6 | 22.4 | 20.5 | 23.3 | 20.4 | 23.3 | 23.1 | 23.7 |
| | (cm) | (0.80) | (0.59) | (0.88) | (0.53) | (1.04) | (0.47) | (0.42) | (0.39) |
| | Root | 10.1 | 11.1 | 10.2 | 11.7 | 11.2 | 10.9 | 9.8 | 111.4 |
| | (cm) | (0.49) | (0.52) | (0.42) | (0.67) | (0.50) | (0.51) | (0.55) | (0.72) |
| | Total | 30.8 | 33.5 | 30.5 | 35.0 | 31.7 | 32.2 | 33.0 | 35.1 |
| | (cm) | (0.95) | (0.60) | (0.75) | (0.73) | (0.95) | (0.48) | (0.72) | (0.87) |
| 10 | Shoot | 22.9 | 20.72 | 19.5 | 20.4 | 23.5 | 20.6 | 22.5 | 21.1 |
| | (cm) | (0.48) | (0.55) | (0.98) | (1.14) | (0.64) | (1.15) | (0.37) | (0.58) |
| | Root | 11.0 | 11.0 | 8.0 | 11.0 | 12.3 | 9.7 | 8.7 | 11.0 |
| | (cm) | (0.54) | (0.41) | (0.64) | (0.46) | (0.43) | (0.42) | (0.71) | (0.48) |
| | Total | 34.0 | 31.7 | 27.5 | 31.4 | 35.8 | 30.3 | 31.2 | 32.0 |
| | (cm) | (0.81) | (0.95) | (1.27) | (1.29) | (0.93) | (1.16) | (0.92) | (0.63) |

Note:
Concentration in the medium is in μl of oil/100 ml base medium.
Values in parenthesis indicate standard deviation.

Dry biomass weight/10 seedlings at the end of 5 days is reported in Table 12J below.

TABLE 12J
Effect of plant extracts on P. radiatus germination
activity

| | | | CROT | CROT | TVUL | TVUL | ZOFF | CAMA | |
|---|---|---|---|---|---|---|---|---|---|
| Extract/ | | Sesame | 28.2 | 62.66 | 32.83 | 66.84 | 55.4 | 26.2 | GGLAB |
| Bottle | | Oil | PLUS | MINUS | PLUS | MINUS | PLUS | PLUS | 27.43 |
| 0 | E.A. | 313 | | | | | | | |
| Control | (mg) | | | | | | | | |
| | Root | 71 | | | | | | | |
| | (mg) | | | | | | | | |
| | Shoot | 242 | | | | | | | |
| | (mg) | | | | | | | | |
| 1 | E.A. | 320 | 291 | 317 | 294 | 272 | 303 | 281 | 284 |
| | (mg) | | | | | | | | |
| | Root | 74 | 73 | 86 | 74 | 62 | 67 | 61 | 62 |
| | (mg) | | | | | | | | |
| | Shoot | 246 | 218 | 231 | 220 | 204 | 236 | 220 | 222 |
| | (mg) | | | | | | | | |
| 3 | E.A. | 290 | 279 | 316 | 298 | 307 | 311 | 297 | 244 |
| | (mg) | | | | | | | | |
| | Root | 72 | 67 | 82 | 80 | 67 | 73 | 75 | 72 |
| | (mg) | | | | | | | | |
| | Shoot | 218 | 212 | 234 | 218 | 240 | 238 | 222 | 172 |
| | (mg) | | | | | | | | |
| 10 | E.A. | 314 | 297 | 324 | 327 | 314 | 298 | 308 | 254 |
| | (mg) | | | | | | | | |
| | Root | 77 | 83 | 82 | 84 | 82 | 78 | 73 | 75 |
| | (mg) | | | | | | | | |
| | Shoot | 237 | 214 | 242 | 243 | 232 | 220 | 235 | 179 |
| | (mg) | | | | | | | | |

Note:
Concentration in the medium is in μl of oil/100 ml base medium.
E.A.—Embryo Axis (Root + Shoot)

Cyperous rotundus fractions (CROT 28.2 PLUS and CROT 62.66 MINUS) show differential activity. The PLUS fraction appears to promote shoot+root length at the lower dose; however, this isnot reflected in the biomass growth. The minus fraction does not siginificantly promote shoot+root growth and starts showing inhibitory effects at 10 μl of oil/100 ml base medium. Triticum vulagare fractions (TVUL 32.83 PLUS and TVUL 66.84 MINUS) promote shoot growth at 3 μl of oil/100 ml base medium and 10 μl of oil/100 ml base medium, respectively. This, in terms of starting wheat seeds, is approx. at 1 mg of wheat equivalent/100 ml base medium for the PLUS fraction and 6.7 mg of wheat equivalent/100 ml base medium for the minus fraction. Curcuma amamada (CAMA 26.2 PLUS) promotes particularly shoot growth at the lower concentrations of 1 and 3 μl of oil/100 ml base medium. Glycyrrhiza glabra (GGLAB 27.43) also promotes root+shoot growth at 1 and 3 μl of oil/100 ml base medium. However, this growth is accompanied by a reduction in biomass growth.

TABLE 12K
Effect of plant extracts on P. radiatus seed
germination activity

| Extract/ | | Sesame | A-100 | A-100 | B-100 | B-100 | C-100 | C-100 |
|---|---|---|---|---|---|---|---|---|
| Bottle | | Oil | PLUS | MINUS | PLUS | MINUS | PLUS | MINUS |
| 0 | Shoot | 16.4 | | | | | | |
| Control | (cm) | (0.69) | | | | | | |
| | Root | 8.13 | | | | | | |
| | (cm) | (0.55) | | | | | | |
| | Total | 24.53 | | | | | | |
| | (cm) | (0.86) | | | | | | |
| 1 | Shoot | 16.78 | 14.98 | 18.53 | 17.23 | 16.6 | 15.56 | 17.02 |
| | (cm) | (0.49) | (0.46) | (0.62) | (0.54) | (0.66) | (0.35) | (0.48) |
| | Root | 7.88 | 7.73 | 9.29 | 11.67 | 8.9 | 9.59 | 8.41 |
| | (cm) | (0.33) | (0.64) | (0.67) | (0.31 | (0.44) | (0.40) | (0.55) |
| | Total | 25.18 | 22.71 | 27.82 | 28.9 | 25.5 | 25.15 | 25.42 |
| | (cm) | (0.55) | (0.58) | (1.01) | (0.69) | (0.71) | (0.63) | (0.91) |
| 3 | Shoot | 17.51 | 16.84 | 17.32 | 16.23 | 17.25 | 16.84 | 16.29 |
| | (cm) | (0.57) | (0.16) | (0.34) | (0.42) | (0.40) | (0.54) | (0.51) |
| | Root | 8.4 | 7.43 | 8.79 | 10.92 | 9.21 | 9.12 | 7.69 |
| | (cm) | (0.39) | (0.39) | (0.64) | (0.47) | (0.45) | (0.42) | (0.43) |
| | Total | 25.91 | 24.27 | 26.11 | 27.15 | 26.46 | 25.96 | 23.88 |
| | (cm) | (0.62) | (0.48) | (0.99) | (0.83) | (0.75) | (0.58) | (0.43) |
| 10 | Shoot | 17.99 | 16.89 | 16.48 | 16.33 | 19.34 | 19.69 | 15.5 |
| | (cm) | (0.51) | (0.64) | (0.27) | (0.43) | (0.36) | (0.67) | (0.32) |
| | Root | 8.55 | 7.08 | 7.04 | 9.84 | 11.29 | 10.2 | 6.88 |
| | (cm) | (0.79) | (0.50) | (0.43) | (0.65) | (0.44) | (0.45) | (0.53) |
| | Total | 26.54 | 23.97 | 23.52 | 26.17 | 30.63 | 29.89 | 22.19 |
| | (cm) | (0.87) | (0.84) | (0.40) | (0.74) | (0.50) | (0.95) | (0.70) |

Note:
Extract/Bottle in the medium is in μl of oil/100 ml base medium.
Values in parenthesis indicate standard deviation.

In this set, different compositions show differential activity. The A-100 MINUS and B-100 PLUS both show a strong root and shoot promotional activity at the 1 μl of oil/100 ml level and the activity declines at higher concentrations. The B-100 MINUS and C-100 PLUS show strong promotional activity at the 10 μl of oil/100 ml level.

TABLE 12M
Effect of plant extracts on P. radiatus seed
germination activity

| | | | | RS- | | | | | |
|---|---|---|---|---|---|---|---|---|---|
| | | | | 10 | | | R-25 | R-25 | R-25 |
| Extract/ | | Sesame | R- | Leaf | R-100 | R-100 | 30 Min | 120 Min | 240 Min |
| Bottle | | Oil | 100 | Stem | (J + L/4) | (4J + L/8) | boiling | boiling | boiling |
| 0 | Shoot | 16.4 | | | | | | | |
| Control | (cm) | (0.69) | | | | | | | |
| | Root | 8.13 | | | | | | | |
| | (cm) | (0.55) | | | | | | | |
| | Total | 24.53 | | | | | | | |
| | (cm) | (0.86) | | | | | | | |
| 1 | Shoot | 16.78 | 16.4 | 18.84 | 15.76 | 16.24 | 16.71 | 12.93 | 15.51 |
| | (cm) | (0.49) | (0.41) | (0.51) | (0.61) | (0.37) | (0.44) | (0.46) | (0.30) |
| | Root | 7.88 | 8.63 | 10.07 | 5.32 | 5.96 | 8.53 | 4.98 | 8.73 |
| | (cm) | (0.33) | (0.27) | (0.76) | (0.53) | (0.64) | (0.39) | (0.52) | (0.31) |
| | Total | 25.18 | 25.08 | 28.91 | 21.08 | 22.2 | 25.24 | 17.91 | 24.24 |
| | (cm) | (0.55) | (0.32) | (0.47) | (0.65) | (0.81) | (0.48) | (0.54) | (0.44) |
| 3 | Shoot | 17.51 | 17.03 | 17.51 | 16.31 | 16.16 | 16.65 | 18.18 | 16.27 |
| | (cm) | (0.57) | (0.35) | (0.42) | (0.36) | (0.30) | (0.51) | (0.48) | (0.43) |
| | Root | 8.4 | 10.07 | 8.77 | 10.67 | 6.59 | 9.26 | 8.36 | 11.04 |
| | (cm) | (0.39) | (0.52) | (0.66) | (0.47) | (0.54) | (0.46) | (0.37) | (0.30) |
| | Total | 25.91 | 27.1 | 26.28 | 26.98 | 22.75 | 25.91 | 26.54 | 27.31 |
| | (cm) | (0.62) | (0.61) | (1.0) | (0.60) | (0.58) | (0.62) | (0.63) | (0.56) |
| 10 | Shoot | 17.99 | 18.27 | 12.57 | 15.34 | 16.11 | 18.51 | 16.5 | 19.23 |
| | (cm) | (0.51) | (0.64) | (0.34) | (0.29) | (0.21) | (0.82) | (0.50) | (0.36) |
| | Root | 8.55 | 11.2 | 7.41 | 8.62 | 5.48 | 10.87 | 8.61 | 11.39 |
| | (cm) | (0.79) | (0.37) | (0.33) | (0.47) | (0.53) | (0.33) | (0.36) | (0.35) |
| | Total | 26.54 | 29.47 | 19.98 | 23.96 | 21.69 | 29.38 | 25.11 | 30.62 |
| | (cm) | (0.87) | (0.77) | (0.55) | (0.69) | (0.67) | (0.66) | (0.51) | (0.52) |

Note:
Extract/Bottle in the medium is in μl of oil/100 ml base medium.
Values in parenthesis indicate standard deviation.

In this set, R-100, made as per a typical recipe of this invention is compared with R-100(4J+L/8) made using a typical traditional recipe deacribed in Sharangdharsamhita. The (4J+L/8) indicates that the juice taken for boiling was 4 times the oil and the kalka taken for boiling was ⅛^{th} the weight of oil. Although R-100 shows promotion, R-100(4J+L/8) shows inhibition when compared to both the plain control and sesame oil alone.

RS-10 made just from the leaf stem also shows a strong promotion. The last three sets of columns show that over a boiling time range of 30 minutes to 240 minutes, the compositions made as per the present invention show promotion compared to control.

TABLE 12O
Effect of plant extracts on P. radiatus seed
germination activity

| | | | | CLNG | | | |
|---|---|---|---|---|---|---|---|
| Extract/ | | Sesame | CLNG I | II | EJAM | RSER | VR |
| Bottle | | Oil | 33.75 | 26.47 | 34.16 | 29 | 25 |
| 0 | Shoot | 19.71 | | | | | |
| Control | (cm) | (1.06) | | | | | |
| | Root | 8.3 | | | | | |
| | (cm) | (0.48) | | | | | |
| | Total | 28.01 | | | | | |
| | (cm) | (1.32) | | | | | |
| 1 | Shoot | 22.37 | 13.42 | 20.53 | 17.53 | 19.96 | 19.96 |
| | (cm) | (0.55) | (0.63) | (1.43) | (1.24) | (0.39) | (0.45) |
| | Root | 7.9 | 4.29 | 8.26 | 5.93 | 8.12 | 7.54 |
| | (cm) | (0.76) | (0.70) | (0.46) | (1.14) | (0.55) | (0.81) |
| | Total | 30.32 | 17.17 | 28.79 | 23.4 | 27.45 | 24.5 |
| | (cm) | (0.78) | (0.91) | (1.51) | (1.83) | (1.5) | (1.13) |
| 3 | Shoot | 22.22 | 17.0 | 22.83 | 20.12 | 22.25 | 20.54 |
| | (cm) | (1.71) | (0.71) | (0.54) | (1.36) | (0.37) | (0.96) |
| | Root | 9.31 | 7.9 | 10.14 | 10.03 | 11.42 | 10.29 |
| | (cm) | (0.50) | (0.27) | (0.62) | (0.61) | (0.56) | (0.51) |
| | Total | 31.53 | 24.90 | 32.71 | 30.20 | 33.7 | 30.83 |
| | (cm) | (1.9) | (0.75) | (1.05) | (2.0) | (0.93) | (1.22) |
| 10 | Shoot | 22.33 | 21.92 | 23.1 | 21.63 | 18.52 | 20.99 |
| | (cm) | (1.19) | (0.30) | (0.56) | (2.1) | (1.38) | (0.57) |
| | Root | 10.61 | 6.53 | 10.5 | 10.13 | 7.73 | 8.48 |
| | (cm) | (0.55) | (0.38) | (0.52) | (0.34) | (0.50) | (1.37) |
| | Total | 32.94 | 28.45 | 33.60 | 31.76 | 26.25 | 29.47 |
| | (cm) | (1.42) | (0.60) | (0.74) | (1.47) | (1.78) | (1.44) |

Note:
Extract/Bottle in the medium is in μl of oil/100 ml base medium.
Values in parenthesis indicate standard deviation.

Compositions of this set do not show significant promotion compared to plain sesame oil. However, they show a strong promotion effect in mustard seed germination (see Tables 112 C). Absence of promotion in the P. radiatus seed germination and promotion in mustard seed germination is an illustration of how the extracts from different biomass made as per the present invention can be used to provide selective effects in different target biomass. Such selectivity greatly extends the utility of the present invention.

TABLE 12Q
Effect of plant extracts on P. radiatus seed
germination activity

| Extract/ | | Sesame | | LUST- | BAMO- | PRAD- | R-100 | |
|---|---|---|---|---|---|---|---|---|
| Bottle | | Oil | N-50 | 35.71 | 55.59 | 32.2 | Coconut | CCT |
| 0 | Shoot | 20.40 | | | | | | |
| Control | (cm) | (1.35) | | | | | | |
| | Root | 6.00 | | | | | | |
| | (cm) | (0.71) | | | | | | |
| | Total | 26.40 | | | | | | |
| | (cm) | (1.43) | | | | | | |
| 1 | Shoot | 20.3 | 21.5 | 22.05 | 20.15 | 22.05 | 20.5 | 21.2 |
| | (cm) | (1.57) | (1.22) | (2.14) | (1.35) | (1.3) | 1.15) | (1.44) |
| | Root | 6.30 | 7.10 | 6.9 | 6.25 | 7.5 | 7.05 | 7.20 |
| | (cm) | (0.98) | (0.70) | (1.49) | (0.82) | (1.08) | (0.90) | (1.4) |
| | Total | 26.60 | 28.60 | 28.95 | 26.40 | 29.55 | 27.55 | 28.40 |
| | (cm) | (2.94) | (1.82) | (2.86) | (1.20) | (1.98) | (1.52) | (2.51) |
| 3 | Shoot | 22.20 | 20.55 | 21.30 | 22.60 | 21.80 | 20.50 | 22.84 |
| | (cm) | (1.27) | (1.40) | (1.34) | (1.35) | (0.98) | (1.03) | (1.18) |
| | Root | 6.6 | 6.75 | 6.8 | 5.85 | 6.80 | 6.80 | 7.61 |
| | (cm) | (1.02) | (1.01) | (1.14) | (1.08) | (0.71) | (0.95) | (0.81) |
| | Total | 28.80 | 27.30 | 28.10 | 28.45 | 28.60 | 27.30 | 30.45 |
| | (cm) | (1.95) | (1.74) | (1.45) | (1.83) | (1.26) | (1.75) | (1.35) |
| 10 | Shoot | 22.75 | 20.40 | 20.85 | 20.40 | 20.25 | 21.45 | 22.45 |
| | (cm) | (0.95) | (1.07) | (1.42) | (1.22) | (1.27) | (0.98) | (1.28) |
| | Root | 5.45 | 6.60 | 6.45 | 5.05 | 6.40 | 6.75 | 7.45 |
| | (cm) | (1.09) | (1.20) | (0.60) | (1.01) | (0.97) | (0.68) | (1.12) |
| | Total | 28.20 | 27.0 | 27.30 | 25.45 | 26.65 | 28.20 | 29.90 |
| | (cm) | (1.53) | (1.76) | (1.72) | (1.83) | (1.40) | (1.14) | (2.01) |

Note:
Extract/Bottle in the medium is in μl of oil/100 ml base medium.
Values in parenthesis indicate standard deviation.

TABLE 12S
Effect of plant extracts on P. radiatus seed
germination activity

| Extract/ | | Sesame | TICO | CACO | ACEP | PILO | OSA-T | WISO |
|---|---|---|---|---|---|---|---|---|
| Bottle | | Oil | 35.51 | 37.5 | 74.3 | 36.02 | 48.88 | 38.82 |
| 0 | Shoot | 20.61 | | | | | | |
| Control | (cm) | (1.14) | | | | | | |
| | Root | 6.85 | | | | | | |
| | (cm) | (1.05) | | | | | | |
| | Total | 27.46 | | | | | | |
| | (cm) | (1.24) | | | | | | |
| 1 | Shoot | 21.48 | 22.54 | 21.60 | 22.97 | 22.11 | 22.10 | 23.14 |
| | (cm) | (1.18) | (1.04) | (0.63) | (0.93) | (1.13) | (1.01) | (0.90) |
| | Root | 6.76 | 7.41 | 7.15 | 6.87 | 6.80 | 8.5 | 7.75 |
| | (cm) | (1.22) | (0.75) | (0.52) | (0.68) | (0.66) | (0.65) | (0.87) |
| | Total | 28.24 | 29.95 | 28.75 | 29.84 | 28.91 | 30.60 | 30.89 |
| | (cm) | (1.72) | (1.18) | (0.92) | (0.95) | (1.65) | (0.96) | (1.17) |
| 3 | Shoot | 21.54 | 22.83 | 22.29 | 22.61 | 22.25 | 22.31 | 23.28 |
| | (cm) | (1.22) | (0.83) | (1.31) | (0.66) | (0.98) | (1.1) | (0.86) |
| | Root | 6.8 | 7.68 | 7.26 | 7.53 | 7.97 | 8.39 | 7.79 |
| | (cm) | (0.95) | (0.42) | (0.83) | (0.45) | (0.56) | (0.38) | (0.78) |
| | Total | 28.34 | 30.51 | 29.55 | 30.14 | 30.22 | 30.70 | 31.07 |
| | (cm) | (1.89) | (0.92) | (1.32) | (0.79) | (1.22) | (1.28) | (1.15) |
| 10 | Shoot | 21.21 | 22.03 | 22.58 | 22.36 | 20.75 | 22.91 | 21.65 |
| | (cm) | (0.91) | (1.02) | (0.96) | (0.96) | (0.90) | (0.88) | (0.96) |
| | Root | 7.24 | 8.17 | 7.66 | 8.26 | 7.22 | 7.90 | 7.40 |
| | (cm) | (0.75) | (0.70) | (0.78) | (0.87) | (0.78) | (0.81) | (0.87) |
| | Total | 28.45 | 30.20 | 30.24 | 30.62 | 27.97 | 30.81 | 29.05 |
| | (cm) | (1.26) | (1.55) | (0.99) | (1.16) | (0.88) | (1.21) | (1.16) |

Note:
Extract/Bottle in the medium is in μl of oil/100 ml base medium.
Values in parenthesis indicate standard deviation.

A particular composition may promote only the root length or shoot length or both. Also, each composition may show a maximum effect at one particular dose level and above that dose level the effect may decline again. Thus, the compostions in this set are better than sesame oil alone for either shoot or root growth and at some particular dose level. The improvements are marginal in many cases which mean that the difference in the mean values is greater by an average of the standard deviation. However, when compared to water control, several of the extracts do show a stronger promotion.

TABLE 12U
Effect of biomass extracts on P. radiatus seed
germination activity

| Extract/ | | Sesame | ASAT | CUCY | MUSH-A | MOCH | PRWN | KPMS |
|---|---|---|---|---|---|---|---|---|
| Bottle | | Oil | 68.68 | 36.67 | 98.36 | 77.16 | 67.88 | 51.19 |
| 0 | Shoot | 20.82 | | | | | | |
| Control | (cm) | (1.12) | | | | | | |
| | Root | 6.96 | | | | | | |
| | (cm) | (1.1) | | | | | | |
| | Total | 27.78 | | | | | | |
| | (cm) | (1.26) | | | | | | |
| 1 | Shoot | 21.47 | 21.64 | 21.85 | 21.98 | 22.65 | 22.12 | 22.25 |
| | (cm) | (1.16) | (1.14) | (0.83) | (1.12) | (0.90) | (1.05) | (1.13) |
| | Root | 6.67 | 7.24 | 7.25 | 7.15 | 7.78 | 7.86 | 6.80 |
| | (cm) | (1.02) | (0.78) | (0.82) | (0.92) | (0.87) | (0.85) | (0.96) |
| | Total | 28.14 | 28.88 | 29.10 | 29.13 | 30.43 | 29.98 | 29.05 |
| | (cm) | (1.22) | (1.18) | (1.21) | (1.06) | (1.17) | (1.16) | (1.45) |
| 3 | Shoot | 21.74 | 22.23 | 22.21 | 22.96 | 22.98 | 22.72 | 22.25 |
| | (cm) | (1.02) | (0.84) | (1.01) | (0.82) | (0.96) | (1.12) | (0.98) |
| | Root | 6.78 | 7.56 | 7.36 | 7.82 | 7.85 | 7.62 | 8.02 |
| | (cm) | (0.98) | (0.72) | (0.83) | (0.92) | (0.88) | (0.68) | (0.56) |
| | Total | 28.52 | 29.79 | 29.57 | 30.78 | 30.83 | 30.34 | 30.27 |
| | (cm) | (1.42) | (1.02) | (1.2) | (1.26) | (1.25) | (1.22) | (1.22) |
| 10 | Shoot | 21.23 | 22.13 | 22.88 | 23.16 | 22.15 | 23.11 | 22.96 |
| | (cm) | (0.85) | (1.12) | (1.26) | (1.02) | (0.92) | (0.88) | (0.90) |
| | Root | 7.14 | 8.24 | 7.72 | 8.26 | 7.85 | 8.02 | 8.11 |
| | (cm) | (0.78) | (0.82) | (1.05) | (0.94) | (0.85) | (0.81) | (1.02) |
| | Total | 28.37 | 30.37 | 30.60 | 31.42 | 30.0 | 31.13 | 31.07 |
| | (cm) | (1.14) | (1.24) | (1.41) | (1.06) | (1.06) | (1.01) | (1.25) |

Note:
Extract/Bottle in the medium is in μl of oil/100 ml base medium.
Values in parenthesis indicate standard deviation.

The two non-plant biomass extracts from Mushroom (MUSH-A 98.36) and Prawn (PRWN-67.88) show promotion compared to both plain control and same oil.

TABLE 12W
Effect of biomass extracts on P. radiatus seed germination activity

| Extract/ | | Sesame | ASRA | GLMX | YBD | PE- | C- | PINI | GYSY |
|---|---|---|---|---|---|---|---|---|---|
| Bottle | | Oil | 36.16 | 27.65 | 47.55 | 100 | 100 | 26.4 | 26.4 |
| 0 | Shoot | 20.62 | | | | | | | |
| Control | (cm) | (0.86) | | | | | | | |
| | Root | 9.30 | | | | | | | |
| | (cm) | (0.97) | | | | | | | |
| | Total | 29.92 | | | | | | | |
| | (cm) | (0.61) | | | | | | | |
| 1 | Shoot | 20.77 | 21.35 | 20.88 | 20.69 | 20.77 | 20.93 | 20.50 | 21.55 |
| | (cm) | (0.75) | (0.94) | (0.85) | (0.73) | (0.86) | (0.83) | (0.47) | (0.96) |
| | Root | 9.83 | 12.03 | 10.04 | 9.68 | 10.93 | 10.81 | 10.25 | 10.41 |
| | (cm) | (0.78) | (0.74) | (0.42) | (0.48) | (0.84) | (0.490 | (0.79) | (0.68) |
| | Total | 30.60 | 33.38 | 30.92 | 30.37 | 31.70 | 31.74 | 30.75 | 31.96 |
| | (cm) | (0.87) | (1.39) | (1.03) | (0.87) | (1.07) | (0.84) | (1.03) | (1.12) |
| 3 | Shoot | 21.80 | 20.30 | 21.33 | 20.98 | 22.11 | 21.11 | 20.20 | 21.62 |
| | (cm) | (0.85) | (1.16) | (0.83) | (0.62) | (0.90) | (0.64) | (0.59) | (0.84) |
| | Root | 10.58 | 10.62 | 10.97 | 10.09 | 11.24 | 11.04 | 11.40 | 11.19 |
| | (cm) | (0.81) | (0.69) | (0.66) | (0.81) | (0.52) | (0.67) | (0.84) | (0.88) |
| | Total | 31.66 | 30.92 | 32.30 | 31.07 | 33.35 | 32.25 | 31.60 | 32.82 |
| | (cm) | (0.92) | (1.03) | (1.11) | (1.21) | (1.06) | (1.12) | (0.88) | (1.46) |
| 10 | Shoot | 20.80 | 20.20 | 21.56 | 21.05 | 20.30 | 22.00 | 19.90 | 21.88 |
| | (cm) | (0.78) | (1.09) | (0.75) | (0.86) | (0.92) | (0.85) | (0.70 | (0.97) |
| | Root | 11.14 | 9.75 | 12.15 | 10.57 | 11.36 | 11.95 | 11.95 | 12.02 |
| | (cm) | (0.71) | (0.58) | (0.60) | (0.50) | (0.65) | (0.72) | (1.07) | (0.97) |
| | Total | 31.94 | 29.95 | 33.71 | 31.62 | 31.66 | 33.95 | 30.85 | 33.90 |
| | (cm) | (1.30) | (1.35) | (1.04) | (1.00) | (0.68) | (0.93) | (1.27) | (1.41) |

Note:
Extract/Bottle in the medium is in μl of oil/100 ml base medium.
Values in parenthesis indicate standard deviation.

Asperagus racemosus (ASRA-36.16) shows strong promotion even at 1 μl of oil/100 ml. Others also show moderate activity compared to control and also sesame oil. Yeast extract also shows moderate activity against control.

### Example 17B

### Effect of Various Angiosperm and Gymnosperm Plant Extracts and Non-Plant Biomass Extracts on Germination of Mustard (Brassica nigra)

Agar (0.8 wt. % agar in distilled water) was digested in water bath to get transperant medium and 100 ml of this solution (base medium) was poured in 300 ml culture bottles. Different doses of oil extracts of various Angiosperm-monocotyledon, Angiosperm-dicotyledon and Gymnosperm plants, non-plant biomass or plain base sesame oil were added to each bottle. Bottles were sterilized at 15 lbs for 20 mnutes in an autoclave. Surface sterilized 10 seeds of Phaseolus radiatus were added to each bottle and germinated in dark for 5 days. On the 5^{th} day after initiation of experiment, values of shoot Tenth, root lenth, and dry weight of biomass (dried in an oven at 70-80 ° C. for constant dry weight) were measured for 10 seedlings. Mean values are reported in Table 112A-112K, 112M, 112O, 112Q, 112S, 112U, 112W and 112Y. Several extracts of plants and their fractions and non-plant biomass promoted rooting, shooting and biomass mobilization at low doses.

TABLE 112A
Effect of plant extracts on Brassica nigra seed germination activity

| | | | | | | | | | ZOFF- |
|---|---|---|---|---|---|---|---|---|---|
| Extract/ | | Sesame | GGLAB | MPRU | AZIN- | B-100 | TRIGF- | A-100 | 100 |
| Bottle | | Oil | 27.43 | 27.1 | C-29 | PLUS | 29.62 | PLUS | MINUS |
| 0 | Shoot | 8.56 | | | | | | | |
| Control | (cm) | (0.34) | | | | | | | |
| | Root | 5.29 | | | | | | | |
| | (cm) | (0.37) | | | | | | | |
| | Total | 13.85 | | | | | | | |
| | (cm) | (0.32) | | | | | | | |
| 1 | Shoot | 8.12 | 7.74 | 7.86 | 9.42 | 12.0 | 8.48 | 8.67 | 10.50 |
| | (cm) | (0.51) | (0.51) | (0.30) | (0.58) | (0.41) | (0.43) | (0.54) | (0.55) |
| | Root | 4.82 | 4.87 | 7.15 | 6.55 | 7.18 | 4.95 | 5.74 | 3.72 |
| | (cm) | (0.34) | (0.37) | (0.51) | (0.41) | (1.02) | (0.53) | (0.66) | (0.68) |
| | Total | 12.94 | 12.61 | 15.01 | 15.97 | 19.18 | 13.43 | 14.41 | 14.22 |
| | (cm) | (0.63) | (1.62) | (0.58) | (0.63) | (1.27) | (0.72) | (1.01) | (0.89) |
| 3 | Shoot | 8.69 | 8.26 | 8.97 | 10.25 | 9.49 | 10.86 | 9.19 | 11.73 |
| | (cm) | (0.44) | (0.62) | (0.59) | (0.66) | (0.38) | (0.43) | (0.58) | (0.54) |
| | Root | 5.5 | 5.59 | 5.3 | 6.92 | 5.62 | 8.75 | 8.76 | 6.49 |
| | (cm) | (0.38) | (0.30) | (0.44) | (0.64) | (0.52) | (0.24) | (0.56) | (0.62) |
| | Total | 14.19 | 13.85 | 14.28 | 17.17 | 15.1 | 19.61 | 17.95 | 17.72 |
| | (cm) | (0.47) | (0.77) | (0.66) | (1.04) | (0.90) | (0.54) | (0.71) | (0.88) |
| 10 | Shoot | 8.92 | 9.35 | 12.04 | 7.44 | 7.8 | 6.16 | 9.68 | 10.98 |
| | (cm) | (0.44) | (0.49) | (0.71) | (0.52) | (0.31) | (58) | (0.56) | (0.31) |
| | Root | 7.64 | 7.37 | 10.29 | 5.32 | 5.39 | 9.55 | 5.45 | 5.20 |
| | (cm) | (0.36) | (0.23) | (0.58) | (0.69) | (0.47) | (0.38) | (0.71) | (0.59) |
| | Total | 16.56 | 16.72 | 22.33 | 12.76 | 13.19 | 15.71 | 15.13 | 16.18 |
| | (cm) | (0.67) | (0.49) | (1.19) | (0.66) | (0.43) | (0.61) | (0.51) | (0.65) |

Note:
Extract/Bottle in the medium is in μl of oil/100 ml base medium.
Values in parenthesis indicate standard deviation.

Except for G. glabra (GGLAB-27.43), all other extracts of this set are good promoters of root+shoot growth. However, G. glabra (GGLAB-27.43) is a promoter of mustard seed germination.

Dry biomass weight at the end of 5 days is reported in Table 112B below.

TABLE 112B
Effect of plant fractions on Brassica nigra germination activity

| | | | | | | | | | ZOFF- |
|---|---|---|---|---|---|---|---|---|---|
| Extract/ | | Sesame | GGLAB | MPRU | AZIN- | B-100 | TRIGF- | A-100 | 100 |
| Bottle | | Oil | 27.43 | 27.1 | C-29 | PLUS | 29.62 | PLUS | MINUS |
| 0 | E.A.(mg) | 47 | | | | | | | |
| Control | Root(mg) | 15 | | | | | | | |
| | Shoot(mg) | 32 | | | | | | | |
| 1 | E.A.(mg) | 49 | 52 | 56 | 58 | 60 | 42 | 42 | 45 |
| | Root(mg) | 18 | 22 | 23 | 23 | 28 | 18 | 19 | 19 |
| | Shoot(mg) | 31 | 30 | 33 | 25 | 32 | 24 | 23 | 26 |
| 3 | E.A.(mg) | 52 | 57 | 65 | 53 | 54 | 50 | 50 | 63 |
| | Root(mg) | 18 | 25 | 27 | 25 | 25 | 23 | 22 | 25 |
| | Shoot(mg) | 34 | 32 | 38 | 28 | 29 | 27 | 28 | 38 |
| 10 | E.A.(mg) | 55 | 59 | 67 | 37 | 41 | 41 | 43 | 58 |
| | Root(mg) | 20 | 24 | 29 | 15 | 18 | 19 | 20 | 25 |
| | Shoot(mg) | 35 | 35 | 38 | 22 | 23 | 23 | 23 | 33 |

Note:
Extract/Bottle in the medium is in μl of oil/100 ml base medium.
Cot. - Cotyledon;
E.A. - Embryo Axis (Root + Shoot);
Dry weight/10 seedlings

All extracts show biomass promotion compared to control and some of them show promotion compared to sesame oil as well.

TABLE 112C
Effect of plant extracts on Brassica nigra seed germination activity

| | | | CLNG | CLNG- | | | |
|---|---|---|---|---|---|---|---|
| Extract/ | | Sesame | I- | II | EJAM- | RSER- | VR- |
| Bottle | | Oil | 33.75 | 26.47 | 34.16 | 29 | 25 |
| 0 | Shoot | 7.85 | | | | | |
| Control | (cm) | (0.41) | | | | | |
| | Root | 4.84 | | | | | |
| | (cm) | (0.56) | | | | | |
| | Total | 12.69 | | | | | |
| | (cm) | (0.47) | | | | | |
| 1 | Shoot | 8.77 | 10.36 | 10.3 | 8.69 | 10.61 | 10.47 |
| | (cm) | (0.52) | (0.31) | (0.40) | (0.77) | (0.48) | (0.29) |
| | Root | 4.74 | 6.99 | 8.86 | 7.79 | 9.14 | 8.44 |
| | (cm) | (0.52) | (0.54) | (0.42) | (0.49) | (0.74) | (0.40) |
| | Total | 13.51 | 17.35 | 20.22 | 16.48 | 19.75 | 18.91 |
| | (cm) | (0.60) | (0.61) | (0.56) | (0.78) | (0.90) | (0.47) |
| 3 | Shoot | 9.95 | 11.22 | 11.51 | 8.73 | 9.22 | 10.46 |
| | (cm) | (0.51) | (0.25) | (0.72) | (0.47) | (0.47) | (0.25) |
| | Root | 5.4 | 8.74 | 10.03 | 9.36 | 8.01 | 8.75 |
| | (cm) | (0.61) | (0.46) | (0.47) | (0.44) | (1.23) | (0.54) |
| | Total | 15.35 | 19.96 | 21.54 | 18.09 | 17.2 | 19.21 |
| | (cm) | (0.98) | (0.37) | (1.1) | (0.51) | (1.7) | (0.64) |
| 10 | Shoot | 10.15 | 11.75 | 11.99 | 9.91 | 8.55 | 8.27 |
| | (cm) | (0.39) | (0.24) | (0.59) | (0.45) | (0.37) | (0.68) |
| | Root | 7.65 | 9.55 | 10.71 | 8.74 | 6.84 | 6.85 |
| | (cm) | (0.54) | (0.57) | (0.39) | (0.67) | (0.62) | (0.62) |
| | Total | 17.8 | 21.3 | 22.7 | 18.65 | 15.39 | 15.12 |
| | (cm) | (0.62) | (0.81) | (0.85) | (0.93) | (0.61) | (0.99) |

Note:
Extract/Bottle in the medium is in μl of oil/100 ml base medium.
Values in parenthesis indicate standard deviation.

All extracts of this set show strong promotion.

Dry biomass weight at the end of 5 days is reported in Table 112D below.

TABLE 112D
Effect of plant fractions on Brassica nigra germination activity

| Extract/ | | Sesame | CLNG I- | CLNG-II | | | |
|---|---|---|---|---|---|---|---|
| Bottle | | Oil | 33.75 | 26.47 | EJAM-34.16 | RSER-29 | VR-25 |
| 0 | E.A.(mg) | 52 | | | | | |
| Control | Root(mg) | 18 | | | | | |
| | Shoot(mg) | 34 | | | | | |
| 1 | E.A.(mg) | 54 | 50 | 57 | 44 | 45 | 52 |
| | Root(mg) | 18 | 18 | 22 | 16 | 17 | 20 |
| | Shoot(mg) | 36 | 32 | 35 | 28 | 28 | 32 |
| 3 | E.A.(mg) | 59 | 57 | 62 | 45 | 50 | 57 |
| | Root(mg) | 22 | 20 | 25 | 17 | 19 | 22 |
| | Shoot(mg) | 37 | 37 | 37 | 28 | 31 | 35 |
| 10 | E.A.(mg) | 53 | 62 | 66 | 52 | 47 | 49 |
| | Root(mg) | 26 | 23 | 27 | 21 | 21 | 18 |
| | Shoot(mg) | 27 | 39 | 39 | 31 | 26 | 31 |

Note:
Extract/Bottle in the medium is in μl of oil/100 ml base medium.
Cot. - Cotyledon;
E.A. - Embryo Axis (Root + Shoot);
Dry weight/10 seedlings

All extracts show biomass growth promotion compared to water. The CLNG-I and CLING-II extracts continue to show higher growth even at the highest concention.

TABLE 112E
Effect of plant extracts on Brassica nigra seed germination activity

| Extract/ | | Sesame | PRAD | BAMO | R-100 | LUST | |
|---|---|---|---|---|---|---|---|
| Bottle | | Oil | 32.2 | 55.59 | Coconut | 35.71 | N 50 |
| 0 | Shoot | 7.22 | | | | | |
| Control | (cm) | (0.65) | | | | | |
| | Root | 5.76 | | | | | |
| | (cm) | (0.87) | | | | | |
| | Total | 12.98 | | | | | |
| | (cm) | (1.09) | | | | | |
| 1 | Shoot | 7.41 | 7.35 | 7.56 | 8.94 | 7.79 | 9.18 |
| | (cm) | (0.84) | (0.95) | (0.94) | (1.77) | (0.99) | (0.81) |
| | Root | 6.01 | 5.90 | 5.91 | 7.49 | 6.53 | 6.77 |
| | (cm) | (0.71) | (0.73) | (0.91) | (0.94) | (1.01) | (0.74) |
| | Total | 13.42 | 13.25 | 13.47 | 16.43 | 14.32 | 15.95 |
| | (cm) | (1.36) | (1.12) | (1.23) | (1.79) | (1.58) | (0.94) |
| 3 | Shoot | 7.88 | 7.43 | 7.85 | 9.18 | 7.95 | 8.83 |
| | (cm) | (0.65) | (0.74) | (0.99) | (0.90) | (0.55) | (0.91) |
| | Root | 6.1 | 6.53 | 6.38 | 7.59 | 6.05 | 7.63 |
| | (cm) | (0.39) | (1.05) | (0.90) | (1.07) | (0.55) | (0.53) |
| | Total | 13.98 | 13.96 | 14.23 | 16.77 | 14.0 | 16.46 |
| | (cm) | (0.73) | (1.4) | (1.59) | (1.36) | (0.98) | (0.81) |
| 10 | Shoot | 7.95 | 6.3 | 7.76 | 6.84 | 8.25 | 7.94 |
| | (cm) | (0.60) | (0.88) | (1.1) | (0.83) | (0.82) | (0.53) |
| | Root | 6.41 | 4.44 | 5.16 | 7.19 | 5.37 | 6.92 |
| | (cm) | (0.43) | (0.57) | (1.06) | (1.11) | (0.50) | (0.66) |
| | Total | 14.36 | 10.74 | 12.92 | 14.03 | 13.62 | 14.86 |
| | (cm) | (0.88) | (1.25) | (1.96) | (1.51) | (0.87) | (0.98 |

Note:
Extract/Bottle in the medium is in μl of oil/100 ml base medium.
Values in parenthesis indicate standard deviation.

Flaxseed (LUST-35.71), Neemleaf (N-50) and Kalanchoe boiled in coconut oil show strong promotion.

TABLE 112G
Effect of plant extracts on Brassica nigra seed germination activity

| | | Sesame | TICO | CACO | ACEP | PILO | OSA-T | WISO |
|---|---|---|---|---|---|---|---|---|
| Extract/Bottle | | Oil | 35.51 | 37.5 | 74.3 | 36.02 | 48.88 | 38.82 |
| 0 | Shoot | 7.53 | | | | | | |
| Control | (cm) | (1.0) | | | | | | |
| | Root | 6.35 | | | | | | |
| | (cm) | (0.97) | | | | | | |
| | Total | 13.88 | | | | | | |
| | (cm) | (1.32) | | | | | | |
| 1 | Shoot | 7.59 | 7.29 | 7.9 | 7.44 | 7.37 | 7.78 | 7.58 |
| | (cm) | (0.86) | (1.41) | (0.99) | (0.91) | (1.0) | (0.58) | (0.91) |
| | Root | 6.45 | 5.44 | 5.28 | 6.53 | 6.44 | 7.21 | 6.29 |
| | (cm) | (0.54) | (0.88) | (0.46) | (0.73) | (0.68) | (0.65) | (0.52) |
| | Total | 14.04 | 12.73 | 13.18 | 13.97 | 13.81 | 14.99 | 13.87 |
| | (cm) | (1.16) | (1.63) | (1.05) | (1.11) | (1.16) | (1.01) | (1.07) |
| 3 | Shoot | 7.84 | 8.90 | 8.02 | 7.64 | 7.5 | 10.5 | 6.52 |
| | (cm) | (0.90) | (0.77) | (0.83) | (0.46) | (0.56) | (0.57) | (0.52) |
| | Root | 6.84 | 6.63 | 6.91 | 8.05 | 6.57 | 6.1 | 5.61 |
| | (cm) | (0.67) | (0.72) | (0.64) | (0.89) | (0.52) | (0.27) | (0.86) |
| | Total | 14.68 | 15.53 | 14.93 | 15.69 | 14.07 | 17.39 | 12.12 |
| | (cm) | (1.36) | (1.13) | (1.24) | (1.24) | (0.64) | (0.65) | (1.11) |
| 10 | Shoot | 8.48 | 7.98 | 7.69 | 8.21 | 8.42 | 8.44 | 6.26 |
| | (cm) | (0.99) | (0.88) | (0.78) | (0.64) | (0.96) | (1.09) | (0.30) |
| | Root | 7.00 | 6.7 | 6.59 | 6.82 | 7.83 | 6.56 | 5.27 |
| | (cm) | (0.95) | (0.70) | (1.06) | (0.38) | (1.1) | (0.58) | (0.49) |
| | Total | 15.48 | 14.68 | 14.28 | 15.03 | 16.25 | 15.0 | 11.53 |
| | (cm) | (1.46) | (1.36) | (1.21) | (0.61) | (1.44) | (1.38)) | (0.43) |

Note:
Extract/Bottle in the medium is in μl of oil/100 ml base medium.
Values in parenthesis indicate standard deviation.

TABLE 112I
Effect of biomass extracts on Brassica nigra seed germination activity

| | | Sesame | ASAT | CUCY | MUSH- | MOCH | PRWN | KPMS |
|---|---|---|---|---|---|---|---|---|
| Extract/Bottle | | Oil | 68.68 | 36.67 | A-98.36 | 77.16 | 67.88 | 51.19 |
| 0 | Shoot | 7.63 | | | | | | |
| Control | (cm) | (1.00) | | | | | | |
| | Root | 6.45 | | | | | | |
| | (cm) | (1.02) | | | | | | |
| | Total | 14.08 | | | | | | |
| | (cm) | (1.32) | | | | | | |
| 1 | Shoot | 8.02 | 7.89 | 8.09 | 7.98 | 8.12 | 8.11 | 7.99 |
| | (cm) | (0.82) | (1.21) | (0.92) | (0.98) | (0.88) | (0.78) | (0.98) |
| | Root | 6.55 | 6.54 | 7.38 | 7.03 | 6.54 | 7.28 | 7.29 |
| | (cm) | (0.64) | (0.82) | (0.96) | (0.83) | (0/78) | (0.85) | (0.72) |
| | Total | 14.57 | 14.43 | 15.47 | 15.03 | 14.66 | 15.39 | 15.28 |
| | (cm) | (1.16) | (1.13) | (1.25) | (1.12) | (1.15) | (1.02) | (1.27) |
| 3 | Shoot | 7.94 | 8.92 | 9.22 | 8.94 | 8.5 | 10.52 | 9.82 |
| | (cm) | (0.92) | (0.77) | (0.83) | (0.86) | (0.56) | (0.57) | (0.52) |
| | Root | 6.85 | 6.63 | 7.02 | 7.02 | 7.57 | 6.10 | 6.82 |
| | (cm) | (0.97) | (0.72) | (0.64) | (0.82) | (0.52) | (0.87) | (0.82) |
| | Total | 14.79 | 15.55 | 16.24 | 15.96 | 16.07 | 16.62 | 16.64 |
| | (cm) | (1.22) | (1.13) | (1.21) | (1.04) | (1.22) | (1.02) | (0.98) |
| 10 | Shoot | 8.42 | 8.68 | 8.21 | 8.02 | 8.56 | 8.92 | 7.82 |
| | (cm) | (1.09) | (0.84) | (0.85) | (0.64) | (0.96) | (1.02) | (0.65) |
| | Root | 7.05 | 7.17 | 7.12 | 6.92 | 7.36 | 7.12 | 6.32 |
| | (cm) | (0.90) | (0.72) | (0.92) | (0.78) | (1.1) | (0.62) | (0.89) |
| | Total | 15.47 | 15.85 | 15.33 | 14.94 | 15.92 | 16.04 | 14.40 |
| | (cm) | (1.26) | (1.16) | (1.02) | (1.0) | (1.44) | (1.18) | (1.01) |

Note:
Extract/Bottle in the medium is in μl of oil/100 ml base medium.
Values in parenthesis indicate standard deviation.

Several extracts including the non-plant biomass extracts of mushroom and prawn show activity compared to control.

TABLE 112K
Effect of plant extracts on Brassica nigra seed germination activity

| Extract/ | | Sesame | C-100 | C-100 | | R-100 |
|---|---|---|---|---|---|---|
| Bottle | | Oil | PLUS | MINUS | R-100 | (J + L/4) |
| 0 | Shoot | 7.48 | | | | |
| Control | (cm) | (1.02) | | | | |
| | Root | 6.32 | | | | |
| | (cm) | (0.92) | | | | |
| | Total | 13.8 | | | | |
| | (cm) | (1.01) | | | | |
| 1 | Shoot | 7.84 | 7.98 | 8.02 | 8.14 | 8.08 |
| | (cm) | (0.82) | (0.85) | (0.92) | (0.78) | (0.82) |
| | Root | 6.58 | 7.01 | 7.28 | 7.32 | 6.95 |
| | (cm) | (0.64) | (0.88) | (0.98) | (0.82) | (0.78) |
| | Total | 14.42 | 14.99 | 15.30 | 15.46 | 15.03 |
| | (cm) | (1.16) | (1.18) | (1.02) | (1.14) | (1.18) |
| 3 | Shoot | 8.02 | 8.42 | 8.52 | 9.42 | 8.5 |
| | (cm) | (0.98) | (0.92) | (0.83) | (0.98) | (0.66) |
| | Root | 6.86 | 7.24 | 7.42 | 7.25 | 7.58 |
| | (cm) | (0.92) | (0.48) | (0.84) | (0.88) | (0.72) |
| | Total | 14.88 | 15.66 | 15.94 | 16.67 | 16.08 |
| | (cm) | (1.12) | (1.02) | (1.04) | (1.02) | (1/12) |
| 10 | Shoot | 8.34 | 8.88 | 8.75 | 9.64 | 8.92 |
| | (cm) | (1.0) | (0.72) | (0.88) | (1.02) | (0.96) |
| | Root | 6.98 | 7.56 | 7.82 | 7.72 | 7.66 |
| | (cm) | (0.94) | (0.78) | (1.02) | (0.82) | (1.16) |
| | Total | 15.32 | 16.44 | 16.57 | 17.36 | 16.58 |
| | (cm) | (1.02) | (1.08) | (1.22) | (1.28) | (1.24) |

Note:
Extract/Bottle in the medium is in μl of oil/100 ml base medium.
Values in parenthesis indicate standard deviation.

TABLE 112M
Effect of plant extracts on Brassica nigra seed germination activity

| | | | | TVUL- | | | | |
|---|---|---|---|---|---|---|---|---|
| | | Sesame | A-100 | 66.84 | PE-100 | TBEL- | TBEL- | PE- |
| Extract/Bottle | | Oil | MINUS | MINUS | PLUS | P-50 | R-50 | 100 |
| 0 | Shoot | 13.06 | | | | | | |
| Control | (cm) | (0.80) | | | | | | |
| | Root | 9.14 | | | | | | |
| | (cm) | (1.17) | | | | | | |
| | Total | 22.20 | | | | | | |
| | (cm) | (1.60) | | | | | | |
| 1 | Shoot | 14.14 | 10.76 | 11.48 | 13.60 | 9.08 | 10.99 | 13.10 |
| | (cm) | (1.73) | (0.71) | (1.10) | (1.74) | (1.43) | (0.74) | (0.70) |
| | Root | 9.50 | 10.91 | 9.20 | 9.20 | 8.20 | 10.30 | 8.60 |
| | (cm) | (0.97) | (1.01) | (0.95) | (0.71) | (1.36) | (1.59) | (0.77) |
| | Total | 23.64 | 21.67 | 20.68 | 22.80 | 17.28 | 21.29 | 21.70 |
| | (cm) | (2.28) | (1.41) | (1.56) | (2.10) | (2.35) | (0.20) | (1.14) |
| | Shoot | 13.95 | 11.81 | 12.20 | 12.31 | 9.90 | 12.25 | 10.50 |
| | (cm) | (1.71) | (0.99) | (1.40) | (1.76) | (1.07) | (0.87) | (0.97) |
| | Root | 10.05 | 9.58 | 9.32 | 9.00 | 8.54 | 11.81 | 8.90 |
| | (cm) | (1.01) | (1.99) | (1.30) | (1.05) | (0.56) | (0.76) | (0.94) |
| | Total | 24.00 | 21.39 | 21.52 | 21.31 | 18.44 | 24.06 | 19.40 |
| | (cm) | (2.64) | (2.75) | (1.64) | (2.26) | (1.03) | (1.46) | (1.43) |
| 10 | Shoot | 13.30 | 12.06 | 11.84 | 12.00 | 12.14 | 11.90 | 10.27 |
| | (cm) | (0.71) | (1.69) | (1.98) | (1.73) | (1.26) | (0.97) | (0.93) |
| | Root | 11.5 | 8.64 | 9.48 | 7.70 | 10.24 | 10.19 | 9.88 |
| | (cm) | (0.94) | (1.20) | (1.12) | (1.81) | (1.95) | (0.66) | (0.93) |
| | Total | 24.80 | 20.70 | 21.32 | 19.70 | 22.38 | 22.09 | 20.14 |
| | (cm) | (1.09) | (2.51) | (0.87) | (3.10) | (2.40) | (1.13) | (1.02) |

Note:
Extract/Bottle in the medium is in μl of oil/100 ml base medium.
Values in parenthesis indicate standard deviation.

TABLE 112O
Effect of biomass extracts on Brassica nigra seed
germination activity

| | | | ER- | | | | | |
|---|---|---|---|---|---|---|---|---|
| Extract/ | | Sesame | 28.8 | TABA- | TBEL- | YBD- | PGL- | |
| Bottle | | Oil | MINUS | 28 | 27.7 | 47.55 | C-33 | C-100 |
| 0 | Shoot | 13.06 | | | | | | |
| Control | (cm) | (0.80) | | | | | | |
| | Root | 9.14 | | | | | | |
| | (cm) | (1.17) | | | | | | |
| | Total | 22.20 | | | | | | |
| | (cm) | (1.60) | | | | | | |
| 1 | Shoot | 14.14 | 11.78 | 11.86 | 11.35 | 10.86 | 11.07 | 11.87 |
| | (cm) | (1.73) | (0.95) | (0.65) | (0.90) | (0.58) | (0.73) | (0.72) |
| | Root | 9.5 | 8.67 | 10.10 | 9.04 | 8.70 | 9.60 | 10.02 |
| | (cm) | (0.97) | (0.69) | (0.84) | (0.95) | (0.82) | (0.97) | (0.82) |
| | Total | 23.64 | 20.45 | 21.96 | 20.39 | 19.56 | 20.67 | 21.89 |
| | (cm) | (2.28) | (1.13) | (1.13) | (1.14) | (1.01) | (0.99) | (1.22) |
| 3 | Shoot | 13.95 | 11.06 | 11.04 | 11.38 | 11.09 | 11.24 | 11.17 |
| | (cm) | (1.71) | (0.55) | (1.09) | (1.12) | (0.97) | (0.41) | (0.89) |
| | Root | 10.05 | 8.70 | 9.82 | 9.30 | 8.90 | 9.25 | 10.28 |
| | (cm) | (1.01) | (0.71) | (0.82) | (0.95) | (0.88) | (0.98) | (0.78) |
| | Total | 24.00 | 19.76 | 20.86 | 20.68 | 19.99 | 20.49 | 21.45 |
| | (cm) | (2.64) | (0.95) | (1.54) | (1.24) | (1.24) | (1.14) | (1.13) |
| 10 | Shoot | 13.30 | 10.48 | 10.50 | 11.90 | 11.20 | 12.05 | 11.0 |
| | (cm) | (0.71) | (0.45) | (0.67) | (1.07) | (0.71) | (0.93) | (0.86) |
| | Root | 11.50 | 9.00 | 9.10 | 9.70 | 10.75 | 9.20 | 10.60 |
| | (cm) | (0.94) | (0.78) | (0.70) | (1.34) | (0.95) | (0.97) | (0.97) |
| | Total | 24.80 | 19.48 | 19.60 | 21.60 | 21.95 | 21.25 | 21.64 |
| | (cm) | (1.09) | (1.12) | (0.84) | (1.49) | (0.80) | (1.01) | (1.32) |

Note:
Extract/Bottle in the medium is in μl of oil/100 ml base medium.
Values in parenthesis indicate standard deviation.

TABLE 112Q
Effect of plant extracts on Brassica nigra seed
germination activity

| | | | | | | | FB- | |
|---|---|---|---|---|---|---|---|---|
| Extract/ | | Sesame | GLMX- | TCHEB- | PE-100 | ASRA- | 29 | FB-100 |
| Bottle | | Oil | 27.65 | 27.8 | MINUS | 30.16 | PLUS | MINUS |
| 0 | Shoot | 10.73 | | | | | | |
| Control | (cm) | (0.84) | | | | | | |
| | Root | 8.62 | | | | | | |
| | (cm) | (0.88) | | | | | | |
| | Total | 19.35 | | | | | | |
| | (cm) | (1.39) | | | | | | |
| 1 | Shoot | 10.89 | 11.80 | 12.59 | 12.24 | 12.15 | 11.44 | 10.34 |
| | (cm) | (0.72) | (0.89) | (0.81) | (0.75) | (0.94) | (0.84) | (0.62) |
| | Root | 8.92 | 8.95 | 9.00 | 9.50 | 8.55 | 8.25 | 8.48 |
| | (cm) | (0.82) | (0.64) | (0.88) | (0.94) | (0.86) | (0.79) | (0.54) |
| | Total | 19.81 | 20.75 | 21.94 | 21.74 | 20.70 | 19.69 | 18.82 |
| | (cm) | (1.26) | (1.25) | (1.38) | (1.17) | (1.42) | (1.32) | (0.73) |
| 3 | Shoot | 11.38 | 11.04 | 11.72 | 12.29 | 11.65 | 11.57 | 11.90 |
| | (cm) | (0.86) | (0.76) | (0.69) | (0.79) | (0.78) | (0.95) | (0.91) |
| | Root | 9.34 | 8.45 | 8.80 | 8.75 | 8.45 | 8.50 | 9.55 |
| | (cm) | (0.39) | (0.86) | (0.89) | (0.79) | (0.93) | (0.62) | (0.98) |
| | Total | 20.72 | 19.49 | 20.52 | 21.04 | 20.10 | 20.07 | 21.45 |
| | (cm) | (0.82) | (1.43) | (1.17) | (1.10) | (1.37) | (0.94) | (1.57) |
| 10 | Shoot | 11.86 | 10.80 | 10.44 | 12.14 | 11.57 | 10.64 | 12.59 |
| | (cm) | (0.80) | (0.98) | (0.66) | (0.81) | (0.77) | (0.58) | (0.90) |
| | Root | 10.07 | 8.25 | 7.80 | 8.20 | 8.21 | 10.55 | 10.35 |
| | (cm) | (0.85) | (0.86) | (0.98) | (0.71) | (0.97) | (0.96) | (0.91) |
| | Total | 21.93 | 19.05 | 18.24 | 20.34 | 19.78 | 21.19 | 22.94 |
| | (cm) | (1.33) | (1.71) | (1.13) | (1.14) | (1.534) | (1.00) | (1.38) |

Note:
Extract/Bottle in the medium is in μl of oil/100 ml base medium.
Values in parenthesis indicate standard deviation.

TABLE 112S
Effect of plant extracts on Brassica nigra seed
germination activity

| | | | TVUL- | | | | CAMA- | CAMA- |
|---|---|---|---|---|---|---|---|---|
| Extract/ | | Sesame | 32.83 | ER-100 | R- | R-100 | 26.2 | 98.4 |
| Bottle | | Oil | PLUS | MINUS | 100 | (4J + L/8) | PLUS | MINUS |
| 0 | Shoot | 11.17 | | | | | | |
| Control | (cm) | (0.76) | | | | | | |
| | Root | 9.52 | | | | | | |
| | (cm) | (0.79) | | | | | | |
| | Total | 20.69 | | | | | | |
| | (cm) | (1.03) | | | | | | |
| 1 | Shoot | 11.34 | 14.01 | 11.15 | 11.51 | 12.20 | 11.70 | 12.06 |
| | (cm) | (0.59) | (0.78) | (0.75) | (0.75) | (0.79) | (0.79) | (0.63) |
| | Root | 9.57 | 10.93 | 10.45 | 9.41 | 9.70 | 9.57 | 9.35 |
| | (cm) | (0.55) | (0.67) | (0.98) | (0.61) | (0.67) | (0.51) | (0.50) |
| | Total | 20.91 | 24.94 | 21.60 | 20.92 | 21.90 | 21.27 | 21.41 |
| | (cm) | (0.90) | (0.99) | (1.35) | (1.02) | (1.07) | (0.96) | (0.76) |
| 3 | Shoot | 12.03 | 12.51 | 12.50 | 13.30 | 13.30 | 11.85 | 12.41 |
| | (cm) | (0.65) | (0.67) | (0.97) | (0.71) | (0.71) | (0.73) | (0.82) |
| | Root | 9.74 | 11.01 | 9.55 | 9.91 | 9.26 | 9.60 | 9.56 |
| | (cm) | (0.63) | (0.47) | (0.83) | (0.49) | (0.59) | (0.48) | (0.46) |
| | Total | 21.77 | 23.52 | 22.05 | 23.21 | 22.56 | 21.45 | 21.97 |
| | (cm) | (0.96) | (0.99) | (1.17) | (0.80) | (0.84) | (0.96) | (0.91) |
| 10 | Shoot | 12.13 | 11.76 | 13.10 | 13.90 | 14.05 | 12.99 | 13.02 |
| | (cm) | (0.59) | (0.60) | (0.77) | (0.70) | (0.98) | (0.70) | (0.48) |
| | Root | 10.01 | 11.29 | 9.30 | 10.50 | 10.09 | 9.89 | 10.79 |
| | (cm) | (0.80) | (0.76) | (0.79) | (0.82) | (0.94) | (0.59) | (0.79) |
| | Total | 22.14 | 23.05 | 22.40 | 24.40 | 24.14 | 22.88 | 23.81 |
| | (cm) | (0.78) | (1.14) | (0.70) | (1.02) | (0.81) | (0.73) | (1.13) |

Note:
Extract/Bottle in the medium is in μl of oil/100 ml base medium.
Values in parenthesis indicate standard deviation.

TABLE 112U
Effect of plant extracts on Brassica nigra seed
germination activity

| | | | SCHIR- | SCHIR- | | CROT | CROT | ZOFF |
|---|---|---|---|---|---|---|---|---|
| Extract/ | | Sesame | 28.8 | 55 | B-100 | 28.2 | 62.66 | 55.4 |
| Bottle | | Oil | PLUS | MINUS | MINUS | PLUS | MINUS | PLUS |
| 0 | Shoot | 11.17 | | | | | | |
| Control | (cm) | (0.76) | | | | | | |
| | Root | 9.52 | | | | | | |
| | (cm) | (0.79) | | | | | | |
| | Total | 20.69 | | | | | | |
| | (cm) | (1.03) | | | | | | |
| 1 | Shoot | 11.86 | 13.65 | 133.20 | 13.04 | 13.80 | 12.80 | 13.50 |
| | (cm) | (0.85) | (0.71) | (0.89) | (0.96) | (0.92) | (0.79) | (0.47) |
| | Root | 9.40 | 11.0 | 8.84 | 8.30 | 9.83 | 12.30 | 11.65 |
| | (cm) | (0.58) | (0.75) | (0.47) | (0.79) | (0.94) | (0.92) | (0.91) |
| | Total | 21.26 | 24.65 | 22.04 | 21.34 | 23.63 | 25.10 | 25.15 |
| | (cm) | (1.09) | (1.00) | (1.06) | (1.32) | (1.75) | (1.02) | (1.00) |
| 3 | Shoot | 12.33 | 12.30 | 12.00 | 13.50 | 14.00 | 13.35 | 12.90 |
| | (cm) | (0.71 ) | (0.63) | (0.78) | (0.75) | (0.67) | (0.91) | (0.88) |
| | Root | 10.25 | 10.40 | 9.15 | 11.15 | 11.99 | 12.21 | 9.01 |
| | (cm) | (0.52) | (0.63) | (0.94) | (0.91) | (0.80) | (0.77) | (0.91) |
| | Total | 22.58 | 22.70 | 21.15 | 24.65 | 25.99 | 25.66 | 21.91 |
| | (cm) | (0.77) | (0.82) | (1.23) | (1.31) | (1.07) | (1.07) | (1.59) |
| 10 | Shoot | 12.46 | 12.22 | 11.30 | 13.90 | 14.35 | 14.20 | 12.45 |
| | (cm) | (0.75) | (0.75) | (0.95) | (0.57) | (0.88) | (0.86) | (0.69) |
| | Root | 10.50 | 10.00 | 9.55 | 9.08 | 112.49 | 11.94 | 8.65 |
| | (cm) | (0.80) | (0.75) | (0.96) | (0.89) | (0.98) | (0.77) | (0.47) |
| | Total | 22.97 | 22.0 | 20.85 | 22.98 | 26.84 | 26.14 | 21.10 |
| | (cm) | (0.92) | (1.03) | (0.75) | (0.90) | (1.59) | (1.40) | (0.94) |

Note:
Extract/Bottle in the medium is in μl of oil/100 ml base medium.
Values in parenthesis indicate standard deviation.

TABLE 112W
Effect of plant extracts on Brassica nigra seed
germination activity

| | | | R- | | | | | |
|---|---|---|---|---|---|---|---|---|
| Extract/ | | Sesame | STEM | SIND- | HA- | R-25 | R-25 | R-25 |
| Bottle | | Oil | 10 | 28.8 | 29.7 | 30 min | 120 min | 240 min |
| 0 | Shoot | 11.17 | | | | | | |
| Control | (cm) | (0.76) | | | | | | |
| | Root | 9.52 | | | | | | |
| | (cm) | (0.79) | | | | | | |
| | Total | 20.69 | | | | | | |
| | (cm) | (1.03) | | | | | | |
| 1 | Shoot | 11.86 | 12.05 | 11.85 | 12.78 | 12.40 | 14.02 | 13.18 |
| | (cm) | (0.85) | (0.83) | (0.63) | (0.87) | (0.77) | (0.65) | (0.76) |
| | Root | 9.40 | 11.95 | 9.90 | 11.35 | 10.6 | 10.11 | 10.31 |
| | (cm) | (0.58) | (0.83) | (0.99) | (0.94) | (0.77) | (0.56) | (0.71) |
| | Total | 21.26 | 24.00 | 21.75 | 24.13 | 23.00 | 24.13 | 23.49 |
| | (cm) | (1.09) | (0.97) | (1.34) | (1.34) | (0.85) | (0.74) | (1.03) |
| 3 | Shoot | 12.33 | 12.60 | 12.50 | 13.00 | 12.84 | 13.10 | 13.30 |
| | (cm) | (0.71) | (0.77) | (0.94) | (0.85) | (0.73) | (0.94) | (0.92) |
| | Root | 10.25 | 11.30 | 10.94 | 11.85 | 10.94 | 10.71 | 10.01 |
| | (cm) | (0.52) | (0.92) | (0.90) | (0.85) | (0.67) | (0.57) | (0.80) |
| | Total | 22.58 | 23.90 | 23.44 | 24.85 | 23.78 | 23.81 | 23.31 |
| | (cm) | (0.77) | (1.17) | (1.60) | (1.18) | (1.16) | (1.32) | (0.84) |
| 10 | Shoot | 12.46 | 13.45 | 11.45 | 13.50 | 13.25 | 12.20 | 11.65 |
| | (cm) | (0.75) | (0.96) | (0.93) | (0.75) | (0.89) | (0.82) | (0.75) |
| | Root | 10.50 | 9.05 | 9.05 | 12.55 | 9.98 | 10.70 | 9.30 |
| | (cm) | (0.80) | (0.99) | (0.98) | (0.72) | (0.38) | (0.71) | (0.79) |
| | Total | 22.97 | 22.50 | 20.50 | 26.05 | 23.23 | 22.90 | 20.95 |
| | (cm) | (0.92) | (1.64) | (1.00) | (0.72) | (0.90) | (1.37) | (0.83) |
| | Shoot(mg) | | | | | | | |

Note:
Extract/Bottle in the medium is in μl of oil/100 ml base medium.
Values in parenthesis indicate standard deviation.

TABLE 112Y
Effect of plant extracts on Brassica nigra seed
germination activity

| Extract/ | | Sesame | PINI- | GYSY- |
|---|---|---|---|---|
| Bottle | | Oil | 32.4 | 26.4 |
| 0 | Shoot | 7.89 | | |
| Control | (cm) | (0.85) | | |
| | Root | 6.99 | | |
| | (cm) | (0.6) | | |
| | Total | 14.88 | | |
| | (cm) | (1.08) | | |
| 1 | Shoot | 7.88 | 9.91 | 10.33 |
| | (cm) | (0.50) | (0.68) | (0.73) |
| | Root | 7.17 | 8.08 | 8.46 |
| | (cm) | (0.57) | (0.73) | (0.51) |
| | Total | 15.05 | 17.99 | 18.79 |
| | (cm) | (0.95) | (0.86) | (0.93) |
| | Shoot | 9.29 | 10.24 | 10.60 |
| | (cm) | (0.57) | (0.69) | (0.66) |
| | Root | 7.52 | 8.50 | 8.53 |
| | (cm) | (0.52) | (0.88) | (0.56) |
| | Total | 16.81 | 18.74 | 19.13 |
| | (cm) | (0.44) | (1.18) | (0.93) |
| 10 | Shoot | 9.59 | 10.32 | 10.59 |
| | (cm) | (0.88) | (0.84) | (0.70) |
| | Root | 7.38 | 8.77 | 8.96 |
| | (cm) | (0.63) | (0.68) | (0.49) |
| | Total | 16.97 | 19.09 | 19.55 |
| | (cm) | (0.88) | (1.01) | (1.03) |

Note:
Extract/Bottle in the medium is in μl of oil/100 ml base medium.
Values in parenthesis indicate standard deviation.

Both Gymnema sylvestree (GYSY-26.4) and Piper nigrum (PINI-32.4) are strong promoters of gemination in mustard at the low dose level of 1 μl of oil/100 ml base medium.

Thus, oil compositions of total homogenate as also of PLUS and MINUS fractions of a wide range of Angiosprm-monocotyledonous, Angiosperm-dicotyledonous and Gymnosperm plants and also extracts of non-plant biomass showed significant biological activity with respect to a protein rich dicotyledonous plant seed (Phaseolus radiatus) and an oil rich dicotyledon plant seed (Brassica nigra) at very low dose levels. Several extracts promoted auxin-like (rooting promotion), gibberellin-like (shooting promotion) or cytokine-like (biomass preservation/growth) activity at low doses of 1 μl/100 ml medium to 10 μl/100 ml of medium. Other extracts caused a strong inhibition of rooting, shooting and biomass mobilization at low doses of 3 μl/100 ml medium to 10 μl/100 ml of medium. Thus, a broad range of activity such as promotion, promotion followed by inhibition and inhibition of a wide range of endogenous hormones at a low concentration was observed. These different activities greatly enhance the utility of the compositions of this invention.

Response of any particular extract in case of mungbean (Phaseolus radiatus) may not be identical to its response in mustard seed. As can be seen from the set of Tables 12 and 112, a particvular extract may promote both, promote mungbean and inhibit mustard or promote mustard and inhibit mungbean. Such differential activity can allow more selectivity in directing a specific extract combination towards a specific plant.

Effects on Fruits and Vegetables

### Example 18

### Promotion of Plant Defense, Growth, and Enzymes in Okra (Abelmoschus esculentus L CV Pusa Savani)

Experiments were carried out at Pune, India, using R-5 oil (batch 920814). Plants were cultured in both hydroponic (sand culture with Modified Hoagland media) and normal soil (loamy soil and farmyard manure (3:1), 12 kg/pot) media. The three treatment levels-used were 1 mg, 3 mg and 10 mg per liter of R-5, and were applied at a rate of one liter per pot per application. Thus, the amount of R-100 oil equivalent added per treatment/pot was 0.05 mg, 0.15 mg and 0.50 mg. Plants were treated for 15 and 30 days after sowing. The results are summarized in Table 13.

TABLE 13
Increase in plant height (cm)

| Conc., mg R-5 oil | Initial | @ 40 days | @ 60 days |
|---|---|---|---|
| Control | 8.3 | 42.3 | 52.6 |
| 1 | 8.2 | 51.4 | 61.7 |
| 3 | 8.3 | 53.3 | 67.5 |
| 10 | 8.2 | 54.6 | 69.4 |
| Stand. Error. | 0.41 | 1.02 | 0.83 |
| C.D. @ 1% | 1.52 | 3.78 | 3.09 |

Leaf area (LA), and leaf dry weight (LW) increased in both hydroponics and soil cultures at flowering (mean of three plants) at all concentrations compared to control (Table 14).

TABLE 14
Increase in leaf area and leaf dry weight

| | LA | | LW | |
|---|---|---|---|---|
| | (cm²/plant) | | g/plant | |
| Conc., mg, R-5 oil | Std. Deviation. | | Std. Deviation | |
| HYDROPONICS (54 days) | | | | |
| 0(Control) | 356.0 | 1.85 | 1.49 | 0.5 |
| 1 | 396.3 | 1.62 | 2.15 | 0.1 |
| 3 | 392.1 | 1.68 | 2.01 | 0.47 |
| 10 | 374.0 | 0.87 | 2.35 | 0.67 |
| SOIL (58 days) | | | | |
| 0(Control) | 636.3 | 2.09 | 1.93 | 0.26 |
| 1 | 744.1 | 2.16 | 2.87 | 0.12 |
| 3 | 834.1 | 1.5 | 3.01 | 0.19 |
| 10 | 756.3 | 2.04 | 2.63 | 0.05 |

All treated plants had dark green glossy leaves and higher chlorophyll level, particularly those of chlorophyll-b (mean of three samples), summarized in Table 15.

TABLE 15
Leaf chlorophyll levels

| | | @ 30 days | | @ 60 days |
|---|---|---|---|---|
| Conc., mg, R-5 | chloro-a | chloro-b | chloro-a | chloro-b |
| HYDRO- | | | | |
| PONICS | | | | |
| 0 (Control) | 101.9 (0.56) | 127.6 (0.85) | 97.4 (0.64) | 123.6 (0.55) |
| 1 | 104.2 (1.94) | 129.1 (1.65) | 101.5 (0.52) | 133.9 (0.76) |
| 3 | 106.2 ((1.47) | 143.8 (1.03) | 103.9 (0.77) | 141.8 (0.52) |
| 10 | 93.5 (0.87) | 131.8 (0.68) | 104.0 (0.59) | 130.1 (0.88) |
| SOIL | | | | |
| 0 (Control) | 96.4 (1.26) | 117.4 (1.20) | 103.8 (1.09) | 132.8 (1.09) |
| 1 | 104.9 (0.93) | 127.5 (1.29) | 105.4 (0.76) | 134.7 (0.80) |
| 3 | 115.9 (1.14) | 156.4 (1.05) | 116.8 (0.95) | 172.1 (0.89) |
| 10 | 103.2 (1.76) | 129.5 (1.22) | 101.2 (1.02) | 144.1 (0.67) |
| C.D. (1%) | 5.87 | 5.86 | 3.51 | 4.1 |

NOTE:
Chlorophyll levels are in mg/100 g fresh weight (FW) of leaves.
Numbers in parenthesis are values for standard deviation.

Activity of photosynthetic enzyme, ribulose phosphate (RuBp) and oxidative enzymes (Peroxidase, Polyphenol oxidase (PPO) and IAA oxidase (IAAO)) creased in treated okra plants, as summaraized in Table 16.

TABLE 16
Leaf enzyme activity

| Conc., mg, R-5 | RuBp | IAAO | PPO | Peroxidase |
|---|---|---|---|---|
| HYDROPONICS@ 52 days | | | | |
| 0(Control) | 0.083 | 0.87 | 0.086 | 0.62 |
| 1 | 0.092 | 0.91 | 0.102 | 0.89 |
| 3 | 0.098 | 0.98 | 0.127 | 0.96 |
| 10 | 0.099 | 0.89 | 0.088 | 0.66 |
| SOIL@ 52 days | | | | |
| 0(Control) | 0.092 | 0.94 | 0.087 | 0.88 |
| 1 | 1.025 | 1.29 | 0.092 | 0.93 |
| 3 | 1.058 | 1.44 | 0.111 | 1.27 |
| 10 | 1.098 | 1.21 | 0.102 | 1.14 |

NOTE:
Enzyme activity for IIAO, PPO and Peroxidase is expressed as change in optical density/min/g protein
RuBp enzyme: specific activity = micromoles/min/mg protein.

In addition, the level of carbohydrates, proteins and polyphenols increased in leaves of treated plants, summarized in Table 17.

TABLE 17
Increase in carbohydrates, proteins and
polyphenols in leaves (mean of three plants)

| | Carbohydrates | Proteins | Polyphenol |
|---|---|---|---|
| Conc., mg, R-5 | mg/g Fresh Wt. | mg/g Fresh Wt | mg/g Wt |
| HYDROPONICS@ | | | |
| 62 days | | | |
| 0(Control) | 30.1(1.21) | 56.3(0.81) | 6.2(0.98) |
| 1 | 52.2(1.38) | 57.9(0.98) | 16.0(0.92) |
| 3 | 59.8(0.98) | 58.1(0.76) | 18.0(1.02) |
| 10 | 68.2(1.08) | 58.3(0.56) | 11.1(0.78) |
| SOIL@ 68 days | | | |
| 0(Control) | 38.7 | 61.1 | 8.3 |
| 1 | 98.4 | 65.1 | 10.6 |
| 3 | 88.1 | 66.1 | 14.3 |
| 10 | 102.4 | 65.3 | 12.9 |
| Stand. Error | 4.04 | 0.77 | 0.84 |
| C.D. @ 1% | 14.96 | 2.85 | 3.1 |

NOTE:
Numbers in parenthesis are values of standard deviation.

Qualitative observations included larger flowers and higher fruit yield in treated plants. Thus, R-100 appears to act at a very fundamental level in all stages of plant growth. For example, higher chlorophyll level and altered metabolic activities caused by R-100 might have increased the RuBp-case activity and resulted in a higher carbohydrate level in the leaves.

Induction of endogenous phytohormone synthesis by R-100 may be responsible for increase in height and leaf area and IIA oxidase level.

Induced auxin and cytokine levels and higher peroxidase activity may have reduced hydrogen peroxide levels and delayed senescence.

Effect of R-100 was much more pronounced in soil culture than in hydroponics medium. This may be a result of a synergistic interaction of R-100 with the rhizosphere microflora (fungi, yeast, actinomycetes, etc.)

Growth parameters and biochemical status were also affected. Plant height increased for treated plants in soil culture (mean of 10 plants) at all concentrations compared to control.

### Example 19

### Yield, Productive Life, and Pest Resistance in Tomato, Brinjal and Okra

Trials were carried out near Daund, Maharashtra, India on Tomato (Lycopersicum esculentum), Golden variety; Brinjal (Solanum melangona), Kalptharu variety; and Okra (Abelmoschus esculentus L.) Parbhani kranti variety. The plants were administered a concentration of 1 T-5 (250 mg) tablet/5 liters at 30, 60 and 90 days after transplantation. The solution was used at 3, 4, and 5 liters/100 sq. ft for sprays 1, 2 and 3, respectively. Fifty (50) plants were used per experimental condition; the results are reported in Table 18.

TABLE 18
Yield, productive life, and pest resistance

| OBSERVATIONS | TOMATO | | BRINJAL | | OKRA | |
|---|---|---|---|---|---|---|
| Yield, kg | Test | Control | Test | Control | Test | Control |
| 1^{st} Harvest | 80 | 60 | 55 | 40 | 35 | 35 |
| 2^{nd} Harvest (31 days | 30 | 20 | 15 | 12 | 15 | 04 |
| after 1^{st} harvest) | | | | | | |
| 3^{rd} Harvest, (61 days | 20 | 06 | 10 | 02 | 14 | 07 |
| after 1^{st} harvest) | | | | | | |
| TOTAL | 130 | 86 | 80 | 54 | 64 | 46 |

Other observations relative to control plants were:
Tomato: Sucking pest attack reduced
Brinjal: Fruit borer attack was reduced. Fruit soft, tender.
Okra: Leaf curling reduced.

### Example 20

### Yield in Capsicum Annuum and Okra (Abelmoschus esculenuts L.)

Trials were carried out at Dapoli, Maharashtra, India; 3 replicates were used for each treatment. R-10 oil (Batch 910318) was used, and a total of three sprays were applied. Controls were given water sprays. The results are presented in Table 19.

TABLE 19
Yield

| | RED CHILLIES | | | OKRA | | |
|---|---|---|---|---|---|---|
| | CON- | | | CON- | | |
| OBSERVATIONS | TROL | SET I | SET II | TROL | SET I | SET II |
| R-10 oil | 0 | 10 | 20 | 0 | 10 | 20 |
| Conc., ml/ha | | | | | | |
| Yield/plant, g | 63.0 | 81.3 | 103.1 | | | |
| Yield/Ha, | 35.03 | 45.10 | 57.33 | 168.3 | 180.0 | 188.80 |
| quintals | | | | | | |

### Example 21

### Shelf Life of R-5 Treated Okra

Experiments were carried out at Pune, India in Nov. 1999 using R-5 oil (batch 920814) and R-5 oil (batch 990509) from R-100 oil preparation. Abelmoschus esculentus L CV Lucy was grown in loamy soil and farmyard manure (3:1), 20 kg/pot (Plastic tubs with 25 cm radius and 25 cm high). The three treatment levels used were 1 mg, 3 mg and 10 mg per liter of R-5. Per application, one liter of solution was applied per pot. Thus, the amount of R-100 oil equivalent added per treatment/pot was 0.05 mg, 0.15 mg and 0.50 mg. Plants were treated 15 days and 30 days after sowing.

The growth parameters and biochemical status were examined (Table 20) Plant height increased for treated plants (mean of 10 plants) compared to control at up to 3 mg of R-5. At higher concentrations, there was a reversal observed in both sets. The results of treatment with older (7 years old) and newer batch of R-100 did not show significant differences.

TABLE 20
Increase in plant height (cm)

| R-100 BATCH DATE | |
|---|---|
| 14^{th} August, 1992 | 9^{th} May, 1999 |

| Conc., mg R-5 oil | Initial | @ 60 days | Initial | @ 60 days |
|---|---|---|---|---|
| Control | 8.6 | 49.9 | 8.2 | 46.6 |
| 1 | 8.6 | 53.6 | 8.2 | 52.3 |
| 3 | 8.6 | 54.8 | 8.3 | 56.3 |
| 10 | 8.6 | 51.5 | 8.3 | 48.6 |
| Stand. Error. | 0.26 | 0.38 | 0.35 | 0.91 |
| C.D. (1%) | 1.52 | 1.41 | 1.29 | 3.39 |

Leaf area (LA), and leaf dry weight (LW) increased in both hydroponics and soil culture at flowering (mean of three plants) at all concentrations compared to controls. All treated plants had dark green glossy leaves and higher chlorophyll a and b level particularly up to 3 mg R-5 level (Table 21).

TABLE 21
Chlorophyll levels

| | R-100 DATE | | | |
|---|---|---|---|---|
| | 14^{th} August, 1992 | | 9^{th} May, 1999 | |
| Conc., mg, R-5 | @ 60 days | | @ 60 days | |
| SOIL | chloro-a | chloro-b | chloro-a | chloro-b |
| 0(Control) | 107.4(0.93) | 141.4(0.96) | 105.8(0.63) | 136.5(0.67) |
| 1 | 111.5(0.83) | 152.1(0.35) | 115.5(0.91) | 152.9(0.77) |
| 3 | 114.6(0.76) | 154.3(0.42) | 116.8(0.74) | 172.1(0.70) |
| 10 | 114.0(0.47) | 148.4(0.49) | 107.5(0.39) | 143.3(0.60) |
| C.D. (1%) | 3.72 | 2.56 | 3.0 | 3.46 |

NOTE:
Chlorophyll levels are in mg/100 gm fresh weight (FW) of leaves.
Numbers in parenthesis are values of Standard Deviation.

Levels of reducing sugars increased in treated plants at flowering both with old and new composition. Increases in non-reducing sugars were not highly significant (Table 22).

TABLE 22
Effect on non-reducing and reducing sugars

| | R-100 DATE | | | |
|---|---|---|---|---|
| | 14^{th} August, 1992 | | 9^{th} May, 1999 | |
| | | Non- | | Non- |
| SOIL@ −62 days | Reducing | Reducing | Reducing | Reducing |
| Conc., mg of R-5 oil | mg/g FW | mg/g FW | mg/g FW | mg/g FW |
| 0(Control) | 17.9 | 16.8 | 14.3 | 17.12 |
| 1 | 22.4 | 18.8 | 22.6 | 20.08 |
| 3 | 26.4 | 19.4 | 22.7 | 21.6 |
| 10 | 22.4 | 18.7 | 21.9 | 19.3 |
| Stand. Error | 0.81 | 0.71 | 0.77 | 0.52 |
| C.D. @ 1% | 3.01 | 2.65 | 2.88 | 1.91 |

Other observations included larger flower and higher fruit yield in treated plants with both the new and old composition. Thus, R-100 appeared to act at a very fundamental level in all stages of plant growth. R-100 activity is substantially retained in samples that are 7 years old.

### Example 22

### Fruit: Higher Yield, Pest Resistance and Shelf Life

Trials with T-5 (250 mg) tablets were carried out near Pune, India on a variety of fruit trees. Observations were recorded with respect to control trees. 10 trees of each type were used for measurements at the end of the season. Two T-5 tablets were dissolved in a minimum of 2 liters of water. This solution was used per spray per tree. The results are reported in Table 23.

Mango, Pomegranate, Ber, Sapota: Two sprays were given at a 20 day interval during the flowering.

Lime and Guava: Three sprays were given at 30 day interval during flowering.

TABLE 23
Fruit yield, resistance, quality, ripening and shelf life

| | TEST | CONTROL |
|---|---|---|
| MANGO | | |
| (Mangifera indica) | | |
| Number of fruit | 370 | 300 |
| Fruit quality | Shiny, attractive | |
| Ripening | delayed by 10-12 days | |
| Resistance: | Leaf curling reduced | |
| POMEGRANATE | | |
| (Punica granatum) | | |
| Number of fruit | 197 | 150 |
| Fruit quality | Redness increased | |
| | Black spots reduced | |
| | Fruit drop and decay reduced | |
| Resistance: | Pomegranate butterfly attack | |
| | reduced | |
| BER (Zizyphus jujuba) | | |
| Fruit/tree, kg | 6 | 4 |
| Fruit quality | Shiny, Ready for harvest early | |
| | Longer shelf life | |
| Resistence: | sucking pest/fruit borer attack | |
| | reduced | |
| SAPOTA (Achras sapota) | | |
| Number of fruit | 333 | 300 |
| Fruit quality | healthy looking | |
| | Late ripening and. | |
| | Longer shelf life | |
| LIME | | |
| (Citrus aurantifoliea) | | |
| Yield increase | 25% | |
| Fruit quality | Larger size | |
| | Reduced fruit drop and | |
| | decay of fallen fruit | |
| Resistance: | Black leaf eating caterpillar | |
| | reduced | |
| GUAVA (Psidum guava) | | |
| Yield increase | 20% increase | |
| Fruit quality | Larger | |
| | Attractive color | |
| | Development on ripe fruit | |

### Example 23

### Yield and Size in Strawberry (Fragaria×ananasa) (Chandllar Variety)

Trials with R-5 oil were carried out at Panchgani, Maharashtra, India. Spray volume was 300 liter/ha. Three treatments with R-5 oil rate of 3 ml/ha, 10 ml/ha and 30 ml/ha were used. This corresponds to R-5 concentration of 33 ppm, 100 ppm and 300 ppm solutions. A total of four sprays were given (one at 31 days, 42 days, 138 days and 156 days after planting). Each plot was 1 m² with 5 plants. Randomized Block design with 5 replicates was used.

The first flush was washed out due to rain. The fruit were collected from the next ten flushes. The results are reported in Table 24.

TABLE 24
Yield and size

| Level of application | CONTROL | 10 ppm | 33 ppm | | | 100 ppm |
|---|---|---|---|---|---|---|
| OBSERVATION | | | | | | |
| S.E. CD@ 5% | | | | | | |
| Av. Wt. of Fruit/plot, g | 257.4 | 279.8 | 546.0 | 472.4 | 16.23 | 50.01 |
| Av. No. of Fruit/plot | 23.4 | 23.4 | 25.0 | 29.4 | 1.27 | 3.92 |
| Av. Wt. of Fruit, g | 11.0 | 11.9 | 21.8 | 16.0 | | |
| OTHER OBSERVATIONS | | | | | | |
| LEAVES | — | glossy | glossy | glossy | | |
| FRUIT | | shiny | shiny | shiny | | |

Thus, there was a substantial increase in yield and size at concentrations of 33 and 100 ppm.

### Example 24

### Growth, Chlorophyll, Nutrients, Phenols and Solasodine in Solanum khasianum

Trials were carried out on plants grown in soil at Pune with T-1 (150mg) tablets (batch 930417) and R-5 (batch 920814) from R-100 preparations that were more than 6 years old. Seeds were obtained from Mahatma Phule Agricultureal University, Rahuri, Maharasshtra, India. The plants were cultured in plots 1 m×1.5 m, using the ridges and furrow method. Plants were space 30 cm within rows 60 cm apart. Five plants per treatment, each in a row, were used.

Solution concentrations used: (Control: distilled water)

| Treatment | T-1(150) Tablets/lit. | Treatment | R-5 Oil, μl/liter |
|---|---|---|---|
| Tab 1 | 3 | O 4 | 60 |
| Tab 2 | 1 | O 5 | 20 |
| Tab 3 | 1/3 | O 6 | 7 |

Spray Method: 10 ml/plant, twice a month up to fruiting. Thus, the amount of R-100 or leaf equivalent used per plant per spray was approx. 0.03 mg, 0.01 mg and 0.0033 mg. Treatments were initiated 30 days after seedling (30 day old seedlings) transplanting. The average results of the combined three sets for Tab1, Tab2, O5 and O6 are summarized in Tables 25-27.

TABLE 25
Plant growth parameters:
Observations taken 58-60 days after first treatment;
Average values of three plants
per set for three sets were measured:

| | | | | | |
|---|---|---|---|---|---|
| Treatment | Control | Tab1 | Tab2 | O 5 | O 6 |

PLANT (Average values)

| | | | | | |
|---|---|---|---|---|---|
| Height, cm | 56.0 (2.31) | 77.0 (1.31) | 92.7 (1.3) | 64.5 (65.5) | 66.3 (0.85) |
| Branches | 33.5 (2.07) | 45.0 (1.410) | 56.5 (1.1) | 52.1 (1.49) | 50.4 (0.72) |

LEAF (Average values)

| | | | | | |
|---|---|---|---|---|---|
| Spines (upper) | 30.3 (0.85) | 14.8 (0.30) | 12.8 (0.91) | 13.4 (0.53) | 16.2 (0.66) |
| Spines (lower) | 37.5 (0.70) | 16.4 (1.14) | 14.2 (0.60) | 17.4 (0.53) | 16.2 (0.79) |

Values in parenthesis indicate standard deviations.

TABLE 26
Pigments, proteins and polyphenols in leaves
60 days after transplanting; Average values of three
plants each from three sets are reported:

| | | | | | |
|---|---|---|---|---|---|
| Treatment | Control | Tab1 | Tab2 | O 5 | O 6 |

(mg/100 g of Fresh Wt. of leaves)

| | | | | | |
|---|---|---|---|---|---|
| Chlorophyll, | 132.6 (3.39) | 138.3 (4.62) | 140.4 (1.57) | 133.8 (1.84) | 136.8 (2.1) |

(g/100 g of Fresh Wt. of leaves)

| | | | | | |
|---|---|---|---|---|---|
| Carbohydrates | 2.5 (0.07) | 3.1 (0.11) | 2.9 (0.05) | 2.8 (0.04) | 2.6 (0.05) |
| Proteins | 3.4 (0.06) | 4.1 (0.63) | 3.8 (0.28) | 4.3 (0.20) | 3.7 (0.17) |
| Total Phenols | 2.82 (0.09) | 3.23 (0.12) | 3.1 (0.07) | 3.41 (0.06) | 3.02 (0.15) |

TABLE 27
Fruit and seeds yield
Average value of three plants each for three sets are reported.

| Treatment | Control | Tab1 | Tab2 | O 5 | O 6 |
|---|---|---|---|---|---|
| Fresh Wt. of Fruit/ | 77.3 (0.68) | 82.1 (0.44) | 90.3 (1.3) | 85.7 (1.01) | 80.2 (1.78) |
| plant, g | | | | | |
| Solasodine | 31.4 (0.092) | 40.2 (0.60) | 45.3 (1.23) | 46.1 (0.54) | 45.1 (0.27) |
| (mg/100 g DW of Fruit) | | | | | |
| gm/100 seeds | 0.20 (0.02) | 0.25 (0.02) | 0.30 (0.04) | 0.30 (0.04) | 0.28 (0.02) |
| seeds/fruit | 181.5 (3.62) | 210.2 (1.24) | 215.3 (4.20) | 217.3 (1.41) | 215.2 (3.21) |

Thus, plant height and number of branches were enhanced by application of tablets and oil. At these treatment levels, carbohydrate, protein and phenol levels/FW of leaves increased marginally in treated plants. However, the spines were reduced by more than 50% at all treatment levels used. This makes harvesting easier. Fruit yield was higher and at this higher yield, solasodine levels are 40% to 50% higher in treated plants than in controls. Thus, the medicinally important alkaloid levels have been increased/plant.

### Example 25

### Cotton

Trials were carried out at Dharwad, Karnataka, India in Kharif on cotton. Plants were cultivated on 18.2 m² plots; 3 replicates were used for each set.

R-2 oil (batch 910608) was used, and a total of 3 sprays were applied: 65, 83, 113 days after sowing. The results are reported in Table 28.

TABLE 28
Cotton yield

| | CONTROL | CONTROL | |
|---|---|---|---|
| | Without Water | With Water | TEST |
| R-2 oil Conc., ml/ha | 0 | 0 | 50 |
| Yield/Ha, quintals | 14.27 | 13.73 | 16.98 |
| % Increase | 3.92 | 0 | 23.46 |

### Example 26

### Growth, Resistance, Leaf Active Life, and Yield in Soybean

Trials were carried out at the Pune University campus with T-1 (150 mg) tablets (batch 930417) and R-5 (batch 920814) from R-100 preparations that were more than 6 years old. Plants (Glycine max L. cv Macs) (winter variety) were cultivated in soil in pots 20 cm×30 cm×40 cm; (farmyard manure and garden soil in 1:3 ratio). Seed were obtained from Agharkar Research Institute (Pune, Maharasshtra, India). Four plants were grown in each pot, and each treatment consisted of 3 pots. Control solution was distilled water. Treatments were:

| Treatment | T-1(150) Tablets/lit. | Treatment | R-5 Oil, microlit./lit. |
|---|---|---|---|
| Tab 1 | 3 | O 4 | 60 |
| Tab 2 | 1 | O 5 | 20 |
| Tab 3 | 1/3 | O 6 | 7 |

Spray Method: 100 ml/pot, twice a month up to fruiting. Thus amount of R-100 used per plant per spray was approx. 0.3 mg, 0.1 mg and 0.033 mg. The first treatment was applied 40 days after sowing.

Two sets of treatment were carried out. Average values of the combined set are reported in Tables 29-31. (Values in parentheses indicate standard deviations).

TABLE 29
Growth parameters
(Observations just before flowering)

| | Control | Tab1 | Tab2 | O 5 | O 6 |
|---|---|---|---|---|---|
| | PLANT (Av. Value) | | | | |
| Height, cm | 27.1 (0.28) | 30.3 (1.27) | 32.4 (2.55) | 32.5 (1.27) | 31.3 (0.71) |
| Branches | 5.2 (0.21) | 8.6 (0.24) | 9.2 (0.18) | 9.1 (0.22) | 8.2 (0.24) |
| Leaves | 9.1 (0.42) | 12.3 (0.24) | 12.8 (0.38) | 12.6 (0.35) | 12.5 (0.39) |
| Leaf area (cm²) | 27.5 (1.12) | 36.6 (1.28) | 36.8 (1.35) | 36.3 (1.23) | 36.5 (1.27) |

TABLE 30
Yield and leaf productivity

| | Control | Tab1 | Tab2 | O 5 | O 6 |
|---|---|---|---|---|---|
| | PLANT (Av. Value) | | | | |
| LAD | 60.5 (2.54) | 80.8 (1.81) | 85.6 (2.08) | 85.7 (1.98) | 85.2 (1/56) |
| (Leaf Area | | | | | |
| Duration), | | | | | |
| i.e. average | | | | | |
| number of days | | | | | |
| for which | | | | | |
| leaves remain | | | | | |
| green | | | | | |
| Pods/Plant | 5.5 (0.07) | 10.1 (0.71) | 12.2 (0.49) | 12.1 (0.35) | 11.3 (1.13) |
| Seeds/Pod | 2.4 (0.014) | 2.7 (0.07) | 2.9 (0.09) | 3.2 (9.12) | 2.7 (0.16) |
| Gm/25 seeds | 3.4 (0.15) | 3.9 (0.18) | 4.0 (0.21) | 4.1 (0.13) | 3.8 (0.28) |
| Husk, gm | 0.20 (0.12) | 0.23 (0.03) | 0.23 (0.01) | 0.22 (0.02) | 0.21 (0.04) |
| (Pod-Seeds), | | | | | |
| i.e. the weight | | | | | |
| of husk reported | | | | | |
| is the total | | | | | |
| weight of the | | | | | |
| pods minus the | | | | | |
| weight of the | | | | | |
| seeds. | | | | | |

TABLE 31
Biochemical parameters at flowering

| mg/100 g Fresh | | | | | |
|---|---|---|---|---|---|
| Wt. of Leaves | | | | | |
| Chlorophyll-a | 101.2 (3.12) | 122.7 (3.24) | 134.0 (2.82) | 128.9 (2.08)) | 126.2 (2.58) |
| Chlorophyll-b | 108.5 (3.45) | 137.6 (2.98) | 140.1 (2.54) | 139.3 (2.92) | 130.1 (2.53) |
| Proline | 16.2 (0.65) | 25.2 (0.82) | 30.1 (0.18) | 26.5 (0.62) | 26.6 (0.54) |
| Polyphenols | 23.68 (1.24) | 34.12 (1.02) | 46.98 (1.08) | 46.01 (0.68) | 45.01 (1.15) |
| Chlorophyll | 0.64 (0.03) | 0.72 (0.04) | 0.88 (0.02) | 0.86 (0.03) | 0.83 (0.02) |
| Stability Index | | | | | |
| g/100 g Fresh | | | | | |
| Wt. of leaves | | | | | |
| Reducing Sugars | 0.56 (0.05) | 0.62 (0.04) | 0.74 (0.05) | 0.68 (0.02) | 0.69 (0.04) |
| Non-reducing Sugars | 0.67 (0.08) | 1.28 (0.06) | 1.9 (0.05) | 1.8 (0.05) | 1.7 (0.06) |
| Proteins | 2.26 (0.12) | 2.28 (0.2) | 3.2 (0.18) | 2.6 (0.12) | 2.8 (0.18) |

Thus, R-100 induced increases in a variety of growth parameters, such as height, number of branches, total leaf area, chlorophyll, etc. With an increase in total proteins and carbohydrates and particularly in non-reducing sugars, increase in number of pods and seeds per pod and with a considerable increase in LAD, higher yield of oilseeds can be expected. The increase in LAD or delaying of leaf senescence was particularly significant for legumes as they otherwise suffer from monocarpic senescence leading to lower overall yield in comparison with cereals.

The level of defense chemicals, polyphenols, was also considerably enhanced.

Higher proline levels and chlorophyll stability index are both strong indicators of environmental stress resistance against drought, frost, etc. Thus, an increase in biotic and abiotic stress tolerance was noted in this trial. Plants showed higher resistance to pest damage and also higher tolerance to water stress.

### Example 26A

### Effect of CAM Plant Extracts in Flowering and Seed Production

Trials were carried out at the Pune University campus with R-5 PLUS (batch 000930) and R-5 MINUS (batch 000930) oil. Brassica juncea L. plants were cultivated in soil (farmyard manure and garden soil in 1:3 ratio) in pots 20 cm×20 cm×40 cm. Fifteen plants were grown in each pot, and each treatment group consisted of 2 pots. Control solution was distilled water. Spray solutions of different concentrations were made in 100 ml distilled water. 100 ml of solution was used per spray per pot. Spraying was done on 8^{th} 28^{th} and 48^{th} day after sowing. No spraying was done after flowering.

Two sets of treatment were carried out. Average values of the combined set are reported in Tables B and C.

Values in parentheses below indicate standard deviations.

TABLE B
Plant Height (cm) (days (d) after treatment)

| Spray Conc. | R-5 PLUS | | | R-5 MINUS | | |
|---|---|---|---|---|---|---|
| mg/100 ml | 20 d | 60 d | 90 d | 20 d | 60 d | 90 d |
| 0 | 6.38 | 48.65 | 92.36 | 6.38 | 48.65 | 92.36 |
| | (0.52) | (1.21) | (2.03) | (0.58) | (1.21) | (2.03) |
| 3 | 7.82 | 53.92 | 106.86 | 7.95 | 52.36 | 88.54 |
| | (0.82) | (1.55) | (2.120 | (0.61) | (1.08) | (2.88) |
| 10 | 9.38 | 43.25 | 62.36 | 8.56 | 38.58 | 68.98 |
| | (0.57) | (1.03) | (3.21) | (0.82) | (1.31) | (3.24) |

Note:
Above values are mean of five plants from each set.

TABLE C
Yield

| | R-5 PLUS | | | | R-5 MINUS | | | |
|---|---|---|---|---|---|---|---|---|
| Spray Conc. | | Pods/ | Seed | | | Pods/ | Seed | |
| mg/100 ml | D. F. | Plant | mg. | Yield g | D. F. | Plant | mg | Yield. g |
| 0 | 65.32 | 106.9 | 436.4 | 4.85 | 65.32 | 106.9 | 436.4 | 4.85 |
| | (0.56) | (3.25) | (5.36) | (0.45) | (0.56) | (3.25) | (5.36) | (0.45) |
| 3 | 63.88 | 124.2 | 532.2 | 5.68 | 65.52 | 110.3 | 502.4 | 5.02 |
| | (0.87) | (3.78) | (4.98) | (0.47) | (0.85) | (4.56) | (5.21) | (0.84) |
| 10 | 63.51 | 117.3 | 494.8 | 5.12 | 62.35 | 78.21 | 411.21 | 3.69 |
| | (0.96) | (3.21) | (5.33) | (0.89) | (0.75) | (4.89) | (4.98) | (0.98) |
| 100 | 62.25 | 98.65 | 431.25 | 4.36 | 60.21 | 61.25 | 408.23 | 2.56 |
| | (0.87) | (4.56) | (5.41) | (0.56) | (0.71) | (3.21) | (5.69) | (0.85) |

Note:
D. F. = Days to Flowering; Seed wt is in mg per 100 seeds; Yield is in g/plant
Note:
The above values are mean of five plants from each set. Yield, numbers of pods and seed weight were taken at the time of harvest (110 days after sowing).

The data from the two tables highlight the differential activity possible by fractionating extracts.

The plant height data shows the early onset of toxicity of the R-5 MINUS oil compared to R-5 PLUS oil. This observation is further corroborated by the data on the number of pods per plant, average seed weight and the yield per plant.

What is particularly striking is the strongly negative effect MINUS extract has on flowering, as seen from the number of pods and the detrimental effect on seed size.

However, R-5 PLUS shows excellent promotional effects, even at 10 mg per spray, up to harvesting.

The negative effects with R-5 MINUS would have been even greater if the spraying was continued beyond the third spray.

### Example 26B

### Effect of Non-CAM Plants on Phaseolus radiatus

Experiments were carried out at Pune, India in November 2001 using a variety of oil extract preparations. Phaseolus radiatus was grown in loamy soil and farmyard manure (3:1), 15 kg/pot (earthen pots with 33 cm diam. and 25 cm high). The two dose levels used were 1 μl, and 10 μl of oil extract per treatment applied in one liter of water per pot. Two pots per dose were used. Plants were treated 34 days and 46 days after sowing.

Date of sowing: 14^{th} Nov., 2001

First Treatment: 18^{th} Dec., 2001

Second Treatment: 30^{th} Dec., 2001

Date of Observation: 6^{th} Jan., 2002

The growth parameters were examined (Table 26 A). Values are mean of five plants from each pot.

TABLE 26A
Effect of Non-CAM Plant Extracts on Phaseolus
radiatus

| | Concen- | | | | |
|---|---|---|---|---|---|
| | tration | | | | |
| | μl extract/ | Height of | Number of | Number | Number of |
| Extract | treatment | Plant, cm | Branches | of Leaves | Inflorescences |
| Control | 0 | 18.0 | 1.0 | 10.2 | 1.8 |
| Sesame | 1 | 13.7 | 0.0 | 9.4 | 1.0 |
| Oil | 10 | 21.0 | 1.0 | 11.2 | 1.8 |
| TABA- | 1 | 22.2 | 1.0 | 11.2 | 2.8 |
| 28 | 10 | 26.3 | 1.6 | 13.6 | 3.8 |
| PE-100 | 1 | 24.9 | 1.0 | 11.0 | 1.4 |
| PLUS | 10 | 28.4 | 3.4 | 13.6 | 4.2 |
| CROT- | 1 | 22.4 | 0.6 | 10.2 | 0.6 |
| 28.2 | 10 | 26.0 | 1.0 | 14.0 | 4.6 |
| PLUS | | | | | |
| AZIN- | 1 | 25.1 | 1.2 | 11.0 | 2.2 |
| C-29 | 10 | 20.9 | 0.8 | 9.2 | 1.4 |
| TRIGF- | 1 | 20.8 | 1.6 | 9.4 | 0.8 |
| 29.62 | 10 | 23.3 | 1.0 | 11.0 | 1.2 |
| TVUL- | 1 | 26.3 | 1.8 | 13.4 | 4.2 |
| 32.83 | 10 | 28.6 | 2.0 | 13.4 | 4.8 |
| PLUS | | | | | |
| MPRU- | 1 | 21.3 | 1.0 | 13.6 | 1.4 |
| 27.1 | 10 | 23.3 | 1.0 | 14.0 | 2.2 |

Several extracts show a strong promotion of growth and maturation parameters such as height, number of branches and leaves and inflorescence. Phylanthus emblica (PE-100 PLUS) and Triticum vulgare (TVUL-32.83 PLUS) extracts show strong activity in all parameters even at 1 μl dose per application. AZIN-C-29 also shows strong activity at 1 μl dose. However, the activity reduces at the higher dose. All the other extractst tested also show promotional activity at 10 μl dose.

### Example 26C

### Effect of Plant Extracts and Combinations on Phaseolus radiatus

Field experiments were carried out at Pune, India in May 2002 using a variety of oil extract preparations and their combinations. An RBD design was used. There were three replicates of each treatment dose level for each preparation. The two treatment dose levels used were 1 μl, and 10 μl of oil extract per treatment administered in 100 ml of distilled water by foliar spray.

Each treatment plot was 1.8m×0.45 m and was fertilized with 2.5 kg of farmyard manure. In each treatment a plot of 30 seeds of Phaseolus radiatus was sown. Weak plants were weeded out to leave 20 plants per plot.

Apart from individual extracts, two combinations were also tested.

A mixture titled ‘AMA-15’ oil was prepared. This oil contains equal parts of 1) a mixture of Zingiber officinale, Carum copticum, Cuminum cyminum, Piper longum, 2) Terminalia Bellerica, Terminalia chebula, Phyllanthus emblica, Aloe indica, Glycyrrhiza glabra, and 3) Trigonella, Linum usitatisum, Phaseolus radiatus, Triticum vulgar. The total amount of the herb equivalent was 15 gm per 100 gm of the ‘AMA-15’ oil. The ‘AMA-15’ oil also contained MCT oil( 60/40 mixture of caprylic/capric acid tryglycerides) at the level of 25 gm per 100 gm of ‘AMA-15’ oil.

A mixture titled ‘SPRAIN-20’ oil was prepared. This oil contains 12 parts of 1) a mixture of Trigonella, Linum usitatisum, Phaseolus radiatus, Triticum vulgar, 4 parts of 2) a mixture of Terminalia Bellerica, Terminalia chebula, Phyllanthus emblica, Aloe indica, Glycyrrhiza glabra, and 4 parts of 3) a mixture of Phylanthus emblica, Mucuna pruriens and Glycyrrhiza glabra. The total amount of the herb equivalent was 20 gm per 100 gm of the ‘SPRAIN-20’ oil. The ‘SPRAIN-20’ oil also contained MCT oil (60:40 mixture of caprylic/capric acid tryglycerides) at the level of 25 gm per 100 gm of ‘SPRAIN-20’ oil.

Date of sowing: 5^{th} May, 2002

First Treatment: 24^{th} May, 2002

Second Treatment: 10^{th} Aug., 2002

Date of Observation: 25^{th} Aug., 2002

The growth parameters were examined (Table 26 B). Ten (10) plants from each replicate were observed. Estimated values are mean of 30 (10 per replicate) observations from each treatment.

Values of standard deviation (s.d.) are given in the same box below the mean values.

TABLE 26 B
Effect of Plant Extracts and Combinations on
Phaseolus radiatus
Date of observation: 25th August, 2002.

| Dosage | | | | | | | | | Grain | | Dry | |
|---|---|---|---|---|---|---|---|---|---|---|---|---|
| μl/100 ml | Height | | Branches/ | | Leaves/ | | Pods/ | | Yield | | weight | |
| distilled | cm | | Plant | | Plant | | Plant | | gm/Plant | | gm/Plant | |
| water | 1 μl | 10 μl | 1 μl | 10 μl | 1 μl | 10 μl | 1 μl | 10 μl | 1 μl | 10 μl | 1 μl | 10 μl |
| Control | 67.2 | 67.2 | 4.58 | 4.59 | 8.84 | 8.85 | 21.82 | 21.91 | 7.08 | 7.07 | 30.5 | 30.9 |
| Dist. | 0.72 | 0.72 | 0.06 | 0.06 | 0.05 | 0.07 | 0.37 | 0.27 | 0.06 | 0.05 | 0.70 | 0.91 |
| Water | | | | | | | | | | | | |
| Sesame | 68.9 | 68.74 | 4.78 | 4.84 | 8.90 | 8.87 | 23.21 | 23.24 | 7.17 | 7.18 | 31.7 | 31.8 |
| Oil | 0.63 | 0.62 | 0.08 | 0.07 | 0.03 | 0.02 | 0.31 | 0.52 | 0.05 | 0.05 | 0.46 | 0.65 |
| AMA- | 71.2 | 70.7 | 5.33 | 5.23 | 9.20 | 9.15 | 26.69 | 26.77 | 8.88 | 8.86 | 33.4 | 32.5 |
| 15 | 0.52 | 0.62 | 0.05 | 0.10 | 0.07 | 0.07 | 0.31 | 0.68 | 0.12 | 0.11 | 0.85 | 1.11 |
| RS-10 | 72.3 | 71.6 | 5.93 | 5.80 | 9.32 | 9.26 | 27.98 | 27.52 | 8.98 | 8.95 | 34.0 | 33.3 |
| | 0.58 | 0.44 | 0.05 | 0.07 | 0.04 | 0.06 | 0.52 | 0.39 | 0.14 | 0.05 | 0.44 | 0.59 |
| ZOFF- | 71.5 | 70.4 | 5.48 | 5.31 | 9.35 | 9.31 | 26.23 | 25.98 | 8.76 | 8.73 | 32.5 | 32.3 |
| 55.2 | 0.47 | 0.65 | 0.06 | 0.04 | 0.05 | 0.10 | 0.25 | 0.21 | 0.10 | 0.04 | 0.46 | 0.99 |
| PLUS | | | | | | | | | | | | |
| MPRU- | 70.6 | 69.9 | 5.55 | 5.34 | 9.11 | 9.08 | 27.25 | 27.22 | 8.99 | 8.92 | 33.9 | 33.5 |
| 27.4 | 0.38 | 0.59 | 0.18 | 0.12 | 0.10 | 0.06 | 0.29 | 0.58 | 0.14 | 0.06 | 0.34 | 1.15 |
| SPRAIN- | 72.8 | 72.1 | 5.75 | 5.63 | 9.62 | 9.26 | 28.27 | 27.85 | 9.48 | 9.36 | 34.3 | 34.4 |
| 20 | 0.80 | 0.45 | 0.07 | 0.08 | 0.06 | 0.03 | 0.47 | 0.83 | 0.27 | 0.06 | 0.37 | 0.46 |
| HA- | 71.9 | 71.2 | 5.27 | 5.21 | 9.31 | 9.13 | 25.88 | 25.60 | 8.11 | 7.86 | 34.3 | 33.8 |
| 29.7 | 0.83 | 0.26 | 0.04 | 0.08 | 0.08 | 0.04 | 0.32 | 0.49 | 0.12 | 0.04 | 0.51 | 0.50 |
| GGLAB- | 71.0 | 70.1 | 5.72 | 5.44 | 9.79 | 9.67 | 26.06 | 25.77 | 8.76 | 8.85 | 34.4 | 34.1 |
| 27.43 | 0.66 | 0.56 | 0.20 | 0.13 | 0.09 | 0.03 | 0.43 | 0.42 | 0.22 | 0.10 | 0.73 | 0.52 |
| R-100 | 72.9 | 72.3 | 5.77 | 5.58 | 9.69 | 9.59 | 27.68 | 27.20 | 9.51 | 9.23 | 34.6 | 34.1 |
| PLUS | 0.31 | 0.84 | 0.04 | 0.04 | 0.03 | 0.05 | 0.63 | 0.85 | 0.17 | 0.13 | 0.55 | 0.11 |

In the above Table, several extracts show a promotion of growth parameters such as height, number of branches and leaves and number of pods. They also show an increase in dry biomass. However, increase in the grain yield in case of several extracts is 20% to 30%. The combination extracts also show these gains.

The extracts showing a significant promotion in growth parameters and in grain yield have also shown a good promotion of P. Radiatus shoot and root growth as documented in the Table 12 set of tables. Thus, the germination assay results of growth promotion are further corroborated with the promotion observed in the field trial encompassing the entire crop cycle.

Thus, extracts made as per the methods of this invention are promoters of plant growth and biomass yield throughout the entire life cycle of the plant.

### Example 27

### Post-Harvest Ripening of Banana, Musa paradisii Cv. Basrai

Fruit were selected from mature bunches. Bunch of 14-18 fruit each, uniform in size were selected. Test solutions were made with 0, 20, 60, 100 and 200 micro-liter of R-5 oil per 1000 ml distilled water. One bunch was treated with each test solution for 30 minutes. Green life and yellow life were estimated by visual examination. Acidity and total soluble solids (TSS) were measured at the end of yellow life. Change of peel color from green to yellow indicated end of green life. Weakening of the fruit stalk, causing the fruit to be detachable, indicated end of yellow life.

As shown in Table 32, the shelf life of banana was extended by treatment up to 100 μl/liter, with the maximum extension at 60 gl/liter. Acidity development was slowed, and TSS levels steadily increased with concentration.

TABLE 32
Effect on ripening and shelf life

| | | | Green | Yellow | Total |
|---|---|---|---|---|---|
| Treatment | TSS | | Life (G) | Life(Y) | Life (G + Y) |
| μl/L | % Brix | Acidity % | Day | Day | Day |
| Control | 23.88 | 0.63 | 8 | 5 | 13 |
| 20 | 24.58 | 0.61 | 9 | 6 | 15 |
| 60 | 24.75 | 0.57 | 9 | 7 | 16 |
| 100 | 25.05 | 0.54 | 9 | 6 | 15 |
| 200 | 25.25 | 0.52 | 7 | 5 | 12 |

Life extension was likely due to a progressively slower accumulation of α-amylase activity, with increasing concentrations up to 100 μl/liter. The peak of α-amylase activity coincided with the end of yellow life. Afterwards, the activity declined rapidly. At the highest concentration, this process was reversed: amylase activity peaked early, as did the end of yellow life. Thus, partial inhibition of α-amylase activity results in longer shelf life of fruit (Table 33).

TABLE 33
Effect of R-5 on α-amylase activity during
ripening

| Treatment | 1^{st} | 3^{rd} | 5^{th} | 7^{th} | 9^{th} |
|---|---|---|---|---|---|
| μl/liter | Day | Day | Day | Day | Day |
| Control | 63.5 | 115.3 | 190.5 | 150.5 | 34.5 |
| 20 | 50.4 | 102.7 | 168.5 | 170.6 | 60.3 |
| 60 | 48.6 | 98.7 | 165.1 | 178.3 | 62.4 |
| 100 | 47.6 | 98.0 | 172.2 | 164.8 | 52.3 |
| 200 | 48.7 | 101.3 | 199.5 | 120.2 | 20.3 |

Note:
Days counted during yellow life.
Activity expressed as change of O.D./min/mg protein

Applications to Monocotyledonous Plants

### Example 28

### Effect of Oil Medium on Germination of Sorghum vulgare (cv. M, 35-1)

R-100 was made by the methods in examples described above with commercial grade coconut oil, and safflower oil, respectively. 20 seeds of Sorghum vulgare (Jowar) were placed in a plate with 5ml of distilled water containing various concentrations of R-100 oil or plain base oil (controls). On the 7^{th} day after initiation of the experiments, mean values for several variables of 11 seedlings were taken. As shown in Table 34, R-100 made in coconut and safflower oil media promoted both rooting and shooting in germination up to 1 μl/50 ml distilled water. At the higher concentration of 10 μl/50 ml DW, both R-100 oils showed a marked decline in root and shoot length unlike the plain coconut or safflower oil medium.

TABLE 34
Effect of oil medium on R-100 activity in germination

| BASE: | R-100 | Control R-100 | Control | |
|---|---|---|---|---|
| Conc. | Coconut oil | Coconut oil | Safflower oil | Safflower oil |

Root Length(cm)

| | | | | |
|---|---|---|---|---|
| 0.0 | 5.12(0.78) | 5.12(0.78) | 5.12(0.78) | 5.12(0.78) |
| 0.33 | 6.13(0.75) | 5.28(0.48) | 6.78(0.42) | 5.86(0.82) |
| 1.0 | 7.12(0.65) | 6.24(0.57) | 7.25(0.40) | 6.12(0.67) |
| 3.0 | 8.26(0.76) | 6.76(0.45) | 8.56(0.38) | 6.92(0.82) |
| 10.0 | 5.46(0.72) | 7.02(0.23) | 5.98(0.37) | 7.21(0.0.92) |

Shoot Length(cm)

| | | | | |
|---|---|---|---|---|
| 0.0 | 2.58(0.72) | 2.58(0.72) | 2.58(0.78) | 2.58(0.78) |
| 0.33 | 3.12(0.72) | 2.65(0.45) | 3.12(0.36) | 2.94(0.56) |
| 1.0 | 3.62(0.78) | 3.14(0.38) | 3.84(0.46) | 3.28(0.82) |
| 3.0 | 4.16(0.98) | 3.42(0.56) | 4.56(0.42) | 3.83(0.74) |
| 10.0 | 2.83(0.82) | 3.62(0.31) | 3.23(0.39) | 3.78(0.62) |

Note:
Concentration in the medium is in μl of R-100 oil/50 ml distilled water. Values in parenthesis indicate standard deviation.

### Example 29

### Effect of CAM Plant Extracts in Seed Germination in Monocots

R-100 and C-100 were used. Twenty (20) seeds of Sorghum vulgare (cv. M, 35-1)(Jowar) were placed in a plate with 5ml of distilled water containing different concentrations of R-100 oil. On the 7^{th} day after initiation of experiment, mean values of several variables of 11 seedlings were taken. As shown in Table 35, R-100 and C-100 promoted both rooting and shooting in germination compared to controls (0.0 concentration and sesame oil at 1 μl/50 ml distilled water).

TABLE 35
Effect of R-100 and C-100 on germination

| | | | |
|---|---|---|---|
| Conc. | C-100 oil | R-100 oil | Sesame Oil |

Root Length (cm)

| | | | |
|---|---|---|---|
| 0.0 | 5.12 (0.78) | 5.12 (0.78) | 5.12 (0.78) |
| 0.04 | 6.98 (0.52) | 6.52 (0.74) | |
| 0.20 | 8.45 (0.12) | 7.98 (0.63) | |
| 1.0 | 7.55 (0.23) | 8.02 (0.65) | 6.14 (0.68) |

Shoot Length (cm)

| | | | |
|---|---|---|---|
| 0.0 | 2.58 (0.72) | 2.58 (0.72) | |
| 0.04 | 3.56 (0.51) | 3.29 (0.71) | |
| 0.20 | 4.52 (0.58) | 4.18 (0.74) | 3.14 (0.47) |
| 1.0 | 4.28 (0.56) | 4.12 (0.75) | |

Note:
Concentration in the medium is in μl of oil/50 ml distilled water.
Values in parenthesis indicate standard deviation.

### Example 29 A

### Effect of Plant and Non-Plant Extracts in Seed Germination in Monocots

Agar (0.8 wt. % agar in distilled water) was digested in water bath to get transparent medium and 100 ml of this solution was poured in 300 ml culture bottles. Different doses of oil extracts of various Angiosperm-monocotyledon, Angiosperm-dicotyledon and Gymnosperm plants or plain base sesame oil were added to each bottle. Bottles were sterilized at 15 lbs for 20 minutes in an autoclave. Surface sterilized 10 seeds of Triticum vulgare were added to each bottle and germinated in dark for 7 days. On the 7^{th} day after initiation of experiment, values of shoot length, root length, and dry weight of biomass (dried in an oven at 70-80 deg. C. for constant dry weight) were measured for 10 seedlings. Mean values of 10 seedlings have been reported in Table 29A-29D. Several extracts and fractions promoted both rooting and shooting and biomass mobilization in germination compared to controls (0.0 concentration and sesame oil at 1, 3 and 10 μl/100 ml base medium).

TABLE 29A
Effect of plant extracts on T. vulgare seed
germination activity

| | | | | PE- | PE- | CROT- | CROT- | | PGL- |
|---|---|---|---|---|---|---|---|---|---|
| Extract/ | | Sesame | TABA | 100 | 100 | 28.2 | 62.66 | AZIN- | C- |
| Bottle | | Oil | 28 | PLUS | MINUS | PLUS | MINUS | C-29 | 33 |
| 0 | Shoot | 9.2 | | | | | | | |
| Control | (cm) | (0.48) | | | | | | | |
| | Root | 9.6 | | | | | | | |
| | (cm) | (0.53) | | | | | | | |
| | Total | 18.9 | | | | | | | |
| | (cm) | (0.79) | | | | | | | |
| 1 | Shoot | 10.9 | 12.8 | 11.5 | 11.9 | 11.5 | 11.1 | 13.5 | 12.8 |
| | (cm) | (0.45) | (0.87) | (0.80) | (0.78) | (0.77) | (0.45) | (0.53) | (0.44) |
| | Root | 11.5 | 11.9 | 12.6 | 11.7 | 11.1 | 9.7 | 11.5 | 10.5 |
| | (cm) | (0.50) | (0.62) | (0.62) | (0.72) | (0.71) | (0.48) | (0.44) | (0.40) |
| | Total | 22.4 | 24.7 | 24.0 | 23.7 | 22.6 | 20.8 | 25.0 | 23.2 |
| | (cm) | 0.75) | (1.40) | (0.88) | (1.09) | (1.26) | (0.75) | (0.81) | (0.57) |
| 3 | Shoot | 12.1 | 12.42 | 13.1 | 14.1 | 11.6 | 13.0 | *14.0 | 14.1 |
| | (cm) | (0.85) | (0.86) | (0.56) | (0.66) | (0.50) | (0.77) | (1.00) | (0.70) |
| | Root | 12.9 | 12.6 | 10.9 | 12.9 | 10.8 | 11.5 | *11.6 | 12.2 |
| | (cm) | (1.06) | (0.65) | (0.68) | (0.82) | (0.41) | (0.57) | (0.62) | (0.85) |
| | Total | 25.1 | 25.0 | 24.0 | 27.0 | 22.4 | 24.5 | *25.6 | 26.3 |
| | (cm) | (0.98) | (1.22) | (1.00) | (1.10) | (0.92) | (1.05) | (1.13) | (0.85) |
| 10 | Shoot | 13.7 | 12.2 | 9.71 | 12.9 | 11.9 | 12.0 | *12.0 | 12.6 |
| | (cm) | (0.61) | (1.06) | (0.51) | (0.99) | (0.72) | (0.70) | (0.85) | (0.63) |
| | Root | 13.2 | 13.8 | 10.4 | 10.5 | 10.5 | 10.6 | *12.6 | 11.8 |
| | (cm) | (0.54) | (0.63) | (0.71) | (0.48) | (0.57) | (0.38) | (1.13) | (0.90) |
| | Total | 26.13 | 26.0 | 20.1 | 23.4 | 22.4 | 22.6 | *24.6 | 24.4 |
| | (cm) | (0.76) | (1.50) | (0.77) | (1.20) | (1.10) | (0.64) | (1.54) | (1.21) |

*In this set, there was a liquefaction of the base medium.
Note:
Extract/Bottle in the medium is in μl of oil/100 ml base medium.
Values in parenthesis indicate standard deviation.

Dry biomass weight at the end of 7 days is reported in Table 29 B below.

Cot.—Cotyledon; E.A.—Embryo Axis (Root+Shoot); Dry weight/10 seedlings.

TABLE 29B
Effect of plant extracts on T. vulgare germination
activity

| | | | | PE- | PE- | CROT- | CROT- | | PGL- |
|---|---|---|---|---|---|---|---|---|---|
| Extract/ | | Sesame | TABA- | 100 | 100 | 28.2 | 62.66 | AZIN- | C- |
| Bottle | | Oil | 28 | PLUS | MINUS | PLUS | MINUS | C-29 | 33 |
| 0 | E.A.(mg) | 142 | | | | | | | |
| Control | Root(mg) | 61 | | | | | | | |
| | Shoot(mg) | 81 | | | | | | | |
| 1 | E.A.(mg) | 166 | 223 | 165 | 179 | 184 | 147 | 222 | 191 |
| | Root(mg) | 73 | 95 | 76 | 77 | 78 | 56 | 98 | 76 |
| | Shoot(mg) | 93 | 128 | 89 | 102 | 106 | 91 | 124 | 115 |
| 3 | E.A.(mg) | 210 | 187 | 178 | 207 | 187 | 210 | *176 | 183 |
| | Root(mg) | 81 | 86 | 74 | 89 | 79 | 90 | *72 | 77 |
| | Shoot(mg) | 129 | 99 | 104 | 118 | 108 | 120 | *104 | 106 |
| 10 | E.A.(mg) | 204 | 194 | 182 | 163 | 187 | 185 | *206 | 192 |
| | Root(mg) | 81 | 77 | 78 | 71 | 70 | 71 | *85 | 78 |
| | Shoot(mg) | 123 | 117 | 104 | 92 | 117 | 114 | *121 | 114 |

*In this set, there was a liquefaction of the base medium
Note:
Extract/Bottle in the medium is in μl of oil/100 ml base medium.

In this set, sesame oil itself shows a steady promotion in both root and shoot growth as also in biomass growth (E.A.). Phyllanthus emblica (PE-100 PLUS and MINUS) fractions and Azadiracta indica (AZIN-C-29) show further enhancement at the lowest dose of 1 in μl of oil/100 ml base medium.

TABLE 29C
Effect of plant extracts on T. vulgare seed germination activity

| | | | | SCHIR- | SCHIR- | TVUL- | TVUL- | | |
|---|---|---|---|---|---|---|---|---|---|
| Extract/ | | Sesame | HA- | 28.8 | 62.66 | 32.8 | 66.4 | MPRU- | GGLAB- |
| Bottle | | Oil | 29.7 | PLUS | MINUS | PLUS | MINUS | 27.1 | 27.43 |
| 0 | Shoot | 10.4 | | | | | | | |
| Control | (cm) | (1.18) | | | | | | | |
| | Root | 11.83 | | | | | | | |
| | (cm) | (0.98) | | | | | | | |
| | Total | 22.26 | | | | | | | |
| | (cm) | (1.95) | | | | | | | |
| 1 | Shoot | 12.2 | 12.4 | 11.2 | 12.1 | 11.7 | 11.1 | 11.1 | 17.2 |
| | (cm) | (0.91) | (0.94) | (0.55) | (0.53) | (0.62) | (0.54) | (0.59) | (0.84) |
| | Root | 12.0 | 12.0 | 17.4 | 16.1 | 12.5 | 11.9 | 12.1 | 13.1 |
| | (cm) | (0.87) | (0.80) | (0.94) | (0.88) | (0.56) | (0.92) | (0.84) | (0.67) |
| | Total | 24.2 | 24.3 | 28.6 | 28.2 | 24.2 | 23.0 | 23.2 | 30.3 |
| | (cm) | (1.41) | (1.09) | (1.19) | (0.71) | (0.76) | (0.91)) | (0.94) | (0.98) |
| 3 | Shoot | 14.7 | 12.5 | 13.0 | 13.4 | 13.1 | 13.1 | 14.4 | 14.9 |
| | (cm) | (0.66) | (0.65) | (0.82) | (0.72) | (0.74) | (0.58)) | (0.92) | (0.82) |
| | Root | 12.2 | 16.3 | 15.5 | 14.2 | 14.1 | 12.4 | 12.4 | 13.6 |
| | (cm) | (0.56) | (0.54) | (1.94) | (0.89) | (0.94) | (0.86) | (0.86) | (0.94) |
| | Total | 26.8 | 28.8 | 28.6 | 27.6 | 27.2 | 25.5 | 26.8 | 28.5 |
| | (cm) | (0.82) | (1.01) | (1.80) | (1.18) | (1.29) | (0.88) | (1.33) | (1.24) |
| 10 | Shoot | 15.1 | 14.1 | 11.8 | 14.0 | 15.3 | 13.6 | 17.5 | 13.4 |
| | (cm) | (0.49) | (0.43) | (0.79) | (0.73) | (0.83) | (0.98) | (1.22) | (0.62) |
| | Root | 12.3 | 17.7 | 9.6 | 12.1 | 17.2 | 12.8 | 12.2 | 14.2 |
| | (cm) | (0.8) | (1.17) | (0.75) | (1.10) | (1.54) | (0.96) | (0.95) | (0.89) |
| | Total | 27.4 | 31.8 | 21.4 | 26.2 | 32.5 | 26.4 | 29.7 | 27.6 |
| | (cm) | 0.91) | (1.22) | (0.92) | (0.77) | (2.0) | (1.13) | (1.91) | (1.34) |

Note:
Extract/Bottle in the medium is in μl of oil/100 ml base medium.
Values in parenthesis indicate standard deviation.

Dry biomass weight at the end of 7 days is reported in Table 29D below.

Cot.—Cotyledon; E.A.—Embryo Axis (Root+Shoot); Dry weight/10 seedlings.

TABLE 29D
Effect of plant extracts on T. vulgare germination activity

| | | | | SCHIR- | SCHIR- | TVUL- | TVUL- | | |
|---|---|---|---|---|---|---|---|---|---|
| Extract/ | | Sesame | HA- | 28.8 | 62.66 | 32.8 | 66.4 | MPRU- | GGLAB- |
| Bottle | | Oil | 29.7 | PLUS | MINUS | PLUS | MINUS | 27.1 | 27.43 |
| 0 | E.A.(mg) | 121 | | | | | | | |
| Control | Root(mg) | 49 | | | | | | | |
| | Shoot(mg) | 72 | | | | | | | |
| 1 | E.A.(mg) | 150 | 168 | 193 | 195 | 198 | 166 | 180 | 208 |
| | Root(mg) | 62 | 64 | 77 | 64 | 79 | 58 | 57 | 76 |
| | Shoot(mg) | 88 | 104 | 116 | 131 | 119 | 108 | 128 | 132 |
| 3 | E.A.(mg) | 171 | 234 | 209 | 184 | 199 | 179 | 197 | 185 |
| | Root(mg) | 73 | 91 | 81 | 66 | 76 | 67 | 73 | 52 |
| | Shoot(mg) | 95 | 143 | 128 | 118 | 123 | 112 | 124 | 133 |
| 10 | E.A.(mg) | 186 | 187 | 151 | 184 | 259 | 173 | 230 | 201 |
| | Root(mg) | 84 | 74 | 58 | 69 | 87 | 69 | 87 | 59 |
| | Shoot(mg) | 102 | 113 | 93 | 115 | 172 | 104 | 143 | 142 |

Note:
Extract/Bottle in the medium is in μl of oil/100 ml base medium.
Values in parenthesis indicate standard deviation.

Extracts pf Swertia chirata (SCHIR PLUS and MONUS) showed a strong root promotion and extract of Glycyrrhiza glabra (GGLAB) showed a very strong shoot promotion even at 1 μl/litre of base medium. Extracts of Holarrhena antidysenterica (HA-29.7), Mucuna pruriens (MPRU-27.1) and Triticum vulgare (TVUL-32.8 PLUS) showed a dose dependant increase in promotion.

TABLE 29E
Effect of plant extracts on T. vulgare seed germination activity

| | | | FBENG- | FBENG- | CAMA- | CAMA- |
|---|---|---|---|---|---|---|
| Extract/ | | Sesame | 29 | 100 | 26.2 | 98.4 |
| Bottle | | Oil | PLUS | MINUS | PLUS | MINUS |
| 0 | Shoot | 11.9 | | | | |
| Control | (cm) | (0.64) | | | | |
| | Root | 8.2 | | | | |
| | (cm) | (0.72) | | | | |
| | Total | 20.1 | | | | |
| | (cm) | (0.82) | | | | |
| 1 | Shoot | 13.7 | 14.1 | 14.2 | 12.9 | 15.1 |
| | (cm) | (0.64) | (1.08) | (0.44) | (0.53) | (0.75) |
| | Root | 9.4 | 15.1 | 15.7 | 14.7 | 15.4 |
| | (cm) | (0.54) | (0.43) | (0.65) | (1.27) | (0.76) |
| | Total | 23.1 | 29.2 | 29.9 | 27.6 | 30.5 |
| | (cm) | (0.75) | (1.01) | (0.75) | (1.36) | (0.89) |
| 3 | Shoot | 14.0 | 14.9 | 14.0 | 13.3 | 13.8 |
| | (cm) | (0.53) | (0.61) | (0.66) | (0.60) | (0.66) |
| | Root | 9.8 | 15.3 | 16.5 | 12.9 | 17.9 |
| | (cm) | (0.51) | (0.83) | (0.73) | (0.49) | (0.60) |
| | Total | 23.6 | 30.2 | 30.5 | 26.2 | 31.6 |
| | (cm) | (0.57) | (1.28) | (1.21) | (0.72) | (0.72) |
| 10 | Shoot | 15.8 | 15.2 | 13.4 | 14.2 | 14.8 |
| | (cm) | (0.71) | (0.54) | (0.38) | (0.62) | (1.25) |
| | Root | 10.2 | 9.8 | 15.4 | 11.4 | 13.7 |
| | (cm) | (0.40) | (0.61) | (0.59) | (0.64) | (0.84) |
| | Total | 25.9 | 25.0 | 28.8 | 25.6 | 28.4 |
| | (cm) | (1/00) | (0.91) | (0.81) | (0.74) | (1.47) |

Note:
Extract/Bottle in the medium is in μl of oil/100 ml base medium.
Values in parenthesis indicate standard deviation.

Dry biomass weight at the end of 5 days is reported in Table 29F below.

TABLE 29F
Effect of plant extracts on T. vulgare germination activity

| | | | FBENG- | FBENG- | CAMA- | CAMA- |
|---|---|---|---|---|---|---|
| Extract/ | | Sesame | 29 | 100 | 26.2 | 98.4 |
| Bottle | | Oil | PLUS | MINUS | PLUS | MINUS |
| 0 | E.A.(mg) | 121 | | | | |
| Control | Root(mg) | 52 | | | | |
| | Shoot(mg) | 69 | | | | |
| 1 | E.A.(mg) | 144 | 184 | 177 | 204 | 195 |
| | Root(mg) | 59 | 73 | 68 | 85 | 71 |
| | Shoot(mg) | 85 | 111 | 109 | 119 | 124 |
| 3 | E.A.(mg) | 163 | 214 | 214 | 178 | 213 |
| | Root(mg) | 64 | 81 | 89 | 80 | 88 |
| | Shoot(mg) | 99 | 133 | 125 | 98 | 125 |
| 10 | E.A.(mg) | 180 | 158 | 209 | 211 | 192 |
| | Root(mg) | 71 | 54 | 78 | 75 | 77 |
| | Shoot(mg) | 109 | 104 | 121 | 136 | 115 |

Note:
Extract/Bottle in the medium is in μl of oil/100 ml base medium.
Cot. - Cotyledon;
E.A. - Embryo Axis (Root + Shoot);
Dry weight/10 seedlings.

Both fractions of Ficus bengalensis and Curcuma amada show a very considerable promotion of root growth even compared to sesame oil. The biomass growth is considerably higher both for root and shoot. Thus, all four fractions are very strong promoters up to 3 μl of oil/100 ml base medium. Many of them continue to be promoters even at 10 μl of oil/100 ml base medium.

TABLE 29G
Effect of plant extracts on T. vulgare seed germination activity

| | | | | | ZOFF- | ZOFF- | ER- | |
|---|---|---|---|---|---|---|---|---|
| | | Sesame | TRIGF- | SIND- | 55.4 | 100 | 28.8 | ER-100 |
| Extract/Bottle | | Oil | 29.62 | 28.8 | PLUS | MINUS | PLUS | MINUS |
| 0 | Shoot | 11.62 | | | | | | |
| Control | (cm) | (0.60) | | | | | | |
| | Root | 10.04 | | | | | | |
| | (cm) | (0.36) | | | | | | |
| | Total | 21.66 | | | | | | |
| | (cm) | (0.79) | | | | | | |
| 1 | Shoot | 12.23 | 13.99 | 12.1 | 11.19 | 12.32 | 12.55 | 14.47 |
| | (cm) | (0.49) | (0.40) | (0.73) | (0.65) | (0.51) | (0.38) | (0.41) |
| | Root | 10.35 | 14.72 | 11.16 | 13.54 | 11.37 | 15.14 | 11.25 |
| | (cm) | (0.46) | (0.61) | (0.58) | (0.87) | (0.63) | (1.22) | (0.44) |
| | Total | 22.58 | 28.71 | 23.26 | 24.73 | 23.69 | 27.69 | 25.72 |
| | (cm) | (0.68) | (0.39) | (0.98) | (1.38) | (0.61) | (1.39) | (0.37) |
| 3 | Shoot | 13.15 | 13.46 | 14.28 | 12.86 | 13.23 | 14.7 | 15.75 |
| | (cm) | (0.39) | (0.90) | (0.71) | (0.41) | (0.37) | (0.38) | (0.51) |
| | Root | 11.33 | 14.63 | 12.45 | 12.53 | 12.60 | 16.19 | 13.38 |
| | (cm) | (0.75) | (0.88) | (0.39) | (0.78) | (0.59) | (0.56) | (0.55) |
| | Total | 24.48 | 28.09 | 26.73 | 25.39 | 25.83 | 30.89 | 29.13 |
| | (cm) | (0.93) | (1.5) | (080) | (0.74) | (0.53) | (0.51) | (0.68) |
| 10 | Shoot | 13.85 | 10.99 | 13.66 | 15.48 | 11.99 | 12.92 | 13.34 |
| | (cm) | (0.24) | (0.42) | (0.36) | (0.66) | (0.48) | (0.54) | (0.50) |
| | Root | 12.12 | 13.66 | 12.41 | 12.17 | 16.78 | 16.69 | 12.30 |
| | (cm) | (0.67) | (0.50) | (1.14) | (1.24) | (0.76) | (0.54) | (0.52) |
| | Total | 25.97 | 24.65 | 26.07 | 27.65 | 28.77 | 29.61 | 25.64 |
| | (cm) | (0.72) | (0.68) | (1.4) | (1.39) | (1.2) | (0.95) | (0.61) |

Note:
Extract/Bottle in the medium is in μl of oil/100 ml base medium.
Values in parenthesis indicate standard deviation.

All plants and fractions in this set are strong promoters of overall growth. Except Sesamum indicum, the other extracts show the activity even at 1 μl of oil/100 ml base medium.

Dry biomass weight at the end of 5 days is reported in Table 29H below.

TABLE 29 H
Effect of plant extracts on T. vulgare germination activity

| | | | | | ZOFF- | ZOFF- | ER- | |
|---|---|---|---|---|---|---|---|---|
| Extract/ | | Sesame | TRIGF- | SIND- | 55.4 | 100 | 28.8 | ER-100 |
| Bottle | | Oil | 29.62 | 28.8 | PLUS | MINUS | PLUS | MINUS |
| 0 | E.A.(mg) | 133 | | | | | | |
| Control | Root(mg) | 52 | | | | | | |
| | Shoot(mg) | 81 | | | | | | |
| 1 | E.A.(mg) | 155 | 303 | 146 | 161 | 159 | 141 | 208 |
| | Root(mg) | 57 | 98 | 56 | 59 | 58 | 56 | 76 |
| | Shoot(mg) | 98 | 205 | 90 | 102 | 101 | 85 | 132 |
| 3 | E.A.(mg) | 168 | 198 | 201 | 164 | 165 | 174 | 185 |
| | Root(mg) | 62 | 74 | 87 | 66 | 64 | 62 | 52 |
| | Shoot(mg) | 106 | 124 | 114 | 98 | 101 | 112 | 133 |
| 10 | E.A.(mg) | 170 | 172 | 148 | 171 | 188 | 149 | 203 |
| | Root(mg) | 72 | 56 | 46 | 67 | 82 | 56 | 59 |
| | Shoot(mg) | 98 | 110 | 102 | 104 | 106 | 93 | 142 |

Note:
Extract/Bottle in the medium is in μl of oil/100 ml base medium.
Cot. - Cotyledon;
E.A. - Embryo Axis (Root + Shoot);
Dry weight/10 seedlings.

All plants and fractions from this set show faster biomass accumulation.

TABLE 29I
Effect of plant extracts on T. vulgare seed germination activity

| Extract/ | | Sesame | TCHEB- | TBEL- | TBEL- |
|---|---|---|---|---|---|
| Bottle | | Oil | 27.8 | R-50 | P-50 |
| 0 | Shoot | 11.62 | | | |
| Control | (cm) | (0.60) | | | |
| | Root | 10.04 | | | |
| | (cm) | (0.36) | | | |
| | Total | 21.66 | | | |
| | (cm) | (0.79) | | | |
| 1 | Shoot | 12.23 | 12.85 | 10.33 | 16.71 |
| | (cm) | (0.49) | (0.66) | (0.61) | (0.43) |
| | Root | 10.35 | 13.99 | 13.66 | 11.22 |
| | (cm) | (0.46) | (0.86) | (0.82) | (0.53) |
| | Total | 22.58 | 26.34 | 23.99 | 27.93 |
| | (cm) | (0.68) | (1.48) | (1.28) | (0.57) |
| 3 | Shoot | 13.15 | 13.94 | 13.79 | 13.79 |
| | (cm) | (0.39) | (0.66) | (0.57) | (0.70) |
| | Root | 11.33 | 14.94 | 11.01 | 12.18 |
| | (cm) | (0.75) | (0.68) | (0.42) | (0.59) |
| | Total | 24.48 | 28.88 | 24.80 | 25.97 |
| | (cm) | (0.93) | (1.22) | (0.77) | (0.67) |
| 10 | Shoot | 13.85 | 14.59 | 14.97 | 9.73 |
| | (cm) | (0.24) | (0.82) | (0.45) | (0.87) |
| | Root | 12.12 | 14.85 | 12.14 | 10.38 |
| | (cm) | (0.67) | (1.1) | (0.65) | (0.68) |
| | Total | 25.67 | 29.44 | 27.11 | 20.11 |
| | (cm) | (0.72) | (1.55) | (0.69) | (0.63) |

Note:
Extract/Bottle in the medium is in μl of oil/100 ml base medium.
Values in parenthesis indicate standard deviation.

All fractions in this set show promotion of shoot/root growth.

TABLE 29K
Effect of plant extracts on T. vulgare seed germination activity

| | | Sesame | A-100 | A-100 | R-100 | R-100 | C-100 | C-100 |
|---|---|---|---|---|---|---|---|---|
| Extract/Bottle | | Oil | PLUS | MINUS | PLUS | MINUS | PLUS | MINUS |
| 0 | Shoot | 11.44 | | | | | | |
| Control | (cm) | (0.55) | | | | | | |
| | Root | 8.99 | | | | | | |
| | (cm) | (0.61) | | | | | | |
| | Total | 20.43 | | | | | | |
| | (cm) | (0.69) | | | | | | |
| 1 | Shoot | 11.99 | 13.73 | 13.93 | 12.57 | 12.83 | 11.11 | 11.3 |
| | (cm) | (0.40) | (0.65) | (0.30) | (0.51) | (0.55) | (0.52) | (0.38) |
| | Root | 10.76 | 11.78 | 14.71 | 15.12 | 12.78 | 10.0 | 9.46 |
| | (cm) | (0.82) | (0.58) | (0.74) | (1.0) | (0.72) | (0.47) | (0.59) |
| | Total | 22.76 | 25.51 | 28.64 | 27.69 | 25.61 | 21.11 | 20.76 |
| | (cm) | (0.83) | (0.99) | (0.91) | (1.01) | (1.1) | (0.86) | (0.73) |
| 3 | Shoot | 12.63 | 14.94 | 15.01 | 12.94 | 11.82 | 12.51 | 11.54 |
| | (cm) | (0.65) | (0.45) | (0.57) | (0.68) | (0.85) | (0.52) | (0.97) |
| | Root | 10.89 | 12.24 | 15.94 | 11.70 | 11.17 | 12.34 | 12.01 |
| | (cm) | (0.77) | (0.38) | (0.74) | (0.49) | (0.83) | (0.52) | (0.46) |
| | Total | 23.42 | 27.18 | 30.95 | 24.64 | 22.99 | 24.85 | 23.55 |
| | (cm) | (1.22) | (0.66) | (0.88) | (0.57) | (1.05) | (0.84) | (0.79) |
| 10 | Shoot | 13.99 | 16.29 | 13.88 | 9.45 | 11.87 | 12.5 | 12.86 |
| | (cm) | (0.35) | (0.76) | (0.35) | (0.56) | (0.58) | (0.50) | (0.82) |
| | Root | 11.88 | 14.66 | 9.87 | 10.79 | 10.95 | 11.21 | 14.63 |
| | (cm) | (0.54) | (0.95) | (0.36) | (0.53) | (0.56) | (0.38) | (0.81) |
| | Total | 25.87 | 30.97 | 23.75 | 20.24 | 22.82 | 23.71 | 27.49 |
| | (cm) | (0.66) | (1.29) | (0.38) | (0.99) | (0.69) | (0.75) | (1.22) |

Note:
Extract/Bottle in the medium is in μl of oil/100 ml base medium.
Values in parenthesis indicate standard deviation.

Both PLUS and MINUS fractions of A-100 (Aloe) and B-100 (Kalanchoe pinnata) show promotional activity at 1 μl of oil/100 ml base medium. C-100 MINUS fraction also shows moderate promotional activity at the higher dose of 10 μl of oil/100 ml base medium.

TABLE 29 M
Effect of plant extracts on T. vulgare seed germination activity

| | | | | RS- | | | | | |
|---|---|---|---|---|---|---|---|---|---|
| | | | | 10 | | | R-25 | R-25 | R-25 |
| Extract/ | | Sesame | R- | Leaf | R-100 | R-100 | 30 Min | 120 Min | 240 Min |
| Bottle | | Oil | 100 | Stem | (J + L/4) | (4J + L/8) | Boiling | Boiling | Boiling |
| 0 | Shoot | 11.44 | | | | | | | |
| Control | (cm) | (0.55) | | | | | | | |
| | Root | 8.99 | | | | | | | |
| | (cm) | (0.61) | | | | | | | |
| | Total | 20.43 | | | | | | | |
| | (cm) | (0.69) | | | | | | | |
| 1 | Shoot | 11.99 | 12.43 | 13.82 | 11.27 | 15.21 | 12.75 | 14.79 | 13.25 |
| | (cm) | (0.40) | (0.39) | (0.67) | (0.48) | (0.71) | (0.55) | (0.29) | (0.49) |
| | Root | 10.76 | 9.64 | 12.02 | 13.1 | 10.0 | 11.54 | 15.77 | 15.44 |
| | (cm) | (0.82) | (0.51) | (0.54) | (0.24) | (0.21) | (0.70) | (0.53) | (0.33) |
| | Total | 22.76 | 22.07 | 25.84 | 24.37 | 25.30 | 24.29 | 30.58 | 28.69 |
| | (cm) | (0.83) | (0.76) | (1.1) | (0.51) | (0.84) | (0.64) | (0.51) | (0.52) |
| 3 | Shoot | 12.63 | 13.01 | 12.02 | 13.23 | 15.65 | 15.80 | 14.49 | 14.37 |
| | (cm) | (0.65) | (0.41) | (1.34) | (0.65) | (0.62) | (0.55) | (0.35) | (0.48) |
| | Root | 10.89 | 12.61 | 13.09 | 14.05 | 11.97 | 14.28 | 12.24 | 14.81 |
| | (cm) | (0.77) | (1.01) | (0.59) | (0.82) | (0.85) | (0.53) | (0.40) | (0.36) |
| | Total | 23.42 | 25.62 | 25.11 | 27.28 | 27.62 | 30.08 | 26.73 | 29.18 |
| | (cm) | (1.22) | (1.31) | (1.39) | (1.23) | (1.09) | (1.0) | (0.56) | (0.75) |
| 10 | Shoot | 13.99 | 14.21 | 11.66 | 14.68 | 14.92 | 14.77 | 12.5 | 12.63 |
| | (cm) | (0.35) | (0.81) | (0.51) | (0.56) | (0.28) | (0.65) | (0.64) | (0.59) |
| | Root | 11.88 | 9.84 | 8.87 | 14.3 | 14.42 | 14.43 | 11.9 | 11.94 |
| | (cm) | (0.54) | (0.65) | (0.49) | (0.62) | (0.77) | (0.57) | (0.33) | (0.40) |
| | Total | 25.87 | 24.05 | 20.53 | 28.98 | 29.14 | 29.2 | 24.4 | 24.57 |
| | (cm) | (0.66) | (0.86) | (0.75) | (0.81) | (0.85) | (0.75) | (0.68) | (0.84) |

Note:
Extract/Bottle in the medium is in μl of oil/100 ml base medium.
Values in parenthesis indicate standard deviation.

These are all various methods of preparing Kalanchoe pinnata based extracts. All of them are active compared to control. R-100 is marginally better than sesame oil particularly for root promotion at 3 μl of oil/100 ml base medium.

TABLE 29O
Effect of plant extracts on T. vulgare seed germination activity

| | | | | CLNG | | | |
|---|---|---|---|---|---|---|---|
| Extract/ | | Sesame | CLNG I | II | EJAM | RSER | VR |
| Bottle | | Oil | 33.75 | 26.47 | 34.16 | 29 | 25 |
| 0 | Shoot | 10.76 | | | | | |
| Control | (cm) | (0.33) | | | | | |
| | Root | 8.98 | | | | | |
| | (cm) | (0.65) | | | | | |
| | Total | 19.34 | | | | | |
| | (cm) | (0.81) | | | | | |
| 1 | Shoot | 11.29 | 12.05 | 12.81 | 10.45 | 11.57 | 11.0 |
| | (cm) | (0.73) | (0.51) | (0.53) | (0.64) | (0.33) | (0.63) |
| | Root | 10.45 | 14.68 | 13.47 | 12.53 | 13.90 | 12.45 |
| | (cm) | (0.45) | (0.92) | (0.77) | (0.56) | (0.65) | (0.37) |
| | Total | 22.46 | 26.73 | 26.77 | 22.98 | 25.47 | 23.45 |
| | (cm) | (0.84) | (1.36) | (1.49) | (0.91) | (0.76) | (0.94) |
| 3 | Shoot | 13.02 | 12.64 | 11.92 | 12.49 | 12.64 | 12.38 |
| | (cm) | (0.42) | (0.25) | (0.64) | (0.28) | (0.34) | (0.38) |
| | Root | 11.17 | 14.24 | 15.26 | 13.72 | 14.72 | 14.38 |
| | (cm) | (0.63) | (0.50) | (1.24) | (0.34) | (0.60) | (0.85) |
| | Total | 24.19 | 26.88 | 27.18 | 26.2 | 27.4 | 27.21 |
| | (cm) | (0.66) | (0.56) | (1.14) | (0.39) | (0.94) | (0.78) |
| 10 | Shoot | 14.06 | 10.63 | 14.56 | 14.56 | 11.47 | 13.65 |
| | (cm) | (0.65) | (0.33) | (0.31) | (0.93) | (0.32) | (0.47) |
| | Root | 11.77 | 12.43 | 13.63 | 17.17 | 12.67 | 12.74 |
| | (cm) | (0.64) | (0.37) | (0.37) | (1.3) | (0.30) | (0.37) |
| | Total | 25.83 | 23.06 | 28.22 | 31.73 | 24.14 | 26.39 |
| | (cm) | (1.01) | (0.58) | (0.66) | (1.36) | (0.39) | (0.58) |

Note:
Extract/Bottle in the medium is in μl of oil/100 ml base medium.
Values in parenthesis indicate standard deviation.

All of extracts of this set show activity at 1 to 10 μl of oil/100 ml base medium.

TABLE 29Q
Effect of plant extracts on T. vulgare seed
germination activity

| Extract/ | | Sesame | PRAD | BAMO | R-100 | LUST | |
|---|---|---|---|---|---|---|---|
| Bottle | | Oil | 32.2 | 55.59 | Coconut | 35.71 | N-50 |
| 0 | Shoot | 13.54 | | | | | |
| Control | (cm) | (0.74) | | | | | |
| | Root | 9.42 | | | | | |
| | (cm) | (0.73) | | | | | |
| | Total | 22.96 | | | | | |
| | (cm) | (1.23) | | | | | |
| 1 | Shoot | 14.51 | 14.0 | 14.18 | 14.72 | 14.90 | 14.81 |
| | (cm) | (0.72) | (1.11) | (1.19) | (1.06) | (0.83) | (1.19) |
| | Root | 8.71 | 9.2 | 8.64 | 10.03 | 10.11 | 9.89 |
| | (cm) | (0.91) | (0.54) | (1.44) | (0.71) | (0.51) | (0.57) |
| | Total | 23.22 | 23.20 | 22.82 | 24.75 | 25.01 | 24.70 |
| | (cm) | (0.99) | (1.58) | (1.41) | (1.25) | (1.25) | (1.37) |
| 3 | Shoot | 14.52 | 13.46 | 14.73 | 14.75 | 15.01 | 14.63 |
| | (cm) | (0.83) | (1.46) | (0.99) | (0.84) | (0.60) | (0.79) |
| | Root | 8.11 | 8.90 | 7.91 | 9.88 | 11.27 | 10.40 |
| | (cm) | (0.98) | (1.22) | (0.99) | (0.60 | (0.78) | (0.49) |
| | Total | 22.63 | 22.36 | 22.64 | 24.63 | 26.28 | 25.03 |
| | (cm) | (1.15) | (2.16) | (1.5) | (1.14) | (1.02) | (1.04) |
| 10 | Shoot | 14.60 | 13.77 | 12.1 | 14.5 | 13.9 | 16.20 |
| | (cm) | (0.90) | (1.33) | (1.13) | (0.94) | (0.51) | (0.87) |
| | Root | 7.98 | 6.7 | 7.82 | 9.1 | 7.91 | 10.77 |
| | (cm) | (0.94) | (0.78) | (1.5) | (1.26) | (0.92) | (0.77) |
| | Total | 22.58 | 20.47 | 19.92 | 23.60 | 21.81 | 26.97 |
| | (cm) | (1.58) | (1.44) | (1.56) | (1.87) | (1.17) | (1.06) |

Note:
Extract/Bottle in the medium is in μl of oil/100 ml base medium.
Values in parenthesis indicate standard deviation.

Flaxseed (LUST-35.71), Neemleaf(N-50) and R-100 made using coconut oil as cooking oil show activity.

TABLE 29S
Effect of plant extracts on T. vulgare seed
germination activity

| Extract/ | | | OSA-T | CUCY | PILO | TICO | WISO | GLMX |
|---|---|---|---|---|---|---|---|---|
| Bottle | | Sesame Oil | 48.68 | 36.67 | 36.02 | 35.51 | 38.82 | 27.65 |
| 0 | Shoot | 13.57 | | | | | | |
| Control | (cm) | (0.88) | | | | | | |
| | Root | 11.85 | | | | | | |
| | (cm) | (0.99) | | | | | | |
| | Total | 25.42 | | | | | | |
| | (cm) | (1.33) | | | | | | |
| 1 | Shoot | 13.95 | 15.51 | 14.13 | 14.41 | 14.34 | 14.81 | 14.31 |
| | (cm) | (1.06) | (0.61) | (0.49) | (0.71) | (0.94) | (0.94) | (0.91) |
| | Root | 11.99 | 12.70 | 12.04 | 11.83 | 12.01 | 12.92 | 12.13 |
| | (cm) | (0.82) | (0.82) | (0.77) | (0.50) | (0.67) | (0.67) | (0.87) |
| | Total | 25.94 | 27.81 | 26.17 | 26.24 | 26.35 | 27.73 | 26.44 |
| | (cm) | (1.22) | (0.85) | (1.02) | (0.90) | (1.31) | (1.48) | (1.43) |
| 3 | Shoot | 14.57 | 15.55 | 15.01 | 15.05 | 14.64 | 14.90 | 14.42 |
| | (cm) | (1.57) | (0.77) | (0.80) | (0.84) | (0.84) | (0.97) | (0.75) |
| | Root | 12.10 | 13.11 | 12.43 | 12.53 | 12.71 | 12.08 | 12.29 |
| | (cm) | (0.71) | (0.91) | (0.64) | (0.73) | (0.56) | (0.97) | (1.01) |
| | Total | 26.57 | 28.66 | 27.44 | 27.58 | 27.35 | 26.98 | 26.71 |
| | (cm) | (1.59) | (1.12) | (1.05) | (0.79) | (1.08) | (1.09) | (1.12) |
| 10 | Shoot | 14.53 | 15.88 | 15.66 | 15.47 | 14.47 | 14.72 | 14.24 |
| | (cm) | (1.03) | (0.76) | (0.66) | (0.86) | (0.94) | (0.58) | (0.81) |
| | Root | 12.19 | 13.60 | 12.61 | 13.14 | 11.58 | 10.53 | 11.38 |
| | (cm) | 0.61) | (0.92) | (0.58) | (0.50) | (0.66) | (0.58) | (0.72) |
| | Total | 26.72 | 29.48 | 28.27 | 28.61 | 26.05 | 25.25 | 25.62 |
| | (cm) | (1.37) | (1.15) | (0.88) | (0.97) | (0.95) | (0.99) | (1.27) |

Note:
Extract/Bottle in the medium is in μl of oil/100 ml base medium.
Values in parenthesis indicate standard deviation.

All extracts from this set show activity.

TABLE 29U
Effect of plant fractions on T. vulgare seed
germination activity

| Extract/ | | | ASRA | KPMS | ACEP | ASAT | CACO | MOCH |
|---|---|---|---|---|---|---|---|---|
| Bottle | | Seasme Oil | 30.16 | 51.9 | 74.3 | 68.68 | 37.5 | 77.16 |
| 0 | Shoot | 13.57 | | | | | | |
| Control | (cm) | (0.88) | | | | | | |
| | Root | 11.85 | | | | | | |
| | (cm) | (0.99) | | | | | | |
| | Total | 25.42 | | | | | | |
| | (cm) | (1.33) | | | | | | |
| 1 | Shoot | 13.95 | 15.88 | 13.98 | 14.44 | 14.61 | 13.65 | 15.41 |
| | (cm) | (1.06) | (0.99) | (0.97) | (0.95) | (0.93) | (0.88) | (0.53) |
| | Root | 11.99 | 11.99 | 12.29 | 11.96 | 12.08 | 11.96 | 12.47 |
| | (cm) | (0.82) | (0.74) | (0.41) | (0.86) | (0.40) | (0.28) | (0.51) |
| | Total | 25.94 | 27.87 | 26.27 | 26.40 | 26.69 | 25.63 | 27.88 |
| | (cm) | (1.22) | (1.15) | (0.98) | (1.40) | (0.97) | (0.93) | (0.62) |
| 3 | Shoot | 14.57 | 16.13 | 14.98 | 14.46 | 14.96 | 14.19 | 15.54 |
| | (cm) | (1.57) | (0.90) | (0.84) | (0.95) | (0.76) | (0.94) | (0.75) |
| | Root | 12.10 | 12.73 | 12.32 | 12.96 | 12.48 | 12.23 | 12.69 |
| | (cm) | (0.71) | (0.96) | (0.82) | (0.75) | (0.64) | (0.59) | (0.55) |
| | Total | 26.57 | 28.86 | 27.30 | 27.42 | 27.44 | 26.42 | 28.23 |
| | (cm) | (1.59) | (1.75) | (1.20) | (1.12) | (0.99) | (1.04) | (0.86) |
| 10 | Shoot | 14.53 | 16.17 | 15.22 | 14.32 | 15.05 | 14.85 | 15.78 |
| | (cm) | (1.03) | (0.84) | (0.77) | (0.96) | (1.06) | (0.72) | (0.56) |
| | Root | 12.19 | 13.02 | 12.88 | 11.02 | 12.51 | 12.50 | 12.68 |
| | (cm) | (0.61) | (1.01) | (0.80) | (0.47) | (0.37) | (0.51) | (0.42) |
| | Total | 26.72 | 29.19 | 28.07 | 25.34 | 27.56 | 27.35 | 28.16 |
| | (cm) | (1.37) | (1.16) | (1.05) | (1.25) | (1.08) | (0.90) | (0.62) |

Note:
Extract/Bottle in the medium is in μl of oil/100 ml base medium.
Values in parenthesis indicate standard deviation.

TABLE 29W
Effect of biomass extracts on T. vulgare seed
germination activity

| | | | | MUSH- | | | | |
|---|---|---|---|---|---|---|---|---|
| Extract/ | | | PRWN- | A- | YBD- | TB- | | |
| Bottle | | Sesame Oil | 67.88 | 98.36 | 47.55 | 27.7 | PE-100 | C-100 |
| 0 | Shoot | 13.61 | | | | | | |
| Control | (cm) | (0.83) | | | | | | |
| | Root | 11.90 | | | | | | |
| | (cm) | (0.95) | | | | | | |
| | Total | 25.51 | | | | | | |
| | (cm) | (1.25) | | | | | | |
| 1 | Shoot | 13.99 | 16.57 | 13.84 | 15.88 | 16.19 | 16.55 | 16.12 |
| | (cm) | (1.03) | (0.72) | (0.87) | (0.63) | (0.66) | (0.89) | (0.56) |
| | Root | 12.04 | 12.39 | 12.29 | 13.06 | 12.19 | 12.53 | 13.01 |
| | (cm) | (0.83) | (0.78) | (0.54) | (0.77) | (0.66) | (0.67) | (0.88) |
| | Total | 26.03 | 28.96 | 26.13 | 28.94 | 28.38 | 29.08 | 29.13 |
| | (cm) | (1.17) | (0.98) | (1.06) | (0.94) | (0.68) | (1.27) | (1.01) |
| 3 | Shoot | 14.64 | 16.45 | 14.48 | 16.12 | 16.38 | 16.65 | 16.53 |
| | (cm) | (0.97) | (0.56) | (1.03) | (0.73) | (0.71) | (0.85) | (0.88) |
| | Root | 12.67 | 12.67 | 12.45 | 13.04 | 12.69 | 13.11 | 13.18 |
| | (cm) | (0.67) | (0.72) | (0.54) | (0.67) | (0.76) | (0.59) | (0.88) |
| | Total | 26.83 | 29.12 | 26.93 | 29.16 | 29.07 | 29.73 | 29.72 |
| | (cm) | (1.39) | (0.88) | (1.27) | (1.03) | (1.07) | (1.18) | (1.18) |
| 10 | Shoot | 14.83 | 16.35 | 14.84 | 15.32 | 16.30 | 16.36 | 16.24 |
| | (cm) | (0.89) | (0.79) | (0.70) | (0.63) | (0.79) | (0.81) | (0.98) |
| | Root | 12.25 | 13.01 | 12.66 | 12.71 | 13.53 | 12.67 | 13.11 |
| | (cm) | (0.40) | (0.50) | (0.59) | (0.70) | (0.75) | (0.47) | (0.96) |
| | Total | 27.80 | 29.36 | 27.50 | 28.03 | 29.83 | 29.03 | 29.35 |
| | (cm) | (1.10) | (1.15) | (1.06) | (0.95) | (1.07) | (1.15) | (1.25) |

Note:
Extract/Bottle in the medium is in μl of oil/100 ml base medium.
Values in parenthesis indicate standard deviation.

Except mushroom (MUSH-A-98.36), all other extracts including the prawn and yeast extract show promotional activity.

TABLE 29Y
Effect of biomass extracts on T. vulgare seed
germination activity

| Extract/ | | Sesame | PINI- | GYSY- |
|---|---|---|---|---|
| Bottle | | Oil | 32.4 | 26.4 |
| 0 | Shoot | 13.65 | | |
| Control | (cm) | (0.75) | | |
| | Root | 11.81 | | |
| | (cm) | (0.66) | | |
| | Total | 25.46 | | |
| | (cm) | (0.95) | | |
| 1 | Shoot | 13.93 | 14.46 | 14.12 |
| | (cm) | (0.87) | (0.69) | (1.03) |
| | Root | 12.02 | 12.63 | 11.84 |
| | (cm) | (0.79) | (0.71) | (0.60) |
| | Total | 25.95 | 27.09 | 25.96 |
| | (cm) | (1.30) | (0.96) | (1.32) |
| 3 | Shoot | 13.60 | 13.75 | 13.04 |
| | (cm) | (0.52) | (0.40) | (0.89) |
| | Root | 12.84 | 12.18 | 10.93 |
| | (cm) | (0.90) | (0.85) | (0.62) |
| | Total | 26.44 | 25.93 | 23.9 |
| | (cm) | (1.23) | (0.85) | (1.29) |
| 10 | Shoot | 14.71 | 13.42 | 12.67 |
| | (cm) | (0.74) | (0.69) | (0.75) |
| | Root | 12.25 | 11.54 | 10.39 |
| | (cm) | (0.42) | (0.46) | (0.65) |
| | Total | 26.96 | 24.96 | 23.06 |
| | (cm) | (0.93) | (0.65) | (1.07) |

Note:
Extract/Bottle in the medium is in μl of oil/100 ml base medium.
Values in parenthesis indicate standard deviation.

Thus, extracts of total homogenate as also of PLUS and MINUS fractions of a wide range of Angiosprm-monocotyledonous, Angiosperm-dicotyledonous dicotyledonous and Gymnosperm plants and also extracts of non-plant biomass showed significant biological activity with respect to a monocotyledonous plant (Triticum vulgare) at very low dose levels. Several extracts promoted auxin-like (rooting promotion), gibberellin-like (shooting promotion) or cytokine-like (biomass preservation/growth) activity at low doses of 1 μl/100 ml medium to 10 μl/100 ml of medium. Other extracts caused a strong inhibition of rooting, shooting and biomass mobilization at low doses of 3 μl/100 ml medium to 10 μl/100 ml of medium. Thus, a broad range of activity such as promotion, promotion followed by inhibition and inhibition of a wide range of endogenous hormones at a low concentration was observed. These different activities greatly enhance the utility of the compositions of this invention.

There are important and unexpected differences between the effect of oils and oil extracts on wheat (T. vulgare), mustard (Brassica nigra) and mungbean (Phaseolus radiatus). For comparison please see complete Tables 12, 29 and 112. Thus, the sesame oil promotion is much stronger on the monocot than on the dicot. P. emblica had a significant biomass promotion effect (E.A.) in mungbean germination but does not have such an effect in wheat germination. Taxus baccata (TABA-28) showed a strong promotion of root+shoot and biomass in mungbean up to 3 in μl of oil/100 ml base medium but does not show such a strong effect in wheat. A. indica and Pongamia glabra showed a strong inhibitory effect at and above 3 in μl of oil/100 ml base medium in mungbean germination but show, if at all, a promotional effect in the germination of wheat at 1 in μl of oil/100 ml base medium and the inhibitory effect at the higher concentrations are weak. There are extracts which promote mustard and also wheat but are weak or inhibitors in mungbean germination (Rauwolfia serpentina, Curcuma ionga, Eugenia Jambolina, Vinca rosea). There are other extracts which strongly promote mungbean germinationr but are weak or inhibit mustard germination (G. glabra, T. bellerica, Taxus baccata, Phaseilus radiatus). There are some extracts which promote all three germinations quite well (Triticum vulgare-PLUS, Kalanchoe pinnata-PLUS, Kalanchoe leaf stem, Mucuna pruriens).

While the variety of biomass extracts show germination activity in the germination of different seeds, the same extract can give different and sometimes opposite effect in the germination of different seeds. The interaction of the compositions of this invention with a particular biological specie therefore provides scope for selective and differential activity. This factor greatly extends the utility range of the compositions of the present invention.

### Example 30

### Effects on Rice

Trials were carried out at Kalyani, W. Bengal in the Kharif. Rice plants were cultivated on 40 m² plots; 3 replicates were used for each treatment. R-2 oil (batch 910608) was used, and a total of 2 sprays were applied on the 30^{th} and 60^{th} day after sowing. Results are shown in Table 36.

TABLE 36
Yield

| | UNTREATED CONTROL | TEST |
|---|---|---|
| R-2 oil Conc., ml/ha | 0 | 50 |
| Yield/Ha, quintals | 44.4 | 55.5 |
| % Increase | 0 | 25 |

### Example 31

### Wheat

Trials were carried out at Dakore, Gujrat, India on wheat cultivated in 100 m² plots. R-2 oil (batch 910608) was used, and a total of 2 sprays were applied on the 29^{th} and 60^{th} day after sowing.

TABLE 37
Yield

| | CONTROL | CONTROL | | |
|---|---|---|---|---|
| | w/o WATER | w/WATER | TEST | |
| R-2 oil Conc., ml/ha | 0 | 0 | 25 | 50 |
| Yield/Ha, quintals | 21.8 | 22.5 | 37.62 | 32.75 |
| % Increase | −0.7 | 0 | 67 | 46 |

Plants grown for flowers (monocots and dicots)

### Example 32

### Marigold

Trials with R-5 oil were carried out near Pune, India on marigold, Tagates erecta. Each set had 5 plants; measurements were the average for each set. A concentration of 1 T-5 tablet (250 mg) per 5 liters was used, and 25 ml/spray/plant were applied on mature, flowering plants. Results are reported in Table 38.

TABLE 38
Marigold yield and quality

| | |
|---|---|
| Test | Control |
| Flowering starts | Flowering starts |

Plant height, cm

| Spray Date | 45 | 45 |
|---|---|---|
| 1^{st} application | | |
| No. of flowers | 50 | 30 |
| Size of flowers | Large | Small |
| Branches | 17 | 12 |
| Insect attack | Low | High (Aphids/jassids) |
| 2^{nd} application | | |
| (31 days after 1^{st}) | | |
| No. of flowers | 120 | 60 |
| Av. Flower wt., g | 8 | 6 |

The number and size of flowers were increased by 25%. Resistance to sucking pests was also noted.

### Example 33

### Flowering Plants

Trials with T-5 (250 mg) tablets were taken at Daund, near Pune, Maharashtra, India. Dosages and other details are given below.

JASMINE (Jasminum sambac)

Concentration 1 T-5 (250 mg) tablet per 5 liter; spray volume of 5 liters/20 plants

Frequency: Twice a month. Total sprays=6.

First spray was applied 15 days after pruning.

Plant spacing: 1 m×1 m (20 plants each in TEST and CONTROL groups)

GLADIOLA (Gladiolus sp)

Concentration: 1 T-5 (250 mg) tablet per 5 liter; spray volume was 10 liter/100 m²(1000 plants)

Frequency: 30, 50 and 70 days after bulb opening.

Plant spacing: 30 cm×30 cm (1000 plants each in TEST and CONTROL groups)

ROSES (Rosa indica)

Concentration: 1 T-5 (250 mg) tablet per 5 liter; spray volume was 5 liter/100 m² (100 plants)

Frequency: once every 10 days (total of 9 sprays)

Plant spacing: 90 cm×90 cm (100 plants each in TEST and CONTROL groups)

The observations compared to control are reported in Table 39.

TABLE 39
Yield and quality of flowers

| OBSERVATIONS | TEST | CONTROL |
|---|---|---|
| JASMINE | | |
| Harvesting | 10 to 15 days early | normal |
| Flowering span | within 6 days | 15 days |
| Total flower wt., kg | 2 | 1.5 |
| Other observations | Large, uniform | |
| Compared to control | Complete opening | |
| | Longer shelf life | |
| | Export quality | |
| GLADIOLA | | |
| Number of flowers | 7500 | 6000 |
| Other observations | Attractive, longer stick | |
| Compared to control | Opens completely | |
| ROSES | | |
| Number of flowers | 1400 | 890 |

### Example 34

### Growth Rate in Forest Trees (Monocots and Dicots)

A trial was carried out at Thane-belapur road, Maharashtra, India using T-5 tablets (250 mg) on a variety of forest species. Both foliar spray and root zone application were employed.

T-5 tablets were dissolved in water to the indicated solution concentration and used as a foliar spray. Solution was sprayed once every two months, commencing on 19^{th} Nov. 1990. Data from two plants are reported for each set in Table 40.

TABLE 40
Growth of forest trees

| | 100 ppm | | 50 ppm | | | |
|---|---|---|---|---|---|---|
| | solution | | solution | | control | |
| | Height | Branch | Height | Branch | Height | Branch |
| Date | cm | number | cm | number | cm | number |
| 1990 | | | | | | |
| 19. Nov | 35, 40 | 2, 1 | 30, 40 | 1, 2 | 41, 40 | 3, 2 |
| 1991 | | | | | | |
| 19. Jan | 52, 64 | 5, 3 | 41, 63 | 3, 4 | 52, 59 | 6, 5 |
| 19. Mar | 94, 79 | 12, 13 | 69, 89 | 12, 13 | 69, 78 | 11, 12 |
| 19. May | 138, 135 | 22, 28 | 113, 128 | 41, 49 | 119, 128 | 28, 31 |
| 19. Jul | 235, 199 | 62, 69 | 179, 189 | 51, 63 | 150, 168 | 33, 40 |

LEAVES YELLOW DARK GREEN LIGHT GREEN

Thus, after 19^{th} Mar. 1991, the 50 ppm solution set (1 ppm of R-100 oil) demonstrated an excellent spurt in growth and branching along with the development of dark green foliage. However, the growth spurt with 100 ppm solution was even higher, although leaves were yellow.

ROOT ZONE APPLICATION

T-5 tablets were kept in a small basin 15 cm away from the tree and 5 cm deep. Only one application of tablets was made. Control (0), 1, 2 and 3 tablets were used. Two plants were used in each set. Plant height was recorded in cm and is reported in Table 4 1.

TABLE 41
Tree height

| | | | | |
|---|---|---|---|---|
| Date | Control | 1 Tablet | 2 Tablets | 3 Tablets |

Eucalyptus hybrid

| | | | | |
|---|---|---|---|---|
| 1991 | | | | |
| 1. Jun | 30, 30 | 30, 30 | 30, 30 | 30, 30 |
| 1. Sept | 60, 62 | 70, 82 | 58, 65 | 45, 51 |
| 1. Dec | 71, 79 | 100, 125 | 84, 97 | 58, 60 |
| 1992 | | | | |
| 1. Jan | 90, 100 | 145, 155 | 110, 130 | 70, 84 |
| 1. Feb | 120, 156 | 189, 190 | 145, 155 | 89, 121 |

Tectona grandis

| | | | | |
|---|---|---|---|---|
| 1991 | | | | |
| 1. Jun | 20, 20 | 20, 20 | 20, 20 | 20, 20 |
| 1992 | | | | |
| 1. Feb | 59, 62 | 60, 69 | 74, 70 | 54, 47 |

Dendrocalamus strictus

| | | | | |
|---|---|---|---|---|
| 1991 | | | | |
| 1. Jun | 60, 60 | 60, 60 | 60, 60 | 60, 60 |
| 1992 | | | | |
| 1. Feb | 120, 129 | 120, 138 | 135, 140 | 132, 128 |

Leucena leucephalia

| | | | | |
|---|---|---|---|---|
| 1991 | | | | |
| 1. Jun | 64, 64 | 64, 64 | 64, 64 | 64, 64 |
| 1992 | | | | |
| 1. Feb | 89, 92 | 94, 105 | 78, 72 | 71, 80 |

Thus, depending on the plant system, one and/or two T-5 tablets applied at the root zone lead to a considerable acceleration of height gain in forest trees and at optimal concentrations, the foliage turned dark green.

Root Zone and Terminal Bud Application

One T-5 tablet was applied to the root zone. In addition, a 5 g piece of cotton soaked in a 500 ppm solution of T-5 tablet was also placed on top of the terminal bud once every 3 months. Five (5) test and 5 control plants were used. The results are given in Table 42.

TABLE 42
Tree growth with terminal bud soaking
Casuarina equisetifolia

| | Test | | Control | |
|---|---|---|---|---|
| | Height Range, | | Height Range, | |
| Date | cm | Average, cm | cm | Average, cm |
| 1^{st} app. | 45-60 | 51.8 | 42-60 | 52.6 |
| 3 mos. | 65-80 | 74.0 | 65-84 | 73.6 |
| 5 mos. | 84-98 | 92.2 | 74-92 | 84.6 |
| 7 mos. | 115-130 | 120.4 | 91-105 | 99.2 |

Thus, for the first three months there was no difference. However, during the next four months the test trees grew at a much faster rate than the control trees.

### Example 35

### Mushroom Yield—Pleurotus osteatus

Trials were carried out at Chunchale, near Nashik, Maharastra, India on Pleurotus osteatus in rows of 15 beds each. Each bed was started with 1.5 kg of straw, dal and mushroom spawn tied up in polyethylene bags. The bags were cut open at the end of 3 weeks. From this point onwards, each bed was watered twice a day. 20 ppm solution of R-10 powder was sprayed once in three days (approx. 150 ml solution per bed). Mushrooms were harvested from the control and treated beds over the next 45 days. The total weight of the mushrooms from the control row of beds was 0.85 kg/bed. The weight of mushrooms from each of the tree treated rows was 1.35 to 1.45 kg/bed.

Mushrooms from the treated beds were large, more uniform in size and with a thicker stalk.

### V. MICROBIAL AND MAMMLIAN TOXICITY/PROMOTION

### Example 37

### Bacterial Toxicity

R-100 oil (batch 881206) did not show any antibacterial activity against Staphylococcus aereus and Proteus vulgaris, even at the high ratio of 1:10 of R-100; nutrient broth.

### Example 37

### A Promotion in Bacteria and Yeast

Several extracts were tested at the Garware college, Pune in 2001 for their ability to affect yeast (Candida albicans), gm+ve Bacteria (Staphylococcus aureus), and gm−ve bacteria (Pseudomonas aeruginosa).

The growth media used is described below:

Nutrient Broth (for S. aureus, P. aeruginosa) Composition:

Peptone: 1.0 gm %

NaCl: 0.5 gm %

Beef Extract: 0.3 gm %

PH: 7.2

Glucose Yeast Extract (for Candida albicans)

Peptone-1.0 gm %

Glucose-1.0 gm %

Yeast Extract-0.5 gm %

Water-100 ml

PH-6.5

The following procedure was followed:

1. 10 μl of oil based extract was added to 10 ml of distilled water.

2. This was kept on a rotary shaker for one hour, then it was allowed to settle and 9 ml of water under the oil layer was separated.

3. μl of the aqueous phase was added to 100 ml of distilled water.

4. The respective media (100 ml each) were prepared and autoclaved in 250 ml side tube flasks.

5. 30 μl sample was added in the 100 ml growth medium to get 300 ppm concentration in terms of the original oil extract equivalent.

6. Suspension of organisms was prepared in saline.

7. Above suspension (0.1 ml) was inoculated in each of the 100 ml growth medium.

8. Positive control—growth medium containing organism.

9. Negative control—growth medium.

10. The flasks were incubated for 48 hours at room temperature.

11. Absorbance was noted at 530 nm wavelength using negative control as blank.

TABLE 37
A Effect of Plant Extracts on Bacteria and Yeast

| | P. aeruginosa | | |
|---|---|---|---|
| | Gram | S. aureus | C. albicans |
| Plant Extract | −ve Bacteria | Gram +ve Bacteria | Yeast |
| Positive Control | 0.98 | 0.57 | 1.07 |
| SESAME OIL 110 | 0.36 | 0.46 | 0.48 |
| Angiosperm- | | | |
| Monocots | | | |
| ZOFF 100 | 0.36 | 0.92 | 1.08 |
| MINUS | | | |
| ZOFF 55.4 PLUS | 0.57 | 0.81 | 0.73 |
| CAMA 98.4 | 0.80 | 1.36 | 0.86 |
| MINUS | | | |
| CAMA 26.2 | 0.42 | 0.81 | 1.13 |
| PLUS | | | |
| CROT 62.7 | 0.70 | 0.71 | 1.36 |
| MINUS | | | |
| CROT 28.2 PLUS | 0.67 | | 1.04 |
| A 100 PLUS | 0.86 | | 0.62 |
| Gymnosperm | | | |
| TABA 27 | 0.57 | 1.05 | 1.20 |
| Angiosperm- | | | |
| Dicots | | | |
| AZIN-C-29 | 0.42 | 1.02 | 1.01 |
| PGL 33 | 1.21 | 0.89 | 0.73 |
| PE 100 PLUS | 0.84 | 1.50 | 1.15 |
| TB 27.7 | 060 | 1.12 | 1.24 |
| TC 27.8 | 0.65 | 1.13 | 0.94 |
| B 100 PLUS | 0.50 | 1.67 | 1.03 |
| TRIGF 29.6 | 0.95 | 0.95 | 1.02 |
| FB 29 PLUS | 0.80 | 0.90 | 1.11 |
| HA 29 | 0.42 | 0.84 | 1.13 |

Note 1:
All Extracts are used at 300 ppm level in the medium.
Note 2:
Numbers represent optical density. Variation of absorbance at 530 nm was measured.
Note 3:
Gram negative (gram −ve); gram positive (gram +ve).

The above results show that the base sesame oil boiled with water for 110 minutes (Sesame Oil 110) is inhibitory compared to positive control to all the three organisms studied. The results with all other extracts have to be understood against the backdrop of positive control and ‘sesame oil 110’ results.

Each oil extract is also with sesame oil as the base and has been subjected to boiling with water for some time as part of preparation. However, none of the oil extracts are more inhibitory than ‘sesame oil 110’. On the other hand, each extract studied is a growth promoter of candida yeast (eukaryote) and both gram positive and gram negative bacteria (prokaryotes) to different extent in comparison with ‘sesame oil 110’.

Several extracts are promoters of growth (increase in optical density of the medium) even compared to positive control for candida yeast, an eukaryote. This is particularly so in case of CROT 62.7 MINUS, TABA 27, TB 27.7, and PE 100 PLUS.

All extracts studied are found to be promoters compared to positive control in respect of staphylococcus, a gram positive bacteria and a prokaryote. Some extracts are also promoters for pseudomonas, a gram negative bacteria and a prokaryote.

Thus, several compositions of this invention are promoters not just for plants but also for other eukaryotes such as yeast and also for prokaryotes such as staphylococcus and pseudomonas.

### Example 38

### Anti-Mutagenicity in Bacteria

In a standard Ames test, the following results were obtained: AMES TEST: Salmonella typhimurium (S9)

Tested against the carcinogen B(a)P (Benz(o)pyrene) at 2microg/plate according to the methods of (Ames et al., 1973; Ames et al., 1975).

R-100 oil (batch 910217) diluted 1:200 or 1:500 in water

Concentration of water extract tested: 2 μl/plate

Results expressed as mean number of revertants/plate in Table 43.

TABLE 43
Anti-mutagenicity in bacteria

| | | DILUTION | DILUTION |
|---|---|---|---|
| STRAIN | CONTROL | 1:500 | 1:200 |
| TA98 + S9 | 25 | 23.5 ± 1.6 | 35.1 ± 3.6 |
| TA98 + S9 + B(a)P | 235.5 ± 11.0 | 119 ± 11.8 | 145.3 ± 12.9 |
| TA100 + S9 | 161 | 142.8 | 76 |
| TA100 + S9 + B(a)P | 489.5 ± 5.0 | 124.4 ± 13.2 | 162.5 ± 2.5 |

Thus, R-100 oil did not act as a mutagen when added at 1:200 or 1:500 solution; R-100 oil was anti-mutagenic or prophylactic for the mutagenicity of Benz(o)pyrene in both TA98 and TA100 type of mutations.

### Example 39

### Low Mammalian Toxicity and Faster Growth

A chronic toxicity study of R-10 powder was carried out on Albino rats (Wister strain). There were 20 animals in each group, evenly distributed by sex. Dose levels of 0, 500, 1000 and 2000 mg/kg/day of R-10(P) powder were used. These are equivalent to 0, 50, 100 and 200 mg/kg/day of leaf equivalent or R-100 oil.

Hematology, blood biochemistry and histopathology of all major organs were performed at the end of 41 weeks; no toxic effects were observed. There is no remarkable change in gross pathology or in the histopathology.

The average body weight (g) and standard error of estimate (number in parenthesis) at 0, 14, and 41 weeks in the study for all dose levels are reported in Table 44.

TABLE 44
Growth rate and toxicity in rats

| DOSE | | | | | | | | |
|---|---|---|---|---|---|---|---|---|
| Mg/kg/ | | | | | | | | |
| day of | MALE | | | | FEMALE | | | |
| R-10(P) | 0 | 500 | 1000 | 2000 | 0 | 500 | 1000 | 2000 |
| START | 92.0 | 91.8 | 96.8 | 92.4 | 88.2 | 95.8 | 86.4 | 91.4 |
| | (1.71) | (1.87) | (1.77) | (2.18) | (2.20) | (1.48) | (1.63) | (2.50) |
| 14 WEEKS | 176.4 | 193.4 | 193.0 | 200.8 | 143.0 | 151.4 | 144.0 | 150.0 |
| | (6.20) | (6.48) | (4.78) | (6.67) | (3.51) | (2.72) | (4.00) | (5.30) |
| 41 WEEKS | 220.8 | 227.8 | 209.2 | 217.5 | 156.8 | 165.6 | 157.6 | 159.2 |
| | (8.31) | (14.64) | (8.13) | (12.91) | (5.4) | (3.49) | (6.72) | (5.7) |

Note:
The numbers in parenthesis indicate standard deviation.

The intake of R-10(P) did not resulted in any adverse chronic effects and did not affect weight after full maturity (41 weeks). However, between the 7^{th} to the 21^{th} week or during the early development, the intake accelerated the rate of weight gain or caused an increased, but healthy growth, in experimental animals.

### Example 40

### Faster Cartilage Tissue Growth and Low Toxicity

A chronic toxicity study of R-100 oil (batch 930425) was carried out on in Sprague Dawley rats. There were 10 animals in each group (5 males and 5 females). Dose levels of 0, 5, 50 and 500 mg/kg/day of leaf equivalent in the form of R-100 oil were used. R-100 oil was mixed with corn and administered to rats for 180 days. This was followed by a recovery period of 28 days. Hematology, blood biochemistry, urine analysis and histopathology of all major organs were performed at the end of 180 days, showing no toxic effects. There was no remarkable change in gross pathology or no remarkable changes in the histopathology. Dose levels of 5 and 50 mg/kg/day did not induce any toxicity. At 500 mg/kg/day, nasal secretions, polyurea, diarrhoea, drowsiness, ataxia, alopecia were observed for some male and female animals. These signs of intoxication subsided during the recovery period of 28 days.

During a four week period, 6^{th} to 10^{th} week of study, there was a faster increase in tail length of test animals (both male and female) from various treatment groups compared to controls, as shown in Table 45.

TABLE 45
Tail length gain in rats (6^{th} to 10^{th} week of study), mm

| Dose, mg/kg/day of R-100 | Male | Female |
|---|---|---|
| Control | 4.8 | 5.8 |
| 5 | 6.5 | 10.9 |
| 50 | 10.0 | 9.0 |
| 500 | 12.2 | 7.3 |

This period coincided with the onset of sexual maturity. Faster increase in tail length test animals indicated a faster growth of cartilage tissue due to the intake of R-100 oil.

### Example 41

### Anti-Mutagenicity in Mammals

Mice bone marrow micronucleus test, R-100 oil (batch 910217) was given to mice in drinking water at 2 ppm (v/v) level for 15 days as a prophylactic before challenging them with B(a)P (Benz(o)pyrene). Results are reported as per cent micronucleated cells (% MNPCE) in Table 46.

TABLE 46
Anti - mutagenicity in mammals

| | R-100, ppm | | |
|---|---|---|---|
| | In drinking water | % MNCPE | |
| Solvent Control | nil | 0.7 ± 0.04 | n = 4 |
| Solvent Control | 2 | 0.48 ± 0.025 | n = 4 |
| Solvent Control + B(a)P | nil | 2.0 ± 0.1 | n = 4 |
| Solvent Control + B(a)P | 2 ppm | 0.7 ± 0.04 | n = 4 |

Thus, R-100 exhibited prophylactic activity against an important carcinogen, Benz(0)pyrene in a mammalian system as well as in bacteria.

### Example 42

### Low Toxicity in Topical Application (Skin Irritation and Dermal Toxicity)

R-100 (batch 920814) in the amount of 0.5 ml was applied to the shorn back skin both intact and abraded site of three rabbits per sex. Each site was observed and reaction recorded by Draize method (States, 1979).

No erythema or edema of skin was observed in rabbits after application of test substance. Thus, R-100 oil did not cause any irritation to the skin of rabbits.

The R-100 oil, 0.1 ml, was introduced on the penile and vaginal mucous membrane of male and female rabbits. No erythema or edema was observed as scored by the Draize method after 24, 48 and 72 hours.

R-100, R-5 and R-1 oil (batch 920814) were applied to the shaven back skin of New Zealand White rabbits at the rate of 3 ml/kg body weight. Control animals were treated with sesame oil. 6 animals (3 males and 3 females) were used at each dose level. The extract was kept in contact with the shaven intact skin for 6 hours per day, 5 days a week for 3 weeks. The following results were observed at 21 days:

1. Elevated alkaline phosphatase levels was observed with the R-100 dose set

2. Moderate to severe, well defined, and very slight erythema was observed with R-100, R-5 and R-1 oil, respectively.

3. Higher platelet values were observed in the blood of animals treated with R-100 and male animals treated with R-5 oil.

At the end of 14 day recovery period,

1. Serum alkaline phosphatase levels returned to normal

2. Erythema in all cases subsided

3. However, elevation of platelet level persisted.

Except for these effects, no other macroscopic effects were observed during necropsy.

Thus, R-100 oil showed no observable effects at the dose levels tested.

### Example 43

### Low Cytotoxicity: In Vitro Cancer Cell Line Screen

R-100 oil sample was screened at the Frederick Cancer Research and Development Center of the National Cancer Institute, (Bethesda, MD; USA) according to (Boyd and Pauli, 1995). There was no cell mortality up to a high concentration of 250 mg/liter of R-100 tested in vitro in 60 different cancer cell lines. Thus, the extract showed very low cytotoxicity.

### Example 43A

### Phagocytosis Promotion in Human PMN Leucocytes (Neutrophils) and Monocytres

Several of the extracts were tested for their phagocytosis activity in human PMN (Polymorphonuclear) Leukocytes and in human Monocytes (Lehrer R. I .et al 1969; Lehrar R. I. 1970; Boyum A. 1969; Rege et al. 1993.) at T. N. Nair Medical College, Mumbai.

Procedure for PMN Leucocytes Function Test is given below:

A method described by Lehrer R. I., and M. J. Cline (1969)., “specific cell assay” for phagocytic activity of neutrophil was used with few modifications. From normal healthy volunteer, 10 ml of peripheral venous blood was collected in sterile heparinised tube. Two (2) ml of this blood was centrifuged at 2000 rpm for 10 minutes and plasma was separated. To the rest 8 ml of blood, 8 ml of sterile saline was added. The diluted blood was overlaid on Ficoll Hypaque (2 ml of Ficoll Hypaque for 8 ml of diluted blood was used) and subjected to centrifugation at 2000 rpm for 40 mins (Boyum 1969). After the density gradient centrifugation, the RBC-PMN pellet was separated and mixed with 1 ml of autologous plasma (obtained as described earlier) and 1 ml of 5% dextran. The mixture was allowed to stand at 37° C. for 1 hour for sedimentation of RBCs. The supernatant, which contains more than 90% of PMN, was collected. The percentage of PMN in total cell population was confirmed by differential leukocyte count of the suspension after Giemsa staining. Cell density (count/ml) of PMN was found out using Neubar's chamber. Cell count was adjusted to 1×10⁶ cells/ml in MEM (minimum essential medium). Then 250 μl of the PMN suspension (1×10⁶ cells/ml), 250 μl of Candida cell suspension (1×10⁶ cells/ml) and 500 μl of MEM were added in a siliconised tube and incubated at 37° C. for 1 hour under 5% CO₂ atmosphere as a control. For evaluation of in vitro activity of the test substance, 10 μl/50 μl of the test extract was incorporated in the incubation mixture by replacing the same quantity of MEM. Cytosmears were prepared at 1000 rpm for 8 minutes. The smears were then fixed with methanol, air-dried and stained with Giemsa stain. The smears were observed for assessment of % phagocytosis and phagocytic index as follows:

Procedure for Monocytes Function Test is given below:

From normal healthy volunteer, 10 ml of peripheral venous blood was collected in sterile heparinised tube. Two (2) ml of this blood was centrifuged at 2000 rpm for 10 minutes and plasma was separated. To the rest 8 ml of heparinised blood, 8 ml of sterile saline was added. The diluted blood was overlaid on Ficoll Hypaque and subjected to centrifugation at 2000 rpm for 40 minutes (Boyum 1969). After the density gradient centrifugation, buffy ring containing monocytes cells were separated & count was adjusted to 1×10⁶ cells/ml in MEM (minimum essential medium) and serum in a ratio of 4:1. Then, 375 μl of monocytes (1×10⁶ cells/ml), 125 μl of Candida cells (1×10⁶ cells/ml), 375 μl of MEM (supplemented with 125 μl of serum) was added and incubated at 37° c. for 90 minutes under 5% CO₂ atmosphere as control. For evaluation of in vitro activity of the test substance, 10 μl/50 μl of the test extract was incorporated in the incubation mixture by replacing the same quantity of MEM. Cytosmear was prepared at 1000 rpm for 8 minutes. The smear was then fixed with methanol, air-dried and stained with Giemsa stain. The smear was observed for assessment of percent phagocytosis, phagocytic index and the percent intracellular killing.

Typically, 100 cells were scored and data recorded with the following definitions.

Percentage phagocytosis is defined as percentage of monocytes or PMN Leucocytes involved in phagocytosis

Source of Candida albicans Culture (ATCC Number 10231)

Candida albicans culture has been obtained from the National Chemical Laboratory (NC) Pune. It is subcultured aseptically after 15 days on Sabaroud's agar. A loopful of the spores of C. albicans is inoculated in Sabaroud's broth and incubated at 37° C. for 18 hours before the assay.

In each case of an extract treatment, typically 100 cells were scored. The results are given below: average values of % phagocytosis, and the standard deviation are reported. The results of the Paired ‘T’ test are given both compared to plain control and also against sesame oil at the same concentration as the extract (10 and 50 μl/100 ml of the medium).

Sesame oil alone has shown moderate to high phagocytosis promotion both in case of neutrophils and monocytes. In the table below the biomass extracts in oil (with sesame oil as the oil medium) are compared with the sesame oil results. At least 6 blood samples were tested in each case. Percent phagocytosis was compared with sesame oil and with particular oil extract. A summary of ‘p’ values from the paired Student's ‘T’ Test comparing the Extract with Sesame Oil ‘Negative Control’ are given in Table 43A below.

Values of p <0.0500 are highlighted in bold.

Numbers in parenthesis indicate extracts where the phagocytosis function has been inhibited compared to Sesame Oil ‘Negative Control’. Concentration of Extract in the test medium is indicated at the head of appropriate column as 10 or 50 μl/100 ml

TABLE 43A
Effect of Biomass Extracts on PMN and Monocytes
Phagocytosis

| | Samples | PMN | PMN | Samples | Monocyte | Monocyte |
|---|---|---|---|---|---|---|
| | Tested- | 10 μl/ | 50 μl/ | Tested | 10 μl/ | 50 μl/ |
| Extract | PMN- | 100 ml | 100 ml | Monocytes | 100 ml | 100 ml |
| B-100 Plus | 10 | 0.3903 | 0.0334 | 6 | 0.0004 | 5.675E−05 |
| Exp. Set 1 | | | | | | |
| B-100 Minus | 10 | 0.0214 | 0.0412 | 6 | 0.0925 | 1.262E−05 |
| PE-100 | 10 | 0.7705 | 0.1309 | 6 | 0.0580 | 0.0006 |
| TICO-35.51 | 10 | 0.4035 | 0.0304 | 6 | 0.0034 | 4.644E−06 |
| AZIN-C-29 | 10 | (0.8791) | (0.4405) | 6 | 0.1067 | (0.7412) |
| HA-29.7 | 10 | (0.0321) | (0.0209) | 6 | 0.0896 | 0.1345 |
| ER-100 Minus | 10 | (0.0528) | (0.1533) | 6 | 0.2504 | 0.0108 |
| ZOFF-55.2 Plus | 10 | (0.5621) | (0.1533) | 6 | 1.18E−05 | 1.000 |
| YBD-47.55 | 10 | (0.1142) | (0.7935) | 6 | 0.0335 | 0.0478 |
| MPRU-27.4 | 7 | 0.0161 | 0.0126 | 6 | 0.5549 | 0.0026 |
| ASAT-68.68 | 7 | 0.0618 | 0.0557 | 6 | 0.0323 | 0.0008 |
| PILO-36.02 | 7 | 0.2673 | 0.0041 | 6 | 0.2242 | 0.0003 |
| TCHEB-27.8 | 7 | 0.0090 | 0.2111 | 6 | 0.0525 | 0.0002 |
| WISO-38.82 | 7 | 0.0605 | 0.0313 | 6 | 0.0001 | 0.1412 |
| ASRA-30.2 | 7 | 0.0195 | 0.1192 | 6 | 0.0756 | 0.0004 |
| A-100 Plus | 6 | 0.0026 | 0.0113 | 6 | 0.3522 | 0.6793 |
| A-100 Minus | 6 | 0.0003 | 0.0004 | 6 | 0.2242 | 0.0235 |
| GGLAB-27.43 | 6 | 0.0044 | 0.0128 | 6 | (0.0395) | (0.0009) |
| C-100 | 6 | 0.0003 | 0.0001 | 6 | 0.0013 | 3.658E−05 |
| MUSH-A 98.36 | 6 | 0.0019 | 0.0149 | 6 | 0.5965 | 0.5671 |
| OSA-T 48.88 | 6 | 0.0026 | 0.0160 | 6 | 0.0301 | 0.0335 |
| TVUL32.8 Plus | 6 | 0.0159 | 0.0531 | 6 | 0.8560 | 6.545E−05 |
| PINI-32.4 | 6 | 0.0392 | 0.0466 | 6 | 0.0245 | 0.314 |
| CACO-37.5 | 6 | 0.0451 | 0.0005 | 6 | 0.0018 | 0.4150 |
| PRWN-67.88 | 6 | 0.0067 | 0.0098 | 6 | 0.7926 | 0.0010 |
| EJAM-34.16 | 6 | 0.0981 | 0.0100 | 6 | 0.6952 | 0.0616 |
| B-100 Plus | 6 | 0.0016 | 0.0018 | 6 | 0.0004 | 0.0159 |
| Exp. Set 5 | | | | | | |
| ER-28.8 Plus | 6 | 0.0076 | 0.0197 | 6 | 0.1580 | 0.00737 |
| Extract Combinations | | | | | | |
| AMAVATA-10 | 6 | 0.0004 | 0.0162 | 6 | 0.0002 | 7.443E−06 |
| ARTHRITIS-5 | 6 | 0.0006 | 0.0017 | 6 | 0.0300 | 3.726E−05 |
| FEVER-10 | 6 | 0.0002 | 0.0003 | 6 | 0.1852 | 5.682E−06 |
| PRAMEHA-20 | 6 | 0.0040 | 0.0002 | 6 | 0.2031 | 9.989E−05 |
| PREG-15 | 6 | 0.0006 | 0.0013 | 6 | 0.0822 | 0.0004 |
| STABILIZER-30 | 6 | 0.0004 | 0.0010 | 6 | 0.0172 | 0.0264 |

Several extracts show significant activity of phagocytosis promotion even with respect to sesame oil. Enhancement of activity with respect to water control is higher still. The dose levels at which activity is shown is 10 to 50 μl/100 ml of medium. In terms of the starting biomass equivalent this level is typically <200 mg/liter of the medium. This is a very low dosage level.

Even extracts of food materials and non-plant biomass which are normally consumed in quantities from 10 to 50 gm such as button mushroom (MUSH-A-98.36), yeast (YBD-47.55), wheat (TVUL-32.8 PLUS) and prawn (PRWN-0.67.88) have shown significant phagocytosis promotion of PMN Leucocytes and/or monocytes at a low dose of 10 to 50 μl/100 ml.

Some of the extracts show inhibition of phagocytosis. Such an effect is related to the anti-inflammatory activity of the extract.

Combination extracts also show very high activity. A description of the composition is given below. All combination extracts also contain 20 gm MCT Oil (a 70:30 micture of Capryllic: Capric tryglycerides obtained from Subhash Chemicals, Pune). A 10 μl/100 ml combination extract of stength-10 is equivalent to 1 mg of starting herbs/100 ml of medium. This is indeed a very low concentration in the medium. Combination extracts are also given typically at the rate of 2 to 4 drops per day. A strength-10 extract has only 4 mg of total herbal material per drop.

AMAVAT-10 oil is based on the extract of 10 gms of total herbal material/100 gm of oilextract. The herbal mixture contains equal parts of: 1) an equal part mixture of Zingiber officinale, Carum copticum, Cuminum cyminum, Piper longum, 2) a mixture of Terminalia Bellerica (1 part), Terminalia chebula (3 parts), Phyllanthus emblica (6 parts), Aloe indica (10 parts), Glycyrrhiza glabra (10 parts), 3) an equal part mixture of Trigonella, Linum usitatisum, Phaseolus radiatus, Triticum vulagare and 4) an equal part mixture of Holarrhena antidysenterica, Embelia ribes, Tinospora cordifolia, Swertia chirata. This extract is found useful in G.I. tract problems such as gassess, tendancy for loose bowels and colitis.

ARTHRITIS-5 based on the extract of 50 gms of total herbal material/100 gm of oilextract. The herbal mixture contains equal parts of: 1) an equal part mixture of Kalanchoe pinnata, Cissus quadrangularis, Mucuna Pruriens and 2) a mixture of Terminalia Bellerica (1 part), Terminalia chebula (3 parts), Phyllanthus emblica (6 parts), Aloe indica (10 parts), Glycyrrhiza glabra (10 parts). Each drop of this ARTHRITIS-5 oil contains 2 mg equivalent of all the above herbs combined. This oil is useful in both topical applications and oral intake for pain and inflammation due to osteoarthritis and in conditions such as frozen shoulder, i.e. a joint stiffness conditionw here e.g. raising an arm above the shoulder level may be painful.

FEVER-10 oil is based on the extract of 10 gm of total herbal material/100 gm of oilextract. The herbal mixture is a mixture of 6.25 parts of Swertia chirata and 1.25 parts each of Tinospora cordifolia, Cyperus rotundus, and Ocinum sanctum. Each drop of this FEVER-10 oil contains 4 mg equivalent of all the above herbs combined. This combination is useful as an anti-pyretic.

PRAMEHA-20 is based on the extract of 20 gm of total herbal material/100 gm of oil extract. The herbal mixture is equal parts of Ficus bengalenis, Terminalia chebula, Eugenia jambolana and Momordica charantia. Each drop of this PRAMEHA-20 oil contains 8 mg equivalent of all the above herbs combined amd is useful in diabetes-II with a tendancy for loss of weight.

PREG-15 is based on the extract of 15 gm of total herbal material/100 gm of oil extract. The herbal mixture is equal parts of Emblica officinalis, Asperagus racemosus and Gycyrrhiza galbra. Each drop of this PREG-15 oil contains 6 mg equivalent of all the above herbs combined amd is useful as an uterotonic, promotes healthy growth of foetus and is a good lactating agent post-delivery.

STABILIZER-30 is based on the extract of 30 gm of total herbal material/100 gm of oil extract. The herbal mixture is a mixture of Terminalia Bellerica (1 part), Terminalia chebula (3 parts), Phyllanthus emblica (6 parts), Aloe indica (10 parts), and Glycyrrhiza glabra (10 parts). Each drop of this STABILIZER-30 oil contains 12 mg equivalent of all the above herbs combined and is found useful in headaches due to exposure to sun and in reducing inflammation of the G.I. tract. VI.

Applications to Livestock and Poultry

### Example 44

### Reduced Feed Conversion Ratio (FCR) and Low Mortality in Poultry (Layer Birds)

Experiments were carried out with BABCOCK brand (BV300) layer birds near Panvel, Maharashtra, India. In the 20^{th} week after hatching, the birds were transferred to layer cages. In one typical experiment, one row of 168 birds served as test birds, whereas 2250 remaining birds served as controls. Test birds were fed 100 g/day/bird, and the control birds were given 110 g/day/bird of feed consisting of 33% red maize, 35% of de-oiled soya and groundnut cakes, 15% de-oiled rice polish, 5% rice polish and calciferous material, fish meal, etc. Feed of test birds contained 400 ppm (v/w) R-10 oil (approx. 40 mg/kg feed of leaf equivalent).

At the end of 47^{th} week, the test birds had produced 23683 eggs with a feed consumption of 2842 kg, i.e., with an FCR of 120 g feed/egg. The control birds had produced 258074 eggs with a total feed consumption of 40272 kg, i.e., with an FCR of 156 g feed/egg. Thus, there was a 23% reduction in the FCR.

Mortality in the test group during this 26 week period was 9.5%, whereas mortality in the control group during this period was 12.2%. Thus, survival of the test birds was definitely improved.

### Example 45

### Lower Feed Conversion Ratio (FCR) and Higher Egg Production in Poultry

Experiments were conducted at Sangli, Maharashtra, India, with HISEX brand layer birds. Five (5) mg R-100 oil/kg feed and 2 mg R-100/kg feed were used for test birds. The birds were moved to layer cages after 20 weeks. Control and test birds were fed identical feed, except that the test birds received the R-100 oil.

The average weekly feed intake during the laying period was approximately 0.8 kg. Hence, the approximate weekly consumption of R-100 at the higher dose of 5 mg/kg feed was 4 mg./bird. The average weight of birds during the laying period was 1.6 to 1.8 kg. Hence the approximate daily dose in test birds was 4/(1.7×7)=0.335 mg of R-100/kg body weight per day. At the lower dose, the intake was 0.135 mg R-100/kg body weight per day. These results are summarized in Table 47.

The first trial where 5 mg/kg feed of R-100 oil (5 mg leaf equivalent/kg feed) was given only after attaining 20 weeks, the reduction in feed consumption/egg (FCR ratio) was 7.0%, along with a 4.7% increase in the number of eggs produced.

In the second trial R-100 was given from birth. In this case, improvement in FCR was much higher: 13.4 % with a lower (2%) increase in total egg production.

In the third trial at the lower dose of 2 mg/kg feed of R-100 equivalent, there was still a 7.75% improvement in FCR ratio.

TABLE 47
Summary of ‘layer’ bird trials
TYPE OF BIRDS: HISEX
DOSE: R-10 OIL 50 ml/MT Feed, for TRIALS 1, 2 and 3
R-10 OIL 20 ml/MT Feed for TRIAL 4

| | TRIAL NO | | |
|---|---|---|---|
| | 1 | 2 | 3 |
| Date of Hatch | Mar 1, 90 | Sep 17, 90 | Dec 22, 90 |
| Trial Started on | Jun 22, 90 | Sep 17, 90 | Dec 22, 90 |
| From - week | 20 | 0 | 0 |
| To - week | 82 | 61 | 52 |
| Duration, weeks | 62 | 61 | 52 |
| Test Birds. (T) | 1350 | 1514 | 1399 |
| Control Birds, (C) | 1744 | 3000 | 3200 |

CUMULATIVE RESULTS

| | Test | Control | Test | Control | Test | Control |
|---|---|---|---|---|---|---|
| Feed, kg/Bird | 49.246 | 50.925 | 38.912 | 43.196 | 31.244 | 33.739 |
| Eggs/Bird | 296.15 | 282.92 | 210.09 | 205.96 | 157.87 | 157.0 |
| Feed/Egg, g | 166.3 | 178.0 | 147.6 | 167.4 | 153.5 | 165.4 |
| | | | (>20 weeks) | | (>20 weeks) | |
| FCR, % | 7.03 | | 13.4 | | 7.75 | |
| reduction | | | | | | |
| Eggs, % | 4.67 | | 2.00 | | 0.55 | |
| increase | | | | | | |

The reported use of direct leaf extract is at the level of 70 leaves per day per 2000 birds. Assuming 5 g weight per leaf (these leaves were generally turgid), and 1.7 kg as the average weight per bird, this is approx. 350 g per 3400 kg per day or 100 mg/kg body weight per day. Thus, use of R-100 in layer birds results in a combination of improvement in FCR ratio and increase in egg production. This effect is novel and is produced at a leaf equivalent or R-100 dose levels considerably lower (0.335 and 0.135 mg/kg) than the reported use (100 mg/kg) of direct leaf extract dose in the literature.

### Example 46

### Broiler Productivity and Mortality

The experiment was conducted near Panvel, Maharashtra, India. One (1) g of R-100 oil (batch 910316) was solubilized in one liter of 10% polysorbate. This solution (1000 ppm of R-100) was added to drinking water to the test batch at the rate of 1 ml/liter, creating approximately a 1 ppm solution of R-100. During the study, each batch consumed approximately 20000 liters of drinking water, or 20 gm of R-100 oil.

The feed was 50% red maize, 20% roasted soybean, 10% groundnut cake, 7% fish meal and 3% minerals. The feed was given ad lib. Three consecutive batches, two controls and one experimental, were run. The results are given in Table 48.

TABLE 48
Broiler productivity and mortality

| | CONTROL I | TEST | CONTROL II |
|---|---|---|---|
| Total Days | 56 | 49 | 53 |
| Initial No. of Birds | 1529 | 1530 | 1500 |
| Mortality | 120 | 72 | 217 |
| Total Feed, kg | 4425 | 5025 | 4575 |
| Total Broiler Wt., kg | 1969 | 2223 | 1709 |
| Ave Broiler(end), kg | 1.40 | 1.52 | 1.33 |
| Feed Con. Ratio, kg/kg | 2.25 | 2.26 | 2.68 |
| Productivity, kg meat/day | 35.16 | 45.37 | 32.24 |

Thus, there was a considerable reduction in the mortality of birds. Also, the growth rate of broilers was much faster resulting in shed productivity for the test group being 30 to 40% higher than the two control runs.

Total consumption of R-100 was 20000 mg on a total feed of 5000 kg. Thus, the average level was 4 mg/kg feed. This is a range similar to that used in Example 45 for Layers. The total weight of broilers is about 2000 kg at the end of 50 days. Thus, using an average weight of 1000 kg for 50 days, the average R-100 dose was 0.4 mg/kg body wt/day.

### Example 47

### Lower Acidity and Bacterial Count in Buffalo Milk

Four Murrah buffaloes were given 2 drops per day of R-5 oil in drinking water over a 2.5 month period. The milk quality was tested on the day following the final administration of R-5. Dosage was approximately 3.5 mg per day of R-100 oil or leaf equivalent per animal.

The acidity (expressed as wt % lactic acid equivalent) of 10 ml milk after incubation at 37° C. for 4 hours was 0.18 acidity units for the control animal and 0.14 to 0.15 for the test animals.

The SPC by standard plate count was 54000 for control vs. 43000 to 49000 for the test animals. A coliform test (Durham) showed gas formation in the control animal sample and no visible gas formation in all the test animal samples. Thus, the coliform level in the milk of treated animals was reduced and shelf life of milk improved (acidity formation slowed down).

Characterization of Extracts

### Example 48

### TLC Comparison with Commercial Extracts of the Same Plant

A TLC (Thin Layer Chromatography) based comparison of the oil extracts of this invention was made with commercial total plant extracts of the same plant of Saiba Industries, Mumbai. The database of these commercial standard extracts was provided by Saiba Industries, Mumbai.

Standard Merck Aluminum sheets (20 cm×20 cm) coated with silica gel stationery phase (60F-254) were used.

In Table 49,

The TLC spot Rf values are shown as fractions.

The solvent system used for TLC is indicated for each plant extract.

Common spot Rf values are indicated by bold letters. Additional spot Rf values are indicated in bold letters within parenthesis. All other spot Rf values shown by commercial extracts are absent from the oil extracts of the Invention.

TABLE 49
TLC spot Rf value comparison with commercial
extracts of the same plant.

| | | Rf values of |
|---|---|---|
| Plant Extract/Code | Rf values of | Commercial |
| TLC | Plant extracts | Extract |
| Solvent system | Of Invention | of the same plant |
| Azadiracta. indica cake | 0.96, | 0.96, 0.91, 0.72, 0.68, 0.63, 0.52, 0.45, |
| AZIN-C-29 | (0.94), | 0.40, 0.33, 0.27, 0.18, 0.11 |
| Ethanol:chloroform:ammonia/ | 0.84, 0.72, 0.68 | |
| 8:5:2 | | |
| Azadiracta. indica Leaf | 0.96, | 0.96, 0.86, 0.80, 0.72, 0.65, 0.56, 0.44, |
| N-50 | (0.92), | 0.42, 0.37, 0.34, 0.26, 0.19, 0.10 |
| Ethanol:chloroform:ammonia/ | 0.72, 0.40 | |
| 8:5:2 | | |
| Allium sativum | 0.96, | 0.96, 0.90, 0.86, 0.66, 0.60, 0.53, 0.47, |
| ASAT-68.68 | (0.74), | 0.38, 0.30, 0.26, 0.14, 0.10 |
| Ethanol:chloroform:ammonia/ | 0.66 | |
| 8:5:2 | | |
| Allium cepa | 0.95, 0.75 | Not available |
| ACEP-74.3 | | |
| Ethanol:chloroform:ammonia/ | | |
| 8:5:2 | | |
| Aloe vera | 0.92, | 0.97, 0.95, 0.89, 0.79, 0.66, 0.61, |
| A-50 | (0.79) | 0.59, 0.54, 0.49, 0.44, 0.37, 0.24, |
| Ethyl acetate:Methanol:water/ | | 0.06 |
| 100:16.5:13.5 | | |
| Asperagus racemosus | 0.71 | 0.97, 0.93, 0.83, |
| ASRA-30.16 | | 0.71 |
| Ethanol:chloroform:ammonia/ | | 0.58, 0.51, 0.43, 0.34, 0.27, 0.14 |
| 8:5:2 | | |
| Bacopa monnieri | 0.94, 0.84, 0.74, | 0.94, 0.91, 0.84, 0.79, 0.74, 0.70, |
| BAMO-53.59 | (0.48), (0.28) | 0.63, 0.59, 0.55, 0.49, 0.26, 0.13, |
| Ethanol:chloroform:ammonia/ | | 0.05 |
| 8:5:2 | | |
| Cissus quadrangularis | 0.95, 0.81 | Not available |
| C-100 | | |
| Ethanol:chloroform:ammonia/ | | |
| 8:5:2 | | |
| Curcuma amada | 0.92, 0.81, 0.65 | 0.97, 0.92, 0.88, 0.81, 0.73, 0.65 |
| CAMA-20.69 | | 0.60, 0.58, 0.40, 0.36, 0.24, 0.13, 0.06 |
| Ethanol:chloroform:ammonia/ | | |
| 8:5:2 | | |
| Glycyrriza glabra | 0.65 | 0.85, 0.74 |
| GGLAB-29.76 | | 0.65 |
| Chloroform:methanol/ | | 0.54, 0.45 |
| 90:10 | | |
| Holarrhena | 0.96, | 0.96 |
| antidysenterica | 0.71, 0.64 | 0.91, 0.83, 0.77 |
| HA-29.7 | (0.42) | 0.71, 0.64 |
| Ethanol:chloroform:ammonia/ | | 0.58, 0.53, 0.48, 0.45, 0.20, 0.07 |
| 8:5:2 | | |
| Mucuna pruriens | 0.80, 0.68 | 0.94, 0.85, 0.83 |
| MPRU-27.13 | | 0.80, 0.68 |
| Ethanol:chloroform:ammonia/ | | 0.45, 0.35, 0.22, 0.13, 0.08 |
| 8:5:2 | | |
| Ocimum sanctum | 0.94, 0.83, 0.72, 0.69, 0.63 | 0.97 |
| OSA-T-48.88 | 0.38 | 0.94, 0.83, 0.72, 0.69, 0.63 |
| Ethanol:chloroform:ammonia/ | | 0.55, 0.46, 0.38, |
| 8:5:2 | | 0.32, 0.25, 0.17, 0.12, 0.09 |
| Phyllanthus emblica | (0.91), (0.84) | 0.86, 0.67, 0.47, 0.31, 0.28, 0.11 |
| PE-100 | | |
| Benzene:Methanol: | | |
| Acetone:Acetic acid/ | | |
| 70:20:5:5 | | |
| Tinospora cordifolia | 0.97, | 0.98, 0.97, 0.96, 0.91, 0.89, 0.86 |
| TICO-35.51 | 0.71 | 0.71, 0.66, 0.61, 0.56, 0.50, |
| Ethanol:chloroform:ammonia/ | | 0.45, 0.39, 0.35, 0.31, 0.27, 0.20, 0.12, |
| 8:5:2 | | 0.07 |
| Withania somnifera | 0.61, 0.54 | 0.92, 0.88, 0.81, |
| WISO-38.82 | | 0.61, 0.54 |
| Chloroform:Methanol:water/ | | 0.49, 0.33, 0.23, 0.10 |
| 64:50:10 | | |

Compositions of this invention are quite different from the conventional total extracts in terms of their chemical characteristics as shown by the considerable difference in the TLC comparison above. Thus,

The compositions of this invention do not show several of the TLC spots that are normally present in the commercial herbal extracts of the same herb. This indicates that several chemical structural types are absent in the extracts of this invention.

In most cases, the low fractional value spots or the slow moving spots are absent in the extracts of this invention.

The compositions of this invention also show some new spots. This indicates that new structural classes are present in the extracts of this invention.

Therefore, the extracts and compostions of this invention are novel product compostions.

References

General and Cam Plant References

1973. Perry's chemical engineer's handbook. McGraw-Hill, New York.

1979. Spray-Drying handbook. Goodwin Ltd, London.

Ali, M., N. Shalaby, M. Elgamal, and A. Moussa. 1999. Antifungal effects of different plant extracts and their major components of selected Aloe species. Phytother Res. 13:401-407.

Ames, B. N., W. E. Durston, E. Yamasaki, and F. D. Lee. 1973. Carcinogens are mutagens: a simple test system combining liver homogenates for activation and bacteria for detection. Proc Natl Acad Sci USA. 70:2281-5.

Ames, B. N., J. McCann, and E. Yamasaki. 1975. Proceedings: carcinogens are mutagens: a simple test system. Mutat Res. 33:27-8.

Avila, H., J. rivero, F. Herrera, and G. Fraile. 1997. Cytotoxicity of low molecular weight fractions from Aloe vera. Toxicon. 35:1423-1430.

Balachandran, B., S. Sivaswamy, and V. Sivaramsrishnan. 1991. Genotoxic effects of some foods and food components in Swiss mice. Indian J. Med. Res. 94:378-383.

Barakat, S., S. Adam, M. Maglad, and I. Wasfi. 1985. Effect of Cissus quadrangularis on goats and sheep in Sudan. Rev. Elev. Met. Vet. Pays Trop. 38:185-194.

Blazovics, A., J. Feher, E. Feher, A. Kery, et al. 1993. Liver protecting and lipid lowering effects of Sempervivum tectorum extract in the rat. Phytotherapy Research. 7:98-199.

Boikova, V., and Z. Akulova. 1995. Effect of infusion of some medicinal plants on ovulation in experimental animals. Rastitel'nye Resursy. 31:57-60.

Botha, C., J. Van der Lugt, G. Erasmus, T. Kellerman, et al. 1997. Neurotoxicity of cardiac glycosides—Bufadienolide Krimpsiekte. A paretic condition of small stock poisoned by bufadienolide-containing plants of Crassulaceae. In Proc. int. Symp. Poisonous Plants, 5th meeting: Toxic plants and other natural toxicants. T. Garland and A. Barr, editors. CAB International, Wallingford: UK. 407-412.

Boyd, M., and K. Pauli. 1995. Some practical considerations and applications of the National Cancer Institute in vitro anticancer drug discovery screen. Drug Devel Rsrch. 34:91-109.

Boyum A. Isolation of Mononuclear cells and granulocytes from human blood, Scand J Clin Lab Invest, 1968; 21. 77.8

Chopra, S., M. Patel, and R. Awadhiya. 1975. Studies of Cissus quadrangularis in experimental fracture repair: Effect on chemical parameters in blood. Indian J Med. Res. 63:824-828.

Chopra, S., M. Patel, and R. Awadhiya. 1976. Studies of Cissus quadrangularis in experimental fracture repair: A histopathological study. Indian J Med. Res. 64:1365-1368.

Da Silva, S., S. Costa, S. Mendonca, E. Silva, el al. 1995. Therapeutic effect of oral Kalanchoe pinnata leaf extact on murine leishmaniasis. Acta Trop. 60:201-210.

Da Silva, S. A., S. S. Costa, and B. Rossi-Bergmann. 1999. The anti-leishmanial effect of Kalanchoe is mediated by nitric oxide intermediates. Parasitology. 118:575-82.

Davies, R., M. Leitner, J. Russo, and M. Byrne. 1989. Wound healing, oral and topical activity of Aloe vera. J. Am. Pod. Med. Assoc. 79:559-562.

Davies, R., G. Strewart, and P. Bregman. 1992. Aloe vera and the inflamed synovial pouch model. J. Am. Pod. Med. Assoc. 82:140-148.

Djeraba, A., and P. Quere. 2000. In vivo macrophage activation with chickens with Acemannan, a complex carbohydrate extracted from aloe vera. Int. J. Immunopharmacol. 22:365-372.

U.S. Pat. No. 4,522,811. Serial injection of muramyldipeptides and liposomes enhances the anti-infective activity of muramyldipeptides Serial injection of muramyldipeptides and liposomes enhances the anti-infective activity of muramyldipeptides. 1985.

Gennaro, A. R. 2000. Remington: The science and practice of pharmacy. Lippincott, Williams & Wilkins, Philadelphia, Pa.

Gogte V. M. 2000.Ayurvedic Pharmacology & Therapeutic Uses Of Medicinal Plants (Dravyagunavignyan). Translation: The Academic Team of Bharatiya Vidya Bhavan's Swami Prakashananad Ayurveda Research Centre. Bharatiya Vidya Bhavan, Mumbai.

Hifny, A., A. Hemmoda, and R. Berg. 1984. Anatomical studies on the cerebellum of the donkey of Egypt. Gegenbaurs Mrphol. Jahrb. 130:707-717.

Lans, C., and G. Brown. 1998. Ethnoveterinary medicine. Prev Vet Med. 35:125-142.

Lehrer R. I., M. J. Cline. (1969): Interactioon of C. albicans with human leucocytes and serum, J Bacteriol, 1969; 98 (3) :0996-1004

Lehrer R I. Measurement of candidacidal activity of specific leucocyte type in mixed cell populations, Infection & Immunity, 1970; 42-7.

Meenakumari, S. 1995. Mitoplastic and clastogenic properties of analgin. J. Cytol. Gen. 30:27-33.

Mercykutty, S. 1980. Adriamycin induced genetic toxicity as demonstrated by the Allium test. Cytologia. 45:769-777.

Mueller, S., and H. Stopper. 1999. Characterization of the genotoxicity of anthraquinones in mammalian cells. Biochim biophys Acta. 1428:2-3, 406-414.

Nadkarni, A. 1982. Dr. K. M. Nadkami's Indian materia medica: with Ayurvedic, Unani-tibbi, Siddha, allopathic, homeopathic, naturopathic & home remedies, appendicces & indexes. Sangam Books, Ltd., London.

Nanal, V. M. “Fundamental principles of Ayurveda and their applications in the study of Parnabeeja (Kalanchoe pinnata) in Glimpses of Indian Ethnopharmacology. Ed. P. Pushpangadan et al.) pp 123-128 1995 (English)

Nassis, C., E. Haebisch, and A. ; Giesbrecht. 1992. Antihistamine activity of Bryophyllum calycinum. Braz. J. Med. Biol. Res. 25:929-936.

Obaseiki-Ebor, E. 1985. Preliminary report onthe in vitro activity of Bryophyllum pinnatum. Afr. J. Med. Sci. 14: 199-202.

Pal, S., A. Nag, and N. Choudhary. 1992. Further studies on the anti-inflammatory profile of the methanolic fraction of the fresh leaf extract of Bryophyllum pinnatum. Fitotherapia. 63:451-459.

Paturmaj, S. 2000. Therapeutic effects of Aloe vera on cutaneous micrcirculation and wound healing in second degree burn model in rats. J. Med. Assoc. Thai. 83:417-425.

Qiu, Z., K. Jones, M. Wylie, Q. Jia, et al. 2000. Modified Aloe barbadensis polysaccharide with immunoregulatory activity. Planta Med. 66:152-156.

Raven, P., R. Evert, and S. Eichhorn. 1999. Biology of Plants. W. H. Freeman and Company, New York, N.Y. 944 pp.

Rege N. N., S. A. Dahanukar. Quantitation of microbicidal activity of mononuclear phagocytes: an in vitro technique, J Postgrad Med. 1993: 399: 25-55

Reynolds, T., and A. Dweck. 1999. Aloe vera leaf gel: A review update. J. Ethnopharmac. 68:1-37.

Sendl, A., N. Mulinacchi, F. Vincieri, and H. Wagner. 1993. Anti-inflammatory and immunologically active polysaccharides of Sedum telephium. Phytochemistry. 34:1357-1362.

Shrisharangdharacharyavirachit Sharangdharsamhita (1961). A Hindi Translation with commentary by Acharya Shri Radhakrishna Parashar. Calcutta, India: Baidyanath Ayurveda Bhavan Pvt. Ltd.

Sivaswamy, S., B. Balachandran, S. Balanehru, and V. Sivaramsrishnan. 1991. Mutagenic activity of South Indian food items. Indian J. Exp. Biol. 29:730-737.

States, A.o.F.a.D.O.o.t.U. 1979. Dermal toxicity. In Appraisal of the safety of chemicals in foods, Drugs and cosmetics. 46-59.

Verma, R., B. Garg, and A. Ahmad. 1985. Preliminary report on the in vitro antibacterial activity of Bryophyllum pinnatum leaf juice. Indian J. Pharmac. 18:78-83.

Verma, R., B. Garg, and A. Ahmad. 1986. Pharmacodynamic studies on Kalanchoe integra—An indigenous plant. Indian J. Pharmac. 18:78-83.

Vogler, B., and E. Ernst. 1999. Aloe vera: A systematic review of its clinical effectiveness. Br. J Gen. Pract. 49:823-828.

Yoshikawa, M., J. Yamahara, and H. Matsuda. 1997. Bioactive constituent of Chinese natural medicine. IV. Rhodiolae Radix. 2. On the histamine release inhibitors from the underground parts of Rhodiola sacra (Prain ex Hamet) S.H.Fu (Crassulaceae). Chem. Charm. Bull. 45:1498-1503.

Angiosperm-Monocotyledon Plant References:

Cyperous Rotundus

Gupta M B, T K Palit, N. Singh, K P Bhargava. 1971. Pharmacological studies to isolate the active constituents from Cyperus rotundus possessing anti-inflammatory, anti-pyretic and analgesic activities. Indian J. Med. Res. 59(1):76-82.

Seo W., H. Pae, G. Oh, K. Chai, T. Kwon, Y. Yun, N. Kim, H. Chung. 2001. Inhibitory effects of methanol extracts of Cyperus rotundus rhizomes on nitric oxide and superoxide production by murine macrophage cell line, RAW 264.7 cells. J. Ethnopharmacology 76(1): 59-64.

Thebtaranonth C., Y. Thebtaranonth, S. Wanauppathamkul, Y. Yuthavong. 1995. Antimalrial sequiterpenes from tubers of Cyperus rotundus structure of 10, 12-peroxycalamenene, a sequiterpene endoperoxide. J. Phytochemistry, 40(1):125-28.

Weenan H., M H Nkuma, D H Bray, L B Mwasumbi, L S Kinabo, V A Kilimali, J B Wijnberg. 1990. Antimalarial compounds containing an alpha, beta-unsaturated moiety from Tanzanian medicinal plants. Planta Med. 56(4): 371-3.

Triticum vulgare

Stoika R., N. Kashchak, M. Lutsik-Kordovsky, M. Boyko, A. Tasyrulnik. 2001. In vitro response of phagocytic cells to immunomodulating agents. Med. Sci. Monit. 7(4). 652-8.

Curcuma amada

Ghosh S B, S. Gupta, A K Chandra. 1980. Antifungal activity in rhizomes of Curcuma amada Roxb. Indian J. Exp. Biol. 18(2):174-6.

Zingiber officinale

Agarwal A K, C V Rao, K. Sairam, V K Joshi, R K Goel. 2000. Effect of Piper longum Linn., Zingiber officinale and Ferula species on gastric ulcerations and secretions in rats. Indian J. Exp. Biol. 38(10).:994-8.

Agarwal M., S. Walia, S. Dhingra, B P Khambay. 2001. Insect growth inhibition, antifeedant and antifungal activity of compounds isolated/derived from Zingiber officinale Rosc. (ginger) rhizomes. J. Pest Manag. Sci. 5 7(3) :289-300.

Ahmed R S, V. Seth, B D Banerjee. 2000. Influence of dietary ginger (Zingiber officinales Rosc) on antioxidant defense system in rat:comparison with ascorbic acid. Indian J. Exp. Biol. 38(6).604-6.

Bhandari U., J N Sharma, R. Zafar. 1998. The protective action of ethanolic ginger (Zingiber officinale) extract in cholesterol fed rabbits. J. Ethnopharmacol. 61(2):167-71.

Datta A., N C Sukul. 1987. Antifilarial effect of Zingiber officinale on Dirofilaria imimitis. J. Helminthot 61(3).268-70.

Katiyar S K, R. Agarwal, H. Mukhtar. 1996. Inhibition of tumor promotion in SENCAR mouse skin by ethanol extract of Zingiber officinale rhizome. Cancer Res.56(5):1023-30.

Puri A., R. Sahai, K L Singh, R P Saxena, J S Tandon, K C Saxena. 2000. Immunostimulant activity of dry fruits and plant materials used in Indian traditional medical system for mothers after child birth and invalids. J. Ethnopharmacol. 71(1-2):89-92.

Singh R., B. Rai. 2000. Antifungal potential of some higher plants against Fusarium udum causing wilt disease of Cajanus cajan. Microbios. 102(403):165-73.

Singh K., D K Singh. 2000. Effect of different combinations of MGK-24 or piperonyl butoxide with plant-derived molluscicides on snail reproduction. Arch. Environ. Contam. Toxicol. 38(2):182-90.

Srivastava K C., T. Mustafa. 1992. Ginger (Zingiber oficinale) in rheumatism and musculoskeletal disorders. Med. Hypothesis 39(4):342-8.

Al-Yahya M A., S. Rafatullah, J S Mossa, A N Ageel, N S Parmar, M. Tariq. 1989. Gastroprotective activity of ginger (Zingiber officinale rocs.) in albino rats. Am. J. Chin. Med.17(1-2):51-6.

Angiosperm-Dictyledon Plant References

Holarrhena genus

Abreu P M., E S Martins, O. Kayser, K U Bindseil, K. Siems, A. Seemann, A. Fevert. 1999. Antimicrobial, antitumor and antileishmania screening of medicinal plants from Guinea-Bissau. Phytomedicine 6(3);187-95.

Ahmed I., Z. Mehmood, F. Mohammad. 1998. Screening of some Indian medicinal plants for their antimicrobial properties. J. Ethnopharmacol. 62(2).183-93.

Arseculeratne S N, A A Gunatilaka, R G Panabokke. 1981. Studies on medicinal plants of Sri Lanka: occurrence of pyrolizidine alkaloids and hepatotoxic properties in some traditional medicinal herbs. J. Ethnopharmacol. 4(2):159-77.

Atal C K, M L Sharma, A. Kaul, A. Khajuria. 1986. Immunomodulating agents of plant origin. 1: Preliminary screening. J. Ethnopharmacol. 18(2):133-41.

Chakraborty A., A H Brantner. 1999. Antibacterial steroid alkaloids from the stem bark of Holarrhena pubescens. J Ethnopharmacol. 68(1-3):339-44.

Loukaci A., O. Kayser, K. Bindseil, K. Siems, J. Frevert, P M Abreu. 2000. New trichothecenes isolated from Holarrhena floribunda. J. Nat. Prod. 63(1):52-6.

Swertia chirata

Karan M., K. Vasisht, S S Handa. 1999. Antihepatotoxic activity of Swertia chirata on paracetamol and galactosamine induced hepatotoxicity in rats. Phytother Res. 13(2):95-101.

Khanom F., H. Kayahara, K. Tadasa. April 2000. Superoxide-scavenging and prolyl endopeptidase inhibitory activities of Bangladeshi indigenous medicinal plants. Biosci. Biotechnol. Biochem.64(4).837-40.

Khanom F., H. Kayahara, K. Tadasa. Sept. 2000. Tyrosinase inhibitory activity of Bangladeshi indigenous medicinal plants. Biosci. Biotechnol. Biochem. 64(9):1967-9.

Rafatullah S., M. Tariq, J S Mossa, M A al-Yahya, M S al-Said. M A Ageel. 1993. Protective effect of Sivertial chirata against indometbacin and other ulcerogenic agent-induced gastric ulcers. Drugs Exp. Clin. Res. 19(2).68-73.

Reen R K, N. Karan, K. Singh, V. Karan, R K Johri, J. Singh. 2001. Screening of various swertia species extracts in primary monolayer cultures of rat hepatocytes against carbon tetrachloride and paracetamol induced toxicity. J. Ethnopharmacology, 75(2-3):239-47.

Phyllanthus Genus

Calixto J B, A R Santos, F V Cechinel, R A Yunes. 1998. Med. Res. Rev 18(4):225-58.

Ishizaki T., A. Ushirosako, F. Kimizuka, T. Kawabe, H. Morita (Takara Shuzo Co. Ltd.). 1999. Hyaluronidase inhibitors from Phyllanthus emblica and foods and beverages containing them. JP 11071290 A2 16 Mar. 1999 Khanom et al ( see under Sweretia chirata: Khanom et al., April 2000)

Liu K C, M T Lin, S S Lee, J F Chiou, S Ren, E J Lien. 1999. Antiviral tannins from two Phyllanthus species. Planta Med 65(]).43-46.

Sur P., D K Ganguly, Y. Hara, Y. Matsuo. 1998. Antitumor activity of Emblica officinalis Gaerin fruit extract. ACS Symp. Ser., 701(Functional Foods for Disease Prevention 1:Fruits, Vegetables and Teas), 104-113(English) American Chemical Society.

Linum usitatissimum

Cunnane S C, S. Ganguli, C. Menard, A C Liede, M J Hamadeh, Z Y Chen, T M Wolever, D J Jenkins. 1993. High alpha-linolenic acid flaxseed (Linum usitatissimum): Some nutritional properties in humans. Br. J Nutr. 69(2):443-53.

Harris W S. 1997. n-3 fatty acids and serum lipoproteins: human studies. Am. J Clin. Nutr. 65(5 Suppl): 1645S-1654S.

James M J, R A Gibson, L G Cleland. 2000. Dietary polyunsaturated fatty acids and inflammatory mediator production. Am. J. Clin. Nuir. 71(1 Suppl):343S-8S.

Azadiracta indica

Singh K. A. Singh, D K Singh. 1996. Molluscicidal activity of neem (Azadirachta indica A. Juss). J. Ethnopharmacol. 52(1):35-40.Rao DR, R. Reuben, M S Venugopal, B A

Nagasampagi, H. Schumutterer. 1992. Evaluation of neem, Azadiracta indica, with and without water management, for the control of culicine mosquito larvae in rice fields. Med Vet Entomol.6(4).318-24.

Virk A S, H. Steingass, K H Menke. 1989. Studies on in vitro degradation and in vivo digestion of a slow ammonia releasing urea product. Arch Tierernahr 39(1-2).167-76

Terminalia bellerica/Terminalia chebula

Ahmad I., Z. Mehmood, F. Mohammad. 1998. Screening of some Indian Medicinal Plants for their antimicrobial properties. J. Ethnopharmacol 62(2).183-93.

Jagtap A. G., S. G. Karkera. 1999. Potential of the aqeous extacts of Terminalia chebula as an anticaries agent. J. Ethnopharmacol.68(1-3):299-306.

el-Mekkawt S., M R Meselhy, I T Kusumoto, S. Kadota, M. Hattori, T. Namba. Inhibitory effects of Egyptian folk medicines on human immunodeficiency virus (HIV) reverse transcriptase. Chem Pharm Bull(Tokyo) 43(4):641-8.

Tamhane M D, S P Thorat, N N Rege, S P Dahanukar. 1997. Effect of oral administration of Terminalia chebula on gastric emptying: an experimental study. J. Postgrad Med 43(1).12-3.

Embelia ribes

Chitra M., E. Sukumar, C S Devi. (3-H)-thymidine uptake of and lipid peroxidation by tumour cells on embelin treatment: an in vitro study. Oncology 52(1):62-8.

Gupta S., S N Sanyal, U. Kanwar. 1989. Antispermatogenic effect of embelin, a plant benzoquinone, on male albino rats in vivo and in vitro. Contraception 39(3): 307-20.

Low G., L J Rogers, S P Brumley, D Erlich. 1985. Visual deficits and retinotoxicity caused by the naturally occurring anthelmintics, Embelia ribes and Hagenia Abyssinia. Toxicol. Appl. Pharmacol 81(2):220-30.

Glycyrrhiza glabra

Khanom et al ( see under Sweretia chirata: Khanom et al., September, 2000)

Konovalova G G, A K Tikhaze, Y Z Lankin. 2000. Antioxidant activity parapharmaceutics containing natural inhibitors of free radicle processes. Bull Exp Biol Med. 130(7):658-60.

Kroes B H, C J Beukelman, A J van den Berg, G J Wolbink, H. can Dijk, R P Labadie. 1997. inhibition of human complement by beta-glycyrrhetinic acid. Immunology 90(1):115-20.

Nose M., K. Terawaki, K. Oguri, Y. Ogihara, K. Yoshimatsu, K. Shimomura. 1998. Activation of macrophages by crude polysaccharide fractions obtained from shoots of Glycyrrhiza glabra and hairy roots of Glycyrrhiza uralensis. Bio. Pharm Bul.21(10)1110-12.

Mucuna pruriens

Akhtar M S, A Q Quereshi, J Iqual. 1990. Antidiabetic evaluation of Mucuna pruriens Linn. Seeds. J. Pak Med Assoc. 40(7):147-50.

Guerranti R., J C Aguiyi, E. Errico, R. Pagani, E. Marinello. 2001. Effect of Mucuna pruriens extract on activation of prothrombin by Echscarinatus venom. J Ethnopharmacol. 75(2-3):175-80.

Nagashayana N., P. Sankarankutty, M R Nampoothiri, P K Mohan, K P Mohanakumar. 2000. Association of L-DOPA with recovery following Ayurveda medication in Parkinson's disease. J. Neurol Sci. 176(2): 124-7.

Pomgamia glabra

Ravi U. P. Singh, A K Garg, D K Agarwal. 2000. Performance of lambs fed expeller pressed and sovent extracted karanj (P. glabra) oil cake. 0377-8401 88(1-2).121-128.

Sagar S K, S S Sehgal. 1996. Effects of aqeous extract of deoiled neem(Azadirachta indica A. juss) seed kernal and Karanj (Pongamia glabra vent) seed kernel against Culex quinquefasciatus. J. Commun Dis. 28(4):260-69. Trigonella foenum-graecum

Zia T., S N Hasnain, S K Hasan. 2001. Evaluation of the oral hypoglycaemic effect of Trigonella foenum-gaecum L. (methi) in normal mice. J. Ethnopharmacol. 75(2-3):191-95.

Weder J K, K. Haussner. 1991. Inhibitors of human and bovine trypsin and chymotrypsin in fenugreek (Trigonella foenum-graecum) seeds. Reaction with the human and bovine proteinases. Z Lebensm Unters Forch. 193(4).321-325.

Ficus bengalensis

Kumar R V, K T Augusti. 1989. Antidiabetic effect of leucocyanidin derivative isolated from the bark of of Ficus bengalensis. Indian J. Biochem Biophys. 26(6):400-4.

Mousa O., P. Vuorela, J. Kiviranta, S A Wahab, R. Hiltunen, H. Vuorela. 1994. Bioactivity of certain Egyptian Ficus species. J Ethnopharmacol. 41(1-2): 71-6

Gymnosperm Plant References:

Taxus baccata/Abies webbiana Lindle.

Kite G C, T J Lawrence, E A Dauncey.2000. Detecting Taxus poisoning in horses using liquid chromatography/mass spectrometry. VET Hum Toxicol. 42(3):151-4.

Mantle D., T W Lenard, A T Pickering. 2000. Therapeutic applications of medicinal plants in the treatment of breast cancer: a review of their pharmacology, efficacy and tolerability. Adverse Drug React Toxicol Rev 2000. 19(3):223-40.

Other Biologicals References

Yeast

Bacon J, et al. 1969. The glucan component of the cell wall of baker's yeast (Saccharomyces cerevisiae) considered in relation to its ultrastructure. Biochem J 1969,1114:557-67.

Mushroom

Lombardi R. M. 2002. Mycological Medicine. Functional Foods and Nutraceuticals. January 2002.

I claim:

1. An oil extract of a monocotyledonous or dicotyledonous Angiosperm or Gymnosperm plants biomass and any other non-plant biomass other than Cyperous rotundus, Triticum vulgare, Allium sativum, Allium cepa, Curcuma longa, Curcuma amada, Zingiber officinalis, Momordica charantia, Holarrhena antydysenterica, Rauwolfia serpentina, Vinca rosea, Hemedesmus indicus, Swertia chirata, Phyllanthus emblica, Linum usitatissimum, Azadiracta indica, Gymnema sylvestree, Terminalia chebula, Terminalia bellerica, Eugenia jamnolana, Piper longa, Piper nigrum, Embelia ribes, Tinospora cordifolia, Glycine max, Glycyrrhiza glabra, Mucuna pruriens, Phaseolus radiatus, Pongamia glabra, Trigonella foenum-graecum, Santalum alba, Ocimum sanctum, Sesamum indicum, Bacopa monnieri, Withania somnifera, Carum copticum, Cuminum cyminum, Ficus bengalensis, Taxus baccata, Cissus quadrangularis, Kalanchoe pinnata, Aloe vera, Agaricus spinosus, Sacharimyces cereviceae, or Prawn produced by the process of boiling said Angiosperm or Gymnosperm plant biomass or non-plant biomass with oil and water.

2. The oil extract of claim 1, wherein the plant and non-plant biomass is Cyperous rotundus, Triticum vulgare, Allium sativum, Allium cepa, Curcuma longa, Curcuma amada, Zingiber officinalis, Momordica charantia, Holarrhena antydysenterica, Rauwolfia serpentina, Vinca rosea, Hemedesmus indicus, Gymnema sylvestree, Swertia chirata, Phyllanthus emblica, Linum usitatissimum, Azadiracta indica, Terminalia chebula, Terminalia bellerica, Eugenia jamnolana, Piper longa, Piper nigrum, Embelia ribes, Tinospora cordifolia, Glycine max, Glycyrrhiza glabra, Mucuna pruriens, Phaseolus radiatus, Pongamia glabra, Trigonella foenum-graecum, Santalum alba, Ocimum sanctum, Sesamum indicum, Bacopa monnieri, Withania somnifera, Carum copticum, Cuminum cyminum, Ficus bengalensis, Taxus baccata, Cissus quadrangularis, Kalanchoe pinnata, Aloe vera, Agaricus spinosus, Sacharimyces cereviceae, or Prawn.

3. An oil extract of Cyperous rotundus, Triticum vulgare, Allium sativum, Allium cepa, Curcuma longa, Curcuma amada, Zingiber officinalis, Momordica charantia, Holarrhena antydysenterica, Rauwolfia serpentina, Vinca rosea, Hemedesmus indicus, Gymnema sylvestree, Swertia chirata, Phyllanthus emblica, Linum usitatissimum, Azadiracta indica, Terminalia chebula, Terminalia bellerica, Eugenia jamnolana, Piper longa, Piper nigrum, Embelia ribes, Tinospora cordifolia, Glycine max, Glycyrrhiza glabra, Mucuna pruriens, Phaseolus radiatus, Pongamia glabra, Trigonella foenum-graecum, Santalum alba, Ocimum sanctum, Sesamum indicum, Bacopa monnieri, Withania somnifera, Carum copticum, Cuminum cyminum, Ficus bengalensis, Taxus baccata, Cissus quadrangularis, Kalanchoe pinnata, Aloe vera, Agaricus spinosus, Sacharimyces cereviceae, or Prawn, produced by the process of boiling an Angiosperm or Gymnosperm plant biomass or non-plant biomass in oil and water, wherein the ratio of water to kalka is less than 16:1.

4. An oil extract of Cyperous rotundus, Triticum vulgare, Allium sativum, Allium cepa, Curcuma longa, Curcuma amada, Zingiber officinalis, Momordica charantia, Holarrhena antydysenterica, Rauwolfia serpentina, Vinca rosea, Hemedesmus indicus, Gymnema sylvestree, Swertia chirata, Phyllanthus emblica, Linum usitatissimum, Azadiracta indica, Terminalia chebula, Terminalia bellerica, Eugenia jamnolana, Piper longa, Piper nigrum, Embelia ribes, Tinospora cordifolia, Glycine max, Glycyrrhiza glabra, Mucuna pruriens, Phaseolus radiatus, Pongamia glabra, Trigonella foenum-graecum, Santalum alba, Ocimum sanctum, Sesamum indicum, Bacopa monnieri, Withania somnifera, Carum copticum, Cuminum cyminum, Ficus bengalensis, Taxus baccata, Cissus quadrangularis, Kalanchoe pinnata, Aloe vera, Agaricus spinosus, Sacharimyces cereviceae, or Prawn, made by the process of boiling an Angiosperm or Gymnosperm plant biomass or non-plant biomass in oil and water, wherein the ratio of decoction of plant or non-plant biomass part in water to kalka is less than 24:1.

5. An oil extract of Cyperous rotundus, Triticum vulgare, Allium sativum, Allium cepa, Curcuma longa, Curcuma amada, Zingiber officinalis, Momordica charantia, Holarrhena antydysenterica, Rauwolfia serpentina, Vinca rosea, Hemedesmus indicus, Gymnema sylvestree, Swertia chirata, Phyllanthus emblica, Linum usitatissimum, Azadiracta indica, Terminalia chebula, Terminalia bellerica, Eugenia jamnolana, Piper longa, Piper nigrum, Embelia ribes, Tinospora cordifolia, Glycine max, Glycyrrhiza glabra, Mucuna pruriens, Phaseilus radiatus, Pongamia glabra, Trigonella foenum-graecum, Santalum alba, Ocimum sanctum, Sesamum indicum, Bacopa monnieri, Withania somnifera, Carum copticum, Cuminum cyminum, Ficus bengalensis, Taxus baccata, Cissus quadrangularis, Kalanchoe pinnata, Aloe vera, Agaricus spinosus, Sacharimyces cereviceae, or Prawn, made by the process of boiling an Angiosperm or Gymnosperm plant biomass or non-plant biomass in oil and water, wherein the ratio of juice to kalka is less than 32:1.

6. The oil extract of claim 1, wherein the oil is a vegetable oil, mineral oil, animal oil, or coconut oil.

7. The oil extract of claim 2, wherein the oil is a vegetable oil, mineral oil, animal oil, or coconut oil.

8. The oil extract of claim 3, wherein the oil is a vegetable oil, mineral oil, animal oil, or coconut oil.

9. The oil extract of claim 4, wherein the oil is a vegetable oil, mineral oil, animal oil, or coconut oil.

10. The oil extract of claim 5, wherein the oil is a vegetable oil, mineral oil, animal oil, or coconut oil.

11. A composition comprising the oil extract of claim 1 and a carrier.

12. A composition comprising the oil extract of claim 2 and a carrier.

13. A composition comprising the oil extract of claim 3 and a carrier.

14. A composition comprising the oil extract of claim 4 and a carrier.

15. A composition comprising the oil extract of claim 5 and a carrier.

16. The oil extract of claim 1 wherein the non-plant biomass includes those of microbial, mycological, bird, marine including fish, amphibian, veterinary or human origin.

17. A method of preparing an oil extract of an Angiosperm or Gymnosperm plant or any other non-plant biomass or biomass part comprising:
soaking in water and/or pounding and/or cutting a biomass or biomass part thereof, and then
mixing the biomass or biomass part thereof with water to form an aqueous mixture,
homogenizing the aqueous mixture to form a homogenate,
adding the homogenate to an oil to form an oil mixture,
boiling the oil mixture, and
filtering the oil mixture to produce a first extract and a first residue, such that the first extract is the oil extract of a biomass or biomass part.

18. The method of claim 17, wherein Angiosperm or Gymnosperm plant biomass or biomass part is a leaf, rhizome, seed or root.

19. The method of claim 17, wherein the Angiosperm or Gymnosperm plant or non-plant biomass or biomass part is a decoction of the Angiosperm or Gymnosperm plant or non-plant biomass or biomass part in water with kalka in the ratio of decoction of the Angiosperm or Gymnosperm plant or non-plant biomass or biomass part in water to kalka of less than 24:1.

20. The method of claim 17, wherein the entire Angiosperm or Gymnosperm plant or non-plant biomass comprises soaking in water, pounding and cutting, and then mixed with water.

21. The method of claim 17, wherein the Angiosperm or Gymnosperm plant or non-plant biomass or biomass part is selected from one member of the group consisting of a rhizome, seed, stem, leaf, shoot, flower, root, cotyledon from plants, milk, bacterial or fungal or yeast or organs or meat cell mass, blood and bone or cartilage tissue from non-plant biomass.

22. The method of claim 21, wherein the Angiosperm or Gymnosperm plant or non-plant biomass or biomass part is selected from two members of the group.

23. The method of claim 2 1, wherein the Angiosperm or Gymnosperm plant or non-plant biomass or biomass part is selected from three members of the group.

24. The method of claim 21, wherein the Angiosperm or Gymnosperm plant or non-plant biomass or biomass part is selected from four members of the group.

25. The method of claim 21, wherein the Angiosperm or Gymnosperm plant or non-plant biomass or biomass part is selected from at least five members.

26. The method of claim 21, wherein the Angiosperm or Gymnosperm plant or non-plant biomass or biomass part is selected from six members of the group.

27. The method of claim 17, wherein the boiling is performed for about 15 to about 360 minutes.

28. The method of claim 17, wherein wherein the Angiosperm or Gymnosperm plant or non-plant biomass or biomass part is Cyperous rotundus, Triticum vulgare, Allium sativum, Allium cepa, Curcuma longa, Curcuma amada, Zingiber officinalis, Momordica charantia, Holarrhena antydysenterica, Rauwolfia serpentina, Vinca rosea, Hemedesmus indicus, Gymnema sylvestree, Swertia chirata, Phyllanthus emblica, Linum usitatissimum, Azadiracta indica, Terminalia chebula, Terminalia bellerica, Eugenia jamnolana, Piper longa, Piper nigrum, Embelia ribes, Tinospora cordifolia, Glycine max, Glycyrrhiza glabra, Mucuna pruriens, Phaseolus radiatus, Pongamia glabra, Trigonella foenum-graecum, Santalum alba, Ocimum sanctum, Sesamum indicum, Bacopa monnieri, Withania somnifera, Carum copticum, Cuminum cyminum, Ficus bengalensis, Taxus baccata, Cissus quadrangularis, Kalanchoe pinnata, Aloe vera, Agaricus spinosus, Sacharimyces cereviceae, or Prawn.

29. The method of claim 17, wherein a ratio of the homogenate to the oil is 0.1 to 1.5.

30. A method of preparing an oil extract of wherein the Angiosperm or Gymnosperm plant or any other non-plant biomass comprising:
soaking in water and/or pounding and/or cutting the biomass or a biomass part thereof to reduce size, and then
mixing at least one biomass or biomass part thereof with water to form an aqueous mixture,
homogenizing the aqueous mixture,
filtering the aqueous mixture to obtain a biomass part residue fraction and a juice fraction,
combining the biomass residue fraction and the juice fraction in any proportion with an oil to create an oil mixture,
boiling the oil mixture, and
filtering the oil mixture to produce a first extract and a first residue, such that the first extract is the oil extract of an Angiosperm or Gymnosperm plant biomass or any other non-plant biomass.

31. The method of claim 30, wherein the entire biomass is mixed with water.

32. The method of claim 30, wherein the Angiosperm or Gymnosperm plant or non-plant biomass or biomass part is selected from one member of the group consisting of a rhizome, seed, stem, leaf, shoot, flower, root, cotyledon from plants, milk, bacterial or fungal or yeast or organs or meat cell mass, blood, or bone or cartilage tissue.

33. The method of claim 32, wherein the Angiosperm or Gymnosperm plant or non-plant biomass or biomass part is selected from two members.

34. The method of claim 32, wherein wherein the Angiosperm or Gymnosperm plant or non-plant biomass or biomass part is selected from three members of the group.

35. The method of claim 32, wherein the Angiosperm or Gymnosperm plant or non-plant biomass or biomass part is selected from four members of the group.

36. The method of claim 32, wherein the Angiosperm or Gymnosperm plant or non-plant biomass or biomass part is selected from five members of the group.

37. The method of claim 32, wherein the Angiosperm or Gymnosperm plant or non-plant biomass or biomass part is selected from six members of the group.

38. The method of claim 32, wherein the boiling is performed for about 25 to about 360 minutes.

39. The method of claim 30, wherein the Angiosperm or Gymnosperm plant or non-plant biomass or biomass part is Cyperous rotundus, Triticum vulgare, Allium sativum, Allium cepa, Curcuma longa, Curcuma amada, Zingiber officinalis, Momordica charantia, Holarrhena antydysenterica, Rauwolfia serpentina, Vinca rosea, Hemedesmus indicus, Gymnema sylvestree, Swertia chirata, Phyllanthus emblica, Linum usitatissimum, Azadiracta indica, Terminalia chebula, Terminalia bellerica, Eugenia jamnolana, Piper longa, Piper nigrum, Embelia ribes, Tinospora cordifolia, Glycine max, Glycyrrhiza glabra, Mucuna pruriens, Phaseolus radiatus, Pongamia glabra, Trigonella foenum-graecum, Santalum alba, Ocimum sanctum, Sesamum indicum, Bacopa monnieri, Withania somnifera, Carum copticum, Cuminum cyminum, Ficus bengalensis, Taxus baccata, Cissus quadrangularis, Kalanchoe pinnata, Aloe vera, Agaricus spinosus, Sacharimyces cereviceae, or Prawn.

40. The method of claim 30, wherein said proportion of the biomass part residue fraction to the juice fraction is 1:0.

41. The method of claim 30, wherein said proportion of the biomass part residue fraction to the juice fraction is 0:1.

42. The method of claim 30, wherein the ratio of first residue to the oil is 0.05 to 0.5.

43. A method to treat a human disease or condition comprising administering an oil extract of a monocotyledonous or dicotyledonous Angiosperm or Gymnosperm plant or any other non-plant biomass to a human in need thereof.

44. The method of claim 43, wherein the human disease or condition is concerning a system and/or organ including nervous, respiratory, high stress, energy level, digestive and immune system, regulation of phagocytosis, high blood lipid levels, wound, inflammatory, general pain, swelling, neuromuscular junction, reproductive, hair loss, skincare, hyperpigmentation, cancer, tumors or vision.

45. The method of claim 43, wherein the human disease or condition is cough, cold, congestion, allergy-induced asthma, stress-induced asthma, sleeplessness, poor feet circulation, stomach acidity, stomach upsets, poor appetite, colitis, poor growth in children, poor height growth in children, poor weight gain, high blood LDL cholesterol, high blood triglycerides, bruises, cuts, diabetic ulcers, leprotic ulcers, varicose ulcers, bedsores, burns, piles, fistula, spondulitis, arthritis, sciatica, gingivitis, toothache, oligospermia, poor sperm motility, poor ovulation, menstrual discomfort, menstrual pain, menorrahgia, skin inflammation, pimples, lichenplanus, eczema, dermatitis, psoriasis, poor hair growth, neuralgia, neuropathy, diabetes, neuromuscular coordination, sluured speech, loss of mental concentration and memory, hyperpigmentation, macular surgery, dry cornea or sty, tremors.

46. A method to enhance plant germination, vigor and defenses, or to enhance from a plant the yield of a seed, tuber, rhizome, vegetable, fruit, flower or secondary metabolite, or to extend the shelf life of a fruit or flower, comprising applying an oil extract of an Angiosperm or Gymnosprm plant or any other non-plant biomass to a seed or a portion of a plant.

47. The method of claim 46, wherein the plant, seed, tuber, rhizome, vegetable, fruit, flower or secondary metabolite is produced by a monocotyledonous or dicotyledonous Angiosperm or Gymnosperm.

48. The method of claim 46, wherein the plant is okra, tomato, brinjal, red chili, marigold, jasmine, gladiola, rose, mango, pomegranate, ber, sapota, lime, guava, strawberry, Solanum khasianum, rice, wheat, cotton, soybean, mustard, Dendrocalamus strictus, Eucalyptus sp., Leucina leucephalia, Casuarina equisetifolia, Brassica juncea, Jowar, Sorghum vulgare sugar cane or mung bean.

49. The method of claim 46, wherein said applying comprises spraying on foliage, applying to terminal buds, fruits, flowers, injecting into phloem, or applying to soil at root zone.

50. A method to enhance the growth of a mushroom, applying an oil extract of an Angiosperm or Gymnosperm plant biomass or any other non-plant biomass to the mushroom.

51. A method to decrease mutations in an individual, comprising administering an Angiosperm or Gymnosperm plant biomass or any other non-plant biomass oil extract to the individual.

52. The method of claim 51, wherein the individual is human, animal or plant.

53. A method to control the growth of or kill plants comprising applying an Angiosperm or Gymnosperm plant biomass or any other non-plant biomass extract to said plants.

54. The method of claims 53, wherein the concentration of the Angiosperm or Gymnosperm plant biomass or any other non-plant biomass extract is 25 to 500 g/hectare.

55. The method of claim 53, wherein said applying comprises spraying on foliage, applying to terminal buds, fruits, flowers, injecting into phloem, or applying to soil at root zone.

56. A method to control growth of gram positive or gram negative bacteria, yeast and fungi comprising applying an Angiosperm or Gymnosperm plant extract or any other non-plant biomass extract to said bacteria, yeast, fungi growing in a medium.

57. A method of claim 56, wherein the bacteria and yeast are pseudomonas spp., staphylococcus spp. and candida spp.

58. The method of claim 56, wherein said medium comprises a liquid, gel, soil or carrier solids containing a culture of the said bacteria, yeast, fungi.

59. The method of claims 56, wherein the concentration of the Angiosperm or Gymnosperm plant biomass or any other non-plant biomass extract is 1 to 1000 mg/litre of medium.
